(19) United States
(12) Patent Application Publication
(10) Pub. No.: US 2020/0291355 A1
(43) Pub. Date: Sep. 17, 2020

(54) ENGINEERED ERYTHROID CELLS INCLUDING LOADABLE ANTIGEN-PRESENTING POLYPEPTIDES AND METHODS OF USE

(71) Applicant: Rubius Therapeutics, Inc., Cambridge, MA (US)

(72) Inventors: Tiffany Fen-Yi Chen, Cambridge, MA (US); Thomas Joseph Wickham, Groton, MA (US); Christopher Lawrence Moore, Boston, MA (US); Shamael Rabia Dastagir, Somerville, MA (US); Douglas Charles Mclaughlin, Quincy, MA (US); Regina Sophia Salvat, Cambridge, MA (US); Alex Richard Nanna, Cambridge, MA (US)

(21) Appl. No.: 16/796,801

(22) Filed: Feb. 20, 2020

Related U.S. Application Data
(60) Provisional application No. 62/938,839, filed on Nov. 21, 2019.
(60) Provisional application No. 62/926,222, filed on Oct. 25, 2019.
(60) Provisional application No. 62/877,190, filed on Jul. 22, 2019.
(60) Provisional application No. 62/808,253, filed on Feb. 20, 2019.

(51) Int. Cl. C12N 5/078; C07K 14/74

(52) U.S. Cl. CPC C12N 5/0641; C07K 2319/03; C12N 2510/00; C07K 14/70539

(57) ABSTRACT

The present disclosure provides customizable enucleated erythroid cells or enucleated cells that can be engineered to include, on their surface, a loadable exogenous antigen-presenting polypeptide, wherein the loadable exogenous antigen-presenting polypeptide comprises one or more amino acid substitutions. In some embodiments, the one or more amino acid substitutions stabilize the loadable exogenous antigen-presenting polypeptide on the cell surface. In some embodiments, the loadable exogenous antigen-presenting polypeptide is stabilized on the cell surface in the absence of a polypeptide bound to the loadable exogenous antigen-presenting polypeptide. In some embodiments, the loadable exogenous antigen-presenting polypeptide comprises an exogenous displaceable polypeptide bound to the loadable exogenous antigen-presenting polypeptide. In some embodiments, the loadable exogenous antigen-presenting polypeptide is stabilized on the cell surface upon release of the displaceable polypeptide.

### RELATED APPLICATIONS

This application claims the benefit of priority to U.S. Provisional Patent Application No. 62/808,253, filed Feb. 20, 2019, U.S. Provisional Patent Application No. 62/877,190, filed Jul. 22, 2019, U.S. Provisional Patent Application No. 62/926,222, filed Oct. 25, 2019, and U.S. Provisional Patent Application No. 62/938,839, filed Nov. 21, 2019. The contents of each of these applications are incorporated herein by reference in its entirety.

### SEQUENCE LISTING

The instant application contains a Sequence Listing which has been submitted electronically in ASCII format and is hereby incorporated by reference in its entirety. Said ASCII copy, created on Feb. 20, 2020, is named 129267-01205_SL.txt and is 680,946 bytes in size.

### BACKGROUND

Active immune responses depend on efficient presentation of antigens and co-stimulatory signals by antigen-presenting cells (APCs). Upon internalization of an antigen, the APCs can display antigen-class I and II major histocompatibility complex (MHC) on the membrane together with co-stimulatory signals to activate antigen-specific T cells, which play a key role in the adaptive immune response. In vivo, induction of T cell responses is highly dependent on interactions with professional APCs, in particular dendritic cells (DCs), which present, for example, tumor-specific antigens. Generally, antigen-specific T cells can be primed and amplified ex vivo before they are transferred back to the subject. For example, in adoptive cell transfer (ACT), tumor-specific T cells are isolated then expanded ex vivo to obtain a large number of cells for transfusion. As one of the APCs, DCs are usually used to maximize T cell stimulation ex vivo. However, the use of natural APCs, such as DCs, has been met with certain challenges, including lack of knowledge of the optimal antigen-loaded DC, and mixed results have been found in clinical trials (Steenblock et al. (2009) Expert Opin. Biol. Ther. 9: 451-64; Melief (2008) Immunity 29: 372-83; Palucka and Banchereau (2013) Immunity 39: 38-48). In addition, isolation and ex vivo stimulation of autologous DCs is time-consuming and expensive, and the quality of ex vivo-generated DCs can be variable (Steenblock et al. (2009); Kim et al. (2004) Nat. Biotechnol. 22: 403-10). The use of subject-derived autologous DCs therefore limits standardization of DC-based treatment protocols (see Steenblock et al. (2009); and Kim et al. (2004)).

Artificial APCs (aAPCs) are engineered platforms for T cell activation and expansion that aim to avoid the aforementioned obstacles while mimicking the interaction between DCs and T cells. They include multiple systems, including synthetic biomaterials that have been engineered to activate and/or expand desirable immune cell populations (e.g., T cells). These systems may act by mimicking the interaction between DCs and T cells. For instance, several cell-sized, rigid, beads, such as latex microbeads, polystyrene-coated magnetic microbeads and biodegradable poly(lactic-co-glycolic acid) microparticles, have been developed. The efficacy of these beads in inducing activation and/or expansion of immune cells appears to be highly dependent on the properties of the materials used. For example, beads greater than 200 nm are typically retained at the site of inoculation, while smaller particles may be taken up by DCs (see, e.g., Reddy et al. (2006) J. Control. Release 112: 26-34). In contrast, the membrane of natural APCs is much more dynamic than the outer surface of these beads.

There remains a need for improved ways to stimulate T cells and to promulgate sufficient numbers of therapeutic T cells for adoptive immunotherapy. In particular, there is a need for aAPCs capable of presenting exogenous antigenic polypeptides that are customizable so as to present any antigenic polypeptide of interest to induce a desired response.

### SUMMARY

The present disclosure relates to customizable engineered erythroid cells (e.g., engineered enucleated erythroid cells) or enucleated cells (e.g., modified enucleated cells), that are engineered to include, on their surface (e.g., on the plasma membrane) a loadable exogenous antigen-presenting polypeptide and are capable of, inter alia, activating, expanding or differentiating/de-differentiating T cells, suppressing T cell activity, suppressing T effector cells, and/or stimulating and expanding T regulatory cells. The engineered erythroid cells or enucleated cells described herein offer numerous advantages over other cells that present antigens to activate an immune response. For example, provided herein are engineered erythroid cells or enucleated cells that are readily customizable to present a selected exogenous antigenic polypeptide of interest on a loadable exogenous antigen-presenting polypeptide, for administration to a subject in need thereof.

In some aspects, the present disclosure provides an engineered enucleated erythroid cell comprising a loadable exogenous antigen-presenting polypeptide on the cell surface, wherein the loadable exogenous antigen-presenting polypeptide comprises one or more amino acid substitutions which stabilize the loadable exogenous antigen-presenting polypeptide on the cell surface. In some embodiments, the loadable exogenous antigen-presenting polypeptide is stabilized in the absence of a polypeptide bound to (e.g., the antigen-binding cleft of) the exogenous antigen-presenting polypeptide.

In some embodiments, the engineered enucleated erythroid cell further comprises an exogenous antigenic polypeptide bound to the loadable exogenous antigen-presenting polypeptide.

In some embodiments, the loadable exogenous antigen-presenting polypeptide has a higher affinity for the exogenous antigenic polypeptide than for an exogenous displaceable polypeptide.

In some embodiments, the exogenous antigenic polypeptide binds to the loadable exogenous antigen-presenting polypeptide with a K_{D} of from about 1 picomolar to about 100 nanomolar.

In some embodiments, the exogenous antigenic polypeptide comprises an amino acid sequence provided in any one of Tables 7-8, 16-26, or B. In some embodiments, the exogenous antigenic polypeptide comprises an amino acid sequence provided in Table 7.

In some embodiments, the exogenous antigenic polypeptide is non-covalently attached to the loadable exogenous antigen-presenting polypeptide. In other embodiments, the exogenous antigenic polypeptide is covalently attached to the loadable exogenous antigen-presenting polypeptide.

In some embodiments, the exogenous antigenic polypeptide is covalently attached to the loadable exogenous antigen-presenting polypeptide by a linker.

In some embodiments, the loadable exogenous antigen-presenting polypeptide comprises a linker, wherein the linker comprises an acceptor sequence for conjugation of an exogenous antigenic polypeptide.

In some embodiments, the linker is between about 10 amino acids and 30 amino acids in length. In other embodiments, the linker is about 15 amino acid residues in length.

In some embodiments, wherein the loadable exogenous antigen-presenting polypeptide comprises a transmembrane domain. In some embodiments, the transmembrane domain comprises a transmembrane domain of a Type 1 membrane protein. In some embodiments, the Type 1 membrane protein comprises glycophorin A (GPA).

In some embodiments, the engineered enucleated erythroid cell further comprises a displaceable exogenous polypeptide bound to the loadable exogenous antigen-presenting polypeptide.

In some embodiments, the exogenous displaceable polypeptide is capable of being displaced from the loadable exogenous antigen-presenting polypeptide by an exogenous antigenic polypeptide.

In some embodiments, the exogenous displaceable polypeptide is between about 6 amino acids in length to about 30 amino acids in length.

In some embodiments, the exogenous displaceable polypeptide binds to the loadable exogenous antigen-presenting polypeptide with a K_{D} of from about 1 nM to about 100 μM.

In some embodiments, the exogenous displaceable polypeptide comprises an amino acid sequence provided in Table 6.

In some embodiments, the loadable exogenous antigen-presenting polypeptide and the exogenous displaceable polypeptide are comprised in a single chain fusion protein. In some embodiments, the single chain fusion protein comprises a linker disposed between the loadable exogenous antigen-presenting polypeptide and the exogenous displaceable polypeptide.

In some embodiments, the linker comprises an enzymatic cleavage site. In some embodiments, the enzymatic cleavage site comprises the amino acid sequence LEVLFQGP (SEQ ID NO: 1). In some embodiments, the enzymatic cleavage site comprises the amino acid sequence ENLYFQG (SEQ ID NO: 2). In some embodiments, the enzymatic cleavage site is within 10 amino acids or less from a GGG motif. In some embodiments, the enzymatic cleavage site is within 10 amino acids or less from an LPTXG motif (SEQ ID NO: 3). In some embodiments, the enzymatic cleavage site is within 10 amino acids or less from the amino acid sequence AHIVMVDAYKPTK (SEQ ID NO: 4). In some embodiments, the enzymatic cleavage site is within 10 amino acids or less from the amino acid sequence ATHIKFSKRD (SEQ ID NO: 5).

In some embodiments, the linker is between about 10 amino acids and 30 amino acids in length. In some embodiments, the linker is about 15 amino acid residues in length.

In some embodiments, the single chain fusion protein comprises a transmembrane domain. In some embodiments, the transmembrane domain comprises a transmembrane domain of a Type 1 membrane protein. In some embodiments, the Type 1 membrane protein comprises glycophorin A (GPA).

In some embodiments, the loadable exogenous antigen-presenting polypeptide comprises a human leukocyte antigen a (HLA) heavy chain polypeptide and a beta-2-microglobulin (β2M) polypeptide, or a fragment thereof.

In some embodiments, the loadable exogenous antigen-presenting polypeptide comprises a linker disposed between the HLAα heavy chain polypeptide and the (32M polypeptide.

In some embodiments, the linker is a GlySer linker.

In some embodiments, the linker is about 2 to about 30 amino acid residues in length.

In some embodiments, the linker is about 18 amino acid residues in length.

In some embodiments, the HLA heavy chain polypeptide is derived from an HLA class I polypeptide.

In some embodiments, the HLA class I polypeptide is selected from the group consisting of: HLA-A, HLA-B, HLA-C, HLA-E, and HLA-G.

In some embodiments, the HLA-A polypeptide comprises an HLA-A allele selected from the group consisting of: A*01:01, A*02:01, A *03:01, A*24:02, A*11:01, A*29:02, A*32:01, A*68:01, A*31:01, A*25:01, A*26:01, A*23:01, and A*30:01.

In some embodiments, the HLA-B polypeptide comprises an HLA-B allele selected from the group consisting of: B*08:01, B*07:02, B*44:02, B*15:01, B*40:01, B*44:03, B*35:01, B*51:01, B*27:05, B*57:01, B*18:01, B*14:02, B*13:02, B*55:01, B*14:01, B*49:01, B*37:01, B*38:01, B*39:01, B*35:03, and B*40:02.

In some embodiments, HLA-C polypeptide comprises an HLA-C allele selected from the group consisting of: C*07:01, C*07:02, C*05:01, C*06:02, C*04:01, C*03:04, C*03:03, C*02:02, C*16:01, C*08:02, C*12:03, C*01:02, C*15:02, C*07:04, and C*14:02.

In some embodiments, the HLA-E polypeptide comprises an HLA-E allele selected from the group consisting of: E*01:01:01:01, E*01:01:01:02, E*01:01:01:03, E*01:01:01:04, E*01:01:01:05, E*01:01:01:06, E*01:01:01:07, E*01:01:01:08, E*01:01:01:09, E*01:01:01:10, E*01:01:02, E*01:03:01:01, E*01:03:01:02, E*01:03:01:03, E*01:03:01:04, E*01:03:02:01, E*01:03:02:02, E*01:03:03, E*01:03:04, E*01:03:05, E*01:04, E*01:05, E*01:06, E*01:07, E*01:08N, E*01:09 and E*01:10.

In some embodiments, the HLA-G polypeptide comprises an HLA-G allele selected from the group consisting of: G*01:01:01:01, G*01:01:01:02, G*01:01:01:03, G*01:01:01:04, G*01:01:01:05, G*01:01:01:06, G*01:01:01:07, G*01:01:01:08, G*01:01:02:01, G*01:01:02:02, G*01:01:03:01, G*01:01:03:02, G*01:01:03:03, G*01:01:03:04, G*01:01:04, G*01:01:05, G*01:01:06, G*01:01:07, G*01:01:08, G*01:01:09, G*01:01:11, G*01:01:12, G*01:01:13, G*01:01:14, G*01:01:15, G*01:01:16, G*01:01:17, G*01:01:18, G*01:01:19, G*01:01:20, G*01:01:21, G*01:01:22, G*01:02, G*01:03:01:01, G*01:03:01:02, G*01:04:01:01, G*01:04:01:02, G*01:04:02, G*01:04:03, G*01:04:04, G*01:04:05, G*01:04:06, G*01:05N, G*01:06, G*01:07, G*01:08:01, G*01:08:02, G*01:09, G*01:10 and G*01:11.

In some embodiments, the loadable exogenous antigen-presenting polypeptide comprises an amino acid substitution to an alanine at the amino acid residue corresponding to position 84 of the alpha chain.

In some embodiments, the loadable exogenous antigen-presenting polypeptide comprises an amino acid substitution to cysteine at each of the amino acid residues corresponding to positions 84 and 139 of the alpha chain.

In some embodiments, the loadable exogenous antigen-presenting polypeptide comprises an amino acid substitution to cysteine at each of the amino acid residues corresponding to positions 51 and 175 of the alpha chain.

In some embodiments, wherein the loadable exogenous antigen-presenting polypeptide comprises at least one pair of amino acid substitutions to cysteine at amino acid residues corresponding to the following positions of the alpha chain: 84 and 139; 51 and 175; 5 and 168; 130 and 157; 135 and 140; 11 and 74; 45 and 63; and 33 and 49.

In some embodiments, the loadable exogenous antigen-presenting polypeptide comprises an amino acid substitution to cysteine at an amino acid residue corresponding to position 84 of the alpha chain and a cysteine at a second amino acid residue of the linker disposed between the (32M polypeptide and the displaceable exogenous polypeptide.

In some embodiments, the loadable exogenous antigen-presenting polypeptide comprises an alpha chain derived from an HLA-A*02:01 allele, and wherein the alpha chain comprises an amino acid substitution to glutamic acid at the amino acid residue corresponding to position 115.

In other embodiments, the HLA heavy chain polypeptide is derived from an HLA class II polypeptide.

In some embodiments, the HLA class II polypeptide is selected from the group consisting of: HLA-DPα, HLA-DPβ, HLA-DMA, HLA-DMB, HLA DOA, HLA DOB, HLA DQα, HLA DQβ, HLA DRα, and HLA

In some embodiments, the HLA-DPα polypeptide comprises an allele selected from the group consisting of: DPA1*01:03, DPA1*02:01, DPA1*02:07.

In some embodiments, the HLA-Dβ polypeptide comprises an allele selected from the group consisting of: DPB1*04:01, DPB1*02:01, DPB1*04:02, DPB1*03:01, DPB1*01:01, DPB1*11:01, DPB1*05:01, DPB1*10:01, DPB1*06:01, DPB1*13:01, DPB1*14:01, and DPB1*17:01.

In some embodiments, the HLA-DQα polypeptide comprises an allele selected from the group consisting of: DQA1*05:01, DQA1*03:01, DQA1*01:02, DQA1*02:01, DQA1*01:01, DQA1*01:03, and DQA1*04:01.

In some embodiments, the HLA-DQβ polypeptide comprises an allele selected from the group consisting of: DQB1*03:01, DQB1*02:01, DQB1*06:02, DQB1*05:01, DQB1*02:02, DQB1*03:02, DQB1*06:03, DQB1*03:03, DQB1*06:04, DQB1*05:03, and DQB1*04:02.

In some embodiments, the HLA-DRβ polypeptide comprises an allele selected from the group consisting of: DRB1*07:01, DRB1*03:01, DRB1*15:01, DRB1*04:01, DRB1*01:01, DRB1*13:01, DRB1*11:01, DRB1*04:04, DRB1*13:02, DRB1*08:01, DRB1*12:01, DRB1*11:04, DRB1*09:01, DRB1*14:01, DRB1*04:07, and DRB1*14:04.

In some aspects, the present disclosure provides a method of treating a subject in need of an altered immune response, comprising determining an HLA status of the subject, selecting an engineered enucleated erythroid cell comprising a loadable exogenous antigen-presenting polypeptide on the cell surface, wherein the antigen-presenting polypeptide is immunologically compatible with the subject, and wherein the exogenous antigen-presenting polypeptide comprises one or more amino acid substitutions which stabilize the loadable exogenous antigen-presenting polypeptide on the cell surface, contacting the engineered enucleated erythroid cell with an exogenous antigenic polypeptide and administering the engineered enucleated erythroid cell to the subject, thereby treating the subject.

In some embodiments, the loadable exogenous antigen-presenting polypeptide is stabilized in the absence of a polypeptide bound to the exogenous antigen-presenting polypeptide.

In some embodiments, the method further comprises conjugating the exogenous antigenic polypeptide to the loadable exogenous antigen-presenting polypeptide.

In some embodiments, a displaceable exogenous polypeptide is bound to the loadable exogenous antigen-presenting polypeptide.

In other embodiments, the method further comprises displacing the displaceable exogenous polypeptide from the loadable exogenous antigen-presenting polypeptide with the exogenous antigenic polypeptide prior to administering the engineered enucleated erythroid cell to the subject.

In other embodiments, the method further comprises conjugating the exogenous antigenic polypeptide to the loadable exogenous antigen-presenting polypeptide.

In other embodiments, the method further comprises selecting an exogenous antigenic polypeptide.

In some embodiments, the subject has or is at risk of developing cancer. In other embodiments, the subject has or is at risk of developing an autoimmune disease. In other embodiments, the subject has or is at risk of developing an infectious disease.

In some aspects, the present disclosure provides a method of making an engineered enucleated erythroid cell comprising an antigen-loaded exogenous antigen-presenting polypeptide, comprising obtaining an engineered enucleated erythroid cell comprising a loadable exogenous antigen-presenting polypeptide on the cell surface, wherein the loadable exogenous antigen-presenting polypeptide comprises one or more amino acid substitutions which stabilize the loadable exogenous antigen-presenting polypeptide on the cell surface, and contacting the engineered enucleated erythroid cell with an exogenous antigenic polypeptide, thereby preparing an engineered enucleated erythroid cell comprising an antigen-loaded exogenous antigen-presenting polypeptide.

In some embodiments, the loadable exogenous antigen-presenting polypeptide is stabilized in the absence of a polypeptide bound to the exogenous antigen-presenting polypeptide.

In some embodiments, the method further comprises conjugating the exogenous antigenic polypeptide to the loadable exogenous antigen-presenting polypeptide.

In some embodiments, the method further comprises selecting an exogenous antigenic polypeptide.

In some embodiments, a displaceable exogenous polypeptide is bound to the loadable exogenous antigen-presenting polypeptide

In some embodiments, the method further comprises displacing the displaceable exogenous polypeptide from the loadable exogenous antigen-presenting polypeptide with the exogenous antigenic polypeptide.

In some aspects, the present disclosure provides a method of making an engineered enucleated erythroid cell comprising a loadable exogenous antigen-presenting polypeptide on the cell surface, comprising introducing an exogenous nucleic acid encoding the loadable exogenous antigen-presenting polypeptide into a nucleated erythroid precursor cell, wherein the loadable exogenous antigen-presenting polypeptide comprises one or more amino acid substitutions which stabilize the loadable exogenous antigen-presenting polypeptide on the cell surface, and culturing the nucleated erythroid precursor cell under conditions suitable for enucleation and production of the loadable exogenous antigen-presenting polypeptide, thereby making an engineered enucleated erythroid cell comprising a loadable exogenous antigen-presenting polypeptide on the cell surface, thereby making the engineered enucleated erythroid cell.

In some embodiments, the loadable exogenous antigen-presenting polypeptide is stabilized in the absence of a polypeptide bound to the exogenous antigen-presenting polypeptide.

In some embodiments, the loadable exogenous antigen-presenting polypeptide comprises a linker, wherein the linker comprises an acceptor sequence for conjugation of an exogenous antigenic polypeptide.

In some embodiments, the linker is between about 10 amino acids and 30 amino acids in length. In some embodiments, the linker is about 15 amino acid residues in length.

In some embodiments, the loadable exogenous antigen-presenting polypeptide comprises a transmembrane domain,

In some embodiments, a linker is disposed between the transmembrane domain and the loadable exogenous antigen-presenting polypeptide.

In some embodiments, the transmembrane domain comprises a transmembrane domain of a Type 1 membrane protein. In some embodiments, the Type I membrane protein comprises a GPA

In some embodiments, the method further comprises contacting the engineered enucleated erythroid cell with at least one exogenous antigenic polypeptide.

In some embodiments, the method further comprises conjugating the exogenous antigenic polypeptide to the loadable exogenous antigen-presenting polypeptide.

In some embodiments, the exogenous nucleic acid further encodes an exogenous displaceable polypeptide.

In some embodiments, the exogenous loadable antigen-presenting polypeptide and exogenous displaceable polypeptide are comprised in a single chain fusion protein.

In some embodiments, the single chain fusion protein comprises a linker disposed between the loadable exogenous antigen-presenting polypeptide and the exogenous displaceable polypeptide.

In some embodiments, the linker comprises an enzymatic cleavage site.

In some embodiments, the enzymatic cleavage site comprises the amino acid sequence LEVLFQGP (SEQ ID NO: 1). In other embodiments, the enzymatic cleavage site comprises the amino acid sequence ENLYFQG (SEQ ID NO: 2). In other embodiments, the enzymatic cleavage site is within 10 amino acids or less from a GGG motif. In other embodiments, the enzymatic cleavage site is within 10 amino acids or less from an LPTXG motif (SEQ ID NO: 3). In other embodiments, the enzymatic cleavage site is within 10 amino acids or less from the amino acid sequence AHIVMVDAYKPTK (SEQ ID NO: 4).

In other embodiments, the enzymatic cleavage site is within 10 amino acids or less from the amino acid sequence ATHIKFSKRD (SEQ ID NO: 5).

In some embodiments, the linker is between about 10 amino acids and 30 amino acids in length. In other embodiments, the linker is about 15 amino acid residues in length.

In some embodiments, the loadable exogenous antigen-presenting polypeptide comprises a transmembrane domain. In some embodiments, the transmembrane domain comprises a transmembrane domain of a Type 1 membrane protein. In some embodiments, the Type I membrane protein comprises a GPA.

In some embodiments, the method further comprises contacting the engineered enucleated erythroid cell with an exogenous antigenic polypeptide having a greater binding affinity to the loadable exogenous antigen-presenting polypeptide than the displaceable exogenous polypeptide.

In some embodiments, the method further comprises contacting the engineered enucleated erythroid cell with a dipeptide.

In some embodiments, the loadable exogenous antigen-presenting polypeptide comprises HLA-A:02:01, HLA-A1:01, HLA-A3:01, HLA-A24:02, HLA-A26:01, HLA-B7:02, HLA-B08:01, HLA-B27:05, HLA-B27:05, HLA-B39:01, HLA-B40:01, HLA-B58:01, HLA-B15:01, or HLA-E01:01 and the dipeptide is glycyl-leucine (GL), glycyl-methionine (GM), GG, glycyl-alanine (GA), glycyl-valine (GV), glycyl-phenylalanine (GF), leucyl-glycine (LG), glycyl-cyclohexylalanine (G-Cha), glycyl-homoleucine (GHLe), acetylated leucine, or glycyl-arginine (GR).

In some embodiments, the loadable exogenous antigen-presenting polypeptide comprises HLA-B:27:05 and the dipeptide is GR or G-Cha.

In some embodiments, the method further comprises contacting the cell with an enzyme under conditions suitable for cleavage of the enzymatic cleavage site.

In some embodiments, the method further comprises conjugating the exogenous antigenic polypeptide to the loadable exogenous antigen-presenting polypeptide.

In some embodiments, the exogenous antigenic polypeptide is conjugated to the loadable exogenous antigenic polypeptide using click chemistry.

In some embodiments, the engineered enucleated erythroid cell described herein can be contacted with multiple exogenous antigenic polypeptides, to allow the binding of the multiple exogenous antigenic polypeptides on the loadable exogenous antigen-presenting polypeptide. In some embodiments, the engineered enucleated erythroid cell described herein can be contacted with one or more exogenous antigenic polypeptides, to allow the binding of the one or more exogenous antigenic polypeptides on the loadable exogenous antigen-presenting polypeptide. In some embodiments, the engineered enucleated erythroid cell described herein can be contacted with two or more exogenous antigenic polypeptides, to allow the binding of the two or more exogenous antigenic polypeptides on the loadable exogenous antigen-presenting polypeptide. In some embodiments, the engineered enucleated erythroid cell described herein can be contacted with three or more exogenous antigenic polypeptides, to allow the binding of the three or more exogenous antigenic polypeptides on the loadable exogenous antigen-presenting polypeptide. In some embodiments, the engineered enucleated erythroid cell described herein can be contacted with four or more exogenous antigenic polypeptides, to allow the binding of the four or more exogenous antigenic polypeptides on the loadable exogenous antigen-presenting polypeptide. In some embodiments, the engineered enucleated erythroid cell described herein can be contacted with five or more exogenous antigenic polypeptides, to allow the binding of the five or more exogenous antigenic polypeptides on the loadable exogenous antigen-presenting polypeptide. In some embodiments, the loadable exogenous antigen-presenting polypeptide comprises an MHC class I polypeptide. In some embodiments, the loadable exogenous antigen-presenting polypeptide comprises an MHC class II polypeptide.

In some aspects, the present disclosure provides a method of activating an antigen-specific T cell population, the method comprising contacting the T cell population with the engineered enucleated erythroid cell described herein, thereby activating the antigen-specific T cell population.

In some embodiments, the engineered enucleated erythroid cell described herein can be contacted with multiple antigen-specific T cell populations, to allow the activation of the multiple antigen-specific T cell populations. In some embodiments, the engineered enucleated erythroid cell comprising one or more exogenous antigenic polypeptides can be contacted with one or more antigen-specific T cell populations, to allow the activation of the one or more antigen-specific T cell populations. In some embodiments, the engineered enucleated erythroid cell comprising two or more exogenous antigenic polypeptides can be contacted with two or more antigen-specific T cell populations, to allow the activation of the two or more antigen-specific T cell populations. In some embodiments, the engineered enucleated erythroid cell comprising three or more exogenous antigenic polypeptides can be contacted with three or more antigen-specific T cell populations, to allow the activation of the three or more antigen-specific T cell populations. In some embodiments, the engineered enucleated erythroid cell comprising four or more exogenous antigenic polypeptides can be contacted with four or more antigen-specific T cell populations, to allow the activation of the four or more antigen-specific T cell populations. In some embodiments, the engineered enucleated erythroid cell comprising five or more exogenous antigenic polypeptides can be contacted with five or more antigen-specific T cell populations, to allow the activation of the five or more antigen-specific T cell populations.

### BRIEF DESCRIPTION OF DRAWINGS

The figures are meant to be illustrative of one or more features, aspects, or embodiments provided herein and are not intended to be limiting.

FIG. 1 depicts amino acid sequence alignments of exemplary wild-type alleles of HLA-A (i.e., HLA-A*01:01) (SEQ ID NO: 1593), HLA-B (i.e., HLA-B*51:01) (SEQ ID NO: 1594), HLA-C (i.e., HLA-C*12:02) (SEQ ID NO: 1595), HLA-E (i.e., HLA-E*01:03) (SEQ ID NO: 1596), and HLA-G (i.e., HLA-G*01:01) (SEQ ID NO: 922). Each of the depicted amino acid sequences are of the mature protein excluding the N-terminal leader sequence. Amino acid residues that are predicted to stabilize the protein when substituted to cysteine are highlighted in boxes. The consensus shows conserved amino acid residues in greater than half of the exemplary wild-type alleles shown.

FIGS. 2A-B depict schematics showing exemplary constructs as described herein. FIG. 2A depicts a construct which comprises a loadable exogenous antigen-presenting polypeptide, e.g., an HLA Class I molecule, wherein the HLA Class I molecule comprises an HLA (32M subunit linked to the HLA a subunit, which comprises a transmembrane domain, such as a Type 1 membrane protein transmembrane domain (e.g., a GPA transmembrane domain), wherein the HLA Class I molecule comprises one or more mutations which allow the HLA Class I molecule to be present in a stable conformation on the cell surface in the absence of a bound polypeptide, and wherein the HLA Class I molecule is capable of binding an exogenous antigenic polypeptide. FIG. 2B depicts the exemplary construct of FIG. 2A, further comprising an exogenous displaceable polypeptide bound to the loadable exogenous antigen-presenting polypeptide. In some embodiments, the exogenous displaceable polypeptide can be displaced, e.g., by cleaving an enzyme cleavage site as described herein, to allow for the binding of an exogenous antigenic polypeptide.

FIGS. 3A-3C are line graphs showing the activation of reporter T lymphocyte cell lines including an HPV E7-specific T-cell receptor after being contacted with engineered enucleated erythroid cells including either (1) an antigen-presenting polypeptide comprising wild-type HLA*02:01 polypeptide, β2M polypeptide, GPA, and FLAG-tag (“wt HLA-A2”); (2) a loadable antigen-presenting polypeptide comprising mutant HLA*02:01 polypeptide having the amino acid substitutions Y84C and A139C, β2M polypeptide, GPA, and a FLAG-tag (“ds HLA-A2”); or (3) a fusion polypeptide comprising an HPV E7 peptide, mutant HLA*02:01 polypeptide having the amino acid substitution Y84A, β2M polypeptide, GPA, and FLAG-tag (“sc trimer”). FIG. 3A is a line graph showing the activation of the reporter T lymphocyte cell line after being contacted with engineered enucleated erythroid cells that had been cultured for 0 days. FIG. 3B is a line graph showing the activation of the reporter T lymphocyte cell line after being contacted with engineered enucleated erythroid cells that had been cultured for 3 days. FIG. 3C is a line graph showing the activation of the reporter T lymphocyte cell line after being contacted with engineered enucleated erythroid cells that had been cultured for 6 days. RLU=relative light units.

FIGS. 4A and 4B depict the stability of antigen-presenting polypeptides on engineered erythroid cells over a 10-day period. FIG. 4A is a bar graph showing the mean fluorescence intensity (MFI) detected using anti-β2M antibody staining and flow cytometry for engineered enucleated erythroid cells including: (1) a loadable antigen-presenting polypeptide comprising mutant HLA*02:01 polypeptide having the amino acid substitutions Y84C and A139C, 132M polypeptide, GPA, and a FLAG-tag, which was loaded with HPV E7 peptide (“ds +peptide”); (2) an antigen-presenting polypeptide comprising wild-type HLA*02:01 polypeptide, β2M polypeptide, GPA, and FLAG-tag, which was loaded with HPV E7 peptide (“wt +peptide”); (3) a loadable antigen-presenting polypeptide comprising mutant HLA*02:01 polypeptide having the amino acid substitutions Y84C and A139C, β2M polypeptide, GPA, and a FLAG-tag, unloaded (“ds”); or (4) an antigen-presenting polypeptide comprising wild-type HLA*02:01 polypeptide, β2M polypeptide, GPA, and FLAG-tag, unloaded (“wt”). FIG. 4B is a bar graph showing the MFI detected using anti-HLA-A2 antibody staining and flow cytometry for engineered enucleated erythroid cells including: (1) a loadable antigen-presenting polypeptide comprising mutant HLA*02:01 polypeptide having the amino acid substitutions Y84C and A139C, β2M polypeptide, GPA, and a FLAG-tag, which was loaded with HPV E7 peptide (“ds +peptide”); (2) an antigen-presenting polypeptide comprising wild-type HLA*02:01 polypeptide, β2M polypeptide, GPA, and FLAG-tag, which was loaded with HPV E7 peptide (“wt +peptide”); (3) a loadable antigen-presenting polypeptide comprising mutant HLA*02:01 polypeptide having the amino acid substitutions Y84C and A139C, β2M polypeptide, GPA, and a FLAG-tag, unloaded (“ds”); or (4) an antigen-presenting polypeptide comprising wild-type HLA*02:01 polypeptide, β2M polypeptide, GPA, and FLAG-tag, unloaded (“wt”). The MFI for cells cultured during 0, 3, 5, 7, or 10 days is shown.

FIGS. 5A-5F are line graphs showing the activation of reporter T lymphocyte cell lines including either an HPV E7-specific TCR (FIGS. 5A-5C) or an HPV E6-specific TCR (FIGS. 5D-5F) after being contacted with engineered enucleated erythroid cells including either (1) a fusion polypeptide comprising an HPV E7 peptide, mutant HLA*02:01 polypeptide having the amino acid substitution Y84A, β2M polypeptide, GPA, and FLAG-tag (“sc trimer”); (2) a loadable antigen-presenting polypeptide comprising mutant HLA*02:01 polypeptide having the amino acid substitutions Y84C and A139C, β2M polypeptide, and GPA (without a FLAG-tag) (“ds”); (3) a loadable antigen-presenting polypeptide comprising mutant HLA*02:01 polypeptide having the amino acid substitutions Y84C and A139C, β2M polypeptide, GPA, and a FLAG-tag (“ds+FLAG”); (4) an antigen-presenting polypeptide comprising wild-type HLA*02:01 polypeptide, β2M polypeptide, GPA, and FLAG-tag (“wt+FLAG”); or (5) engineered enucleated erythroid cells generated from untransduced erythroid precursor cells (“Untransduced”). Enucleated erythroid cells including antigen-presenting polypeptides were contacted with both HPV E7 and HPV E6 antigenic polypeptides to load the antigen-presenting polypeptides at the concentrations indicated. FIG. 5A is a line graph showing the activation of the reporter T lymphocyte cell line including an HPV E7-specific TCR after being contacted with the indicated enucleated erythroid cells. Cells including the ds, ds+FLAG, or wt+FLAG antigen-presenting polypeptides were loaded using 10 μg/mL of each of HPV E6 antigenic polypeptide and HPV E7 antigenic polypeptide. FIG. 5B is a line graph showing the activation of the reporter T lymphocyte cell line including an HPV E7-specific TCR after being contacted with the indicated enucleated erythroid cells. Cells including the ds, ds+FLAG, or wt+FLAG antigen-presenting polypeptides were loaded using 1 μg/mL of each of the HPV E6 antigenic polypeptide and the HPV E7 antigenic polypeptide. FIG. 5C is a line graph showing the activation of the reporter T lymphocyte cell line including an HPV E7-specific TCR after being contacted with the indicated enucleated erythroid cells. Cells including the ds, ds+FLAG, or wt+FLAG antigen-presenting polypeptides were loaded using 100 ng/mL of each of the HPV E6 antigenic polypeptide and the HPV E7 antigenic polypeptide. FIG. 5D is a line graph showing the activation of the reporter T lymphocyte cell line including an HPV E6-specific TCR after being contacted with the indicated enucleated erythroid cells. Cells including the ds, ds+FLAG, or wt+FLAG antigen-presenting polypeptides were loaded using 10 μg/mL of each of the HPV E6 antigenic polypeptide and the HPV E7 antigenic polypeptide. FIG. 5E is a line graph showing the activation of the reporter T lymphocyte cell line including an HPV E6-specific TCR after being contacted with the indicated enucleated erythroid cells. Cells including the ds, ds+FLAG, or wt+FLAG antigen-presenting polypeptides were loaded using 1 μg/mL of each of the HPV E6 antigenic polypeptide and the HPV E7 antigenic polypeptide. FIG. 5F is a line graph showing the activation of the reporter T lymphocyte cell line including an HPV E6-specific TCR after being contacted with the indicated enucleated erythroid cells. Cells including the ds, ds+FLAG, or wt+FLAG antigen-presenting polypeptides were loaded using 100 ng/mL of each of the HPV E6 antigenic polypeptide and the HPV E7 antigenic polypeptide. RLU=relative light units.

FIG. 6 is a plot showing the activation of reporter T lymphocyte cell lines including either an HPV E7-specific TCR or an HPV E6-specific TCR after being contacted with engineered enucleated erythroid cells including either (1) a fusion polypeptide comprising an HPV E7 peptide, mutant HLA*02:01 polypeptide having the amino acid substitution Y84A, β2M polypeptide, GPA, and FLAG-tag (“sc trimer (E7)”); (2) a loadable antigen-presenting polypeptide comprising mutant HLA*02:01 polypeptide having the amino acid substitutions Y84C and A139C, 132M polypeptide, and GPA (without a FLAG-tag) (“ds”); (3) a loadable antigen-presenting polypeptide comprising mutant HLA*02:01 polypeptide having the amino acid substitutions Y84C and A139C, 132M polypeptide, GPA, and a FLAG-tag (“ds+FLAG”); or (4) an antigen-presenting polypeptide comprising wild-type HLA*02:01 polypeptide, β2M polypeptide, GPA, and FLAG-tag (“wt+FLAG”); or with untransduced K562 cells (“UNT K562s”).

FIGS. 7A-7C are line graphs depicting the binding kinetics of HPV E7 fluorescent antigenic polypeptides onto engineered enucleated erythroid cells including either (1) a loadable antigen-presenting polypeptide comprising mutant HLA*02:01 polypeptide having the amino acid substitutions Y84C and A139C, β2M polypeptide, GPA, and a FLAG-tag (“ds”); or (2) an antigen-presenting polypeptide comprising wild-type HLA*02:01 polypeptide, β2M polypeptide, GPA, and FLAG-tag (“wt”). Cells were loaded with 10 ng/mL of one of the tetramethylrhodamine (TAMRA)-labeled versions of the HPV E7 antigenic polypeptides: HPV E7-GGK or HPV E7-E18K. FIG. 7A is a line graph showing the binding kinetics of HPV E7-GGK or HPV E7-E18K over 90 minutes onto engineered erythroid cells including the ds loadable antigen-presenting polypeptide or the wt antigen-presenting polypeptide at 4° C. FIG. 7B is a line graph showing the binding kinetics of HPV E7-GGK or HPV E7-E18K over 90 minutes onto engineered erythroid cells including the ds loadable antigen-presenting polypeptide or the wt antigen-presenting polypeptide at room temperature. FIG. 7C is a line graph showing the binding kinetics of HPV E7-GGK or HPV E7-E18K over 90 minutes onto engineered erythroid cells including the ds loadable antigen-presenting polypeptide or the wt antigen-presenting polypeptide at 37° C.

FIGS. 8A-8C are bar graphs showing the activation or expansion of CMV-specific T cells after being contacted with engineered enucleated erythroid cells including either (1) an antigen-presenting polypeptide comprising wild-type HLA*02:01 polypeptide, β2M polypeptide, GPA, and a FLAG-tag (“wt HLA-A2”); or (2) a loadable antigen-presenting polypeptide comprising mutant HLA*02:01 polypeptide having the amino acid substitutions Y84C and A139C, β2M polypeptide, GPA, and a FLAG-tag (“ds HLA-A2”), which were loaded with a CMV antigenic polypeptide (3) erythroid cells generated from untransduced erythroid precursor cells contacted with CMV antigenic polypeptide (UNT-pulsed); or (4) erythroid cells generated from untransduced erythroid precursor cells not contacted with CMV antigenic polypeptide (UNT-non-pulsed). FIG. 8A is a bar graph showing the activation of CMV-specific CD8⁺ T cells after being contacted with the indicated erythroid cells for 4 hours at the specified erythroid cells:T cell ratios, as indicated by the upregulation of NFAT. FIG. 8C is a bar graph showing the activation of CMV-specific CD8⁺ T cells after being contacted with the indicated enucleated erythroid cells for 4 hours at the specified erythroid cells:T cell ratios, as indicated by the upregulation of Nur77. FIG. 8D is a bar graph showing the expansion of the T lymphocytes from PBMCs contacted with the indicated enucleated erythroid cells during 5 days.

FIG. 9A depicts an exogenous loadable antigen-presenting polypeptides comprising an HLA class II a polypeptide chain (excluding the native transmembrane domain and cytosolic region), and HLA class II β polypeptide chain (excluding the native transmembrane domain and cytosolic region), and a GPA transmembrane domain.

FIGS. 9B and 9C are plots showing the expression in K562 cells of exogenous antigen-presenting polypeptides comprising either (1) a HLA-DRA*01:01 polypeptide (excluding the native transmembrane domain and cytosolic region), a GlySer linker, HLA-DRB1*01:01 polypeptide (excluding the native transmembrane domain and cytosolic region) and a GPA transmembrane domain (FIG. 9B), or (2) a HLA-DPA1*01:03 polypeptide (excluding the native transmembrane domain and cytosolic region), a GlySer linker, HLA-DPB1*04:01 polypeptide (excluding the native transmembrane domain and cytosolic region), and a GPA transmembrane domain (FIG. 9C).

### DETAILED DESCRIPTION

The present disclosure is based on the development of readily customizable enucleated erythroid cells or enucleated cells that can be engineered to include, on their surface (e.g., on the plasma membrane), a loadable exogenous antigen-presenting polypeptide, e.g., an HLA polypeptide, and are capable of, inter alia, activating, expanding or differentiating/de-differentiating T cells, suppressing T cell activity, suppressing T effector cells, and/or stimulating and expanding T regulatory cells. In particular, the engineered erythroid cells or enucleated cells provided herein comprise a loadable exogenous antigen-presenting polypeptide on the cell surface which comprises one or more amino acid substitutions. In some embodiments, the one or more amino acid substitutions stabilize the loadable exogenous antigen-presenting polypeptide on the cell surface. In some embodiments, the loadable exogenous antigen-presenting polypeptide is stabilized on the cell surface in the absence of a polypeptide bound to the exogenous antigen-presenting polypeptide.

The present disclosure also provides enucleated erythroid cells or enucleated cells that can be engineered to include, on their surface (e.g., on the plasma membrane) a wild-type exogenous antigen-presenting polypeptide, e.g., an HLA polypeptide, and are capable of, inter alia, activating, expanding or differentiating/de-differentiating T cells, suppressing T cell activity, suppressing T effector cells, and/or stimulating and expanding T regulatory cells.

In some embodiments, an exogenous antigenic polypeptide of interest can be selected and bound to the loadable exogenous antigen-presenting polypeptide or wild-type exogenous antigen-presenting polypeptide prior to administration to a subject, thus allowing for customizable therapy specific to the particular subject.

In some embodiments, the loadable exogenous antigen-presenting polypeptide comprises an exogenous displaceable polypeptide bound to the loadable exogenous antigen-presenting polypeptide (e.g., at the antigen-binding cleft of the loadable exogenous antigen-presenting polypeptide). In some embodiments, the exogenous displaceable polypeptide can be replaced with a selected exogenous antigenic polypeptide of interest prior to administration to a subject.

In some embodiments of the present disclosure, the engineered erythroid cells are engineered enucleated erythroid cells, e.g., reticulocytes or erythrocytes. In some embodiments of the present disclosure, the enucleated cell (e.g., modified enucleated cell) is a reticulocyte, an erythrocyte or a platelet.

### Definitions

As used in this specification and the appended claims, the singular forms “a”, “an” and “the” include plural references unless the content clearly dictates otherwise.

The use of the alternative (e.g., “or”) should be understood to mean either one, both, or any combination thereof of the alternatives.

As used herein, the term “about,” when referring to a measurable value such as an amount, a temporal duration, and the like, encompasses variations of ±20% or ±10%, more preferably ±5%, even more preferably ±1%, and still more preferably ±0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “comprise,” “comprising,” and “comprises” and “comprised of” are meant to be synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

As used herein, the terms “such as”, “for example” and the like are intended to refer to exemplary embodiments and not to limit the scope of the present disclosure.

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the embodiments provided herein, preferred materials and methods are described herein.

As used herein, the terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” refers to a primary response induced by ligation of a cell surface moiety. For example, in the context of receptors, such stimulation entails the ligation of a receptor and a subsequent signal transduction event. With respect to stimulation of a T cell, such stimulation refers to the ligation of a T cell surface moiety that in some embodiments subsequently induces a signal transduction event, such as binding the TCR/CD3 complex. Further, the stimulation event may activate a cell and upregulate or downregulate expression or secretion of a molecule. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cellular responses. “Activation” includes activation of CD8+ T cells, activation of CD4+ T cells, stimulation of cytotoxic activity of T cells, stimulation of cytokine secretion by T cells, detectable effector functions, modification of the differentiation state of a T cell (e.g. promote expansion and differentiation from T effector to T memory cell), and/or any combination thereof The term “activated T cells” refers to, among other things, T cells that are undergoing cell division.

As used herein, “altered immune response” refers to changing the form or character of the immune response, for example stimulation or inhibition of the immune response, e.g., as measured by ELISPOT assay (cellular immune response), ICS (intracellular cytokine staining assay) and major histocompatibility complex (MHC) tetramer assay to detect and quantify antigen-specific T cells, quantifying the blood population of antigen-specific CD4+ T cells, or quantifying the blood population of antigen specific CD8+ T cells by a measurable amount, or where the increase is by at least 10%, at least 20%, at least 30%, at least 40%, at least 50%, at least 60%, at least 70%, at least 80%, at least 90%, at least 95%, at least 96%, at least 97%, at least 98%, at least 99%, at least 100%, when compared to a suitable control (e.g., a control composition where DCs are not loaded with tumor-specific cells, or not loaded with peptide derived from tumor-specific cells).

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition.

As used herein, the terms “suppressing immune cells” or “inhibiting immune cells” refer to a process (e.g., a signaling event) causing or resulting in the inhibition or suppression of one or more cellular responses or activities of an immune cell, selected from: proliferation, differentiation, cytokine secretion, cytotoxic effector molecule release, cytotoxic activity, and expression of activation markers, or resulting in anergizing of an immune cell or induction of apoptosis of an immune cell. Suitable assays to measure immune cell inhibition or suppression are known in the art and are described herein.

The term “specifically binds,” as used herein refers to the ability of a polypeptide or polypeptide complex to recognize and bind to a ligand in vitro or in vivo while not substantially recognizing or binding to other molecules in the surrounding milieu. In some embodiments, the polypeptide (e.g., an exogenous antigenic polypeptide or a displaceable exogenous polypeptide) binds the antigen-binding cleft of the loadable exogenous antigen-presenting polypeptide. Methods for determining whether two molecules specifically bind are well known in the art and include, for example, equilibrium dialysis, surface plasmon resonance, and the like.

The term “exogenous antigen-presenting polypeptide” as used herein, refers to a cell surface protein selected from an HLA class I polypeptide (e.g., HLA-A, HLA-B, HLA-C, HLA-E, or HLA-G), or an HLA class II polypeptide (e.g., HLA-DPα, HLA-DPβ, HLA-DMA, HLA-DMB, HLA DOA, HLA DOB, HLA DQα, HLA DQβ, HLA DRα, and HLA DRβ), comprised in a fusion construct, that is capable of binding an antigen and displaying the antigen on the cell surface for recognition by the appropriate immune cells. As used herein, an HLA class I polypeptide includes classical and non-classical HLA class I polypeptides. HLA class I molecules include β2 subunits and are thus only be recognized by CD8 co-receptors. HLA class II molecules include β1 and β2 subunits and thus can be recognized by CD4 co-receptors.

The term “wild-type exogenous antigen-presenting polypeptide” refers to an exogenous antigen-presenting polypeptide, as described herein, which does not contain one or more amino acid substitution(s) which stabilize the exogenous antigen-presenting polypeptide on a cell surface.

The term “loadable exogenous antigen-presenting polypeptide” or “loadable antigen-presenting polypeptide” as used herein refers to an exogenous antigen-presenting polypeptide comprised in a fusion construct, comprising one or more amino acid substitutions (including one or more pairs of amino acid substitutions) in the ectodomain of the fusion construct, as compared to the wild-type exogenous antigen-presenting polypeptide from which it was derived, which stabilizes the loadable exogenous antigen-presenting polypeptide on the cell surface. In some embodiments, the loadable exogenous antigen-presenting polypeptide is stabilized on the cell surface in the absence of a bound polypeptide. In some embodiments, the loadable exogenous antigen-presenting polypeptide binds an exogenous polypeptide, e.g., an exogenous antigenic polypeptide, and displays the exogenous polypeptide on the cell surface for recognition by the appropriate T-cells. In some embodiments, the loadable exogenous antigen-presenting polypeptide binds an exogenous displaceable polypeptide. In some embodiments, the exogenous displaceable polypeptide is displaced from the loadable exogenous antigen-presenting polypeptide, and replaced by an exogenous antigenic polypeptide. In some embodiments, the loadable exogenous antigen-presenting polypeptide is stabilized on the cell surface upon release of the displaceable polypeptide.

As used herein, the term “exogenous” in reference to a polypeptide (e.g., “exogenous polypeptide”) refers to a polypeptide that is introduced into or onto a cell, or is caused to be expressed by the cell by introducing an exogenous nucleic acid encoding the exogenous polypeptide into the cell or into a progenitor of the cell. In some embodiments, an exogenous polypeptide is a polypeptide encoded by an exogenous nucleic acid that was introduced into the cell or a progenitor of the cell, which nucleic acid is optionally not retained by the cell. In some embodiments, the exogenous polypeptide is a loadable exogenous antigen-presenting polypeptide, a wild-type exogenous antigen-presenting polypeptide, an exogenous antigenic polypeptide, an exogenous displaceable polypeptide, a cytokine, a coinhibitory polypeptide or a Treg costimulatory polypeptide. In some embodiments, an exogenous polypeptide is a polypeptide conjugated to the surface of the cell by chemical or enzymatic means.

The term “exogenous antigenic polypeptide” as used herein refers to an exogenous polypeptide that, together with the exogenous antigen-presenting polypeptide, is capable of inducing an immune response. In some embodiments, an exogenous antigenic polypeptide is bound to a loadable exogenous antigen-presenting polypeptide. In some embodiments, an exogenous antigenic polypeptide is bound to a wild-type exogenous antigen-presenting polypeptide. In some embodiments, a loadable exogenous antigen-presenting polypeptide has a higher affinity for an exogenous antigenic polypeptide than for an exogenous displaceable polypeptide. In some embodiments, an exogenous antigenic polypeptide binds to a loadable exogenous antigen-presenting polypeptide with a K_{D} of from about 1 picomolar to about 100 nanomolar. In some embodiments, a loadable exogenous antigen-presenting polypeptide has a higher affinity for an exogenous antigenic polypeptide than for an exogenous displaceable polypeptide. In some embodiments, the exogenous antigenic polypeptide binds to the loadable exogenous antigen-presenting polypeptide with a K_{D} of from about 1 picomolar to about 100 nanomolar.

The term “exogenous displaceable polypeptide” as used herein, refers to an exogenous polypeptide that is capable of binding and unbinding from the antigen-binding cleft of a loadable exogenous antigen-presenting polypeptide. In some embodiments, the loadable exogenous antigen-presenting polypeptide has a lower affinity for the exogenous displaceable polypeptide than for an exogenous antigenic polypeptide. In some embodiments, the exogenous displaceable polypeptide binds to the loadable exogenous antigen-presenting polypeptide with a K_{D} of from about 1 nM to about 100 μM. In some embodiments, the exogenous displaceable polypeptide binds to the loadable exogenous antigen-presenting polypeptide with a K_{D} of from about 10 nM to about 100 μM.

The term “exogenous T cell costimulatory polypeptide” as used herein, includes a polypeptide on an engineered erythroid cell or enucleated cell that specifically binds a cognate co-stimulatory molecule on a T cell (e.g., an HLA molecule, B and T lymphocyte attenuator (CD272), and a Toll like receptor), thereby providing a signal which, in addition to the primary signal provided by, for instance, binding of a TCR/CD3 complex with a wild-type or loadable exogenous antigen-presenting polypeptide loaded with peptide, mediates a T cell response, including, but not limited to, proliferation, activation, differentiation, and the like. A co-stimulatory polypeptide also encompasses, inter alia, an antibody that specifically binds with a co-stimulatory molecule present on a T cell. Exemplary exogenous co-stimulatory polypeptides are described in more detail below.

The term “exogenous T cell co-inhibitory polypeptide” as used herein refers to any polypeptide that suppresses a T cell, including inhibition of T cell activity, inhibition of T cell proliferation, anergizing of a T cell, or induction of apoptosis of a T cell. Exemplary exogenous co-inhibitory polypeptides are described in more detail below.

The term “Treg costimulatory polypeptide” as used herein refers to an exogenous polypeptide that expands regulatory T-cells (Tregs). In some embodiments, a Treg costimulatory polypeptide stimulates Treg cells by stimulating at least one of three signals involved in Treg cell development. Exemplary exogenous Treg co-stimulatory polypeptides are described in more detail below.

As used herein, the terms “click reaction” or “click chemistry” are interchangeably used to refer to a range of reactions used to covalently attach a first and a second moiety, for convenient production of linked products. It typically has one or more of the following characteristics: it is fast, is specific, is high-yield, is efficient, is spontaneous, does not significantly alter biocompatibility of the linked entities, has a high reaction rate, produces a stable product, favors production of a single reaction product, has high atom economy, is chemoselective, is modular, is stereoselective, is insensitive to oxygen, is insensitive to water, is high purity, generates only inoffensive or relatively non-toxic by-products that can be removed by nonchromatographic methods (e.g., crystallization or distillation), needs no solvent or can be performed in a solvent that is benign or physiologically compatible, e.g., water, stable under physiological conditions. Examples include an alkyne/azide reaction, a diene/dienophile reaction, or a thiol/alkene reaction. Other reactions can be used. In some embodiments, the click reaction is fast, specific, and high-yield.

As used herein, the terms “click handle” or “click chemistry handle” refer to a chemical moiety that is capable of reacting with a second click chemistry handle in a click reaction to produce a click signature. In embodiments, a click chemistry handle is comprised by a coupling reagent, and the coupling reagent may further comprise a substrate reactive moiety.

As used herein, the term “cytokine” refers to small soluble protein substances secreted by cells which have a variety of effects on other cells. Cytokines mediate many important physiological functions including growth, development, wound healing, and the immune response. Cytokines can act both locally and distantly from a site of release. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of other cytokines. Non limiting examples of cytokines include e.g., IL-1, IL-2, IL-3, IL-4, IL-5, IL-6, IL-7, IL-8, IL-9, IL-10, IL-11, IL-12/IL-23 P40, IL13, IL-15, IL-15/IL-15-RA, IL-17, IL-18, IL-21, IL-23, TGF-β, IFNγ, GM-CSF, Groα, MCP-1 and TNF-α.

As used herein, the term “endogenous” is meant to refer to a native form of a compound (e.g., a small molecule) or process. For example, in some embodiments, the term “endogenous” refers to the native form of a nucleic acid or polypeptide in its natural location in an organism or a cell or in the genome of an organism or a cell.

As used herein, the term “exogenous nucleic acid” refers to a nucleic acid (e.g., a gene) which is not native to a cell, but which is introduced into the cell or a progenitor of the cell. An exogenous nucleic acid may include a region or open reading frame (e.g., a gene) that is homologous to, or identical to, an endogenous nucleic acid native to the cell. In some embodiments, the exogenous nucleic acid comprises RNA. In some embodiments, the exogenous nucleic acid comprises DNA. In some embodiments, the exogenous nucleic acid is integrated into the genome of the cell. In some embodiments, the exogenous nucleic acid is processed by the cellular machinery to produce an exogenous polypeptide. In some embodiments, the exogenous nucleic acid is not retained by the cell or by a cell that is the progeny of the cell into which the exogenous nucleic acid was introduced.

As used herein, the term “stabilize” refers to an increased or prolonged presence of a loadable exogenous antigen-presenting polypeptide on the cell surface when not bound to an exogenous antigenic polypeptide as compared to the presence of the corresponding wild type antigen-presenting polypeptide when not bound to an exogenous antigenic polypeptide. For example, the loadable exogenous antigen-presenting polypeptide may be displayed on the surface when not bound to an exogenous antigenic polypeptide for an amount of time comparable to the presence of a wild-type exogenous antigen-presenting polypeptide on the cell surface when bound to an exogenous polypeptide. In some embodiments, the loadable exogenous antigen-presenting polypeptide may be displayed on the surface when not bound to an exogenous polypeptide for 10%, 20%, 30%, 40%, 50%, 60%, 70%, 80%, 90% or 100% the amount of time a wild-type exogenous antigen-presenting polypeptide is present on the cell surface when bound to an exogenous polypeptide. Methods of determining levels of exogenous antigen-presenting polypeptide are known in the art and include, for example, flow cytometry.

As used herein, the term “immunologically compatible” refers to a nucleic acid, polypeptide, a cell, or any combination thereof that is recognized as self by a subject's immune system, or that is not capable of eliciting an immune response in a subject.

As used herein, the term “immunologically incompatible” refers to a nucleic acid, polypeptide, a cell, or any combination thereof that is recognized as non-self by a subject's immune system, or that is capable of eliciting an immune response in a subject.

As used herein, the term “acceptor sequence” refers to a polymeric sequence which conditionally alters the state of another polymeric sequence. An acceptor sequence can comprise a polypeptide sequence, nucleic acid sequence (DNA sequence, aptamer sequence, RNA sequence, ribozyme sequence, hybrid sequence, modified or analogous nucleic acid sequence, etc.), carbohydrate sequence, and the like. Nucleic acid and amino acid sequences for use as acceptor sequences can be naturally occurring sequences, engineered sequences (for example, modified natural sequences), or sequences designed de novo.

As used herein, the term “engineered cell” refers to a genetically-modified cell or progeny thereof.

As used herein, the term “enucleated cell” refers to a cell that lacks a nucleus (e.g., due to a differentiation process such as erythropoiesis). In some embodiments, an enucleated cell is incapable of expressing a polypeptide. In some embodiments, an enucleated cell is an erythrocyte, a reticulocyte, or a platelet.

As used herein, “engineered enucleated cell” refers to a cell that originated from a genetically-modified nucleated cell or progeny thereof, and lacks a nucleus (e.g., due to differentiation). In some embodiments, the engineered enucleated cell includes an exogenous polypeptide that was produced by the genetically-modified nucleated cell or progeny thereof (e.g., prior to enucleation) from which the engineered enucleated cell originated.

As used herein, “engineered erythroid cell” refers to a genetically-modified erythroid cell or progeny thereof. Engineered erythroid cells include engineered nucleated erythroid cells (e.g., a genetically-modified erythroid precursor cell) and engineered enucleated erythroid cells (e.g., reticulocytes and erythrocytes that originated from a genetically modified erythroid precursor cell).

As used herein, “engineered enucleated erythroid cell” refers to an erythroid cell that originated from a genetically-modified nucleated erythroid cell or progeny thereof, and lacks a nucleus (e.g., due to differentiation). In some embodiments, an engineered enucleated erythroid cell comprises an erythrocyte or a reticulocyte that originated from a genetically-modified nucleated erythroid cell or progeny thereof. In some embodiments, the engineered enucleated erythroid cell did not originate from an immortalized nucleated erythroid cell or progeny thereof.

An “erythroid precursor cell”, as used herein, refers to a cell capable of differentiating into a reticulocyte or erythrocyte. Generally, erythroid precursor cells are nucleated. Erythroid precursor cells include a cord blood stem cell, a CD34⁺ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), an induced pluripotent stem cell (iPSC), a mesenchymal stem cell (MSC), a polychromatic normoblast, and an orthochromatic normoblast. In some embodiments, an erythroid precursor cell is an immortal or immortalized cell. For example, immortalized erythroblast cells can be generated by retroviral transduction of CD34⁺ hematopoietic progenitor cells to express Oct4, Sox2, Klf4, cMyc, and suppress TP53 (e.g., as described in Huang et al. (2014) Mol. Ther. 22(2): 451-63, the entire contents of which are incorporated by reference herein).

As used herein, the term “express” or “expression” refers to processes by which a cell produces a polypeptide, including transcription and translation. The expression of a particular polypeptide in a cell may be increased using several different approaches, including, but not limited to, increasing the copy number of genes encoding the polypeptide, increasing the transcription of a gene, and increasing the translation of an mRNA encoding the polypeptide.

As used herein, the terms “first”, “second”, and “third”, etc., with respect to exogenous polypeptides or nucleic acids are used for convenience of distinguishing when there is more than one type of exogenous polypeptide or nucleic acid. Use of these terms is not intended to confer a specific order or orientation of the exogenous polypeptides or nucleic acid unless explicitly so stated.

As used herein, the term “gene” is used broadly to refer to any segment of nucleic acid associated with expression of a given RNA or protein. Thus, genes include regions encoding expressed RNAs (which typically include polypeptide coding sequences) and, often, the regulatory sequences required for their expression. Genes can be obtained from a variety of sources, including cloning from a source of interest or synthesizing from known or predicted sequence information, and may include sequences designed to have specifically desired parameters.

As used herein the term “nucleic acid molecule” refers to a single or double-stranded polymer of deoxyribonucleotide and/or ribonucleotide bases. It includes, but is not limited to, chromosomal DNA, plasmids, vectors, mRNA, tRNA, siRNA, etc. which may be recombinant and from which exogenous polypeptides may be expressed when the nucleic acid is introduced into a cell.

As used herein, the terms “polypeptide”, “peptide” and “protein” are used interchangeably herein to refer to a polymer of amino acid residues. The terms “polypeptide”, “peptide” and “protein” also are inclusive of modifications including, but not limited to, glycosylation, phosphorylation, lipid attachment, sulfation, gamma-carboxylation of glutamic acid residues, hydroxylation, and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally.

As used herein, polypeptides referred to herein as “recombinant” refers to polypeptides which have been produced by recombinant DNA methodology, including those that are generated by procedures which rely upon a method of artificial recombination, such as the polymerase chain reaction (PCR) and/or cloning into a vector using restriction enzymes.

As used herein, the term “variant” of a polypeptide refers to a polypeptide having at least one amino acid residue difference as compared to a reference polypeptide, e.g., one or more substitutions, insertions, or deletions. In some embodiments, a variant has at least about 70%, 75%, 80%, 85%, 90%, 91%, 92%, 93%, 94% 95%, 96%, 97%, 98%, or 99% identity to that polypeptide. A variant may include a fragment (e.g., an enzymatically active fragment of a polypeptide (e.g., an enzyme). In some embodiments, a fragment may lack up to about 1, 2, 3, 4, 5, 10, 20, 30, 40, 50, or 100 amino acid residues on the N-terminus, C-terminus, or both ends (each independently) of a polypeptide, as compared to the full-length polypeptide. Variants may occur naturally or be non-naturally occurring. Non-naturally occurring variants may be generated using mutagenesis methods known in the art. Variant polypeptides may comprise conservative or non-conservative amino acid substitutions, deletions or additions.

As used herein, the term “sequence identity” or “identity,” in reference to nucleic acid and amino acid sequences refers to the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482; by means of the local homology algorithm of Neddleman and Wunsch, 1970, J. Mol. Bio1.48, 443; by means of the similarity search method of Pearson and Lipman, 1988, Proc. Natl. Acad. Sci. USA 85, 2444; or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

As used herein, the term “pharmaceutically acceptable carrier” includes any of the standard pharmaceutical excipients, carrier or stabilizer which are not toxic or deleterious to a mammal being exposed thereto at the dosage and/or concentration employed.

As used herein, the terms “subject”, “individual” and “patient” are used interchangeably herein and refer to any mammalian subject for whom diagnosis, treatment, or therapy is desired, particularly humans. The methods described herein are applicable to both human therapy and veterinary applications. In some embodiments, the subject is a mammal (e.g., a human subject).

As used herein, “administration,” “administering” and variants thereof refers to introducing a composition or agent into a subject and includes concurrent and sequential introduction of a composition or agent. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, or topically. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

As used herein, the terms “dose” or “dosage” are used interchangeably to refer to a specific quantity of a pharmacologically active material for administration to a subject for a given time. Unless otherwise specified, the doses recited refer to a plurality of engineered erythroid cells (e.g., engineered enucleated erythroid cells) or enucleated cells (e.g., modified enucleated cells) comprising a polypeptide(s) of interest as described herein. In some embodiments, a dose of engineered erythroid cells or enucleated cells refers to an effective amount of engineered erythroid cells or enucleated cells. When referring to a dose for administration, in an embodiment of any one of the methods, compositions or kits provided herein, any one of the doses provided herein is the dose as it appears on a label/label dose.

As used herein, the terms “therapeutically effective amount” and “effective amount” are used interchangeably to refer to an amount of an active agent (e.g. an engineered erythroid cell or an enucleated cell described herein) that is sufficient to provide the intended benefit (e.g. prevention, prophylaxis, delay of onset of symptoms, or amelioration of symptoms of a condition, e.g., a cancer, autoimmune disease, or infectious disease. In prophylactic or preventative applications, an effective amount may be administered to a subject susceptible to, or otherwise at risk of developing a disease, disorder or condition (e.g., a cancer, autoimmune disease, or infectious disease) to eliminate or reduce the risk, lessen the severity, or delay the onset of the disease, disorder or condition, including a biochemical, histologic and/or behavioral symptoms of the disease, disorder or condition, its complications, and intermediate pathological phenotypes.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

As used herein, the terms “treat,” “treating,” and/or “treatment” include abrogating, substantially inhibiting, slowing or reversing the progression of a disorder, disease or condition (e.g., a cancer, autoimmune disease, or infectious disease), substantially ameliorating clinical symptoms of a disorder, disease or condition, or substantially preventing the appearance of clinical symptoms of a disorder, disease or condition, obtaining beneficial or desired clinical results. Treating further refers to accomplishing one or more of the following: (a) reducing the severity of the disorder, disease or condition (e.g., a cancer, autoimmune disease, or infectious disease); (b) limiting development of symptoms characteristic of the disorder, disease or condition(s) being treated; (c) limiting worsening of symptoms characteristic of the disorder, disease or condition(s) being treated; (d) limiting recurrence of the disorder, disease or condition(s) in subjects that have previously had the disorder, disease or condition(s); and (e) limiting recurrence of symptoms in subjects that were previously asymptomatic for the disorder, disease or condition(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, the term “cancer” refers to diseases in which abnormal cells divide without control. In certain embodiments, the cancer is selected from cancers including, but not limited to, acute lymphoblastic leukemia (ALL), acute myeloid leukemia (AML), anal cancer, bile duct cancer, bladder cancer, bone cancer, bowel cancer, brain tumour, breast cancer, cancer of unknown primary, cancer spread to bone, cancer spread to brain, cancer spread to liver, cancer spread to lung, carcinoid, cervical cancer, choriocarcinoma, chronic lymphocytic leukemia (CLL), chronic myeloid leukemia (CML), colon cancer, colorectal cancer, endometrial cancer, eye cancer, gallbladder cancer, gastric cancer, gestational trophoblastic tumour (GTT), hairy cell leukemia, head and neck cancer, Hodgkin lymphoma, kidney cancer, laryngeal cancer, leukemia, liver cancer, lung cancer, lymphoma, melanoma skin cancer, mesothelioma, men's cancer, molar pregnancy, mouth and oropharyngeal cancer, myeloma, nasal and sinus cancers, nasopharyngeal cancer, non Hodgkin lymphoma (NHL), oesophageal cancer, ovarian cancer, pancreatic cancer, penile cancer, prostate cancer, rare cancers, rectal cancer, salivary gland cancer, secondary cancers, skin cancer (non melanoma), soft tissue sarcoma, stomach cancer, testicular cancer, thyroid cancer, unknown primary cancer, uterine cancer, vaginal cancer, and vulval cancer.

As used herein, the term “autoimmune disease” refers generally to diseases or conditions in which a subject's immune system attacks the body's own cells, causing tissue destruction or damage. In some embodiments, the term “autoimmune disease” includes any autoimmune disease that is modulated by Follicular helper T (Tfh), Thl cells, and/or T helper 17 (Th17) T cells (see, e.g., Zhang et al. (2017) J. Immunol. 198(1 Suppl.) 55.13; Jeon et al. (2016) Immune Netw. 16(4): 219-32; and Noack et al. (2014) Autoimmunity Reviews 13(6): 668-77; the contents of each of which are incorporated by reference herein). For example, autoimmune diseases include, but are not limited to, rheumatoid arthritis (RA), juvenile idiopathic arthritis, rheumatoid spondylitis, ankylosing spondylitis, osteoarthritis, gouty arthritis, psoriatic arthritis, juvenile rheumatoid arthritis, type I diabetes (T1D), multiple sclerosis (MS), mixed connective tissue disorder, graft versus host disease (GVHD), autoimmune uveitis, nephritis, psoriasis, systemic lupus erythematosus (SLE), herpetic stromal keratitis (HSK), asthma, Crohn's disease, ulcerative colitis, spondylarthritis, active axial spondyloarthritis (active axSpA) and non-radiographic axial spondyloarthritis (nr-axSpA), pemphigus vulgaris, bullous pemphigoid, membranous glomerulonephritis, neuromyelitis optica, autoimmune encephalomyelitis, autoimmune hepatitis, chronic inflammatory demyelinating polyradiculoneuropathy, dermatomyositis, giant cell arteritis, granulomatosis with polyangiitis, Kawasaki disease, lupus nephritis, polyarteritis nodosa, pyoderma gangrenosum, and Takayasu's arteritis. Autoimmune diseases may be diagnosed using blood tests, cerebrospinal fluid analysis, electromyogram (measures muscle function), and magnetic resonance imaging of the brain, but antibody testing in the blood, for self-antibodies (or auto-antibodies) is particularly useful. Usually, IgG class antibodies are associated with autoimmune diseases.

### I. Engineered Erythroid Cells or Enucleated Cells Including Exogenous Antigen-Pesenting Polypeptides

The present disclosure features engineered erythroid cells (e.g., engineered enucleated erythroid cells) or enucleated cells (e.g., modified enucleated cells) that are engineered to include wild-type or loadable exogenous antigen-presenting polypeptides. In some embodiments, the engineered erythroid cells or enucleated cells provided herein comprise a loadable exogenous antigen-presenting polypeptide on the cell surface which comprises one or more amino acid substitutions which stabilize the loadable exogenous antigen-presenting polypeptide on the cell surface. In some embodiments, the loadable exogenous antigen-presenting polypeptide is stabilized on the cell surface in the absence of a polypeptide bound to (e.g., to the antigen-binding cleft of) the exogenous antigen-presenting polypeptide.

In some embodiments, an exogenous antigenic polypeptide of interest can be selected and loaded onto a wild-type or loadable exogenous antigen-presenting polypeptide present on an engineered erythroid or enucleated cell described herein prior to administration to a subject, thus allowing for customizable therapy specific to the particular subject.

In some embodiments, the loadable exogenous antigen-presenting polypeptide comprises a displaceable polypeptide bound to the loadable exogenous antigen-presenting polypeptide. In some embodiments, the displaceable polypeptide can be replaced with a selected exogenous antigenic polypeptide of interest prior to administration to a subject.

The skilled artisan would appreciate, based upon the disclosure provided herein, that numerous immunoregulatory molecules can be used to produce an almost limitless variety of engineered erythroid cells (e.g., engineered enucleated erythroid cells) or enucleated cells (e.g., modified enucleated cells) once armed with the teachings provided herein. That is, there is extensive knowledge in the art regarding the events and molecules involved in activation and induction of immune cells, e.g., activation of T regulatory cells, or inhibition of suppression of immune cells, e.g., natural killer (NK) cells, T cells, B cells, macrophages, and/or DCs.

In some aspects, the present disclosure provides an engineered erythroid cell (e.g., engineered enucleated erythroid cell) or enucleated cell (e.g., modified enucleated cell) comprising an exogenous polypeptide (e.g., presenting the exogenous polypeptide on the cell surface). Exogenous polypeptides include, but are not limited to, wild-type exogenous antigen-presenting polypeptides, loadable exogenous antigen-presenting polypeptides, exogenous antigenic polypeptides, exogenous displaceable polypeptides, exogenous costimulatory polypeptides, exogenous coinhibitory polypeptides, and exogenous Treg costimulatory polypeptides.

### Exogenous Antigen-Presenting Polypeptides

In one aspect, the disclosure provides engineered erythroid cells or enucleated cels including a loadable exogenous antigen-presenting polypeptide. Loadable exogenous antigen-presenting polypeptides of the present disclosure include polypeptides comprising human leukocyte antigen (HLA) class I and HLA class II polypeptides comprising one or more amino acid substitutions as compared to the corresponding wild-type exogenous antigen-presenting polypeptide, which stabilize the loadable exogenous antigen-presenting polypeptide on the cell surface, e.g., in the absence of a bound exogenous polypeptide.

In some embodiments, a wild-type or loadable exogenous antigen-presenting polypeptide, as described herein is bound to an exogenous antigenic polypeptide. In other embodiments, the loadable exogenous antigen-presenting polypeptide is bound to an exogenous displaceable polypeptide. In other embodiments, the exogenous displaceable polypeptide is removed and replaced with an exogenous antigenic polypeptide, and the loadable exogenous antigen-presenting polypeptide maintains a stable conformation on the cell surface.

HLA class I polypeptides are heterodimers that consist of two polypeptide chains, an a chain and a β2-microglobulin (β2M) chain. The HLA class I a chains consist of a single polypeptide composed of three extracellular domains named α1, α2, and α3, a transmembrane domain that anchors it in the plasma membrane, and a short intracytoplasmic tail. The β2M chain is a single polypeptide non-covalently bound to the a chain. Only the a chain is polymorphic and encoded by an HLA gene, while the β2m subunit is not polymorphic and encoded by the β2M gene. HLA class I polypeptides have β2 subunits and can only be recognized by CD8 co-receptors. As used herein, HLA class I polypeptides include HLA-A, HLA-B, HLA-C, HLA-E and HLA-G.

In some embodiments, the wild-type or loadable exogenous antigen-presenting polypeptide comprises a HLA a heavy chain polypeptide and a β2M polypeptide, or a fragment thereof. In some embodiments, the HLA a heavy chain comprises one or more HLAa heavy domains (e.g., one or more of the alphal, alpha2, and alpha3 domains).

In some embodiments of the present disclosure, the wild-type or loadable exogenous antigen-presenting polypeptide comprises a HLA class I polypeptide and includes a signal sequence. In some embodiments, the HLA heavy chain polypeptide is derived from an HLA class I polypeptide. In some embodiments, the wild-type or loadable exogenous antigen-presenting polypeptide is derived from a class I HLA polypeptide, and does not include a signal sequence. In some embodiments, the wild-type or loadable exogenous antigen-presenting polypeptide comprises an ectodomain from a class I HLA polypeptide. In some embodiments, the wild-type or loadable exogenous antigen-presenting polypeptide comprises one or more of the alphal, alpha2, and alpha3 domains from a class I HLA polypeptide.

HLA class II polypeptides are also heterodimers that consist of an a and 13 polypeptide chain. The subdesignation of chains as e.g., α1, α2, and β1 and β2, refers to separate domains (or subunits) within the HLA α gene and β polypepide. CD4 binds to the β2 region. As used herein, HLA class II polypeptides include HLA-DPα, HLA-DPβ, HLA-DMA, HLA-DMB, HLA DOA, HLA DOB, HLA DQα, HLA DQβ, HLA DRα, and HLA DRβ.

In some embodiments, the wild-type or loadable exogenous antigen-presenting polypeptide comprises a HLA class II polypeptide and includes a signal sequence. In some embodiments, the HLA heavy chain polypeptide is derived from an HLA class II polypeptide. In some embodiments, the wild-type or loadable exogenous antigen-presenting polypeptide is derived from a HLA class II polypeptide, and does not include a signal sequence. In some embodiments, the wild-type or loadable exogenous antigen-presenting polypeptide comprises an ectodomain from a HLA class II polypeptide. In some embodiments, the wild-type or loadable exogenous antigen-presenting polypeptide comprises one or more of the α1 domain and α2 domain from a HLA class II a chain. In some embodiments, the wild-type or loadable exogenous antigen-presenting polypeptide comprises one or more of the β1 domain and β2 domain from a HLA class IIβ chain. In some embodiments, the wild-type or loadable exogenous antigen-presenting polypeptide comprises one or more of the α1 domain and α2 domain from a HLA class II a chain, and one or more of the β1 domain and β2 domain from a HLA class II β chain.

The wild-type or loadable exogenous antigen-presenting polypeptides of the present disclosure can include subunits of a cell surface complex or cell surface molecule, e.g., a HLA class I or HLA class II polypeptide, and bind to an exogenous antigenic polypeptide. In some embodiments, the wild-type or loadable exogenous antigen-presenting polypeptides include subunits of HLA class II polypeptides, and a bind to an exogenous antigenic polypeptide. In some embodiments, the wild-type or loadable exogenous antigen-presenting polypeptides include subunits of HLA class I and bind to an exogenous antigenic polypeptide. In some embodiments, the exogenous antigen-presenting polypeptide comprises a leader (signal) sequence. In some embodiments, the exogenous antigen-presenting polypeptide does not comprise a leader (i.e., signal) sequence. In some embodiments, a leader sequence is fused to a wild-type or loadable exogenous antigen-presenting polypeptide (e.g., including a HLA class I or HLA class II polypeptide lacking its leader (i.e., signal sequence)). In some embodiments, the exogenous wild-type or loadable antigen-presenting polypeptide is a fusion polypeptide comprising a leader sequence and a HLA class I polypeptide. In some embodiments, the exogenous wild-type or loadable antigen-presenting polypeptide is a fusion polypeptide comprising a leader sequence and a HLA class II polypeptide. In some embodiments, the leader sequence is selected from the sequences set forth in Table 1.

TABLE 1
Leader Sequences

| SEQ ID | | |
|---|---|---|
| NO: | Sequence Description | Amino Acid Sequence |
| 6 | β2m leader sequence | MSRSVALAVLALLSLSGLEA |
| 7 | GPA signal peptide | MYGKIIFVLLLSEIVSISA |

In some embodiments, a wild-type or loadable exogenous antigen-presenting polypeptide comprises one or more of alpha domains 1-3 of an HLA class I polypeptide and a β2m polypeptide, or fragments or variants thereof. In some embodiments, a wild-type or loadable exogenous antigen-presenting polypeptide comprises the transmembrane domain of a Type I membrane protein (e.g., GPA), the ectodomain of an HLA class I polypeptide, and a β2m polypeptide. In some embodiments, a wild-type or loadable exogenous antigen-presenting polypeptide comprises the transmembrane domain of a Type I membrane protein (e.g., GPA), the following extracellular domains of an HLA class I polypeptide: alpha domain 1, alpha domain 2, and alpha domain 3, and a β2m polypeptide. In some embodiments, a wild-type or loadable exogenous antigen-presenting polypeptide comprises the transmembrane domain of a Type I membrane protein (e.g., GPA), an HLA class I polypeptide that has been truncated to exclude both the native transmembrane domain and the native cytosolic region, and further includes a β2m polypeptide.

In some embodiments a wild-type or loadable exogenous antigen-presenting polypeptide comprises a HLA class II α chain, or fragments thereof (e.g., one or more of alpha domains 1 and 2), and a HLA class II β chain, or fragments thereof (e.g., one or more of beta domains 1 and 2), or fragments or variants thereof. In some embodiments, a wild-type or loadable exogenous antigen presenting polypeptide comprises the transmembrane domain of a Type I membrane protein (e.g., GPA), the ectodomain of an HLA class II α chain (e.g., one or more of alpha domains 1 and 2), and the ectodomain of an HLA class II β chain (e.g., one or more of beta domains 1 and 2). In some embodiments, a wild-type or loadable exogenous antigen presenting polypeptide comprises the transmembrane domain of a Type I membrane protein (e.g., GPA), an HLA class II α chain that has been truncated to exclude both the native transmembrane domain and the native cytosolic region, and an HLA class II β chain that has been truncated to exclude both the native transmembrane domain and the native cytosolic region.

In some embodiments, the engineered erythroid cell (e.g., engineered enucleated erythroid cell) or enucleated cell (e.g., modified enucleated cell) comprises a wild-type or loadable antigen-presenting polypeptide fused to an exogenous antigenic polypeptide (e.g., as a single-chain construct).

In some embodiments, the engineered erythroid cell (e.g., engineered enucleated erythroid cell) or enucleated cell (e.g., modified enucleated cell) comprises a wild-type or loadable antigen-presenting polypeptide fused to an exogenous displaceable polypeptide.

In some embodiments, the wild-type or loadable exogenous antigen-presenting polypeptide comprises both an HLA class I α chain and a β2M polypeptide. In some embodiments, the wild-type or loadable exogenous antigen-presenting polypeptide comprises only an HLA class I α chain. In some embodiments, the wild-type or loadable exogenous antigen-presenting polypeptide comprises only a β2M polypeptide. In some embodiments, the wild-type or loadable exogenous antigen-presenting polypeptide comprises both an HLA class I α chain and a β2M polypeptide, and the HLA class I α chain and β2m polypeptide are non-covalently attached. In some embodiments, the wild-type or loadable exogenous antigen-presenting polypeptide comprises both an HLA class I α chain and a β2M polypeptide, and the HLA class I α chain and β2m polypeptide are covalently attached or fused. In some embodiments, the wild-type or loadable exogenous antigen-presenting polypeptide comprises an exogenous antigenic polypeptide linked to an HLA class I a chain. In some embodiments, the wild-type or loadable exogenous antigen-presenting polypeptide comprises an exogenous antigenic polypeptide linked to a β2M polypeptide. In some embodiments, the wild-type or loadable exogenous antigen-presenting polypeptide comprises an exogenous antigenic polypeptide linked to a β2M polypeptide, which is linked to a HLA class I α chain.

In some embodiments, the wild-type or loadable exogenous antigen-presenting polypeptide comprises both a HLA class II α chain and a HLA class II β chain. In some embodiments, the wild-type or loadable exogenous antigen-presenting polypeptide comprises only a HLA class II α chain. In some embodiments, the wild-type or loadable exogenous antigen-presenting polypeptide comprises only a HLA class II β chain. In some embodiments, the wild-type or loadable exogenous antigen-presenting polypeptide comprises both a HLA class II α chain and a HLA class II β chain, and the HLA class II α chain and HLA class II β chain are non-covalently attached. In some embodiments, the wild-type or loadable exogenous antigen-presenting polypeptide comprises both a HLA class II α chain and a HLA class II β chain, and the HLA class II α chain and HLA class II β chain are covalently attached or fused. In some embodiments, the wild-type or loadable exogenous antigen-presenting polypeptide comprises an exogenous antigenic polypeptide linked to a HLA class II α chain. In some embodiments, the wild-type or loadable exogenous antigen-presenting polypeptide comprises an exogenous antigenic polypeptide linked to a HLA class II β chain. In some embodiments, the wild-type or loadable exogenous antigen-presenting polypeptide comprises an exogenous antigenic polypeptide linked to a HLA class II β-chain, which is linked to a HLA class II α-chain.

In some embodiments, the wild-type or loadable exogenous antigen-presenting polypeptide comprises an anchor or transmembrane domain. In some embodiments, the the wild-type or loadable exogenous antigen-presenting polypeptide comprises an anchor or transmembrane domain from a Type I membrane protein. In some embodiments, the wild-type or loadable exogenous antigen-presenting polypeptide comprises an HLA class I polypeptide that does not comprise a native transmembrane domain nor a native cytosolic region, and instead comprises a non-native transmembrane domain (e.g., the transmembrane domain of a Type I membrane protein). In some embodiments, the wild-type or loadable exogenous antigen-presenting polypeptide comprises an HLA class II α chain and an HLA class II β chain, each of which lack a native transmembrane domain and/or a native cytosolic region, and instead the the wild-type or loadable exogenous antigen-presenting polypeptide comprises a non-native transmembrane domain (e.g., the transmembrane domain of a Type I membrane protein). In some embodiments, the wild-type or loadable exogenous antigen-presenting polypeptide comprises a transmembrane domain from a Type I membrane protein selected from glycophorin A (GPA); glycophorin B (GPB); Basigin (also known as CD147); CD44; CD58 (also known as LFA3); Intercellular Adhesion Molecule 4 (ICAM4); Basal Cell Adhesion Molecule (BCAM); CR1; CD99; Erythroblast Membrane Associated Protein (ERMAP); junctional adhesion molecule A (JAM-A); neuroplastin (NPTN); AMIGO2; and DS Cell Adhesion Molecule Like 1 (DSCAML1). In some embodiments, the Type 1 membrane protein comprises GPA. In some embodiments, the transmembrane domain is a glycophorin anchor, and in particular GPA, or a fragment thereof. In some embodiments, the transmembrane domain comprises an amino acid sequence listed in Table 2.

TABLE 2
Transmembrane Domain Sequences

| SEQ | | | |
|---|---|---|---|
| ID | Sequence | Sequence | |
| NO: | name | description | Amino acid sequence |
| 8 | GPA | Full length GPA | MYGKIIFVLLLSAIVSISALSTTEVAMHT |
| | | | STSSSVTKSYISSQTNDTHKRDTYAATP |
| | | | RAHEVSEISVRTVYPPEEETGERVQLAH |
| | | | HFSEPEITLIIFGVMAGVIGTILLISYGIRR |
| | | | LIKKSPSDVKPLPSPDTDVPLSSVEIENP |
| | | | ETSDQ |
| 9 | GPA | Fragment of | LSTTEVAMHTSTSSSVTKSYISSQTNDT |
| | | GPA comprising | HKRDTYAATPRAHEVSEISVRTVYPPEE |
| | | a transmembrane | ETGERVQLAHHFSEPEITLIIFGVMAGVI |
| | | domain | GTILLISYGIRRLIKKSPSDVKPLPSPDTD |
| | | | VPLSSVEIENPETSDQ |

In some embodiments, the wild-type or loadable exogenous antigen-presenting polypeptide comprises a HLA class I or HLA class II polypeptide (or fragment thereof) which is connected to a transmembrane domain (e.g., non-endogenous transmembrane domain, e.g., GPA) via a linker. In some embodiments, the linker is disposed between the alpha3 chain of a HLA class I polypeptide and a transmembrane domain. In some embodiments, the linker is a GlySer linker. In some embodiments, the linker is about 18 amino acids in length. In other embodiments, the linker is between about 2 amino acids in length and 30 amino acids in length. In some embodiments, the linker is about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more amino acids in length. In some embodiments, the linker is a cleavable linker. In some embodiments, the linker is selected from an amino acid sequence listed in Table 3.

In some embodiments, the wild-type or loadable exogenous antigen-presenting polypeptide comprises a linker disposed between the one or more HLAa heavy chain polypeptide and the β2M polypeptide. In some embodiments, the linker is a GlySer linker. In some embodiments, the linker is about 20 amino acids in length. In other embodiments, the linker is between about 2 amino acids in length and 30 amino acids in length. In some embodiments, the linker is about 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more amino acids in length. In some embodiments, the linker is about 18 amino acid residues in length. In some embodiments, the a linker is a cleavable linker. In some embodiments, the linker is selected from an amino acid sequence listed in Table 3.

TABLE 3
Linker Sequences

| SEQ | Sequence | |
|---|---|---|
| ID | Descrip- | |
| NO: | tion | Amino Acid Sequence |
| 31 | Linker | GGGGSGGGGSGGGGS |
| 10 | Linker | GGGGSGGGGSGGGGSGGGGS |
| 11 | Linker | GSGSGSGSEDGSGSGSGS |
| 12 | Linker | GSGSGSGSGSGSGSGSGS |
| 13 | Linker | GCGGSGGGGSGGGGS |
| 14 | Linker | TGSGGGGSGGGGSGGGGSGGGGSGT |
| 15 | Linker | GGSGGSGGGGGSGGGSGGGSGGGS |
| 16 | Linker | SGRGGGGSGGGGSGGGGSGGGGSSPA |
| 17 | Linker | GGGGSGGGGSGGGGSGGGGSGGGG |
| 18 | Snorkel | SGRGASSGSSGSGSQKKPRYEIRWKVVVISAILALV |
| | linker | VLTVISLIILIMLWGSGMQSPA (“snorkel |
| | | linker”) |

In some embodiments, an exogenous antigenic polypeptide or an exogenous displaceable polypeptide is connected to the wild-type or loadable exogenous antigen-presenting polypeptide via a linker. In some embodiments, the linker is about 15 amino acids in length. In other embodiments, the linker is between about 10 amino acids in length and 30 amino acids in length. In some embodiments, the linker is about 10, 11, 12, 13, 14, 15, 16, 17, 18, 19, 20, 21, 22, 23, 24, 25, 26, 27, 28, 29, or 30 or more amino acids in length.

In some embodiments, an enzymatic cleavage site is disposed in the linker connecting the wild-type or loadable exogenous antigen-presenting polypeptide and the exogenous antigenic polypeptide or exogenous displaceable polypeptide. In some embodiments, the cleavage site can be used to free the exogenous displaceable polypeptide (e.g., upon displacement from the HLA polypeptide). In other embodiments, the cleavage site can expose, upon cleavage, an N-terminal amino acid sequence that can be used to conjugate a moiety (e.g., a click handle) or a peptide. In some embodiments, the enzymatic cleavage site is a human rhinovirus (HRV) 3C protease cleavage site. In some embodiments, the enzymatic cleavage site comprises the amino acid sequence LEVLFQ/GP (SEQ ID NO: 1), wherein the backslash indicates the cleavage site. In some embodiments, the enzymatic cleavage site is a tobacco etch virus (TEV) protease cleavage site. In some embodiments, the enzymatic cleavage site comprises the amino acid sequence ENLYFQ/G (SEQ ID NO: 2), wherein the backslash indicates the cleavage site.

In some embodiments, the enzymatic cleavage site is within 10 amino acids or less from a GGG motif in the linker, and is located between the GGG motif and a displaceable polypeptide. In some embodiments, the GGG motif is C terminal to the enzymatic cleavage site. In some embodiments, the enzymatic cleavage site is within 10 amino acids or less from an LPTXG motif (SEQ ID NO: 3), and is located between the LPTXG motif (SEQ ID NO: 3) and a displaceable polypeptide. In some embodiments, the LPTXG motif (SEQ ID NO: 3) is C terminal to the enzymatic cleavage site.

In some embodiments, the enzymatic cleavage site is adjacent to a SpyTag sequence (AHIVMVDAYKPTK (SEQ ID NO: 4)), and is located between the SpyTag sequence and the displaceable polypeptide. In some embodiments, the SpyTag sequence is C terminal to the enzymatic cleavage site. In some embodiments, the enzymatic cleavage site is within 10 amino acids or less from the amino acid sequence AHIVMVDAYKPTK (SEQ ID NO: 4).

In some embodiments, the enzymatic cleavage site is adjacent to a KTag sequence (ATHIKFSKRD (SEQ ID NO: 5)), and is located between the KTag sequence and a displaceable polypeptide. In some embodiments, the KTag sequence is C terminal to the enzymatic cleavage site. In some embodiments, the enzymatic cleavage site is within 10 amino acids or less from the amino acid sequence ATHIKFSKRD (SEQ ID NO: 5).

In some embodiments, a loadable exogenous antigen-presenting polypeptide provided herein comprises one or more amino acid substitutions which stabilize the loadable exogenous antigen-presenting polypeptide on the cell surface. In some embodiments, the loadable exogenous antigen-presenting polypeptide is stabilized on the cell surface of a cell (e.g., an engineered erythroid cell or an enucleated cell comprising the loadable exogenous antigen-presenting polypeptide) in the absence of a polypeptide (e.g., an exogenous antigenic polypeptide or an exogenous displaceable polypeptide) bound to (e.g., to the antigen-binding cleft of) the loadable exogenous antigen-presenting polypeptide.

In some embodiments, an exogenous antigenic polypeptide or an exogenous displacebale polypeptide is loaded on the antigen-binding cleft of a wild-type or loadable exogenous antigen-presenting polypeptide described herein. In some embodiments, the exogenous antigenic polypeptide or exogenous displaceable polypeptide is presented on a wild-type or loadable exogenous antigen-presenting polypeptide, e.g., the exogenous antigenic polypeptide or exogenous displaceable polypeptide is bound to the wild-type or loadable exogenous antigen-presenting polypeptide. The exogenous antigenic polypeptide or exogenous displaceable polypeptide may be attached either covalently or non-covalently to the wild-type or loadable exogenous antigen-presenting polypeptide. In some embodiments, the exogenous antigenic polypeptide or exogenous displaceable polypeptide is free, and can be bound to the wild-type or loadable exogenous antigen-presenting polypeptide present on cell surface of an engineered erythroid cell (e.g., engineered enucleated erythroid cell) or enucleated cell (e.g., modified enucleated cell). In some embodiments, coupling reagents can be used to link (e.g., covalently attach) an exogenous polypeptide or exogenous displaceable polypeptide to a wild-type or loadable exogenous antigen-presenting polypeptide present on the cell surface.

In some embodiments, click chemistry, as described in detail herein, can be used to link an exogenous antigenic polypeptide or a exogenous displaceable polypeptide to a wild-type or loadable exogenous antigen-presenting polypeptide present on the cell surface.

Multiple assays for assessing binding affinity and/or determining whether an exogenous antigenic polypeptide or exogenous displaceable polypeptide specifically binds to (or is specifically bound to) a particular ligand (e.g., a wild-type or loadable exogenous antigen-presenting polypeptide) are known in the art. For example, surface plasmon resonance (Biacore®) can be used to determine the binding constant of a complex between two polypeptides. In this assay, the dissociation constant for the complex can be determined by monitoring changes in the refractive index with respect to time as buffer is passed over the chip. Other suitable assays for measuring the binding of one polypeptide to another include, for example, immunoassays such as enzyme linked immunosorbent assays (ELISA) and radioimmunoassays (RIA), or determination of binding by monitoring the change in the spectroscopic or optical properties of the proteins using fluorescence, UV absorption, circular dichroism, or nuclear magnetic resonance (NMR). Other exemplary assays include, but are not limited to, Western blot, analytical ultracentrifugation, and spectroscopy (see, e.g., Scatchard et al. (1949) Ann. N.Y. Acad. Sci. 51:660; Wilson (2002) Science 295: 2103; Woffi et al. (1993) Cancer Res. 53: 2560-5; U.S. Pat. Nos. 5,283,173 and 5,468,614; and International Patent Publication No. WO 2018/005559). Alternatively, binding of a polypeptide to a particular ligand (e.g., a wild-type or loadable exogenous antigen-presenting polypeptide) may be determined using a predictive algorithm. For example, methods for predicting HLA class II and class II epitopes are well known in the art, and include TEPITOPE (see, e.g., Meister et al. (1995) Vaccine 13: 581-91), EpiMatrix (De Groot et al. (1997) AIDS Res Hum Retroviruses 13: 529-31), the Predict Method (Yu et al. (2002) Mol. Med. 8: 137-48), the SYFPEITHI epitope prediction algorithm (Schuler et al. (2007) Methods Mol Biol. 409: 75-93, and Rankpep (Reche et al. (2002) Hum. Immunol. 63(9): 701-9). Additional algorithms for predicting HLA class I and class II epitopes are described, for example, in Kessler and Melief (2007) Leukemia 21(9): 1859-74.

In some embodiments, a wild-type or loadable exogenous antigen-presenting polypeptide comprises a HLA-A, a HLA-B, a HLA-C, a HLA-E, a HLA-G, a HLA-DRB1, a HLA-DQA1, a HLA-DQB1, a HLA-DPA1, and/or a HLA-DPB1 polypeptide, or a fragment thereof, and is capable of binding to an exogenous antigenic polypeptide and of displaying it on a cell surface.

Some exemplary contructs comprising the wild-type or loadable exogenous antigen-presenting polypeptides are described in FIGS. 2A and 2B.

In some embodiments, the wild-type or loadable exogenous antigen-presenting polypeptide comprises an HLA class I polypeptide (or a fragment thereof), and optionally a β2M polypeptide, wherein the HLA class I polypeptide is a HLA-A polypeptide. In some embodiments, the HLA-A polypeptide comprises a HLA-A allele selected from the group consisting of A*01:01, A*02:01, A *03:01, A*24:02, A*11:01, A*29:02, A*32:01, A*68:01, A*31:01, A*25:01, A*26:01, A*23:01, and A*30:01. In some embodiments, the HLA-A polypeptide is linked to an exogenous antigenic polypeptide or an exogenous displaceable polypeptide as a single chain fusion polypeptide. In some embodiments, the single chain fusion polypeptide includes a transmembrane domain (e.g., a transmembrane domain set forth in Table 2). In some embodiments, the single chain fusion polypeptide comprises an HLA-A polypeptide that does not comprise a native transmembrane domain nor a native cytosolic region, and instead comprises a non-native transmembrane domain (e.g., the transmembrane domain of a Type I membrane protein). In some embodiments, the single chain fusion polypeptide includes a linker (e.g., between an exogenous antigenic polypeptide or an exogenous displaceable polypeptide and a β2M polypeptide, between a β2M polypeptide and a HLA-A α chain (or fragment thereof), or between a HLA-A α chain (or fragment thereof) and a transmembrane domain). In some embodiments, the linker is selected from a sequence set forth in Table 3. In some embodiments, the single chain fusion polypeptide includes a leader sequence (e.g., a linker sequence set forth in Table 1). In some embodiments, a HLA-A leader sequence is fused to a wild-type or loadable exogenous antigen-presenting polypeptide provided herein that comprises a transmembrane domain.

In some embodiments, the wild-type or loadable exogenous antigen-presenting polypeptide comprises an HLA class I polypeptide (or a fragment thereof), and optionally a (32M polypeptide, wherein the HLA class I polypeptide is a HLA-B polypeptide. In some embodiments, the HLA-B polypeptide comprises a HLA-B allele selected from the group consisting of B*08:01, B*07:02, B*44:02, B*15:01, B*40:01, B*44:03, B*35:01, B*51:01, B*27:05, B*57:01, B*18:01, B*14:02, B*13:02, B*55:01, B*14:01, B*49:01, B*37:01, B*38:01, B*39:01, B*35:03, and B*40:02. In some embodiments, the HLA-B polypeptide is linked to an exogenous antigenic polypeptide or an exogenous displaceable polypeptide as a single chain polypeptide. In some embodiments, the single chain fusion polypeptide includes a transmembrane domain (e.g., a transmembrane domain set forth in Table 2). In some embodiments, the single chain fusion comprises an HLA-B polypeptide that does not comprise a native transmembrane domain nor a native cytosolic region, and instead comprises a non-native transmembrane domain (e.g., the transmembrane domain of a Type I membrane protein). In some embodiments, the single chain fusion polypeptide includes a linker (e.g., between an exogenous antigenic polypeptide or an exogenous displaceable polypeptide and a β2M polypeptide, between a β2M polypeptide and a HLA-B α chain (or fragment thereof), or between a HLA-B α chain (or a fragment thereof) and a transmembrane domain). In some embodiments, the linker is selected from a sequence set forth in Table 3. In some embodiments, the single chain fusion polypeptide includes a leader sequence (e.g., a leader sequence set forth in Table 1). In some embodiments, a HLA-B leader sequence is fused to a wild-type or loadable exogenous antigen-presenting polypeptide provided herein that comprises a transmembrane domain.

In some embodiments, the wild-type or loadable exogenous antigen-presenting polypeptide comprises an HLA class I polypeptide (or a fragment thereof), and optionally a β2M polypeptide, wherein the HLA class I polypeptide is a HLA-C polypeptide. In some embodiments, the HLA-C polypeptide comprises a HLA-C allele selected from the group consisting of C*07:01, C*07:02, C*05:01, C*06:02, C*04:01, C*03:04, C*03:03, C*02:02, C*16:01, C*08:02, C*12:03, C*01:02, C*15:02, C*07:04, and C*14:02. In some embodiments, the HLA-C polypeptide is linked to an exogenous antigenic polypeptide or an exogenous displaceable polypeptide as a single chain fusion polypeptide. In some embodiments, the single chain fusion polypeptide includes a transmembrane domain (e.g., a transmembrane domain set forth in Table 2). In some embodiments, the single chain fusion comprises an HLA-C polypeptide that does not comprise a native transmembrane domain nor a native cytosolic region, and instead comprises a non-native transmembrane domain (e.g., the transmembrane domain of a Type I membrane protein). In some embodiments, the single chain fusion polypeptide includes a linker (e.g., between an exogenous antigenic polypeptide or an exogenous displaceable polypeptide and a β2M polypeptide, between a β2M polypeptide and HLA-C α chain (or fragment thereof), or between a HLA-C α chain (or fragment thereof) and a transmembrane domain). In some embodiments, the linker is selected from a sequence set forth in Table 3. In some embodiments, the single chain fusion polypeptide includes a leader sequence (e.g., a leader sequence set forth in Table 1). In some embodiments, a HLA-C leader sequence is fused to a wild-type or loadable exogenous antigen-presenting polypeptide described herein that comprises a transmembrane domain.

In some embodiments, the wild-type or loadable exogenous antigen-presenting polypeptide comprises an HLA class I polypeptide (or a fragment thereof), and optionally a β2M polypeptide, wherein the HLA class I polypeptide is a HLA-E polypeptide. In some embodiments, the HLA-E polypeptide comprises a HLA-E allele is E*01:01:01:01, E*01:01:01:02, E*01:01:01:03, E*01:01:01:04, E*01:01:01:05, E*01:01:01:06, E*01:01:01:07, E*01:01:01:08, E*01:01:01:09, E*01:01:01:10, E*01:01:02, E*01:03:01:01, E*01:03:01:02, E*01:03:01:03, E*01:03:01:04, E*01:03:02:01, E*01:03:02:02, E*01:03:03, E*01:03:04, E*01:03:05, E*01:04, E*01:05, E*01:06, E*01:07, E*01:08N, E*01:09 or E*01:10 allele. In some embodiments, the HLA-E polypeptide is linked to an exogenous antigenic polypeptide or an exogenous displaceable polypeptide as a single chain fusion polypeptide. In some embodiments, the single chain fusion polypeptide includes a transmembrane domain (e.g.,a transmembrane domain set forth in Table 2). In some embodiments, the single chain fusion polypeptide comprises an HLA-E polypeptide that does not comprise a native transmembrane domain nor a native cytosolic region, and instead comprises a non-native transmembrane domain (e.g., the transmembrane domain of a Type I membrane protein). In some embodiments, the single chain fusion polypeptide includes a linker (e.g., between an exogenous antigenic polypeptide or an exogenous displaceable polypeptide and a β2M polyepeptide, between a β2M polypeptide and a HLA-E α chain (or fragment thereof), or between a HLA-E α chain (or fragment thereof) and a transmembrane domain). In some embodiments, the linker is selected from a sequence set forth in Table 3. In some embodiments, the HLA-E single chain fusion polypeptide includes a leader sequence. In some embodiments, the leader sequence is selected from a sequence set forth in Table 1. In some embodiments, an HLA-E leader sequence is fused to a wild-type or loadable exogenous antigen-presenting polypeptide described herein that comprises a transmembrane domain.

In some embodiments, the wild-type or loadable exogenous antigen-presenting polypeptide comprises an HLA class I polypeptide (or fragment thereof), and optionally a β2M polypeptide, wherein the HLA class I polypeptide is a HLA-G polypeptide. In some embodiments, the HLA-G polypeptide is selected from the group consisting of: HLA-G1, HLA-G2, HLA-G3, HLA-G4, HLA-G5, HLA-G6, and HLA-G7. In some embodiments, the HLA-G is an HLA-G multimer, e.g., a dimer. In some embodiments, the HLA-G polypeptide comprises a HLA-G allele selected from G*01:01:01:01, G*01:01:01:02, G*01:01:01:03, G*01:01:01:04, G*01:01:01:05, G*01:01:01:06, G*01:01:01:07, G*01:01:01:08, G*01:01:02:01, G*01:01:02:02, G*01:01:03:01, G*01:01:03:02, G*01:01:03:03, G*01:01:03:04, G*01:01:04, G*01:01:05, G*01:01:06, G*01:01:07, G*01:01:08, G*01:01:09, G*01:01:11, G*01:01:12, G*01:01:13, G*01:01:14, G*01:01:15, G*01:01:16, G*01:01:17, G*01:01:18, G*01:01:19, G*01:01:20, G*01:01:21, G*01:01:22, G*01:02, G*01:03:01:01, G*01:03:01:02, G*01:04:01:01, G*01:04:01:02, G*01:04:02, G*01:04:03, G*01:04:04, G*01:04:05, G*01:04:06, G*01:05N, G*01:06, G*01:07, G*01:08:01, G*01:08:02, G*01:09, G*01:10 and G*01:11. In some embodiments, the HLA-G polypeptide comprises an unpaired cysteine at residue 42. In some embodiments, the HLA-G polypeptide is linked to an exogenous antigenic polypeptide or an exogenous displaceable polypeptide as a single chain fusion polypeptide. In some embodiments, the single chain fusion polypeptide includes a transmembrane domain (e.g., a transmembrane domain set forth in Table 2). In some embodiments, the single chain fusion comprises an HLA-G polypeptide that does not comprise a native transmembrane domain nor a native cytosolic region, and instead comprises a non-native transmembrane domain (e.g., the transmembrane domain of a Type I membrane protein). In some embodiments, the single chain fusion polypeptide includes a linker (e.g., between an exogenous antigenic polypeptide or an exogenous displaceable polypeptide and a β2M polypeptide, between a β2M polypeptide and a HLA-G α chain (or fragment thereof), or between a HLA-G α chain (or fragment thereof) and a transmembrane domain). In some embodiments, the single chain fusion polypeptide includes a leader sequence (e.g., a leader sequence set forth in Table 1). In some embodiments, an HLA-G leader sequence is fused to a wild-type or loadable exogenous antigen-presenting polypeptide described herein that comprises a transmembrane domain.

In some embodiments, the wild-type or loadable exogenous antigen-presenting polypeptide comprises at least one (e.g., one, two or three) HLA class II polypeptide(s), or a fragment(s) thereof, selected from a HLA-DPα, a HLA-DPβ, a HLA-DMA, HLA-DMB, a HLA DOA, a HLA-DOB, a HLA-DQα, a HLA-DQβ, a HLA-DRα, and a HLA-DRβ polypeptide, or a fragment thereof. In some embodiments, the wild-type or loadable exogenous antigen-presenting polypeptide comprises either: a HLA-DPα polypeptide, or a fragment thereof, and a HLA-DPβ polypeptide, or a fragment thereof, a HLA-DMA polypeptide, or a fragment thereof, and a HLA-DMB polypeptide, or a fragment thereof; a HLA DOA polypeptide, or a fragment thereof, and a HLA-DOB polypeptide, or a fragment thereof; a HLA-DQα polypeptide, or a fragment thereof, and a HLA-DQβ polypeptide, or a fragment thereof; or a HLA-DRα polypeptide, or a fragment thereof, and a HLA-DRβ polypeptide, or a fragment thereof. In some embodiments, the HLA-DPα polypeptide comprises an allele selected from DPA1*01:03, DPA1*02:01, and DPA1*02:07. In some embodiments, the HLA-DPβ polypeptide comprises an allele selected from DPB1*04:01, DPB1*02:01, DPB1*04:02, DPB1*03:01, DPB1*01:01, DPB1*11:01, DPB1*05:01, DPB1*10:01, DPB1*06:01, DPB1*13:01, DPB1*14:01, and DPB1*17:01. In some embodiments, the HLA-DQα polypeptide comprises an allele selected from DQA1*05:01, DQA1*05:03, DQA1*05:05, DQA1*03:01, DQA1*03:02, DQA1*03:03, DQA1*01:02, DQA1*02:01, DQA1*01:01, DQA1*01:03, DQA1*01:04, DQA1*04:01, and DQA1*06:01. In some embodiments, the HLA-DQβ polypeptide comprises an allele selected from DQB1*03:01, DQB1*02:01, DQB1*06:01, DQB1*06:02, DQB1*06:03, DQB1*05:01, DQB1*05:02, DQB1*02:02, DQB1*03:02, DQB1*06:03, DQB1*03:03, DQB1*03:04, DQB1*06:04, DQB1*06:09, DQB1*05:03, DQB1*05:04, and DQB1*04:02. In some embodiments, the HLA-DRβ polypeptide comprises an allele selected from the group consisting of DRB1*07:01, DRB1*03:01, DRB1*15:01, DRB1*04:01, DRB1*01:01, DRB1*13:01, DRB1*11:01, DRB1*04:04, DRB1*13:02, DRB1*08:01, DRB1*12:01, DRB1*11:04, DRB1*09:01, DRB1*14:01, DRB1*04:07, and DRB1*14:04. In some embodiments, the wild-type or loadable exogenous antigen-presenting polypeptide comprises a HLA-DQα polypeptide, or a fragment thereof, and a HLA-DQβ polypeptide, or a fragment thereof, wherein the HLA-DQα polypeptide and the HLA-DQβ polypeptide comprise the following allele combinations, represented as HLA-DQα allele: HLA-DQβ allele: DQA1*05:01:DQB1*2:01; DQA1*2:01:DQB1*2:02; DQA1*03:02:DQB1*2:02; DQA1*3:01:DQB1*4:02; DQA1*03:02:DQB1*4:02; DQA1*4:01:DQB1*4:02; DQA1*1:01:DQB1*5:01; DQA1*1:02:DQB1*5:01; DQA1*1:03:DQB1*5:01; DQA1*1:04:DQB1*5:01; DQA1*1:02:DQB1*5:02; DQA1*1:03:DQB1*5:02; DQA1*1:04:DQB1*5:03; DQA1*1:02:DQB1*5:04; DQA1*1:03:DQB1*6:01; DQA1*1:02:DQB1*6:02; DQA1*1:03:DQB1*6:02; DQA1*1:04:DQB1*6:02; DQA1*1:02:DQB1*6:03; DQA1*1:03:DQB1*6:03; DQA1*1:02:DQB1*6:04; DQA1*1:02:DQB1*6:09; DQA1*2:01:DQB1*3:01; DQA1*3:01:DQB1*3:01; DQA1*03:03:DQB1*3:01; DQA1*3:01:DQB1*3:04; DQA1*03:02:DQB1*3:04; DQA1*4:01:DQB1*3:01; DQA1*05:05:DQB1*3:01; DQA1*6:01:DQB1*3:01; DQA1*3:01:DQB1*3:02; DQA1*03:02:DQB1*3:02; DQA1*2:01:DQB1*3:03; DQA1*03:01:DQB1*03:02; DQA1*03:02:DQB1*03:02; DQA1*04:01:DQB1*03:02; DQA1*05:03:DQB1*03:02 and DQA1*3:02:DQB1*3:03. In some embodiments, the HLA class II polypeptide(s) is linked to an exogenous antigenic polypeptide or an exogenous displaceable polypeptide as a single chain fusion polypeptide. In some embodiments, the single chain fusion polypeptide comprises HLA class II polypeptides that do not comprise a native transmembrane domain nor a native cytosolic domain, and instead the single chain fusion polypeptide comprises a non-native transmembrane domain (e.g., the transmembrane domain of a Type I membrane protein). In some embodiments, the single chain fusion polypeptide includes a linker (e.g., between an exogenous antigenic polypeptide or an exogenous displaceable polypeptide and a HLA class II polypeptide (or fragment thereof), between a first HLA class II polypeptide (or fragment thereof) and a second HLA class II polypeptide (or fragment thereof), or between a HLA class II polypeptide and a transmembrane domain). In some embodiments, the linker is a linker sequence set forth in Table 3. In some embodiments, the single chain fusion polypeptide includes a leader sequence (e.g., a leader sequence set forth in Table 1). In some embodiments, an HLA class II polypeptide leader sequence is fused to a wild-type or loadable exogenous antigen-presenting polypeptide described herein that comprises a transmembrane domain.

In some embodiments, the wild-type or loadable exogenous antigen-presenting polypeptide comprises an HLA class II alpha chain (or fragment thereof) and an HLA class II beta chain (or fragment thereof), each of which lack a native transmembrane domain and/or a native cytosolic region, and instead the wild-type or loadable exogenous antigen-presenting polypeptide comprises a non-native transmembrane domain (e.g., the transmembrane domain of a Type I membrane protein. In some embodiments, the transmembrane domain is selected from a sequence set forth in Table 2. In some embodiments, the wild-type or loadable exogenous antigen-presenting polypeptide comprises includes a linker (e.g., a linker sequence set forth in Table 3. In some embodiments, the wild-type or loadable exogenous antigen-presenting polypeptide comprisesinclude a leader sequence (e.g. a leader sequence set forth in Table 1).

In some embodiments, a wild-type or loadable exogenous antigen-presenting polypeptide comprises an HLA allele polypeptide comprising or consisting of an amino acid sequence set forth in Table 4. It will be readily understood by those of skill in the art that loadable exogenous antigen-presenting polypeptides can include an amino acid sequence set forth Table 4 modified to include one or more amino acid substitutions set forth herein. In some embodiments, the HLA allele polypeptide comprises a signal peptide. In other embodiments, the HLA allele polypeptide does not include a signal peptide. Accordingly, in some embodiments, the wild-type or loadable exogenous antigen-presenting polypeptide comprises the amino acid sequence of any one of the sequences, shown in Table 4, excluding the signal peptide amino acid sequence (shown underlined in the sequences in Table 4). In other embodiments, the wild-type or loadable exogenous antigen-presenting polypeptide comprises the amino acid sequence of any one of the sequences shown in Table 4 including the signal peptide amino acid sequence (shown underlined in Table 4).

TABLE 4
HLA Alleles

| | | |
|---|---|---|
| (SEQ ID | A*01: 01 | MAVMAPRTLLLLLSGALALTQTWAGSHSMRYFF |
| NO: 19) | (IMGT/HLA | TSVSRPGRGEPRFIAVGYVDDTQFVRFDSDAASQ |
| | Accession No. | KMEPRAPWIEQEGPEYWDQETRNMKAHSQTDR |
| | HLA00001) | ANLGTLRGYYNQSEDGSHTIQIMYGCDVGPDGR |
| | * Predicted signal | FLRGYRQDAYDGKDYIALNEDLRSWTAADMAA |
| | peptide underlined | QITKRKWEAVHAAEQRRVYLEGRCVDGLRRYL |
| | | ENGKETLQRTDPPKTHMTHHPISDHEATLRCWA |
| | | LGFYPAEITLTWQRDGEDQTQDTELVETRPAGD |
| | | GTFQKWAAVVVPSGEEQRYTCHVQHEGLPKPLT |
| | | LRWELSSQPTIPIVGIIAGLVLLGAVITGAVVAAV |
| | | MWRRKSSDRKGGSYTQAASSDSAQGSDVSLTAC |
| | | KV |
| (SEQ ID | A*02: 01 | MAVMAPRTLVLLLSGALALTQTWAGSHSMRYF |
| NO: 20) | (IMGT/HLA | FTSVSRPGRGEPRFIAVGYVDDTQFVRFDSDAAS |
| | Accession No. | QRMEPRAPWIEQEGPEYWDGETRKVKAHSQTH |
| | HLA00005) | RVDLGTLRGYYNQSEAGSHTVQRMYGCDVGSD |
| | * Predicted signal | WRFLRGYHQYAYDGKDYIALKEDLRSWTAADM |
| | peptide underlined | AAQTTKHKWEAAHVAEQLRAYLEGTCVEWLRR |
| | | YLENGKETLQRTDAPKTHMTHHAVSDHEATLRC |
| | | WALSFYPAEITLTWQRDGEDQTQDTELVETRPA |
| | | GDGTFQKWAAVVVPSGQEQRYTCHVQHEGLPK |
| | | PLTLRWEPSSQPTIPIVGIIAGLVLFGAVITGAVVA |
| | | AVMWRRKSSDRKGGSYSQAASSDSAQGSDVSLT |
| | | ACKV |
| (SEQ ID | A*03: 01 | MAVMAPRTLLLLLSGALALTQTWAGSHSMRYFF |
| NO: 21) | (IMGT/HLA | TSVSRPGRGEPRFIAVGYVDDTQFVRFDSDAASQ |
| | Accession No. | RMEPRAPWIEQEGPEYWDQETRNVKAQSQTDR |
| | HLA00037) | VDLGTLRGYYNQSEAGSHTIQIMYGCDVGSDGR |
| | * Predicted signal | FLRGYRQDAYDGKDYIALNEDLRSWTAADMAA |
| | peptide underlined | QITKRKWEAAHEAEQLRAYLDGTCVEWLRRYL |
| | | ENGKETLQRTDPPKTHMTHHPISDHEATLRCWA |
| | | LGFYPAEITLTWQRDGEDQTQDTELVETRPAGD |
| | | GTFQKWAAVVVPSGEEQRYTCHVQHEGLPKPLT |
| | | LRWELSSQPTIPIVGIIAGLVLLGAVITGAVVAAV |
| | | MWRRKSSDRKGGSYTQAASSDSAQGSDVSLTAC |
| | | KV |
| (SEQ ID | A*24: 02 | MAVMAPRTLVLLLSGALALTQTWAGSHSMRYF |
| NO: 22) | (IMGT/HLA | STSVSRPGRGEPRFIAVGYVDDTQFVRFDSDAAS |
| | Accession No. | QRMEPRAPWIEQEGPEYWDEETGKVKAHSQTDR |
| | HLA00050) | ENLRIALRYYNQSEAGSHTLQMMFGCDVGSDGR |
| | * Predicted signal | FLRGYHQYAYDGKDYIALKEDLRSWTAADMAA |
| | peptide underlined | QITKRKWEAAHVAEQQRAYLEGTCVDGLRRYL |
| | | ENGKETLQRTDPPKTHMTHHPISDHEATLRCWA |
| | | LGFYPAEITLTWQRDGEDQTQDTELVETRPAGD |
| | | GTFQKWAAVVVPSGEEQRYTCHVQHEGLPKPLT |
| | | LRWEPSSQPTVPIVGIIAGLVLLGAVITGAVVAAV |
| | | MWRRNSSDRKGGSYSQAASSDSAQGSDVSLTAC |
| | | KV |
| (SEQ ID | A*11: 01 | MAVMAPRTLLLLLSGALALTQTWAGSHSMRYF |
| NO: 23) | (IMGT/HLA | YTSVSRPGRGEPRFIAVGYVDDTQFVRFDSDAAS |
| | Accession No. | QRMEPRAPWIEQEGPEYWDQETRNVKAQSQTD |
| | HLA00043) | RVDLGTLRGYYNQSEDGSHTIQIMYGCDVGPDG |
| | * Predicted signal | RFLRGYRQDAYDGKDYIALNEDLRSWTAADMA |
| | peptide underlined | AQITKRKWEAAHAAEQQRAYLEGRCVEWLRRY |
| | | LENGKETLQRTDPPKTHMTHHPISDHEATLRCW |
| | | ALGFYPAEITLTWQRDGEDQTQDTELVETRPAG |
| | | DGTFQKWAAVVVPSGEEQRYTCHVQHEGLPKPL |
| | | TLRWELSSQPTIPIVGIIAGLVLLGAVITGAVVAA |
| | | VMWRRKSSDRKGGSYTQAASSDSAQGSDVSLT |
| | | ACKV |
| (SEQ ID | A*29: 02 | MAVMAPRTLLLLLLGALALTQTWAGSHSMRYF |
| NO: 24) | (IMGT/HLA | TTSVSRPGRGEPRFIAVGYVDDTQFVRFDSDAAS |
| | Accession No. | QRMEPRAPWIEQEGPEYWDLQTRNVKAQSQTD |
| | HLA00086) | RANLGTLRGYYNQSEAGSHTIQMMYGCDVGSD |
| | * Predicted signal | GRFLRGYRQDAYDGKDYIALNEDLRSWTAADM |
| | peptide underlined | AAQITQRKWEAARVAEQLRAYLEGTCVEWLRR |
| | | YLENGKETLQRTDAPKTHMTHHAVSDHEATLRC |
| | | WALSFYPAEITLTWQRDGEDQTQDTELVETRPA |
| | | GDGTFQKWASVVVPSGQEQRYTCHVQHEGLPK |
| | | PLTLRWEPSSQPTIPIVGIIAGLVLFGAVFAGAVV |
| | | AAVRWRRKSSDRKGGSYSQAASSDSAQGSDMS |
| | | LTACKV |
| (SEQ ID | A*32: 01 | MAVMAPRTLLLLLLGALALTQTWAGSHSMRYF |
| NO: 25) | (IMGT/HLA | FTSVSRPGRGEPRFIAVGYVDDTQFVRFDSDAAS |
| | Accession No. | QRMEPRAPWIEQEGPEYWDQETRNVKAHSQTD |
| | HLA00101) | RESLRIALRYYNQSEAGSHTIQMMYGCDVGPDG |
| | * Predicted signal | RLLRGYQQDAYDGKDYIALNEDLRSWTAADMA |
| | peptide underlined | AQITQRKWEAARVAEQLRAYLEGTCVEWLRRY |
| | | LENGKETLQRTDAPKTHMTHHAVSDHEATLRC |
| | | WALSFYPAEITLTWQRDGEDQTQDTELVETRPA |
| | | GDGTFQKWASVVVPSGQEQRYTCHVQHEGLPK |
| | | PLTLRWEPSSQPTIPIVGIIAGLVLFGAMFAGAVV |
| | | AAVRWRRKSSDRKGGSYSQAASSDSAQGSDMS |
| | | LTACKV |
| (SEQ ID | A*68: 01 | MAVMAPRTLVLLLSGALALTQTWAGSHSMRYF |
| NO: 26) | (IMGT/HLA | YTSVSRPGRGEPRFIAVGYVDDTQFVRFDSDAAS |
| | Accession No. | QRMEPRAPWIEQEGPEYWDRNTRNVKAQSQTD |
| | HLA00115) | RVDLGTLRGYYNQSEAGSHTIQMMYGCDVGSD |
| | * Predicted signal | GRFLRGYRQDAYDGKDYIALKEDLRSWTAADM |
| | peptide underlined | AAQTTKHKWEAAHVAEQWRAYLEGTCVEWLR |
| | | RYLENGKETLQRTDAPKTHMTHHAVSDHEATLR |
| | | CWALSFYPAEITLTWQRDGEDQTQDTELVETRP |
| | | AGDGTFQKWVAVVVPSGQEQRYTCHVQHEGLP |
| | | KPLTLRWEPSSQPTIPIVGIIAGLVLFGAVITGAVV |
| | | AAVMWRRKSSDRKGGSYSQAASSDSAQGSDVS |
| | | LTACKV |
| (SEQ ID | A*31: 01 | MAVMAPRTLLLLLLGALALTQTWAGSHSMRYF |
| NO: 30) | (IMGT/HLA | TTSVSRPGRGEPRFIAVGYVDDTQFVRFDSDAAS |
| | Accession No. | QRMEPRAPWIEQERPEYWDQETRNVKAHSQIDR |
| | HLA00092) | VDLGTLRGYYNQSEAGSHTIQMMYGCDVGSDG |
| | * Predicted signal | RFLRGYQQDAYDGKDYIALNEDLRSWTAADMA |
| | peptide underlined | AQITQRKWEAARVAEQLRAYLEGTCVEWLRRY |
| | | LENGKETLQRTDPPKTHMTHHAVSDHEATLRCW |
| | | ALSFYPAEITLTWQRDGEDQTQDTELVETRPAGD |
| | | GTFQKWASVVVPSGQEQRYTCHVQHEGLPKPLT |
| | | LRWEPSSQPTIPIVGIIAGLVLFGAVFAGAVVAAV |
| | | RWRRKSSDRKGGSYSQAASSDSAQGSDMSLTAC |
| | | KV |
| (SEQ ID | A*25: 01 | MAVMAPRTLVLLLSGALALTQTWAGSHSMRYF |
| NO: 46) | (IMGT/HLA | YTSVSRPGRGEPRFIAVGYVDDTQFVRFDSDAAS |
| | Accession No. | QRMEPRAPWIEQEGPEYWDRNTRNVKAHSQTD |
| | HLA00071) | RESLRIALRYYNQSEDGSHTIQRMYGCDVGPDG |
| | * Predicted signal | RFLRGYQQDAYDGKDYIALNEDLRSWTAADMA |
| | peptide underlined | AQITQRKWETAHEAEQWRAYLEGRCVEWLRRY |
| | | LENGKETLQRTDAPKTHMTHHAVSDHEATLRC |
| | | WALSFYPAEITLTWQRDGEDQTQDTELVETRPA |
| | | GDGTFQKWASVVVPSGQEQRYTCHVQHEGLPK |
| | | PLTLRWEPSSQPTIPIVGIIAGLVLFGAVIAGAVVA |
| | | AVMWRRKSSDRKGGSYSQAASSDSAQGSDMSL |
| | | TACKV |
| (SEQ ID | A*26: 01 | MAVMAPRTLVLLLSGALALTQTWAGSHSMRYF |
| NO: 47) | (IMGT/HLA | YTSVSRPGRGEPRFIAVGYVDDTQFVRFDSDAAS |
| | Accession No. | QRMEPRAPWIEQEGPEYWDRNTRNVKAHSQTD |
| | HLA00073) | RANLGTLRGYYNQSEDGSHTIQRMYGCDVGPDG |
| | * Predicted signal | RFLRGYQQDAYDGKDYIALNEDLRSWTAADMA |
| | peptide underlined | AQITQRKWETAHEAEQWRAYLEGRCVEWLRRY |
| | | LENGKETLQRTDAPKTHMTHHAVSDHEATLRC |
| | | WALSFYPAEITLTWQRDGEDQTQDTELVETRPA |
| | | GDGTFQKWASVVVPSGQEQRYTCHVQHEGLPK |
| | | PLTLRWEPSSQPTIPIVGIIAGLVLFGAVIAGAVVA |
| | | AVMWRRKSSDRKGGSYSQAASSDSAQGSDMSL |
| | | TACKV |
| (SEQ ID | A*23: 01 | MAVMAPRTLVLLLSGALALTQTWAGSHSMRYF |
| NO: 48) | (IMGT/HLA | STSVSRPGRGEPRFIAVGYVDDTQFVRFDSDAAS |
| | Accession No. | QRMEPRAPWIEQEGPEYWDEETGKVKAHSQTDR |
| | HLA00048) | ENLRIALRYYNQSEAGSHTLQMMFGCDVGSDGR |
| | * Predicted signal | FLRGYHQYAYDGKDYIALKEDLRSWTAADMAA |
| | peptide underlined | QITQRKWEAARVAEQLRAYLEGTCVDGLRRYLE |
| | | NGKETLQRTDPPKTHMTHHPISDHEATLRCWAL |
| | | GFYPAEITLTWQRDGEDQTQDTELVETRPAGDG |
| | | TFQKWAAVVVPSGEEQRYTCHVQHEGLPKPLTL |
| | | RWEPSSQPTVHIVGIIAGLVLLGAVITGAVVAAV |
| | | MWRRNSSDRKGGSYSQAASSDSAQGSDVSLTAC |
| | | KV |
| (SEQ ID | A*30: 01 | MAVMAPRTLLLLLSGALALTQTWAGSHSMRYFS |
| NO: 49) | (IMGT/HLA | TSVSRPGSGEPRFIAVGYVDDTQFVRFDSDAASQ |
| | Accession No. | RMEPRAPWIEQERPEYWDQETRNVKAQSQTDRV |
| | HLA00089) | DLGTLRGYYNQSEAGSHTIQIMYGCDVGSDGRF |
| | * Predicted signal | LRGYEQHAYDGKDYIALNEDLRSWTAADMAAQ |
| | peptide underlined | ITQRKWEAARWAEQLRAYLEGTCVEWLRRYLE |
| | | NGKETLQRTDPPKTHMTHHPISDHEATLRCWAL |
| | | GFYPAEITLTWQRDGEDQTQDTELVETRPAGDG |
| | | TFQKWAAVVVPSGEEQRYTCHVQHEGLPKPLTL |
| | | RWELSSQPTIPIVGIIAGLVLLGAVITGAVVAAVM |
| | | WRRKSSDRKGGSYTQAASSDSAQGSDVSLTACK |
| | | V |
| (SEQ ID | C*07: 01 | MRVMAPRALLLLLSGGLALTETWACSHSMRYF |
| NO: 50) | (IMGT/HLA | DTAVSRPGRGEPRFISVGYVDDTQFVRFDSDAAS |
| | Accession No. | PRGEPRAPWVEQEGPEYWDRETQNYKRQAQAD |
| | HLA00433) | RVSLRNLRGYYNQSEDGSHTLQRMYGCDLGPD |
| | * Predicted signal | GRLLRGYDQSAYDGKDYIALNEDLRSWTAADT |
| | peptide underlined | AAQITQRKLEAARAAEQLRAYLEGTCVEWLRRY |
| | | LENGKETLQRAEPPKTHVTHHPLSDHEATLRCW |
| | | ALGFYPAEITLTWQRDGEDQTQDTELVETRPAG |
| | | DGTFQKWAAVVVPSGQEQRYTCHMQHEGLQEP |
| | | LTLSWEPSSQPTIPIMGIVAGLAVLVVLAVLGAV |
| | | VTAMMCRRKSSGGKGGSCSQAACSNSAQGSDES |
| | | LITCKA |
| (SEQ ID | C*07: 02 | MRVMAPRALLLLLSGGLALTETWACSHSMRYF |
| NO: 51) | (IMGT/HLA | DTAVSRPGRGEPRFISVGYVDDTQFVRFDSDAAS |
| | Accession No. | PRGEPRAPWVEQEGPEYWDRETQKYKRQAQAD |
| | HLA00434) | RVSLRNLRGYYNQSEDGSHTLQRMSGCDLGPDG |
| | * Predicted signal | RLLRGYDQSAYDGKDYIALNEDLRSWTAADTA |
| | peptide underlined | AQITQRKLEAARAAEQLRAYLEGTCVEWLRRYL |
| | | ENGKETLQRAEPPKTHVTHHPLSDHEATLRCWA |
| | | LGFYPAEITLTWQRDGEDQTQDTELVETRPAGD |
| | | GTFQKWAAVVVPSGQEQRYTCHMQHEGLQEPL |
| | | TLSWEPSSQPTIPIMGIVAGLAVLVVLAVLGAVV |
| | | TAMMCRRKSSGGKGGSCSQAACSNSAQGSDESL |
| | | ITCKA |
| (SEQ ID | C*05: 01 | MRVMAPRTLILLLSGALALTETWACSHSMRYFY |
| NO: 52) | (IMGT/HLA | TAVSRPGRGEPRFIAVGYVDDTQFVQFDSDAASP |
| | Accession No. | RGEPRAPWVEQEGPEYWDRETQKYKRQAQTDR |
| | HLA00427) | VNLRKLRGYYNQSEAGSHTLQRMYGCDLGPDG |
| | * Predicted signal | RLLRGYNQFAYDGKDYIALNEDLRSWTAADKA |
| | peptide underlined | AQITQRKWEAAREAEQRRAYLEGTCVEWLRRY |
| | | LENGKKTLQRAEHPKTHVTHHPVSDHEATLRCW |
| | | ALGFYPAEITLTWQRDGEDQTQDTELVETRPAG |
| | | DGTFQKWAAVVVPSGEEQRYTCHVQHEGLPEPL |
| | | TLRWGPSSQPTIPIVGIVAGLAVLAVLAVLGAVM |
| | | AVVMCRRKSSGGKGGSCSQAASSNSAQGSDESL |
| | | IACKA |
| (SEQ ID | C*06: 02 | MRVMAPRTLILLLSGALALTETWACSHSMRYFD |
| NO: 53) | (IMGT/HLA | TAVSRPGRGEPRFISVGYVDDTQFVRFDSDAASP |
| | Accession No. | RGEPRAPWVEQEGPEYWDRETQKYKRQAQADR |
| | HLA00430) | VNLRKLRGYYNQSEDGSHTLQWMYGCDLGPDG |
| | * Predicted signal | RLLRGYDQSAYDGKDYIALNEDLRSWTAADTA |
| | peptide underlined | AQITQRKWEAAREAEQWRAYLEGTCVEWLRRY |
| | | LENGKETLQRAEHPKTHVTHHPVSDHEATLRCW |
| | | ALGFYPAEITLTWQRDGEDQTQDTELVETRPAG |
| | | DGTFQKWAAVVVPSGEEQRYTCHVQHEGLPEPL |
| | | TLRWEPSSQPTIPIVGIVAGLAVLAVLAVLGAVM |
| | | AVVMCRRKSSGGKGGSCSQAASSNSAQGSDESL |
| | | IACKA |
| (SEQ ID | C*04: 01 | MRVMAPRTLILLLSGALALTETWAGSHSMRYFS |
| NO: 54) | (IMGT/HLA | TSVSWPGRGEPRFIAVGYVDDTQFVRFDSDAASP |
| | Accession No. | RGEPREPWVEQEGPEYWDRETQKYKRQAQADR |
| | HLA00420) | VNLRKLRGYYNQSEDGSHTLQRMFGCDLGPDG |
| | * Predicted signal | RLLRGYNQFAYDGKDYIALNEDLRSWTAADTA |
| | peptide underlined | AQITQRKWEAAREAEQRRAYLEGTCVEWLRRY |
| | | LENGKETLQRAEHPKTHVTHHPVSDHEATLRCW |
| | | ALGFYPAEITLTWQWDGEDQTQDTELVETRPAG |
| | | DGTFQKWAAVVVPSGEEQRYTCHVQHEGLPEPL |
| | | TLRWKPSSQPTIPIVGIVAGLAVLAVLAVLGAMV |
| | | AVVMCRRKSSGGKGGSCSQAASSNSAQGSDESL |
| | | IACKA |
| (SEQ ID | C*03: 04 | MRVMAPRTLILLLSGALALTETWAGSHSMRYFY |
| NO: 55) | (IMGT/HLA | TAVSRPGRGEPHFIAVGYVDDTQFVRFDSDAASP |
| | Accession No. | RGEPRAPWVEQEGPEYWDRETQKYKRQAQTDR |
| | HLA00413) | VSLRNLRGYYNQSEAGSHIIQRMYGCDVGPDGR |
| | * Predicted signal | LLRGYDQYAYDGKDYIALNEDLRSWTAADTAA |
| | peptide underlined | QITQRKWEAAREAEQLRAYLEGLCVEWLRRYLK |
| | | NGKETLQRAEHPKTHVTHHPVSDHEATLRCWAL |
| | | GFYPAEITLTWQWDGEDQTQDTELVETRPAGDG |
| | | TFQKWAAVVVPSGEEQRYTCHVQHEGLPEPLTL |
| | | RWEPSSQPTIPIVGIVAGLAVLAVLAVLGAVVAV |
| | | VMCRRKSSGGKGGSCSQAASSNSAQGSDESLIAC |
| | | KA |
| (SEQ ID | C*03: 03 | MRVMAPRTLILLLSGALALTETWAGSHSMRYFY |
| NO: 56) | (IMGT/HLA | TAVSRPGRGEPHFIAVGYVDDTQFVRFDSDAASP |
| | Accession No. | RGEPRAPWVEQEGPEYWDRETQKYKRQAQTDR |
| | HLA00411) | VSLRNLRGYYNQSEARSHIIQRMYGCDVGPDGR |
| | * Predicted signal | LLRGYDQYAYDGKDYIALNEDLRSWTAADTAA |
| | peptide underlined | QITQRKWEAAREAEQLRAYLEGLCVEWLRRYLK |
| | | NGKETLQRAEHPKTHVTHHPVSDHEATLRCWAL |
| | | GFYPAEITLTWQWDGEDQTQDTELVETRPAGDG |
| | | TFQKWAAVVVPSGEEQRYTCHVQHEGLPEPLTL |
| | | RWEPSSQPTIPIVGIVAGLAVLAVLAVLGAVVAV |
| | | VMCRRKSSGGKGGSCSQAASSNSAQGSDESLIAC |
| | | KA |
| (SEQ ID | C*02: 02 | MRVMAPRTLLLLLSGALALTETWACSHSMRYFY |
| NO: 57) | (IMGT/HLA | TAVSRPSRGEPHFIAVGYVDDTQFVRFDSDAASP |
| | Accession No. | RGEPRAPWVEQEGPEYWDRETQKYKRQAQTDR |
| | HLA00404) | VNLRKLRGYYNQSEAGSHTLQRMYGCDLGPDG |
| | * Predicted signal | RLLRGYDQSAYDGKDYIALNEDLRSWTAADTA |
| | peptide underlined | AQITQRKWEAAREAEQWRAYLEGECVEWLRRY |
| | | LENGKETLQRAEHPKTHVTHHPVSDHEATLRCW |
| | | ALGFYPTEITLTWQRDGEDQTQDTELVETRPAGD |
| | | GTFQKWAAVVVPSGEEQRYTCHVQHEGLPEPLT |
| | | LRWEPSSQPTIPIVGIVAGLAVLAVLAVLGAVVA |
| | | VVMCRRKSSGGKGGSCSQAASSNSAQGSDESLI |
| | | ACKA |
| (SEQ ID | C*16: 01 | MRVMAPRTLILLLSGALALTETWACSHSMRYFY |
| NO: 58) | (IMGT/HLA | TAVSRPGRGEPRFIAVGYVDDTQFVRFDSDAASP |
| | Accession No. | RGEPRAPWVEQEGPEYWDRETQKYKRQAQTDR |
| | HLA00475) | VSLRNLRGYYNQSEAGSHTLQWMYGCDLGPDG |
| | * Predicted signal | RLLRGYDQSAYDGKDYIALNEDLRSWTAADTA |
| | peptide underlined | AQITQRKWEAARAAEQQRAYLEGTCVEWLRRY |
| | | LENGKETLQRAEHPKTHVTHHLVSDHEATLRCW |
| | | ALGFYPAEITLTWQRDGEDQTQDTELVETRPAG |
| | | DGTFQKWAAVVVPSGEEQRYTCHVQHEGLPEPL |
| | | TLRWEPSSQPTIPIVGIVAGLAVLAVLAVLGAVV |
| | | AVVMCRRKSSGGKGGSCSQAASSNSAQGSDESL |
| | | IACKA |
| (SEQ ID | C*08: 02 | MRVMAPRTLILLLSGALALTETWACSHSMRYFY |
| NO: 59) | (IMGT/HLA | TAVSRPGRGEPRFIAVGYVDDTQFVQFDSDAASP |
| | Accession No. | RGEPRAPWVEQEGPEYWDRETQKYKRQAQTDR |
| | HLA00446) | VSLRNLRGYYNQSEAGSHTLQRMYGCDLGPDG |
| | * Predicted signal | RLLRGYNQFAYDGKDYIALNEDLRSWTAADKA |
| | peptide underlined | AQITQRKWEAAREAEQRRAYLEGTCVEWLRRY |
| | | LENGKKTLQRAEHPKTHVTHHPVSDHEATLRCW |
| | | ALGFYPAEITLTWQRDGEDQTQDTELVETRPAG |
| | | DGTFQKWAAVVVPSGEEQRYTCHVQHEGLPEPL |
| | | TLRWGPSSQPTIPIVGIVAGLAVLAVLAVLGAVM |
| | | AVVMCRRKSSGGKGGSCSQAASSNSAQGSDESL |
| | | IACKA |
| (SEQ ID | C*12: 03 | MRVMAPRTLILLLSGALALTETWACSHSMRYFY |
| NO: 60) | (IMGT/HLA | TAVSRPGRGEPRFIAVGYVDDTQFVRFDSDAASP |
| | Accession No. | RGEPRAPWVEQEGPEYWDRETQKYKRQAQADR |
| | HLA00455) | VSLRNLRGYYNQSEAGSHTLQWMYGCDLGPDG |
| | * Predicted signal | RLLRGYDQSAYDGKDYIALNEDLRSWTAADTA |
| | peptide underlined | AQITQRKWEAAREAEQWRAYLEGTCVEWLRRY |
| | | LENGKETLQRAEHPKTHVTHHPVSDHEATLRCW |
| | | ALGFYPAEITLTWQRDGEDQTQDTELVETRPAG |
| | | DGTFQKWAAVVVPSGEEQRYTCHVQHEGLPEPL |
| | | TLRWEPSSQPTIPIVGIVAGLAVLAVLAVLGAVM |
| | | AVVMCRRKSSGGKGGSCSQAASSNSAQGSDESL |
| | | IACKA |
| (SEQ ID | C*01: 02 | MRVMAPRTLILLLSGALALTETWACSHSMKYFF |
| NO: 61) | (IMGT/HLA | TSVSRPGRGEPRFISVGYVDDTQFVRFDSDAASP |
| | Accession No. | RGEPRAPWVEQEGPEYWDRETQKYKRQAQTDR |
| | HLA00401) | VSLRNLRGYYNQSEAGSHTLQWMCGCDLGPDG |
| | * Predicted signal | RLLRGYDQYAYDGKDYIALNEDLRSWTAADTA |
| | peptide underlined | AQITQRKWEAAREAEQRRAYLEGTCVEWLRRY |
| | | LENGKETLQRAEHPKTHVTHHPVSDHEATLRCW |
| | | ALGFYPAEITLTWQWDGEDQTQDTELVETRPAG |
| | | DGTFQKWAAVMVPSGEEQRYTCHVQHEGLPEP |
| | | LTLRWEPSSQPTIPIVGIVAGLAVLAVLAVLGAV |
| | | VAVVMCRRKSSGGKGGSCSQAASSNSAQGSDES |
| | | LIACKA |
| (SEQ ID | C*15: 02 | MRVMAPRTLLLLLSGALALTETWACSHSMRYFY |
| NO: 62) | (IMGT/HLA | TAVSRPGRGEPHFIAVGYVDDTQFVRFDSDAASP |
| | Accession No. | RGEPRAPWVEQEGPEYWDRETQNYKRQAQTDR |
| | HLA00467) | VNLRKLRGYYNQSEAGSHIIQRMYGCDLGPDGR |
| | * Predicted signal | LLRGHDQLAYDGKDYIALNEDLRSWTAADTAA |
| | peptide underlined | QITQRKWEAAREAEQLRAYLEGTCVEWLRRYLE |
| | | NGKETLQRAEHPKTHVTHHPVSDHEATLRCWAL |
| | | GFYPAEITLTWQRDGEDQTQDTELVETRPAGDG |
| | | TFQKWAAVVVPSGEEQRYTCHVQHEGLPEPLTL |
| | | RWEPSSQPTIPIVGIVAGLAVLAVLAVLGAVMAV |
| | | VMCRRKSSGGKGGSCSQAASSNSAQGSDESLIAC |
| | | KA |
| (SEQ ID | C*07: 04 | MRVMAPRALLLLLSGGLALTETWACSHSMRYF |
| NO: 63) | (IMGT/HLA | DTAVSRPGRGEPRFISVGYVDDTQFVRFDSDAAS |
| | Accession No. | PRGEPRAPWVEQEGPEYWDRETQKYKRQAQAD |
| | HLA00406) | RVSLRNLRGYYNQSEDGSHTFQRMYGCDLGPDG |
| | * Predicted signal | RLLRGYDQFAYDGKDYIALNEDLRSWTAADTA |
| | peptide underlined | AQITQRKLEAARAAEQDRAYLEGTCVEWLRRYL |
| | | ENGKKTLQRAEPPKTHVTHHPLSDHEATLRCWA |
| | | LGFYPAEITLTWQRDGEDQTQDTELVETRPAGD |
| | | GTFQKWAAVVVPSGQEQRYTCHMQHEGLQEPL |
| | | TLSWEPSSQPTIPIMGIVAGLAVLVVLAVLGAVV |
| | | TAMMCRRKSSGGKGGSCSQAACSNSAQGSDESL |
| | | ITCKA |
| (SEQ ID | C*14: 02 | MRVMAPRTLILLLSGALALTETWACSHSMRYFS |
| NO: 64) | (IMGT/HLA | TSVSRPGRGEPRFIAVGYVDDTQFVRFDSDAASP |
| | Accession No. | RGEPRAPWVEQEGPEYWDRETQKYKRQAQTDR |
| | HLA00462) | VSLRNLRGYYNQSEAGSHTLQWMFGCDLGPDG |
| | * Predicted signal | RLLRGYDQSAYDGKDYIALNEDLRSWTAADTA |
| | peptide underlined | AQITQRKWEAAREAEQRRAYLEGTCVEWLRRY |
| | | LENGKETLQRAEHPKTHVTHHPVSDHEATLRCW |
| | | ALGFYPAEITLTWQWDGEDQTQDTELVETRPAG |
| | | DGTFQKWAAVVVPSGEEQRYTCHVQHEGLPEPL |
| | | TLRWEPSSQPTIPIVGIVAGLAVLAVLAVLGAVV |
| | | AVVMCRRKSSGGKGGSCSQAASSNSAQGSDESL |
| | | IACKA |
| (SEQ ID | B*08: 01 | MLVMAPRTVLLLLSAALALTETWAGSHSMRYF |
| NO: 65) | (IMGT/HLA | DTAMSRPGRGEPRFISVGYVDDTQFVRFDSDAAS |
| | Accession No. | PREEPRAPWIEQEGPEYWDRNTQIFKTNTQTDRE |
| | HLA00146) | SLRNLRGYYNQSEAGSHTLQSMYGCDVGPDGRL |
| | * Predicted signal | LRGHNQYAYDGKDYIALNEDLRSWTAADTAAQI |
| | peptide underlined | TQRKWEAARVAEQDRAYLEGTCVEWLRRYLEN |
| | | GKDTLERADPPKTHVTHHPISDHEATLRCWALG |
| | | FYPAEITLTWQRDGEDQTQDTELVETRPAGDRTF |
| | | QKWAAVVVPSGEEQRYTCHVQHEGLPKPLTLR |
| | | WEPSSQSTVPIVGIVAGLAVLAVVVIGAVVAAV |
| | | MCRRKSSGGKGGSYSQAACSDSAQGSDVSLTA |
| (SEQ ID | B*07: 02 | MLVMAPRTVLLLLSAALALTETWAGSHSMRYF |
| NO: 66) | (IMGT/HLA | YTSVSRPGRGEPRFISVGYVDDTQFVRFDSDAAS |
| | Accession No. | PREEPRAPWIEQEGPEYWDRNTQIYKAQAQTDR |
| | HLA00132) | ESLRNLRGYYNQSEAGSHTLQSMYGCDVGPDGR |
| | * Predicted signal | LLRGHDQYAYDGKDYIALNEDLRSWTAADTAA |
| | peptide underlined | QITQRKWEAAREAEQRRAYLEGECVEWLRRYLE |
| | | NGKDKLERADPPKTHVTHHPISDHEATLRCWAL |
| | | GFYPAEITLTWQRDGEDQTQDTELVETRPAGDR |
| | | TFQKWAAVVVPSGEEQRYTCHVQHEGLPKPLTL |
| | | RWEPSSQSTVPIVGIVAGLAVLAVVVIGAVVAAV |
| | | MCRRKSSGGKGGSYSQAACSDSAQGSDVSLTA |
| (SEQ ID | B*44: 02 | MRVTAPRTLLLLLWGAVALTETWAGSHSMRYF |
| NO: 67) | (IMGT/HLA | YTAMSRPGRGEPRFITVGYVDDTLFVRFDSDATS |
| | Accession No. | PRKEPRAPWIEQEGPEYWDRETQISKTNTQTYRE |
| | HLA00318) | NLRTALRYYNQSEAGSHIIQRMYGCDVGPDGRL |
| | * Predicted signal | LRGYDQDAYDGKDYIALNEDLSSWTAADTAAQI |
| | peptide underlined | TQRKWEAARVAEQDRAYLEGLCVESLRRYLEN |
| | | GKETLQRADPPKTHVTHHPISDHEVTLRCWALG |
| | | FYPAEITLTWQRDGEDQTQDTELVETRPAGDRTF |
| | | QKWAAVVVPSGEEQRYTCHVQHEGLPKPLTLR |
| | | WEPSSQSTVPIVGIVAGLAVLAVVVIGAVVAAV |
| | | MCRRKSSGGKGGSYSQAACSDSAQGSDVSLTA |
| (SEQ ID | B*15: 01 | MRVTAPRTVLLLLSGALALTETWAGSHSMRYFY |
| NO: 68) | (IMGT/HLA | TAMSRPGRGEPRFIAVGYVDDTQFVRFDSDAASP |
| | Accession No. | RMAPRAPWIEQEGPEYWDRETQISKTNTQTYRES |
| | HLA00162) | LRNLRGYYNQSEAGSHTLQRMYGCDVGPDGRL |
| | * Predicted signal | LRGHDQSAYDGKDYIALNEDLSSWTAADTAAQI |
| | peptide underlined | TQRKWEAAREAEQWRAYLEGLCVEWLRRYLEN |
| | | GKETLQRADPPKTHVTHHPISDHEATLRCWALG |
| | | FYPAEITLTWQRDGEDQTQDTELVETRPAGDRTF |
| | | QKWAAVVVPSGEEQRYTCHVQHEGLPKPLTLR |
| | | WEPSSQSTIPIVGIVAGLAVLAVVVIGAVVATVM |
| | | CRRKSSGGKGGSYSQAASSDSAQGSDVSLTA |
| (SEQ ID | B*40: 01 | MRVTAPRTVLLLLSAALALTETWAGSHSMRYFH |
| NO: 69) | (IMGT/HLA | TAMSRPGRGEPRFITVGYVDDTLFVRFDSDATSP |
| | Accession No. | RKEPRAPWIEQEGPEYWDRETQISKTNTQTYRES |
| | HLA00291) | LRNLRGYYNQSEAGSHTLQRMYGCDVGPDGRL |
| | * Predicted signal | LRGHNQYAYDGKDYIALNEDLRSWTAADTAAQI |
| | peptide underlined | SQRKLEAARVAEQLRAYLEGECVEWLRRYLENG |
| | | KDKLERADPPKTHVTHHPISDHEATLRCWALGF |
| | | YPAEITLTWQRDGEDQTQDTELVETRPAGDRTF |
| | | QKWAAVVVPSGEEQRYTCHVQHEGLPKPLTLR |
| | | WEPSSQSTVPIVGIVAGLAVLAVVVIGAVVAAV |
| | | MCRRKSSGGKGGSYSQAACSDSAQGSDVSLTA |
| (SEQ ID | B*44: 03 | MRVTAPRTLLLLLWGAVALTETWAGSHSMRYF |
| NO: 70) | (IMGT/HLA | YTAMSRPGRGEPRFITVGYVDDTLFVRFDSDATS |
| | Accession No. | PRKEPRAPWIEQEGPEYWDRETQISKTNTQTYRE |
| | HLA00319) | NLRTALRYYNQSEAGSHIIQRMYGCDVGPDGRL |
| | * Predicted signal | LRGYDQDAYDGKDYIALNEDLSSWTAADTAAQI |
| | peptide underlined | TQRKWEAARVAEQLRAYLEGLCVESLRRYLENG |
| | | KETLQRADPPKTHVTHHPISDHEVTLRCWALGF |
| | | YPAEITLTWQRDGEDQTQDTELVETRPAGDRTF |
| | | QKWAAVVVPSGEEQRYTCHVQHEGLPKPLTLR |
| | | WEPSSQSTVPIVGIVAGLAVLAVVVIGAVVAAV |
| | | MCRRKSSGGKGGSYSQAACSDSAQGSDVSLTA |
| (SEQ ID | B*35: 01 | MRVTAPRTVLLLLWGAVALTETWAGSHSMRYF |
| NO: 71) | (IMGT/HLA | YTAMSRPGRGEPRFIAVGYVDDTQFVRFDSDAA |
| | Accession No. | SPRTEPRAPWIEQEGPEYWDRNTQIFKTNTQTYR |
| | HLA00237) | ESLRNLRGYYNQSEAGSHIIQRMYGCDLGPDGRL |
| | * Predicted signal | LRGHDQSAYDGKDYIALNEDLSSWTAADTAAQI |
| | peptide underlined | TQRKWEAARVAEQLRAYLEGLCVEWLRRYLEN |
| | | GKETLQRADPPKTHVTHHPVSDHEATLRCWALG |
| | | FYPAEITLTWQRDGEDQTQDTELVETRPAGDRTF |
| | | QKWAAVVVPSGEEQRYTCHVQHEGLPKPLTLR |
| | | WEPSSQSTIPIVGIVAGLAVLAVVVIGAVVATVM |
| | | CRRKSSGGKGGSYSQAASSDSAQGSDVSLTA |
| (SEQ ID | B*51: 01 | MRVTAPRTVLLLLWGAVALTETWAGSHSMRYF |
| NO: 72) | (IMGT/HLA | YTAMSRPGRGEPRFIAVGYVDDTQFVRFDSDAA |
| | Accession No. | SPRTEPRAPWIEQEGPEYWDRNTQIFKTNTQTYR |
| | HLA00344) | ENLRIALRYYNQSEAGSHTWQTMYGCDVGPDG |
| | * Predicted signal | RLLRGHNQYAYDGKDYIALNEDLSSWTAADTA |
| | peptide underlined | AQITQRKWEAAREAEQLRAYLEGLCVEWLRRHL |
| | | ENGKETLQRADPPKTHVTHHPVSDHEATLRCWA |
| | | LGFYPAEITLTWQRDGEDQTQDTELVETRPAGD |
| | | RTFQKWAAVVVPSGEEQRYTCHVQHEGLPKPLT |
| | | LRWEPSSQSTIPIVGIVAGLAVLAVVVIGAVVAT |
| | | VMCRRKSSGGKGGSYSQAASSDSAQGSDVSLTA |
| (SEQ ID | B*27: 05 | MRVTAPRTLLLLLWGAVALTETWAGSHSMRYF |
| NO: 73) | (IMGT/HLA | HTSVSRPGRGEPRFITVGYVDDTLFVRFDSDAAS |
| | Accession No. | PREEPRAPWIEQEGPEYWDRETQICKAKAQTDRE |
| | HLA00225) | DLRTLLRYYNQSEAGSHTLQNMYGCDVGPDGR |
| | * Predicted signal | LLRGYHQDAYDGKDYIALNEDLSSWTAADTAA |
| | peptide underlined | QITQRKWEAARVAEQLRAYLEGECVEWLRRYLE |
| | | NGKETLQRADPPKTHVTHHPISDHEATLRCWAL |
| | | GFYPAEITLTWQRDGEDQTQDTELVETRPAGDR |
| | | TFQKWAAVVVPSGEEQRYTCHVQHEGLPKPLTL |
| | | RWEPSSQSTVPIVGIVAGLAVLAVVVIGAVVAAV |
| | | MCRRKSSGGKGGSYSQAACSDSAQGSDVSLTA |
| (SEQ ID | B*57: 01 | MRVTAPRTVLLLLWGAVALTETWAGSHSMRYF |
| NO: 74) | (IMGT/HLA | YTAMSRPGRGEPRFIAVGYVDDTQFVRFDSDAA |
| | Accession No. | SPRMAPRAPWIEQEGPEYWDGETRNMKASAQT |
| | HLA00381) | YRENLRIALRYYNQSEAGSHIIQVMYGCDVGPD |
| | * Predicted signal | GRLLRGHDQSAYDGKDYIALNEDLSSWTAADTA |
| | peptide underlined | AQITQRKWEAARVAEQLRAYLEGLCVEWLRRY |
| | | LENGKETLQRADPPKTHVTHHPISDHEATLRCW |
| | | ALGFYPAEITLTWQRDGEDQTQDTELVETRPAG |
| | | DRTFQKWAAVVVPSGEEQRYTCHVQHEGLPKPL |
| | | TLRWEPSSQSTVPIVGIVAGLAVLAVVVIGAVVA |
| | | AVMCRRKSSGGKGGSYSQAACSDSAQGSDVSLT |
| | | A |
| (SEQ ID | B*18: 01 | MRVTAPRTLLLLLWGAVALTETWAGSHSMRYF |
| NO: 75) | (IMGT/HLA | HTSVSRPGRGEPRFISVGYVDGTQFVRFDSDAAS |
| | Accession No. | PRTEPRAPWIEQEGPEYWDRNTQISKTNTQTYRE |
| | HLA00213) | SLRNLRGYYNQSEAGSHTLQRMYGCDVGPDGR |
| | * Predicted signal | LLRGHDQSAYDGKDYIALNEDLSSWTAADTAAQ |
| | peptide underlined | ITQRKWEAARVAEQLRAYLEGTCVEWLRRHLEN |
| | | GKETLQRADPPKTHVTHHPISDHEATLRCWALG |
| | | FYPAEITLTWQRDGEDQTQDTELVETRPAGDRTF |
| | | QKWAAVVVPSGEEQRYTCHVQHEGLPKPLTLR |
| | | WEPSSQSTIPIVGIVAGLAVLAVVVIGAVVATVM |
| | | CRRKSSGGKGGSYSQAASSDSAQGSDVSLTA |
| (SEQ ID | B*14: 02 | MLVMAPRTVLLLLSAALALTETWAGSHSMRYF |
| NO: 76) | (IMGT/HLA | YTAVSRPGRGEPRFISVGYVDDTQFVRFDSDAAS |
| | Accession No. | PREEPRAPWIEQEGPEYWDRNTQICKTNTQTDRE |
| | HLA00158) | SLRNLRGYYNQSEAGSHTLQWMYGCDVGPDGR |
| | * Predicted signal | LLRGYNQFAYDGKDYIALNEDLSSWTAADTAAQ |
| | peptide underlined | ITQRKWEAAREAEQLRAYLEGTCVEWLRRHLEN |
| | | GKETLQRADPPKTHVTHHPISDHEATLRCWALG |
| | | FYPAEITLTWQRDGEDQTQDTELVETRPAGDRTF |
| | | QKWAAVVVPSGEEQRYTCHVQHEGLPKPLTLR |
| | | WEPSSQSTVPIVGIVAGLAVLAVVVIGAVVAAV |
| | | MCRRKSSGGKGGSYSQAASSDSAQGSDVSLTA |
| (SEQ ID | B*13: 02 | MRVTAPRTLLLLLWGAVALTETWAGSHSMRYF |
| NO: 77) | (IMGT/HLA | YTAMSRPGRGEPRFITVGYVDDTQFVRFDSDATS |
| | Accession No. | PRMAPRAPWIEQEGPEYWDRETQISKTNTQTYRE |
| | HLA00153) | NLRTALRYYNQSEAGSHTWQTMYGCDLGPDGR |
| | * Predicted signal | LLRGHNQLAYDGKDYIALNEDLSSWTAADTAA |
| | peptide underlined | QITQLKWEAARVAEQLRAYLEGECVEWLRRYLE |
| | | NGKETLQRADPPKTHVTHHPISDHEATLRCWAL |
| | | GFYPAEITLTWQRDGEDQTQDTELVETRPAGDR |
| | | TFQKWAAVVVPSGEEQRYTCHVQHEGLPKPLTL |
| | | RWEPSSQSTVPIVGIVAGLAVLAVVVIGAVVAAV |
| | | MCRRKSSGGKGGSYSQAACSDSAQGSDVSLTA |
| (SEQ ID | B*55: 01 | MRVTAPRTLLLLLWGALALTETWAGSHSMRYF |
| NO: 78) | (IMGT/HLA | YTAMSRPGRGEPRFIAVGYVDDTQFVRFDSDAA |
| | Accession No. | SPREEPRAPWIEQEGPEYWDRNTQIYKAQAQTD |
| | HLA00368) | RESLRNLRGYYNQSEAGSHTWQTMYGCDLGPD |
| | * Predicted signal | GRLLRGHNQLAYDGKDYIALNEDLSSWTAADTA |
| | peptide underlined | AQITQRKWEAAREAEQLRAYLEGTCVEWLRRYL |
| | | ENGKETLQRADPPKTHVTHHPISDHEATLRCWA |
| | | LGFYPAEITLTWQRDGEDQTQDTELVETRPAGD |
| | | RTFQKWAAVVVPSGEEQRYTCHVQHEGLPKPLT |
| | | LRWEPSSQSTIPIVGIVAGLAVLAVVVIGAVVAT |
| | | VMCRRKSSGGKGGSYSQAASSDSAQGSDVSLTA |
| (SEQ ID | B*14: 01 | MLVMAPRTVLLLLSAALALTETWAGSHSMRYF |
| NO: 79) | (IMGT/HLA | YTSVSRPGRGEPRFISVGYVDDTQFVRFDSDAAS |
| | Accession No. | PREEPRAPWIEQEGPEYWDRNTQICKTNTQTDRE |
| | HLA00157) | SLRNLRGYYNQSEAGSHTLQWMYGCDVGPDGR |
| | * Predicted signal | LLRGYNQFAYDGKDYIALNEDLSSWTAADTAAQ |
| | peptide underlined | ITQRKWEAAREAEQLRAYLEGTCVEWLRRHLEN |
| | | GKETLQRADPPKTHVTHHPISDHEATLRCWALG |
| | | FYPAEITLTWQRDGEDQTQDTELVETRPAGDRTF |
| | | QKWAAVVVPSGEEQRYTCHVQHEGLPKPLTLR |
| | | WEPSSQSTVPIVGIVAGLAVLAVVVIGAVVAAV |
| | | MCRRKSSGGKGGSYSQAASSDSAQGSDVSLTA |
| (SEQ ID | B*49: 01 | MRVTAPRTVLLLLSAALALTETWAGSHSMRYFH |
| NO: 80) | (IMGT/HLA | TAMSRPGRGEPRFITVGYVDDTLFVRFDSDATSP |
| | Accession No. | RKEPRAPWIEQEGPEYWDRETQISKTNTQTYREN |
| | HLA00340) | LRIALRYYNQSEAGSHTWQRMYGCDLGPDGRLL |
| | * Predicted signal | RGYNQLAYDGKDYIALNEDLSSWTAADTAAQIT |
| | peptide underlined | QRKWEAAREAEQLRAYLEGLCVEWLRRYLENG |
| | | KETLQRADPPKTHVTHHPISDHEATLRCWALGF |
| | | YPAEITLTWQRDGEDQTQDTELVETRPAGDRTF |
| | | QKWAAVVVPSGEEQRYTCHVQHEGLPKPLTLR |
| | | WEPSSQSTIPIVGIVAGLAVLAVVVIGAVVATVM |
| | | CRRKSSGGKGGSYSQAASSDSAQGSDVSLTA |
| (SEQ ID | B*37: 01 | MRVTAPRTLLLLLWGAVALTETWAGSHSMRYF |
| NO: 81) | (IMGT/HLA | HTSVSRPGRGEPRFISVGYVDDTQFVRFDSDAAS |
| | Accession No. | PRTEPRAPWIEQEGPEYWDRETQISKTNTQTYRE |
| | HLA00265) | DLRTLLRYYNQSEAGSHTIQRMSGCDVGPDGRL |
| | * Predicted signal | LRGYNQFAYDGKDYIALNEDLSSWTAADTAAQI |
| | peptide underlined | TQRKWEAARVAEQDRAYLEGTCVEWLRRYLEN |
| | | GKETLQRADPPKTHVTHHPISDHEATLRCWALG |
| | | FYPAEITLTWQRDGEDQTQDTELVETRPAGDRTF |
| | | QKWAAVVVPSGEEQRYTCHVQHEGLPKPLTLR |
| | | WEPSSQSTIPIVGIVAGLAVLAVVVIGAVVATVM |
| | | CRRKSSGGKGGSYSQAASSDSAQGSDVSLTA |
| (SEQ ID | B*38: 01 | MLVMAPRTVLLLLSAALALTETWAGSHSMRYF |
| NO: 82) | (IMGT/HLA | YTSVSRPGRGEPRFISVGYVDDTQFVRFDSDAAS |
| | Accession No. | PREEPRAPWIEQEGPEYWDRNTQICKTNTQTYRE |
| | HLA00267) | NLRIALRYYNQSEAGSHTLQRMYGCDVGPDGRL |
| | * Predicted signal | LRGHNQFAYDGKDYIALNEDLSSWTAADTAAQI |
| | peptide underlined | TQRKWEAARVAEQLRTYLEGTCVEWLRRYLEN |
| | | GKETLQRADPPKTHVTHHPISDHEATLRCWALG |
| | | FYPAEITLTWQRDGEDQTQDTELVETRPAGDRTF |
| | | QKWAAVVVPSGEEQRYTCHVQHEGLPKPLTLR |
| | | WEPSSQSTVPIVGIVAGLAVLAVVVIGAVVAAV |
| | | MCRRKSSGGKGGSYSQAASSDSAQGSDVSLTA |
| (SEQ ID | B*39: 01 | MLVMAPRTVLLLLSAALALTETWAGSHSMRYF |
| NO: 83) | (IMGT/HLA | YTSVSRPGRGEPRFISVGYVDDTQFVRFDSDAAS |
| | Accession No. | PREEPRAPWIEQEGPEYWDRNTQICKTNTQTDRE |
| | HLA00271) | SLRNLRGYYNQSEAGSHTLQRMYGCDVGPDGR |
| | * Predicted signal | LLRGHNQFAYDGKDYIALNEDLSSWTAADTAAQ |
| | peptide underlined | ITQRKWEAARVAEQLRTYLEGTCVEWLRRYLEN |
| | | GKETLQRADPPKTHVTHHPISDHEATLRCWALG |
| | | FYPAEITLTWQRDGEDQTQDTELVETRPAGDRTF |
| | | QKWAAVVVPSGEEQRYTCHVQHEGLPKPLTLR |
| | | WEPSSQSTVPIVGIVAGLAVLAVVVIGAVVAAV |
| | | MCRRKSSGGKGGSYSQAASSDSAQGSDVSLTA |
| (SEQ ID | B*35: 03 | MRVTAPRTVLLLLWGAVALTETWAGSHSMRYF |
| NO: 84) | (IMGT/HLA | YTAMSRPGRGEPRFIAVGYVDDTQFVRFDSDAA |
| | Accession No. | SPRTEPRAPWIEQEGPEYWDRNTQIFKTNTQTYR |
| | HLA00239) | ESLRNLRGYYNQSEAGSHIIQRMYGCDLGPDGRL |
| | * Predicted signal | LRGHDQFAYDGKDYIALNEDLSSWTAADTAAQI |
| | peptide underlined | TQRKWEAARVAEQLRAYLEGLCVEWLRRYLEN |
| | | GKETLQRADPPKTHVTHHPVSDHEATLRCWALG |
| | | FYPAEITLTWQRDGEDQTQDTELVETRPAGDRTF |
| | | QKWAAVVVPSGEEQRYTCHVQHEGLPKPLTLR |
| | | WEPSSQSTIPIVGIVAGLAVLAVVVIGAVVATVM |
| | | CRRKSSGGKGGSYSQAASSDSAQGSDVSLTA |
| (SEQ ID | B*40: 02 | MRVTAPRTLLLLLWGAVALTETWAGSHSMRYF |
| NO: 85) | (IMGT/HLA | HTSVSRPGRGEPRFITVGYVDDTLFVRFDSDATSP |
| | Accession No. | RKEPRAPWIEQEGPEYWDRETQISKTNTQTYRES |
| | HLA00293) | LRNLRGYYNQSEAGSHTLQSMYGCDVGPDGRLL |
| | * Predicted signal | RGHNQYAYDGKDYIALNEDLRSWTAADTAAQIT |
| | peptide underlined | QRKWEAARVAEQLRAYLEGECVEWLRRYLENG |
| | | KETLQRADPPKTHVTHHPISDHEATLRCWALGF |
| | | YPAEITLTWQRDGEDQTQDTELVETRPAGDRTF |
| | | QKWAAVVVPSGEEQRYTCHVQHEGLPKPLTLR |
| | | WEPSSQSTVPIVGIVAGLAVLAVVVIGAVVAAV |
| | | MCRRKSSGGKGGSYSQAACSDSAQGSDVSLTA |
| (SEQ ID | DRB1*07: 01 | MVCLKLPGGSCMAALTVTLMVLSSPLALAGDTQ |
| NO: 86) | (IMGT/HLA | PRFLWQGKYKCHFFNGTERVQFLERLFYNQEEF |
| | Accession No. | VRFDSDVGEYRAVTELGRPVAESWNSQKDILED |
| | HLA00719) | RRGQVDTVCRHNYGVGESFTVQRRVHPEVTVYP |
| | * Predicted signal | AKTQPLQHHNLLVCSVSGFYPGSIEVRWFRNGQ |
| | peptide underlined | EEKAGVVSTGLIQNGDWTFQTLVMLETVPRSGE |
| | | VYTCQVEHPSVMSPLTVEWRARSESAQSKMLSG |
| | | VGGFVLGLLFLGAGLFIYFRNQKGHSGLQPTGFL |
| | | S |
| (SEQ ID | DRB1*03: 01 | MVCLRLPGGSCMAVLTVTLMVLSSPLALAGDTR |
| NO: 87) | (IMGT/HLA | PRFLEYSTSECHFFNGTERVRYLDRYFHNQEENV |
| | Accession No. | RFDSDVGEFRAVTELGRPDAEYWNSQKDLLEQK |
| | HLA00671) | RGRVDNYCRHNYGVVESFTVQRRVHPKVTVYPS |
| | * Predicted signal | KTQPLQHHNLLVCSVSGFYPGSIEVRWFRNGQEE |
| | peptide underlined | KTGVVSTGLIHNGDWTFQTLVMLETVPRSGEVY |
| | | TCQVEHPSVTSPLTVEWRARSESAQSKMLSGVG |
| | | GFVLGLLFLGAGLFIYFRNQKGHSGLQPRGFLS |
| (SEQ ID | DRB1*15: 01 | MVCLKLPGGSCMTALTVTLMVLSSPLALSGDTR |
| NO: 88) | (IMGT/HLA | PRFLWQPKRECHFFNGTERVRFLDRYFYNQEESV |
| | Accession No. | RFDSDVGEFRAVTELGRPDAEYWNSQKDILEQA |
| | HLA00865) | RAAVDTYCRHNYGVVESFTVQRRVQPKVTVYPS |
| | * Predicted signal | KTQPLQHHNLLVCSVSGFYPGSIEVRWFLNGQEE |
| | peptide underlined | KAGMVSTGLIQNGDWTFQTLVMLETVPRSGEVY |
| | | TCQVEHPSVTSPLTVEWRARSESAQSKMLSGVG |
| | | GFVLGLLFLGAGLFIYFRNQKGHSGLQPTGFLS |
| (SEQ ID | DRB1*04: 01 | MVCLKFPGGSCMAALTVTLMVLSSPLALAGDTR |
| NO: 89) | (IMGT/HLA | PRFLEQVKHECHFFNGTERVRFLDRYFYHQEEY |
| | Accession No. | VRFDSDVGEYRAVTELGRPDAEYWNSQKDLLEQ |
| | HLA00685) | KRAAVDTYCRHNYGVGESFTVQRRVYPEVTVYP |
| | * Predicted signal | AKTQPLQHHNLLVCSVNGFYPGSIEVRWFRNGQ |
| | peptide underlined | EEKTGVVSTGLIQNGDWTFQTLVMLETVPRSGE |
| | | VYTCQVEHPSLTSPLTVEWRARSESAQSKMLSG |
| | | VGGFVLGLLFLGAGLFIYFRNQKGHSGLQPTGFL |
| | | S |
| (SEQ ID | DRB1*01: 01 | MVCLKLPGGSCMTALTVTLMVLSSPLALAGDTR |
| NO: 90) | (IMGT/HLA | PRFLWQLKFECHFFNGTERVRLLERCIYNQEESV |
| | Accession No. | RFDSDVGEYRAVTELGRPDAEYWNSQKDLLEQR |
| | HLA00664) | RAAVDTYCRHNYGVGESFTVQRRVEPKVTVYPS |
| | * Predicted signal | KTQPLQHHNLLVCSVSGFYPGSIEVRWFRNGQEE |
| | peptide underlined | KAGVVSTGLIQNGDWTFQTLVMLETVPRSGEVY |
| | | TCQVEHPSVTSPLTVEWRARSESAQSKMLSGVG |
| | | GFVLGLLFLGAGLFIYFRNQKGHSGLQPTGFLS |
| (SEQ ID | DRB1*13: 01 | MVCLRLPGGSCMAVLTVTLMVLSSPLALAGDTR |
| NO: 91) | (IMGT/HLA | PRFLEYSTSECHFFNGTERVRFLDRYFHNQEENV |
| | Accession No. | RFDSDVGEFRAVTELGRPDAEYWNSQKDILEDE |
| | HLA00797) | RAAVDTYCRHNYGVVESFTVQRRVHPKVTVYPS |
| | * Predicted signal | KTQPLQHHNLLVCSVSGFYPGSIEVRWFRNGQEE |
| | peptide underlined | KTGVVSTGLIHNGDWTFQTLVMLETVPRSGEVY |
| | | TCQVEHPSVTSPLTVEWRARSESAQSKMLSGVG |
| | | GFVLGLLFLGAGLFIYFRNQKGHSGLQPRGFLS |
| (SEQ ID | DRB1*11: 01 | MVCLRLPGGSCMAVLTVTLMVLSSPLALAGDTR |
| NO: 92) | (IMGT/HLA | PRFLEYSTSECHFFNGTERVRFLDRYFYNQEEYV |
| | Accession No. | RFDSDVGEFRAVTELGRPDEEYWNSQKDFLEDR |
| | HLA00751) | RAAVDTYCRHNYGVGESFTVQRRVHPKVTVYPS |
| | * Predicted signal | KTQPLQHHNLLVCSVSGFYPGSIEVRWFRNGQEE |
| | peptide underlined | KTGVVSTGLIHNGDWTFQTLVMLETVPRSGEVY |
| | | TCQVEHPSVTSPLTVEWRARSESAQSKMLSGVG |
| | | GFVLGLLFLGAGLFIYFRNQKGHSGLQPRGFLS |
| (SEQ ID | DRB1*04: 04 | MVCLKFPGGSCMAALTVTLMVLSSPLALAGDTR |
| NO: 93) | (IMGT/HLA | PRFLEQVKHECHFFNGTERVRFLDRYFYHQEEY |
| | Accession No. | VRFDSDVGEYRAVTELGRPDAEYWNSQKDLLEQ |
| | HLA00689) | RRAAVDTYCRHNYGVVESFTVQRRVYPEVTVYP |
| | * Predicted signal | AKTQPLQHHNLLVCSVNGFYPGSIEVRWFRNGQ |
| | peptide underlined | EEKTGVVSTGLIQNGDWTFQTLVMLETVPRSGE |
| | | VYTCQVEHPSLTSPLTVEWRARSESAQSKMLSG |
| | | VGGFVLGLLFLGAGLFIYFRNQKGHSGLQPTGFL |
| | | S |
| (SEQ ID | DRB1*13: 02 | MVCLRLPGGSCMAVLTVTLMVLSSPLALAGDTR |
| NO: 94) | (IMGT/HLA | PRFLEYSTSECHFFNGTERVRFLDRYFHNQEENV |
| | Accession No. | RFDSDVGEFRAVTELGRPDAEYWNSQKDILEDE |
| | HLA00798) | RAAVDTYCRHNYGVGESFTVQRRVHPKVTVYPS |
| | * Predicted signal | KTQPLQHHNLLVCSVSGFYPGSIEVRWFRNGQEE |
| | peptide underlined | KTGVVSTGLIHNGDWTFQTLVMLETVPRSGEVY |
| | | TCQVEHPSVTSPLTVEWRARSESAQSKMLSGVG |
| | | GFVLGLLFLGAGLFIYFRNQKGHSGLQPRGFLS |
| (SEQ ID | DRB1*08: 01 | MVCLRLPGGSCMAVLTVTLMVLSSPLALAGDTR |
| NO: 95) | (IMGT/HLA | PRFLEYSTGECYFFNGTERVRFLDRYFYNQEEYV |
| | Accession No. | RFDSDVGEYRAVTELGRPSAEYWNSQKDFLEDR |
| | HLA00723) | RALVDTYCRHNYGVGESFTVQRRVHPKVTVYPS |
| | * Predicted signal | KTQPLQHHNLLVCSVSGFYPGSIEVRWFRNGQEE |
| | peptide underlined | KTGVVSTGLIHNGDWTFQTLVMLETVPRSGEVY |
| | | TCQVEHPSVTSPLTVEWSARSESAQSKMLSGVG |
| | | GFVLGLLFLGAGLFIYFRNQKGHSGLQPTGFLS |
| (SEQ ID | DRB1*12: 01 | MVCLRLPGGSCMAVLTVTLMVLSSPLALAGDTR |
| NO: 96) | (IMGT/HLA | PRFLEYSTGECYFFNGTERVRLLERHFHNQEELL |
| | Accession No. | RFDSDVGEFRAVTELGRPVAESWNSQKDILEDRR |
| | HLA00789) | AAVDTYCRHNYGAVESFTVQRRVHPKVTVYPSK |
| | * Predicted signal | TQPLQHHNLLVCSVSGFYPGSIEVRWFRNGQEEK |
| | peptide underlined | TGVVSTGLIHNGDWTFQTLVMLETVPRSGEVYT |
| | | CQVEHPSVTSPLTVEWRARSESAQSKMLSGVGG |
| | | FVLGLLFLGAGLFIYFRNQKGHSGLQPRGFLS |
| (SEQ ID | DRB1*11: 04 | MVCLRLPGGSCMAVLTVTLMVLSSPLALAGDTR |
| NO: 97) | (IMGT/HLA | PRFLEYSTSECHFFNGTERVRFLDRYFYNQEEYV |
| | Accession No. | RFDSDVGEFRAVTELGRPDEEYWNSQKDFLEDR |
| | HLA00756) | RAAVDTYCRHNYGVVESFTVQRRVHPKVTVYPS |
| | * Predicted signal | KTQPLQHHNLLVCSVSGFYPGSIEVRWFRNGQEE |
| | peptide underlined | KTGVVSTGLIHNGDWTFQTLVMLETVPRSGEVY |
| | | TCQVEHPSVTSPLTVEWRARSESAQSKMLSGVG |
| | | GFVLGLLFLGAGLFIYFRNQKGHSGLQPRGFLS |
| (SEQ ID | DRB1*09: 01 | MVCLKLPGGSCMAALTVTLMVLSSPLALAGDTQ |
| NO: 98) | (IMGT/HLA | PRFLKQDKFECHFFNGTERVRYLHRGIYNQEENV |
| | Accession No. | RFDSDVGEYRAVTELGRPVAESWNSQKDFLERR |
| | HLA00749) | RAEVDTVCRHNYGVGESFTVQRRVHPEVTVYPA |
| | * Predicted signal | KTQPLQHHNLLVCSVSGFYPGSIEVRWFRNGQEE |
| | peptide underlined | KAGVVSTGLIQNGDWTFQTLVMLETVPRSGEVY |
| | | TCQVEHPSVMSPLTVEWRARSESAQSKMLSGVG |
| | | GFVLGLLFLGAGLFIYFRNQKGHSGLQPTGFLS |
| (SEQ ID | DRB1*14: 01 | MVCLRLPGGSCMAVLTVTLMVLSSPLALAGDTR |
| NO: 99) | (IMGT/HLA | PRFLEYSTSECHFFNGTERVRFLDRYFHNQEEFV |
| | Accession No. | RFDSDVGEYRAVTELGRPAAEHWNSQKDLLERR |
| | HLA00833) | RAEVDTYCRHNYGVVESFTVQRRVHPKVTVYPS |
| | * Predicted signal | KTQPLQHYNLLVCSVSGFYPGSIEVRWFRNGQEE |
| | peptide underlined | KTGVVSTGLIHNGDWTFQTLVMLETVPRSGEVY |
| | | TCQVEHPSVTSPLTVEWRARSESAQSKMLSGVG |
| | | GFVLGLLFLGAGLFIYFRNQKGHSGLQPRGFLS |
| (SEQ ID | DRB 1*04 : 07 | MVCLKFPGGSCMAALTVTLMVLSSPLALAGDTR |
| NO: 100) | (IMGT/HLA | PRFLEQVKHECHFFNGTERVRFLDRYFYHQEEY |
| | Accession No. | VRFDSDVGEYRAVTELGRPDAEYWNSQKDLLEQ |
| | HLA00693) | RRAEVDTYCRHNYGVGESFTVQRRVYPEVTVYP |
| | * Predicted signal | AKTQPLQHHNLLVCSVNGFYPGSIEVRWFRNGQ |
| | peptide underlined | EEKTGVVSTGLIQNGDWTFQTLVMLETVPRSGE |
| | | VYTCQVEHPSLTSPLTVEWRARSESAQSKMLSG |
| | | VGGFVLGLLFLGAGLFIYFRNQKGHSGLQPTGFL |
| | | S |
| (SEQ ID | DRB1*14: 04 | MVCLRLPGGSCMAVLTVTLMVLSSPLALAGDTR |
| NO: 101) | (IMGT/HLA | PRFLEYSTGECYFFNGTERVRFLDRYFHNQEEFV |
| | Accession No. | RFDSDVGEYRAVTELGRPAAEHWNSQKDLLERR |
| | HLA00836) | RAEVDTYCRHNYGVVESFTVQRRVHPKVTVYPS |
| | * Predicted signal | KTQPLQHHNLLVCSVSGFYPGSIEVRWFRNGQEE |
| | peptide underlined | KTGVVSTGLIHNGDWTFQTLVMLETVPRSGEVY |
| | | TCQVEHPSVTSPLTVEWRARSESAQSKMLSGVG |
| | | GFVLGLLFLGAGLFIYFRNQKGHSGLQPRGFLS |
| (SEQ ID | DQA1*05: 01 | MILNKALMLGALALTTVMSPCGGEDIVADHVAS |
| NO: 102) | (IMGT/HLA | YGVNLYQSYGPSGQYTHEFDGDEQFYVDLGRKE |
| | Accession No. | TVWCLPVLRQFRFDPQFALTNIAVLKHNLNSLIK |
| | HLA00613) | RSNSTAATNEVPEVTVFSKSPVTLGQPNILICLVD |
| | * Predicted signal | NIFPPVVNITWLSNGHSVTEGVSETSFLSKSDHSF |
| | peptide underlined | FKISYLTLLPSAEESYDCKVEHWGLDKPLLKHWE |
| | | PEIPAPMSELTETVVCALGLSVGLVGIVVGTVFII |
| | | RGLRSVGASRHQGPL |
| (SEQ ID | DQA1*03: 01 | MILNKALMLGALALTTVMSPCGGEDIVADHVAS |
| NO: 103) | (IMGT/HLA | YGVNLYQSYGPSGQYSHEFDGDEEFYVDLERKE |
| | Accession No. | TVWQLPLFRRFRRFDPQFALTNIAVLKHNLNIVIK |
| | HLA00608) | RSNSTAATNEVPEVTVFSKSPVTLGQPNTLICLVD |
| | * Predicted signal | NIFPPVVNITWLSNGHSVTEGVSETSFLSKSDHSF |
| | peptide underlined | FKISYLTFLPSADEIYDCKVEHWGLDEPLLKHWE |
| | | PEIPTPMSELTETVVCALGLSVGLVGIVVGTVLIIR |
| | | GLRSVGASRHQGPL |
| (SEQ ID | DQA1*01: 02 | MILNKALLLGALALTTVMSPCGGEDIVADHVAS |
| NO: 104) | (IMGT/HLA | CGVNLYQFYGPSGQYTHEFDGDEQFYVDLERKE |
| | Accession No. | TAWRWPEFSKFGGFDPQGALRNMAVAKHNLNI |
| | HLA00602) | MIKRYNSTAATNEVPEVTVFSKSPVTLGQPNTLI |
| | * Predicted signal | CLVDNIFPPVVNITWLSNGQSVTEGVSETSFLSKS |
| | peptide underlined | DHSFFKISYLTFLPSADEIYDCKVEHWGLDQPLL |
| | | KHWEPEIPAPMSELTETVVCALGLSVGLMGIVVG |
| | | TVFIIQGLRSVGASRHQGPL |
| (SEQ ID | DQA1*02: 01 | MILNKALMLGALALTTVMSPCGGEDIVADHVAS |
| NO: 105) | (IMGT/HLA | YGVNLYQSYGPSGQFTHEFDGDEEFYVDLERKE |
| | Accession No. | TVWKLPLFHRLRFDPQFALTNIAVLKHNLNILIKR |
| | HLA00607) | SNSTAATNEVPEVTVFSKSPVTLGQPNTLICLVD |
| | * Predicted signal | NIFPPVVNITWLSNGHSVTEGVSETSFLSKSDHSF |
| | peptide underlined | FKISYLTFLPSADEIYDCKVEHWGLDEPLLKHWE |
| | | PEIPAPMSELTETVVCALGLSVGLVGIVVGTVLII |
| | | RGLRSVGASRHQGPL |
| (SEQ ID | DQA1*01: 01 | MILNKALLLGALALTTVMSPCGGEDIVADHVAS |
| NO: 106) | (IMGT/HLA | CGVNLYQFYGPSGQYTHEFDGDEEFYVDLERKE |
| | Accession No. | TAWRWPEFSKFGGFDPQGALRNMAVAKHNLNI |
| | HLA00601) | MIKRYNSTAATNEVPEVTVFSKSPVTLGQPNTLI |
| | * Predicted signal | CLVDNIFPPVVNITWLSNGQSVTEGVSETSFLSKS |
| | peptide underlined | DHSFFKISYLTFLPSADEIYDCKVEHWGLDQPLL |
| | | KHWEPEIPAPMSELTETVVCALGLSVGLVGIVVG |
| | | TVFIIQGLRSVGASRHQGPL |
| (SEQ ID | DQA1*01: 03 | MILNKALLLGALALTTVMSPCGGEDIVADHVAS |
| NO: 107) | (IMGT/HLA | CGVNLYQFYGPSGQFTHEFDGDEQFYVDLEKKE |
| | Accession No. | TAWRWPEFSKFGGFDPQGALRNMAVAKHNLNI |
| | HLA00604) | MIKRYNSTAATNEVPEVTVFSKSPVTLGQPNTLI |
| | * Predicted signal | CLVDNIFPPVVNITWLSNGHAVTEGVSETSFLSKS |
| | peptide underlined | DHSFFKISYLTFLPSADEIYDCKVEHWGLDQPLL |
| | | KHWEPEIPAPMSELTETVVCALGLSVGLVGIVVG |
| | | TVFIIQGLRSVGASRHQGPL |
| (SEQ ID | DQA1*04: 01 | MILNKALLLGALALTTVMSPCGGEDIVADHVAS |
| NO: 108) | (IMGT/HLA | YGVNLYQSYGPSGQYTHEFDGDEQFYVDLGRKE |
| | Accession No. | TVWCLPVLRQFRFDPQFALTNIAVTKHNLNILIK |
| | HLA00612) | RSNSTAATNEVPEVTVFSKSPVTLGQPNTLICLVD |
| | * Predicted signal | NIFPPVVNITWLSNGHSVTEGVSETSFLSKSDHSF |
| | peptide underlined | FKISYLTFLPSADEIYDCKVEHWGLDEPLLKHWE |
| | | PEIPAPMSELTETVVCALGLSVGLVGIVVGTVFII |
| | | RGLRSVGASRHQGPL |
| (SEQ ID | DQB1*03: 01 | MSWKKALRIPGGLRAATVTLMLAMLSTPVAEGR |
| NO: 109) | (IMGT/HLA | DSPEDFVYQFKAMCYFTNGTERVRYVTRYIYNR |
| | Accession No. | EEYARFDSDVEVYRAVTPLGPPDAEYWNSQKEV |
| | HLA00625) | LERTRAELDTVCRHNYQLELRTTLQRRVEPTVTI |
| | * Predicted signal | SPSRTEALNHHNLLVCSVTDFYPAQIKVRWFRND |
| | peptide underlined | QEETTGVVSTPLIRNGDWTFQILVMLEMTPQHG |
| | | DVYTCHVEHPSLQNPITVEWRAQSESAQSKMLS |
| | | GIGGFVLGLIFLGLGLIIHHRSQKGLLH |
| (SEQ ID | DQB1*02: 01 | MSWKKALRIPGGLRAATVTLMLSMLSTPVAEGR |
| NO: 110) | (IMGT/HLA | DSPEDFVYQFKGMCYFTNGTERVRLVSRSIYNRE |
| | Accession No. | EIVRFDSDVGEFRAVTLLGLPAAEYWNSQKDILE |
| | HLA00622) | RKRAAVDRVCRHNYQLELRTTLQRRVEPTVTISP |
| | * Predicted signal | SRTEALNHHNLLVCSVTDFYPAQIKVRWFRNDQ |
| | peptide underlined | EETAGVVSTPLIRNGDWTFQILVMLEMTPQRGD |
| | | VYTCHVEHPSLQSPITVEWRAQSESAQSKMLSGI |
| | | GGFVLGLIFLGLGLIIHHRSQKGLLH |
| (SEQ ID | DQB1*06: 02 | MSWKKALRIPGDLRVATVTLMLAMLSSLLAEGR |
| NO: 111) | (IMGT/HLA | DSPEDFVFQFKGMCYFTNGTERVRLVTRYIYNRE |
| | Accession No. | EYARFDSDVGVYRAVTPQGRPDAEYWNSQKEV |
| | HLA00646) | LEGTRAELDTVCRHNYEVAFRGILQRRVEPTVTI |
| | * Predicted signal | SPSRTEALNHHNLLVCSVTDFYPGQIKVRWFRND |
| | peptide underlined | QEETAGVVSTPLIRNGDWTFQILVMLEMTPQRG |
| | | DVYTCHVEHPSLQSPITVEWRAQSESAQSKMLSG |
| | | VGGFVLGLIFLGLGLIIRQRSQKGLLH |
| (SEQ ID | DQB1*05: 01 | MSWKKSLRIPGDLRVATVTLMLAILSSSLAEGRD |
| NO: 112) | (IMGT/HLA | SPEDFVYQFKGLCYFTNGTERVRGVTRHIYNREE |
| | Accession No. | YVRFDSDVGVYRAVTPQGRPVAEYWNSQKEVL |
| | HLA00638) | EGARASVDRVCRHNYEVAYRGILQRRVEPTVTIS |
| | * Predicted signal | PSRTEALNHHNLLICSVTDFYPSQIKVRWFRNDQ |
| | peptide underlined | EETAGVVSTPLIRNGDWTFQILVMLEMTPQRGD |
| | | VYTCHVEHPSLQSPITVEWRAQSESAQSKMLSGV |
| | | GGFVLGLIFLGLGLIIRQRSRKGLLH |
| (SEQ ID | DQB1*02: 02 | MSWKKALRIPGGLRAATVTLMLSMLSTPVAEGR |
| NO: 113) | (IMGT/HLA | DSPEDFVYQFKGMCYFTNGTERVRLVSRSIYNRE |
| | Accession No. | EIVRFDSDVGEFRAVTLLGLPAAEYWNSQKDILE |
| | HLA00623) | RKRAAVDRVCRHNYQLELRTTLQRRVEPTVTISP |
| | * Predicted signal | SRTEALNHHNLLVCSVTDFYPAQIKVRWFRNGQ |
| | peptide underlined | EETAGVVSTPLIRNGDWTFQILVMLEMTPQRGD |
| | | VYTCHVEHPSLQSPITVEWRAQSESAQSKMLSGI |
| | | GGFVLGLIFLGLGLIIHHRSQKGLLH |
| (SEQ ID | DQB1*03: 02 | MSWKKALRIPGGLRVATVTLMLAMLSTPVAEGR |
| NO: 114) | (IMGT/HLA | DSPEDFVYQFKGMCYFTNGTERVRLVTRYIYNR |
| | Accession No. | EEYARFDSDVGVYRAVTPLGPPAAEYWNSQKEV |
| | HLA00627) | LERTRAELDTVCRHNYQLELRTTLQRRVEPTVTI |
| | * Predicted signal | SPSRTEALNHHNLLVCSVTDFYPAQIKVRWFRND |
| | peptide underlined | QEETTGVVSTPLIRNGDWTFQILVMLEMTPQRGD |
| | | VYTCHVEHPSLQNPIIVEWRAQSESAQSKMLSGI |
| | | GGFVLGLIFLGLGLIIHHRSQKGLLH |
| (SEQ ID | DQB1*06: 03 | MSWKKALRIPGDLRVATVTLMLAMLSSLLAEGR |
| NO: 115) | (IMGT/HLA | DSPEDFVYQFKGMCYFTNGTERVRLVTRHIYNR |
| | Accession No. | EEYARFDSDVGVYRAVTPQGRPDAEYWNSQKE |
| | HLA00647) | VLEGTRAELDTVCRHNYEVAFRGILQRRVEPTVT |
| | * Predicted signal | ISPSRTEALNHHNLLVCSVTDFYPGQIKVRWFRN |
| | peptide underlined | DQEETAGVVSTPLIRNGDWTFQILVMLEMTPQR |
| | | GDVYTCHVEHPSLQSPITVEWRAQSESAQSKMLS |
| | | GVGGFVLGLIFLGLGLIIRQRSQKGLLH |
| (SEQ ID | DQB1*03: 03 | MSWKKALRIPGGLRVATVTLMLAMLSTPVAEGR |
| NO: 116) | (IMGT/HLA | DSPEDFVYQFKGMCYFTNGTERVRLVTRYIYNR |
| | Accession No. | EEYARFDSDVGVYRAVTPLGPPDAEYWNSQKEV |
| | HLA00629) | LERTRAELDTVCRHNYQLELRTTLQRRVEPTVTI |
| | * Predicted signal | SPSRTEALNHHNLLVCSVTDFYPAQIKVRWFRND |
| | peptide underlined | QEETTGVVSTPLIRNGDWTFQILVMLEMTPQRGD |
| | | VYTCHVEHPSLQNPIIVEWRAQSESAQSKMLSGI |
| | | GGFVLGLIFLGLGLIIHHRSQKGLLH |
| (SEQ ID | DQB1*06: 04 | MSWKKALRIPGDLRVATVTLMLAMLSSLLAEGR |
| NO: 117) | (IMGT/HLA | DSPEDFVYQFKGMCYFTNGTERVRLVTRHIYNR |
| | Accession No. | EEYARFDSDVGVYRAVTPQGRPVAEYWNSQKE |
| | HLA00648) | VLERTRAELDTVCRHNYEVGYRGILQRRVEPTV |
| | * Predicted signal | TISPSRTEALNHHNLLVCSVTDFYPGQIKVQWFR |
| | peptide underlined | NDQEETAGVVSTPLIRNGDWTFQILVMLEMTPQ |
| | | RGDVYTCHVEHPSLQSPITVEWRAQSESAQSKM |
| | | LSGVGGFVLGLIFLGLGLIIRQRSQKGLLH |
| (SEQ ID | DQB1*05: 03 | MSWKKSLRIPGDLRVATVTLMLAILSSSLAEGRD |
| NO: 118) | (IMGT/HLA | SPEDFVYQFKGLCYFTNGTERVRGVTRHIYNREE |
| | Accession No. | YVRFDSDVGVYRAVTPQGRPDAEYWNSQKEVL |
| | HLA00640) | EGARASVDRVCRHNYEVAYRGILQRRVEPTVTIS |
| | * Predicted signal | PSRTEALNHHNLLICSVTDFYPSQIKVRWFRNDQ |
| | peptide underlined | EETAGVVSTPLIRNGDWTFQILVMLEMTPQRGD |
| | | VYTCHVEHPSLQSPITVEWRAQSESAQSKMLSGV |
| | | GGFVLGLIFLGLGLIIRQRSRKGPQGPPPAGLLH |
| (SEQ ID | DQB1*04: 02 | MSWKKALRIPGGLRVATVTLMLAMLSTPVAEGR |
| NO: 119) | (IMGT/HLA | DSPEDFVFQFKGMCYFTNGTERVRGVTRYIYNR |
| | Accession No. | EEYARFDSDVGVYRAVTPLGRLDAEYWNSQKDI |
| | HLA00637) | LEEDRASVDTVCRHNYQLELRTTLQRRVEPTVTI |
| | * Predicted signal | SPSRTEALNHHNLLVCSVTDFYPAQIKVRWFRND |
| | peptide underlined | QEETTGVVSTPLIRNGDWTFQILVMLEMTPQRGD |
| | | VYTCHVEHPSLQNPIIVEWRAQSESAQSKMLSGI |
| | | GGFVLGLIFLGLGLIIHHRSQKGLLH |
| (SEQ ID | DPA1*01: 03 | MRPEDRMFHIRAVILRALSLAFLLSLRGAGAIKA |
| NO: 120) | (IMGT/HLA | DHVSTYAAFVQTHRPTGEFMFEFDEDEMFYVDL |
| | Accession No. | DKKETVWHLEEFGQAFSFEAQGGLANIAILNNNL |
| | HLA00499) | NTLIQRSNHTQATNDPPEVTVFPKEPVELGQPNT |
| | * Predicted signal | LICHIDKFFPPVLNVTWLCNGELVTEGVAESLFLP |
| | peptide underlined | RTDYSFHKFHYLTFVPSAEDFYDCRVEHWGLDQ |
| | | PLLKHWEAQEPIQMPETTETVLCALGLVLGLVGI |
| | | IVGTVLIIKSLRSGHDPRAQGTL |
| (SEQ ID | DPA1*02: 01 | MRPEDRMFHIRAVILRALSLAFLLSLRGAGAIKA |
| NO: 121) | (IMGT/HLA | DHVSTYAAFVQTHRPTGEFMFEFDEDEQFYVDL |
| | Accession No. | DKKETVWHLEEFGRAFSFEAQGGLANIAILNNNL |
| | HLA00504) | NTLIQRSNHTQAANDPPEVTVFPKEPVELGQPNT |
| | * Predicted signal | LICHIDRFFPPVLNVTWLCNGEPVTEGVAESLFLP |
| | peptide underlined | RTDYSFHKFHYLTFVPSAEDVYDCRVEHWGLDQ |
| | | PLLKHWEAQEPIQMPETTETVLCALGLVLGLVGI |
| | | IVGTVLIIKSLRSGHDPRAQGPL |
| (SEQ ID | DPA1*02: 07 | MRPEDRMFHIRAVILRALSLAFLLSLRGAGAIKA |
| NO: 122) | (IMGT/HLA | DHVSTYAMFVQTHRPTGEFMFEFDEDEQFYVDL |
| | Accession No. | DKKETVWHLEEFGRAFSFEAQGGLANIAILNNNL |
| | HLA15619) | NTLIQRSNHTQAANDPPEVTMFPKEPVELGQPNT |
| | * Predicted signal | LICHIDRFFPPVLNVTWLCNGEPVTEGVAESLFLP |
| | peptide underlined | RTDYSFHKFHYLTFVPSAEDVYDCRVEHWGLDQ |
| | | PLLKHWEAQEPIQMPETTETVLCALGLVLGLVGI |
| | | IVGTVLIIKSLRSGHDPRAQGPL |
| (SEQ ID | DPB1*04: 01 | MMVLQVSAAPRTVALTALLMVLLTSVVQGRAT |
| NO: 123) | (IMGT/HLA | PENYLFQGRQECYAFNGTQRFLERYIYNREEFAR |
| | Accession No. | FDSDVGEFRAVTELGRPAAEYWNSQKDILEEKR |
| | HLA00521) | AVPDRMCRHNYELGGPMTLQRRVQPRVNVSPS |
| | * Predicted signal | KKGPLQHHNLLVCHVTDFYPGSIQVRWFLNGQE |
| | peptide underlined | ETAGVVSTNLIRNGDWTFQILVMLEMTPQQGDV |
| | | YTCQVEHTSLDSPVTVEWKAQSDSARSKTLTGA |
| | | GGFVLGLIICGVGIFMHRRSKKVQRGSA |
| (SEQ ID | DPB1*02: 01 | MMVLQVSAAPRTVALTALLMVLLTSVVQGRAT |
| NO: 124) | (IMGT/HLA | PENYLFQGRQECYAFNGTQRFLERYIYNREEFVR |
| | Accession No. | FDSDVGEFRAVTELGRPDEEYWNSQKDILEEERA |
| | HLA00517) | VPDRMCRHNYELGGPMTLQRRVQPRVNVSPSK |
| | * Predicted signal | KGPLQHHNLLVCHVTDFYPGSIQVRWFLNGQEE |
| | peptide underlined | TAGVVSTNLIRNGDWTFQILVMLEMTPQQGDVY |
| | | TCQVEHTSLDSPVTVEWKAQSDSARSKTLTGAG |
| | | GFVLGLIICGVGIFMHRRSKKVQRGSA |
| (SEQ ID | DPB1*04: 02 | MMVLQVSAAPRTVALTALLMVLLTSVVQGRAT |
| NO: 125) | (IMGT/HLA | PENYLFQGRQECYAFNGTQRFLERYIYNREEFVR |
| | Accession No. | FDSDVGEFRAVTELGRPDEEYWNSQKDILEEKR |
| | HLA00522) | AVPDRMCRHNYELGGPMTLQRRVQPRVNVSPS |
| | * Predicted signal | KKGPLQHHNLLVCHVTDFYPGSIQVRWFLNGQE |
| | peptide underlined | ETAGVVSTNLIRNGDWTFQILVMLEMTPQQGDV |
| | | YTCQVEHTSMDSPVTVEWKAQSDSARSKTLTGA |
| | | GGFVLGLIICGVGIFMHRRSKKVQRGSA |
| (SEQ ID | DPB1*03: 01 | MMVLQVSAAPRTVALTALLMVLLTSVVQGRAT |
| NO: 126) | (IMGT/HLA | PENYVYQLRQECYAFNGTQRFLERYIYNREEFVR |
| | Accession No. | FDSDVGEFRAVTELGRPDEDYWNSQKDLLEEKR |
| | HLA00520) | AVPDRVCRHNYELDEAVTLQRRVQPKVNVSPSK |
| | * Predicted signal | KGPLQHHNLLVCHVTDFYPGSIQVRWFLNGQEE |
| | peptide underlined | TAGVVSTNLIRNGDWTFQILVMLEMTPQQGDVY |
| | | ICQVEHTSLDSPVTVEWKAQSDSARSKTLTGAG |
| | | GFVLGLIICGVGIFMHRRSKKVQRGSA |
| (SEQ ID | DPB1*01: 01 | MMVLQVSAAPRTVALTALLMVLLTSVVQGRAT |
| NO: 127) | (IMGT/HLA | PENYVYQGRQECYAFNGTQRFLERYIYNREEYA |
| | Accession No. | RFDSDVGEFRAVTELGRPAAEYWNSQKDILEEK |
| | HLA00514) | RAVPDRVCRHNYELDEAVTLQRRVQPKVNVSPS |
| | * Predicted signal | KKGPLQHHNLLVCHVTDFYPGSIQVRWFLNGQE |
| | peptide underlined | ETAGVVSTNLIRNGDWTFQILVMLEMTPQQGDV |
| | | YICQVEHTSLDSPVTVEWKAQSDSAQSKTLTGA |
| | | GGFVLGLIICGVGIFMHRRSKKVQRGSA |
| (SEQ ID | DPB1*11: 01 | MMVLQVSAAPRTVALTALLMVLLTSVVQGRAT |
| NO: 128) | (IMGT/HLA | PENYVYQLRQECYAFNGTQRFLERYIYNRQEYA |
| | Accession No. | RFDSDVGEFRAVTELGRPAAEYWNSQKDLLEER |
| | HLA00528) | RAVPDRMCRHNYELDEAVTLQRRVQPKVNVSPS |
| | * Predicted signal | KKGPLQHHNLLVCHVTDFYPGSIQVRWFLNGQE |
| | peptide underlined | ETAGVVSTNLIRNGDWTFQILVMLEMTPQQGDV |
| | | YICQVEHTSLDSPVTVEWKAQSDSARSKTLTGA |
| | | GGFVLGLIICGVGIFMHRRSKKVQRGSA |
| (SEQ ID | DPB1*05: 01 | MMVLQVSAAPRTVALTALLMVLLTSVVQGRAT |
| NO: 129) | (IMGT/HLA | PENYLFQGRQECYAFNGTQRFLERYIYNREELVR |
| | Accession No. | FDSDVGEFRAVTELGRPEAEYWNSQKDILEEKR |
| | HLA00523) | AVPDRMCRHNYELDEAVTLQRRVQPKVNVSPSK |
| | * Predicted signal | KGPLQHHNLLVCHVTDFYPGSIQVRWFLNGQEE |
| | peptide underlined | TAGVVSTNLIRNGDWTFQILVMLEMTPQQGDVY |
| | | ICQVEHTSLDSPVTVEWKAQSDSARSKTLTGAG |
| | | GFMLGLIICGVGIFMHRRSKKVQRGSA |
| (SEQ ID | DPB1*10: 01 | MMVLQVSAAPRTVALTALLMVLLTSVVQGRAT |
| NO: 130) | (IMGT/HLA | PENYVHQLRQECYAFNGTQRFLERYIYNREEFVR |
| | Accession No. | FDSDVGEFRAVTELGRPDEEYWNSQKDILEEERA |
| | HLA00527) | VPDRVCRHNYELDEAVTLQRRVQPKVNVSPSKK |
| | * Predicted signal | GPLQHHNLLVCHVTDFYPGSIQVRWFLNGQEET |
| | peptide underlined | AGVVSTNLIRNGDWTFQILVMLEMTPQQGDVYI |
| | | CQVEHTSLDSPVTVEWKAQSDSARSKTLTGAGG |
| | | FVLGLIICGVGIFMHRRSKKVQRGSA |
| (SEQ ID | DPB1*06: 01 | MMVLQVSAAPRTVALTALLMVLLTSVVQGRAT |
| NO: 131) | (IMGT/HLA | PENYVYQLRQECYAFNGTQRFLERYIYNREEFVR |
| | Accession No. | FDSDVGEFRAVTELGRPDEDYWNSQKDLLEEER |
| | HLA00524) | AVPDRMCRHNYELDEAVTLQRRVQPKVNVSPSK |
| | * Predicted signal | KGPLQHHNLLVCHVTDFYPGSIQVRWFLNGQEE |
| | peptide underlined | TAGVVSTNLIRNGDWTFQILVMLEMTPQQGDVY |
| | | ICQVEHTSLDSPVTVEWKAQSDSARSKTLTGAG |
| | | GFVLGLIICGVGIFMHRRSKKVQRGSA |
| (SEQ ID | DPB1*13: 01 | MMVLQVSAAPRTVALTALLMVLLTSVVQGRAT |
| NO: 132) | (IMGT/HLA | PENYVYQLRQECYAFNGTQRFLERYIYNREEYA |
| | Accession No. | RFDSDVGEFRAVTELGRPAAEYWNSQKDILEEER |
| | HLA00530) | AVPDRICRHNYELDEAVTLQRRVQPKVNVSPSK |
| | * Predicted signal | KGPLQHHNLLVCHVTDFYPGSIQVRWFLNGQEE |
| | peptide underlined | TAGVVSTNLIRNGDWTFQILVMLEMTPQQGDVY |
| | | ICQVEHTSLDSPVTVEWKAQSDSARSKTLTGAG |
| | | GFVLGLIICGVGIFMHRRSKKVQRGSA |
| (SEQ ID | DPB1*14: 01 | MMVLQVSAAPRTVALTALLMVLLTSVVQGRAT |
| NO: 133) | (IMGT/HLA | PENYVHQLRQECYAFNGTQRFLERYIYNREEFVR |
| | Accession No. | FDSDVGEFRAVTELGRPDEDYWNSQKDLLEEKR |
| | HLA00531) | AVPDRVCRHNYELDEAVTLQRRVQPKVNVSPSK |
| | * Predicted signal | KGPLQHHNLLVCHVTDFYPGSIQVRWFLNGQEE |
| | peptide underlined | TAGVVSTNLIRNGDWTFQILVMLEMTPQQGDVY |
| | | ICQVEHTSLDSPVTVEWKAQSDSARSKTLTGAG |
| | | GFVLGLIICGVGIFMHRRSKKVQRGSA |
| (SEQ ID | DPB1*17: 01 | MMVLQVSAAPRTVALTALLMVLLTSVVQGRAT |
| NO: 134) | (IMGT/HLA | PENYVHQLRQECYAFNGTQRFLERYIYNREEFVR |
| | Accession No. | FDSDVGEFRAVTELGRPDEDYWNSQKDILEEER |
| | HLA00534) | AVPDRMCRHNYELDEAVTLQRRVQPRVNVSPSK |
| | * Predicted signal | KGPLQHHNLLVCHVTDFYPGSIQVRWFLNGQEE |
| | peptide underlined | TAGVVSTNLIRNGDWTFQILVMLEMTPQQGDVY |
| | | TCQVEHTSLDSPVTVEWKAQSDSARSKTLTGAG |
| | | GFVLGLIICGVGIFMHRRSKKVQRGSA |
| (SEQ ID | HUMAN HLA- | CSHSMRYFYTAVSRPGRGEPRFIAVGYVDDTQF |
| NO: 135) | C*12: 0 | VRFDSDAASPRGEPRAPWVEQEGPEYWDRETQK |
| | | YKRQAQADRVSLRNLRGYYNQSEAGSHTLQRM |
| | | YGCDLGPDGRLLRGYDQSAYDGKDYIALNEDLR |
| | | SWTAADTAAQITQRKWEAAREAEQWRAYLEGT |
| | | CVEWLRRYLENGKETLQRAEHPKTHVTHHPVSD |
| | | HEATLRCWALGFYPAEITLTWQRDGEDQTQDTE |
| | | LVETRPAGDGTFQKWAAVVVPSGEEQRYTCHV |
| | | QHEGLPEPLTLRWEPSSQPTIPIVGIVAGLAVLAV |
| | | LAVLGAVMAVVMCRRKSSGGKGGSCSQAASSN |
| | | SAQGSDESLIACKA |
| (SEQ ID | HUMAN HLA- | GSHSLKYFHTSVSRPGRGEPRFISVGYVDDTQFV |
| NO: 136) | E*01: 032 | RFDNDAASPRMVPRAPWMEQEGSEYWDRETRS |
| | | ARDTAQIFRVNLRTLRGYYNQSEAGSHTLQWMH |
| | | GCELGPDGRFLRGYEQFAYDGKDYLTLNEDLRS |
| | | WTAVDTAAQISEQKSNDASEAEHQRAYLEDTCV |
| | | EWLHKYLEKGKETLLHLEPPKTHVTHHPISDHEA |
| | | TLRCWALGFYPAEITLTWQQDGEGHTQDTELVE |
| | | TRPAGDGTFQKWAAVVVPSGEEQRYTCHVQHE |
| | | GLPEPVTLRWKPASQPTIPIVGIIAGLVLLGSVVS |
| | | GAVVAAVIWRKKSSGGKGGSYSKAEWSDSAQG |
| | | SESHSL |
| (SEQ ID | HUMAN HLA- | GSHSMRYFSAAVSRPGRGEPRFIAMGYVDDTQF |
| NO: 137) | G*01: 01 | VRFDSDSACPRMEPRAPWVEQEGPEYWEEETRN |
| | | TKAHAQTDRMNLQTLRGYYNQSEASSHTLQWM |
| | | IGCDLGSDGRLLRGYEQYAYDGKDYLALNEDLR |
| | | SWTAADTAAQISKRKCEAANVAEQRRAYLEGTC |
| | | VEWLHRYLENGKEMLQRADPPKTHVTHHPVFD |
| | | YEATLRCWALGFYPAEIILTWQRDGEDQTQDVE |
| | | LVETRPAGDGTFQKWAAVVVPSGEEQRYTCHV |
| | | QHEGLPEPLMLRWKQSSLPTIPIMGIVAGLVVLA |
| | | AVVTGAAVAAVLWRKKSSD |

Additional HLA allele amino acid sequences are known in the art and are available, for example at the IMGT/HLA Database (available on the world wide web at ebi.ac.uk/ipd/imgt/hla/; see Robinson et al. (2015) Nucl. Acids Res. 43: D423-31).

In some embodiments, the engineered erythroid cell (e.g., engineered enucleated erythroid cell) or enucleated cell (e.g., modified enucleated cell) has been derived from an erythroid precursor cell that has been genetically modified to delete and/or alter expression of an endogenous antigen-presenting polypeptide (e.g. a HLA class I or HLA class II polypeptide). In some embodiments, the engineered erythroid cell (e.g., engineered enucleated erythroid cell) or enucleated cell (e.g., modified enucleated cell) has been derived from an erythroid precursor cell that has not been genetically modified to delete and/or alter expression of an endogenous antigen-presenting polypeptide (e.g. a HLA class I or HLA class II polypeptide).

### Amino Acid Substitutions in the Loadable Exogenous Antigen-Presenting Polypeptide

In some embodiments, a loadable exogenous antigen-presenting polypeptide described herein comprises one or more amino acid substitutions (as compared to the wild-type protein from which it was derived) which stabilize the loadable exogenous antigen-presenting polypeptide on a cell surface. In some embodiments, the loadable exogenous antigen-presenting polypeptide is stabilized on a cell surface in the absence of a polypeptide bound to the exogenous antigen-presenting polypeptide.

In some embodiments, the amino acid substitution(s) comprise substitution(s) to cysteine residues, which form disulfide bond(s) that stabilize the exogenous antigen-presenting polypeptide (see, e.g., Hein et al. (2014) J. Cell Sci. 127: 2885-97).

In some embodiments, the loadable exogenous antigen-presenting polypeptide is derived from a wild-type HLA class I polypeptide, e.g., a HLA-A, a HLA-B, a HLA-C, a HLA-E, or a HLA-G polypeptide, and the amino acid residues at the following specific positions of the HLA polypeptide are substituted to cysteines in the following pairs: 84 and 139; 51 and 175; 5 and 168; 130 and 157; 135 and 140; 11 and 74; 45 and 63; 33 and 49, where these positions relate to the alpha chain of the mature HLA class I polypeptide, without the N-terminal leader sequence.

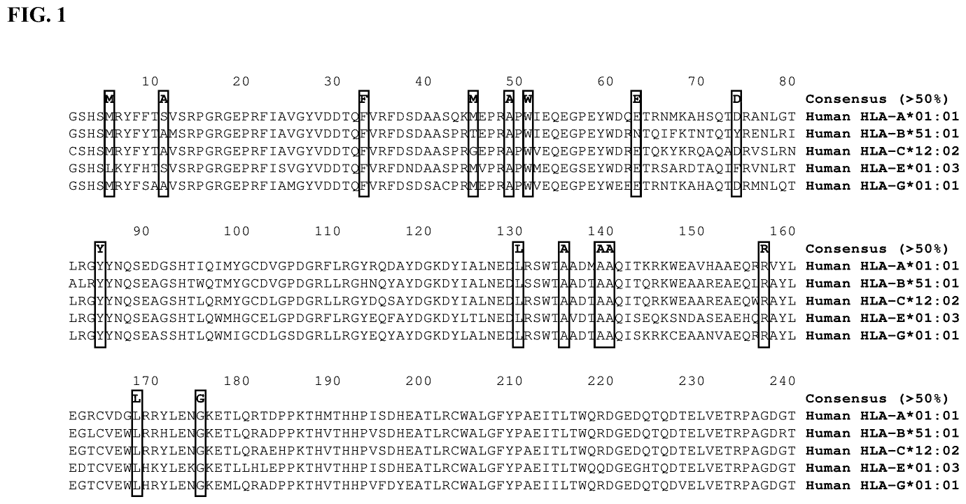

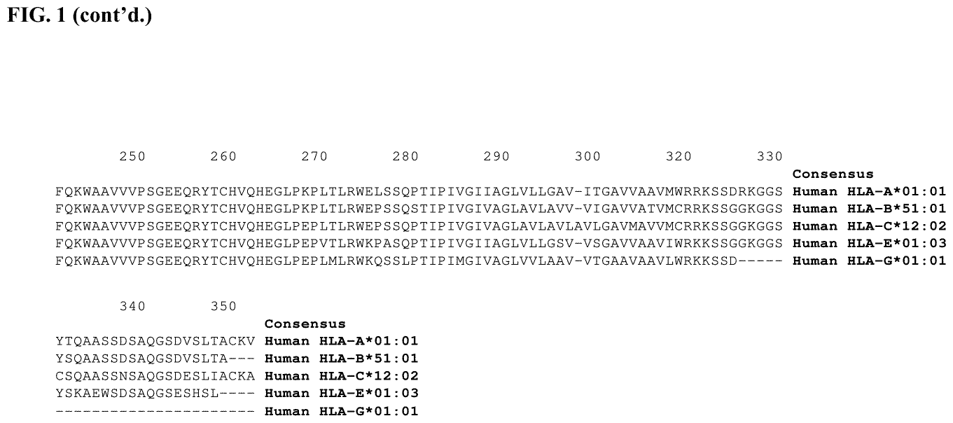

The exemplary pairs of amino acid substitutions are set forth below in Table 5 with respect to exemplary HLA class I (e.g., HLA-A, HLA-B, HLA-C, HLA-G, and HLA-E) alleles. These amino acid substitutions are also set forth in FIG. 1, which depicts amino acid sequence alignments of exemplary wild-type alleles of HLA-A (i.e., HLA-A*01:01), HLA-B (i.e., HLA-B*51:01), HLA-C (i.e., HLA-C*12:02), HLA-E (i.e., HLA-E*01:03), and HLA-G (i.e., HLA-G*01:01).

In some embodiments, the loadable exogenous antigen-presenting polypeptide comprises an amino acid sequence derived from an HLA class I polypeptide comprising amino acid substitutions to cysteine at the amino acid residues 84 and 139 in the alpha chain of the mature HLA class I polypeptide. In other embodiments, the loadable exogenous antigen-presenting polypeptide comprises an HLA class I polypeptide and comprises amino acid substitutions to cysteine at the amino acid residues 51 and 175 in the alpha chain of the mature HLA class I polypeptide. In some embodiments, the loadable exogenous antigen-presenting polypeptide comprises an amino acid sequence derived from an HLA class I polypeptide that does not include amino acid substitutions to cysteine at the amino acid residues 84 and 139 in the alpha chain of the mature HLA class I polypeptide.

In some embodiments, the loadable exogenous antigen-presenting polypeptide, e.g., the HLA polypeptide, comprises at least 1, 2, 3, 4, 5, 6, 7, or 8 of the pairs of amino acid substitutions set forth below in Table 5.

TABLE 5
Exemplary Pairs of HLA Class I Amino Acid Substitutions

| | Amino Acid Substitution Pair |
|---|---|
| Allele | (of the mature HLA polypeptide) |
| HLA-A (e.g., HLA- | Y84C and A139C |
| A*01:01) | W51C and G175C |
| | M5C and L168C |
| | L130C and R157C |
| | A135C and A140C |
| | S11C and D74C |
| | M45C and E63C |
| | F33C and A49C |
| HLA-B (e.g., HLA- | Y84C and A139C |
| B*51:01) | W51C and G175C |
| | M5C and L168C |
| | L130C and R157C |
| | A135C and A140C |
| | A11C and Y74C |
| | T45C and N63C |
| | F33C and A49C |
| HLA-C (e.g., HLA- | Y84C and A139C |
| C*12:02) | W51C and G175C |
| | M5C and L168C |
| | L130C and R157C |
| | A135C and A140C |
| | A11C and D74C |
| | G45C and E63C |
| | F33C and A49C |
| HLA-E (e.g., HLA- | Y84C and A139C |
| E*01:03) | W51C and G175C |
| | L5C and L168C |
| | L130C and R157C |
| | A135C and A140C |
| | S11C and F74C |
| | M45C and E63C |
| | F33C and A49C |
| HLA-G (e.g., HLA- | Y84C and A139C |
| G*01:01) | W51C and G175C |
| | M5C and L168C |
| | L130C and R157C |
| | A135C and A140C |
| | A11C and D74C |
| | M45C and E63C |
| | F33C and A49C |

In other embodiments, the loadable exogenous antigen-presenting polypeptide comprises an HLA class I polypeptide and comprises a mutation to an alanine at amino acid residue 84 in the alpha chain of the mature HLA class I polypeptide. In some embodiments, the loadable exogenous antigen-presenting comprises an amino acid sequence derived from an HLA class I polypeptide that does not include a mutation to an alanine or to a cysteine at amino acid residue 84 in the alpha chain of the mature HLA class I polypeptide.

In some embodiments, the loadable exogenous antigen-presenting polypeptide comprises an HLA class I polypeptide, and comprises a mutation to cysteine at the amino acid residue 84 in the alpha chain of the mature HLA class I polypeptide, and further comprises a cysteine at a second amino acid residue of the linker disposed between the β2M polypeptide and the displaceable exogenous polypeptide.

In some embodiments, the loadable exogenous antigen-presenting polypeptide comprises an HLA-A*02-01 allele, and comprises a Q115E mutation in the alpha chain of the mature HLA class I polypeptide.

The sequences of the wild-type proteins depicted in FIG. 1 are set forth above in Table 4.

Amino acid substitutions in other HLA alleles, but that correspond to the amino acid positions recited herein are also encompassed by the present disclosure.

In some embodiments, the loadable exogenous antigen-presenting polypeptide comprises one or more amino acid substitutions which increase the affinity of the loadable exogenous antigen-presenting polypeptide, e.g., an HLA class I polypeptide, for CD8 TCRs. In some embodiments, a loadable exogenous antigen-presenting polypeptide described herein comprises an amino acid sequence derived from a HLA class I polypeptide, wherein the HLA class I polypeptide comprises at least one amino acid substitution (as compared to the wild-type protein from which it was derived) which increases the affinity of the loadable MHC protein to CD8. In some embodiments, the HLA class I polypeptide is of an HLA-A allele. In some embodiments, the HLA class I polypeptide is of an HLA-A*02-01 allele. In some embodiments, the amino acid substitution is Q115E (e.g., MHCI HLA-A*02-01 Q115E; see e.g., Wooldridge et al. (2005) J. Biol. Chem. 280: 27491-501, the contents of which are hereby incorporated herein by reference).

In some embodiments, provided herein are nucleic acid molecules encoding the loadable exogenous antigen-presenting polypeptides described herein, wherein the nucleic acid molecule encodes one or more of the mutant loadable exogenous antigen-presenting polypeptides described herein.

In some embodiments, a loadable exogenous antigen-presenting polypeptide described herein, e.g., comprising an HLA class II polypeptide, comprises one or more amino acid substitutions (as compared to the wild-type protein from which it was derived) which stabilize the loadable exogenous antigen-presenting polypeptide on the cell surface. In some embodiments, the loadable exogenous antigen-presenting polypeptide is stabilized on the cell surface in the absence of a polypeptide bound to the exogenous antigen-presenting polypeptide. Methods of identifying an HLA class II polypeptide comprising one or more amino acid substitutions of interest are known in the art and include, e.g., directed evolution by yeast display (see e.g., Esteban et al. (2004) J. Mol. Biol. 340: 81-95; U.S. Pat. No. 7,442,773; Starwalt et al. (2003) Protein Eng. 16(2): 147-56; U.S. Patent Application No. 2002/0165149; Brophy et al. (2003) J. Immunol. Methods 272: 235-46; U.S. Patent Application Publication No. 2003/0036506; each of which are incorporated in their entirety herein by reference).

### Exogenous Displaceable Polypeptides

In some embodiments, the loadable exogenous antigen-presented polypeptide described herein is bound to an exogenous displaceable polypeptide. In some embodiments, the loadable exogenous antigen-presenting polypeptide has a lower binding affinity (K_{D}) for the exogenous displaceable polypeptide relative to its binding affinity (K_{D}) for an exogenous antigenic polypeptide. In some embodiments, the exogenous displaceable polypeptide binds to the loadable exogenous antigen-presenting polypeptide with a K_{D} of from about 1 nM to about 100 μM, from about 10 nm to about 100 μm, from about 50 nm to about 100 μm, from about 100 nm to about 100 μm, from about 150 nm to about 100 μm, from about 200 nm to about 100 μm, from about 250 nm to about 100 μm, from about 300 nm to about 100 μm, from about 400 nm to about 100 μm, from about 500 nm to about 100 μm, from about 600 nm to about 100 μm, from about 700 nm to about 100 μm, from about 800 nm to about 100 μm, from about 900 nm to about 100 μm, from about 1 μM to about 100 μm, from about 5 uM to about 100 μm, from about 10 μm to about 100 μm, from about 20 μm to about 100 μm, from about 30 μm to about 100 μm, from about 40 μm to about 100 μm, from about 50 μm to about 100 μm, from about 60 μm to about 100 μm, from about 70 μm to about 100 μm, from about 80 μm to about 100 μm, or from about 90 μm to about 100 μm.

In some embodiments, the exogenous displaceable polypeptide is from about 8 amino acids in length to about 30 amino acids in length, for example 8, 9, 10, 11, 12, 13, 14, 15, 16, 17, 18, 19, 20, 21, 22, 23, 24, 25, 26, 27, 28, 29, or 30 amino acids in length. In further embodiments, the exogenous displaceable polypeptide comprises a cleavable site (e.g., an enzymatic cleavage site (e.g., as described herein)).

In some embodiments, the exogenous displaceable polypeptide is covalently attached to the loadable exogenous antigen-presenting polypeptide. In some embodiments, the exogenous displaceable polypeptide is non-covalently attached to the loadable exogenous antigen-presenting polypeptide. Further details of covalent attachments are described in the methods below.

In certain embodiments, the exogenous displaceable polypeptide comprises or consists of an antigenic polypeptide selected from Table 6, or a fragment or variant thereof

TABLE 6
Exemplary Exogenous Displaceable Polypeptides

| | | Displaceable | | | |
|---|---|---|---|---|---|
| | SEQ | Polypeptide | | | |
| HLAI | ID | Amino Acid | | Polypeptide/HLAI | |
| allele | NO: | Sequence | Description | Kd (nM) | Reference |
| HLA- | 138 | ILKEPVHGV | Human | 2.5 | Saini et al. |
| A*02:01 | 139 | IAKEPVHGV | Immunodeficiency | 7288* | (2015) |
| | 140 | ILKEPVHGA | Virus (HIV)- | 1095* | Proc. |
| | 141 | IAKEPVHGA | derived Reverse | 1911* | Nat'l. |
| | | | Transcriptase | | Acad. Sci. |
| | | | (RT) amino acid | | USA 112: |
| | | | residues 476-484 | | 202-7. |
| | 142 | GLKEPQIQV | | 308* | |
| | 143 | NLVPMVATA | Cytomegalovirus | 171* | |
| | | | (CMV)-derived | | |
| | | | pp65 | | |
| HLA- | 144 | IRAAPPPLA | | 1615.4* | |
| B*27:05 | | | | | |

*Predicted binding affinity determined using NetMHCpan 4.0 (available on the world wide web at cbs.dtu.dk/services/NetMHCpan/; see also Jurtz et al. (2017) J. Immunol. 199: 3360-8)

Methods of determining the affinity of a polypeptide (e.g., an exogenous displaceable polypeptide) to a loadable exogenous antigen-presenting polypeptide, e.g., the HLA molecule are known in the art and include, for example, surface plasmon resonance (SPR).

Suitable exogenous displaceable polypeptides, i.e., polypeptides that can specifically bind to a loadable exogenous antigen-presenting polypeptide and which can be displaced by an exogenous antigenic polypeptide of interest, can be identified using methods known in the art. In some embodiments, the ability of a polypeptide to displace a displaceable polypeptide from a loadable exogenous antigen-presenting polypeptide can be detected in vitro using recombinant proteins as measured using thermal denaturation measured by tryptophan fluorescence (TDTF) as described in Saini et al. (2015) Proc. Nat'l. Acad. Sci. USA 112: 202-7, the contents of which are incorporated by reference herein. In addition, the exchange of an exogenous displaceable peptide for a peptide of interest can be measured for example, by fluorescent anisotropy using fluorescently-labeled peptides (e.g., labelled with FITC or TAMRA).

### Exogenous Antigenic Polypeptides

In some embodiments, the engineered enucleated erythroid cells or enucleated cells described herein comprise a wild-type or loadable exogenous antigen-presenting polypeptide bound to an exogenous antigenic polypeptide. In some embodiments, presentation of the exogenous antigenic polypeptide(s) provided herein are capable of activating one or more antigen-specific T cell populations (e.g., in vitro or in vivo). In some embodiments, the exogenous antigenic polypeptide(s) described herein are capable of inducing an immune response to inhibit a cancer (e.g., reduces or alleviates a cause or symptom of a cancer, or improves a value for a parameter associated with the cancer). In some embodiments, the exogenous antigenic polypeptide(s) described herein are capable of inducing an immune response, inhibit an infectious disease (e.g., reduces or alleviates a cause or symptom of an infectious disease, or improves a value for a parameter associated with the infectious disease.) In some embodiments, the exogenous antigenic polypeptide(s) described herein are capable of inducing an immune response to inhibit an autoimmune disease (e.g., reduces or alleviates a cause or symptom of an autoimmune disease, or improves a value for a parameter associated with the autoimmune disease).

In the present disclosure, exogenous antigenic polypeptide(s) can be chosen based on the specific needs of a subject, and can be bound to a pre-selected wild-type or loadable exogenous antigen-presenting polypeptide, thus allowing for customizable treatment of the subject with a particular exogenous antigenic polypeptide. In some embodiments, multiple different (e.g., two, three, four, five or more) exogenous antigenic polypeptides can be chosen based on the specific needs of a subject, and can be bound to wild-type or loadable exogenous antigen-presenting polypeptides on the same engineered erythroid cell or enucleated cell or on different cells within a population of engineered erythroid cells or enucleated cells, thus allowing for customizable treatment of the subject with multiple particular exogenous antigenic polypeptides.

The exogenous antigenic polypeptide can include any antigenic polypeptide capable of inducing an immune response. In some embodiments, the exogenous antigenic polypeptide comprises or consists of an antigenic polypeptide selected from Table 7, or a fragment or variant thereof, or an antibody molecule thereto. Table 7 also provides non-limiting examples of HLA class I polypeptide alleles that may be incorporated into a wild-type or loadable exogenous antigen-binding polypeptide described herein, for binding to the specified antigenic polypeptide.

TABLE 7
Exogenous Antigenic Polypeptides

| | SEQ | | |
|---|---|---|---|
| | ID | Antigenic | HLA Class I |
| Description | NO: | Polypeptide | Alleles |
| EBNA3A₃₇₉₋₃₈₇ | 145 | RPPIFIRRL | HLA-B:07:01 |
| EBNA3A₆₉₃₋₆₁₁ | 146 | RLRAEAQVK | HLA-A:03 |
| EBNA3B₄₁₆₋₄₂₄ | 147 | IVTDFSVIK | HLA-A:11:01 |
| EBNA 3C ₁₆₃₋₁₇₁ | 148 | EGGVGWRHW | HLA-B:44 |
| EBNA 3C₈₈₁₋₈₉₁ | 149 | QPRAPIRPI | HLA-B:07:02 |
| EBNA 3A₃₂₅₋₃₃₃ | 150 | FRLGRAYGL | HLA-B:08:01 |
| EBNA 3A₄₅₈₋₄₆₆ | 151 | YPLHEQHGM | HLA-B:35 |
| LMP-2₄₂₆₋₄₃₄ | 152 | CLGGLLTMV | HLA-A:02:01 |
| BMLF-1: | 153 | GLCTLVAML | HLA-A:02:01 |
| BZLF-1₁₉₀₋₁₉₇ | 154 | RAKFKQLL | HLA-B:08:01 |
| CMV | 155 | NLVPMVATV | HLA-A:02:01 |
| | 156 | FIAGNSAYEYV | HLA-A:02:01 |
| | 157 | SDEEEAIVAYTL | HLA-B:18:05 |
| Gp100₂₀₉₋₂₁₇ | 158 | ITDQVPFSV | HLA-A:02:01 |
| Tyrosinase₃₆₉₋₃₇₇ | 159 | YMDGTMSQV | HLA-A:02:01 |
| Melan-A₂₆₋₃₅ | 160 | EAAGIGILTV | HLA-A:02:01 |
| MAGEA3 ₁₁₄₋₁₂₂ | 161 | AELVHFLLL | HLA-B:40:01 |
| MAGEA3 ₂₇₁₋₂₇₉ | 162 | FLWGPRALV | HLA-A:02:01 |
| MAGEA10₂₅₄₋₂₆₂ | 163 | GLYDGMEHL | HLA-A:02:01 |
| MAGEC2₃₃₆₋₃₄₄ | 164 | ALKDVEERV | HLA-A:02:01 |
| MAGEC2₄₂₋₅₀ | 165 | ASSTLYLVF | HLA-B:57:02 |
| MAGEC2₁₉₁₋₂₀₀ | 166 | LLGLALIEV | HLA-A:02:01 |
| NYESO-1₁₅₇₋₁₆₅ | 167 | SLLMWITQC | HLA-A:02:01 |
| HPV16E7₁₁₋₂₀ | 168 | YMLDLQPETT | HLA-A:02:01 |
| HPV16E7₁₁₋₁₀ | 41 | YMLDLQPET | HLA-A:02:01 |
| HPV16E6₂₀₋₃₈ | 43 | TIHDIILECV | HLA-A:02:01 |
| ME-1 | 169 | FLDEFMEGV | HLA-A:02:01 |
| FNDC3B | 170 | VVMSWAPPV | HLA-A:02:01 |
| PRDX5 | 171 | LLLDDLLVSI | HLA-A:02:01 |
| GAS7 | 172 | SLADEAEVYL | HLA-A:02:01 |
| KIAA0223 | 173 | VLHDDLLEA | HLA-A:02:01 |
| GAPDH | 174 | GIVEGLITTV | HLA-A:02:01 |
| HSP70 | 175 | SLFEGIDIVT | HLA-A:02:01 |
| ACTININ | 176 | FIASNGVKLV | HLA-A:02:01 |
| HAUS3 | 177 | ILNAMIAKI | HLA-A:02:01 |
| CSNK1A1 | 178 | GLFGDIYLAI | HLA-A:02:01 |
| CLPP | 179 | ILDKVLVHL | HLA-A:02:01 |
| CDK4 | 180 | ACDPHSGHFV | HLA-A:02:01 |
| AHNAK | 181 | FMPDFDLHL | HLA-A:02:01 |
| SRPX | 182 | TLWCSPIKV | HLA-A:02:01 |
| COL18A1 | 183 | VLLGVKLFGV | HLA-A:02:01 |
| ERBB2 | 184 | ALIHHNTYL | HLA-A:02:01 |
| TEAD1 | 185 | VLENFTIFLV | HLA-A:02:01 |
| TEAD1 | 186 | SVLENFTIFL | HLA-A:02:01 |
| NSDHL | 187 | ILTGLNYEV | HLA-A:02:01 |
| GANAB | 188 | ALYGFVPVL | HLA-A:02:01 |
| CDC37L1 | 189 | FLSDHLYLV | HLA-A:02:01 |
| FLNA | 190 | HIAKSLFEV | HLA-A:02:01 |
| SPOP | 191 | FLLDEAIGL | HLA-A:02:01 |
| ACPP | 192 | VLAKKLKFV | HLA-A:02:01 |
| DCAKD | 193 | LLHTELERFL | HLA-A:02:01 |
| CIT | 194 | TLLSQVNKV | HLA-A:02:01 |

In other embodiments, the exogenous antigenic polypeptide comprises or consist of an antigenic polypeptide selected from Table 8, or a fragment or variant thereof, or an antibody molecule thereto. Table 8 provides non-limiting examples of HLA polypeptide alleles that may be incorporated into a wild-type or loadable exogenous antigen-presenting polypeptide described herein, for binding to the specified antigenic polypeptide.

TABLE 8
Exogenous Antigenic Polypeptides

| | | | HLA | | | |
|---|---|---|---|---|---|---|
| | | | Fre- | SEQ | | |
| | | HLA | quency | ID | | |
| Gene/Protein | Tumor | Allele | (%) | NO: | Peptide | Position |
| ABL-BCR alb-b3 | | A2 | | 195 | FVEHDDESPGL | |
| (b2a2) Mutation | | | | | | |
| ABL-BCR alb-b4 | | A3 | | 195 | FVEHDDESPGL | |
| (b3a2) Mutation | | | | | | |
| PPP1R3B | melanoma | A1 | 26 | 196 | YTDFHCQYV | 172-180 |
| alpha-actinin-4 | lung carcinoma | A2 | 44 | 176 | FIASNGVKLV | 118-127 |
| ARTC1 | melanoma | DR1 | 18 | 197 | YSVYFNLPADTIYTNH | |
| CASP-8 | head and neck | B35 | 20 | 198 | FPSDSWCYF | 476-484 |
| | squamous cell | | | | | |
| | carcinoma | | | | | |
| beta-catenin | melanoma | A24 | 20 | 199 | SYLDSGIHF | 29-37 |
| Cdc27 | melanoma | DR4 | 24 | 200 | FSWAMDLDPKGAE | 760-771 |
| CDK4 | melanoma | A2 | 44 | 180 | ACDPHSGHFV | 23-32 |
| CDK12 | melanoma | A11 | 13 | 201 | CILGKLFTK | 924-932 |
| CDKN2A | melanoma | A11 | 13 | 202 | AVCPWTWLRG | 125-133 |
| | | | | | | (p14ARF- |
| | | | | | | ORF3) |
| CLPP | melanoma | A2 | 44 | 179 | ILDKVLVHL | 240-248 |
| COA-1 | colorectal | DR4 | 24 | 203 | TLYQDDTLTLQAAGE | 447-460 |
| | carcinoma | DR13 | 19 | 203 | TLYQDDTLTLQAAGE | 447-460 |
| CSNK1A1 | melanoma | A2 | 44 | 204 | GLFGDIYLA | 26-34 |
| EFTUD2 | melanoma | A3 | 22 | 205 | KILDAVVAQK | 668-677 |
| Elongation factor 2 | lung squamous CC | A68 | 8 | 206 | ETVSEQSNV | 581-589 |
| FN1 | melanoma | DR2 | 25 | 207 | MIFEKHGFRRTTPP | 2050-2063 |
| GAS7 | melanoma | A2 | 44 | 172 | SLADEAEVYL | 141-150 |
| GPNMB | melanoma | A3 | 22 | 208 | TLDWLLQTPK | 179-188 |
| HAUS3 | melanoma | A2 | 44 | 209 | ILNAMIAKIJ | 154-162 |
| HSDL1 | ovarian cancer | Cw14 | 4 | 210 | CYMEAVAL | 20-27 |
| LDLR- | melanoma | DR1 | 18 | 211 | WRRAPAPGA | 315-323 |
| fucosyltransferaseAS | | | | 212 | PVTWRRAPA | 312-320 |
| fusion protein | | | | | | |
| HLA-A2d | renal cell carcinoma | | | | | |
| HLA-A11d | melanoma | | | | | |
| HLA-A0201-R170I | | A2 | | 213 | CVEWLRIYLENG | |
| hsp70-2 | renal cell carcinoma | A2 | 44 | 214 | SLFEGIDIYT | 286-295 |
| | bladder tumor | B44 | 21 | 215 | AEPINIQTW | 262-270 |
| IgGH | | A2 | | 216 | LMISRTPEV | |
| MART2 | melanoma | A1 | 26 | 217 | FLEGNEVGKTY | 446-455 |
| MATN | melanoma | A11 | 13 | 218 | KTLTSVFQK | 226-234 |
| k-ras | non-small cell lung | A2 | 44 | 169 | FLDEFMEGV | 224-232 |
| | carcinoma | A2 | | 219 | KLVVVGAVGV | |
| | | A2 | | 220 | LVVVGAVGV | |
| | | B35 | | 221 | VVVGAVGVG | |
| MUM-1f | melanoma | B44 | 21 | 222 | EEKLIVVLF | 30-38 |
| MUM-2 | melanoma | B44 | 21 | 223 | SELFRSGLDSY | 123-133 |
| | | Cw6 | 18 | 224 | FRSGLDSYV | 126-134 |
| MUM-3 | melanoma | A68 | 8 | 225 | EAFIQPITR | 322-330 |
| neo-PAP | melanoma | DR7 | 25 | 226 | RVIKNSIRLTLE | 724-734 |
| NFYC | lung squamous cell | B52 | 5 | 227 | QQITKTEV | 275-282 |
| | carcinoma | | | | | |
| OS-9 | melanoma | B44 | 21 | 228 | KELEGILLL | 438-446 |
| PTPRK | melanoma | DR10 | 3 | 229 | PYYFAAELPPRNLPEP | 667-682 |
| N-ras | melanoma | A1 | 26 | 230 | ILDTAGREEY | 55-64 |
| BRAF600 | melanoma | B7 | 17 | 231 | RPHVPESAF | 329-337 |
| SIRT2 | melanoma | A3 | 22 | 232 | KIFSEVTLK | 192-200 |
| SNRPD1 | melanoma | B38 | 5 | 233 | SHETVIIEL | 11-19 |
| Triosephosphate | melanoma | DR1 | 18 | 234 | GELIGILNAAKVPAD | 23-37 |
| isomerase | | | | | | |
| Myosin class I | melanoma | A3 | 22 | 235 | KINKNPKYK | 911-919 |
| BCR-ABL fusion | | A2 | | 236 | KLSEQESLL | |
| | | A2 | | 237 | MLTNSCVKL | |
| | | A2 | | 238 | FMVELVEGA | |
| | | A2 | | 239 | GVRGRVEEI | |
| BCR-ABL fusion | chronic myeloid | A2 | 44 | 240 | SSKALQRPV | 926-934 |
| protein (b3a2) | leukemia | A3 | | 241 | ATGFKQSSK | |
| | | A3 | | 242 | HSATGFKQSSK | |
| | | A3 | | 243 | KQSSKALQR | |
| | | A11 | | 242 | HSATGFKQSSK | |
| | | B8 | 14 | 244 | GFKQSSKAL | 922-930 |
| | | DR4 | 24 | 245 | ATGFKQSSKALQRPVAS | 920-936 |
| | | DR9 | 3 | 245 | ATGFKQSSKALQRPVAS | 920-936 |
| B-RAF | melanoma | A2 | | 246 | LATEKSRWS | |
| | | A2 | | 247 | LATEKSRWSG | |
| | | A33 | | 248 | FGLATEKSR | |
| | | B27 | | 249 | GDFGLATEK | |
| | | DR4 | 24 | 250 | EDLTVKIGDFGLATEKSRWSGSH | 586-614 |
| | | | | | QFEQLS | |
| CASP-5 | colorectal, gastric, and | A2 | 44 | 251 | FLIIWQNTM | 67-75 |
| | endometrial carcinoma | | | | | |
| dek-can fusion | myeloid leukemia | DR53 | 49 | 252 | TMKQICKKEIRRLHQY | 342-357 |
| protein | | | | | | |
| ETV6-AML1 fusion | acute lymphoblastic | A2 | 44 | 253 | RIAECILGMI | 334-342 |
| protein | leukemia | DP5 | 3 | 254 | IGRIAECILGMNPSR | 332-346 |
| | | DP17 | 1 | 254 | IGRIAECILGMNPSR | 332-346 |
| FLT3-ITD | acute myelogenous | A1 | 26 | 255 | YVDFREYEYY | 591-600 |
| | leukemia | | | | | |
| FNDC3B | chronic lymphocytic | A2 | 44 | 170 | VVMSWAPPV | 292-300 |
| | leukemia | | | | | |
| OGT | colorectal carcinoma | A2 | 44 | 256 | SLYKFSPFPLG | 28-37 |
| p53 | head and neck | A2 | 44 | 257 | VVPCEPPEV | 217-225 |
| | squamous cell | | | | | |
| | carcinoma | | | | | |
| pml-RARalpha | promyelocytic | DR11 | 25 | 258 | NSNHVASGAGEAAIETQSSSSEEI | |
| fusion protein | leukemia | | | | V | |
| PAX-FKHR fusion | | B7 | | 259 | SPANSIRHNL | |
| | | B7 | | 260 | SPQNSIRHNL | |
| PRDX5 | melanoma | A2 | 44 | 171 | LLLDDLLVSI | 163-172 |
| | | A2 | | 261 | AMAPIKVRL | |
| K-ras | pancreatic | B35 | 20 | 221 | VVVGAVGVG | 4-15 |
| | adenocarcinoma | | | | | |
| SYT-SSX1 or -SSX2 | sarcoma | A24 | | 262 | GYDQIMPKK | |
| fusion protein | | A24 | | 263 | GYDQIMPKI | |
| | | B7 | 17 | 264 | QRPYGYDQIM | 402-410 |
| | | | | | | (SYT) |
| KIAAO205 | mutation | B44 | 21 | 215 | AEPINIQTW | |
| ME1 | mutation | A2 | 44 | 169 | FLDEFMEGV | |
| EGFRvIII | Mutation | A2 | 44 | 265 | LEEKKGNYV | |
| | | A2 | 44 | 266 | RLSSCVPVAG | 131-139 |
| TGF-betaRII | colorectal carcinoma | A2 | 44 | 266 | RLSSCVPVAG | 111-119 |
| | | | | | | (p16INK4a- |
| | | | | | | ORF3) |
| | | | | | | 111-112 |
| | | | | | | (SSX2) |
| BAGE-1 | | Cw16 | 7 | 267 | AARAVFLAL | 2-10 |
| D393-CD20n | | DR4 | 24 | 268 | KPLFRRMSSLELVIA | 28-42 |
| | | A2 | 44 | 269 | FLDRFLSCM | 227-235 |
| Cyclin-A1 | | A2 | 44 | 270 | SLIAAAAFCLA | 341-351 |
| GAGE-1,2,8 | | Cw6 | 18 | 271 | YRPRPRRY | 9-16 |
| GAGE-3,4,5,6,7 | | A29 | 6 | 272 | YYWPRPRRY | 10-18 |
| GnTVf | | A2 | 44 | 273 | VLPDVFIRC(V) | intron |
| GPC3 | | A2 | | 274 | FLAELAYDL | |
| HERV-E | | A11 | | 275 | ATFLGSLTWK | |
| HERV-K-MEL | | A2 | 44 | 276 | MLAVISCAV | 1-9 |
| KK-LC-1 | | B15 | 13 | 277 | RQKRILVNL | 76-84 |
| | | A24 | 20 | 278 | NYNNFYRFL | 196-204 |
| KM-HN-1 | | A24 | 20 | 279 | EYSKECLKEF | 499-508 |
| | | A24 | 20 | 280 | EYLSLSDKI | 770-778 |
| LAGE-1 | | A2 | 44 | 281 | MLMAQEALAFL | ORF2 |
| | | | | | | (1-11) |
| | | A2 | 44 | 167 | SLLMWITQC | 157-165 |
| | | A31 | 5 | 282 | LAAQERRVPR | ORF2 |
| | | | | | | (18-27) |
| | | A68 | 8 | 283 | ELVRRILSR | 103-111 |
| | | B7 | 17 | 284 | APRGVRMAV | ORF2 |
| | | | | | | (46-54) |
| | | DP4 | 75 | 285 | SLLMWITQCFLPVF | 157-170 |
| | | DR3 | 21 | 286 | QGAMLAAQERRVPRAAEVPR | ORF2 |
| | | | | | | (14-33) |
| | | DR4 | 24 | 287 | AADHRQLQLSISSCLQQL | 139-156 |
| | | DR11 | 25 | 288 | CLSRRPWKRSWSAGSCPGMPHL | ORF2 |
| | | | | | | (81-102) |
| | | DR12 | 5 | 288 | CLSRRPWKRSWSAGSCPGMPHL | ORF2 |
| | | | | | | (81-102) |
| | | DR13 | 19 | 289 | ILSRDAAPLPRPG | 108-120 |
| | | DR15 | 20 | 290 | AGATGGRGPRGAGA | 37-50 |
| LY6K | | A24 | 20 | 291 | RYCNLEGPPI | 119-128 |
| | | DP5 | 3 | 292 | KWTEPYCVIAAVKIFPRFFMVAK | 61-84 |
| | | | | | Q | |
| | | DR15 | 20 | 293 | KCCKIRYCNLEGPPINSSVF | 114-133 |
| MAGE-A1 | | A1 | 26 | 294 | EADPTGHSY | 161-169 |
| | | A*02:01 | | 295 | YLEYRQVPV | |
| | | | | 296 | YLEYRQVPD | |
| | | A2 | 44 | 297 | KVLEYVIKV | 278-286 |
| | | A3 | 22 | 298 | SLFRAVITK | 96-104 |
| | | A24 | | 299 | NYKHCFPEI | |
| | | A68 | 8 | 300 | EVYDGREHSA | 222-231 |
| | | B7 | 17 | 301 | RVRFFFPSL | 289-298 |
| | | B35 | 20 | 294 | EADPTGHSY | 161-169 |
| | | B37 | 3 | 302 | REPVTKAEML | 120-129 |
| | | B44 | 21 | 303 | KEADPTGHSY | 160-169 |
| | | B53 | 2 | 304 | DPARYEFLW | 258-266 |
| | | B57 | 8 | 305 | ITKKVADLVGF | 102-112 |
| | | Cw2 | 10 | 306 | SAFPTTINF | 62-70 |
| | | Cw3 | 17 | 307 | SAYGEPRKL | 230-238 |
| | | Cw7 | 41 | 301 | RVRFFFPSL | 289-298 |
| | | Cw16 | 7 | 307 | SAYGEPRKL | 230-238 |
| | | DP4 | 75 | 308 | TSCILESLFRAVITK | 90-104 |
| | | DP4 | 75 | 309 | PRALAETSYVKVLEY | 268-282 |
| | | DR13 | 19 | 310 | FLLLKYRAREPVTKAE | 112-127 |
| | | DR15 | 20 | 311 | EYVIKVSARVRF | 281-292 |

MAGE-A2

| | | | | | | |
|---|---|---|---|---|---|---|
| | | A2 | 44 | 312 | YLQLVFGIEV | 157-166 |
| | | A2 | | 295 | YLEYRQVPV | |
| | | A2 | | 313 | LVHFLLLKY | |
| | | A2 | | 314 | KMVELVHFL | |
| | | A2 | | 315 | LVQENYLEY | |
| | | A24 | 20 | 316 | EYLQLVFGI | 156-164 |
| | | B37 | 3 | 302 | REPVTKAEML | 127-136 |
| | | Cw7 | 41 | 317 | EGDCAPEEK | 212-220 |
| | | DR13 | 19 | 318 | LLKYRAREPVTKAE | 121-134 |
| MAGE-A3 | | A1 | 26 | 319 | EVDPIGHLY | 168-176 |
| | | A2 | 44 | 320 | FLWGPRALVD | 271-279 |
| | | A2 | 44 | 321 | KVAELVHFL | 112-120 |
| | | A2 | | 295 | YLEYRQVPV | |
| | | A2 | | 322 | LVFGIELMEV | |
| | | A24 | | 323 | IMPKAGLLI | |
| | | A24 | 20 | 324 | TFPDLESEF | 97-105 |
| | | A24 | 20 | 325 | VAELVHFLL | 113-121 |
| | | B18 | 6 | 326 | MEVDPIGHLY | 167-176 |
| | | | | 327 | YLEYRQVPG | |
| | | B35 | 20 | 319 | EVDPIGHLY | 168-176 |
| | | B37 | 3 | 302 | REPVTKAEML | 127-136 |
| | | B40 | 6 | 328 | AELVHFLLLI | 114-122 |
| | | B44 | 21 | 326 | MEVDPIGHLY | 167-176 |
| | | B52 | 5 | 329 | WQYFFPVIF | 143-151 |
| | | Cw7 | 41 | 317 | EGDCAPEEK | 212-220 |
| | | DP4 | 75 | 330 | KKLLTQHFVQENYLEY | 243-258 |
| | | DP4 | 75 | 331 | RKVAELVHFLLLKYR | 111-125 |
| | | DQ6 | 63 | 330 | KKLLTQHFVQENYLEY | 243-258 |
| | | DR1 | 18 | 332 | ACYEFLWGPRALVETS | 267-282 |
| | | DR4 | 24 | 331 | RKVAELVHFLLLKYR | 111-125 |
| | | DR4 | 24 | 333 | VIFSKASSSLQL | 149-160 |
| | | DR7 | 25 | 333 | VIFSKASSSLQL | 149-160 |
| | | DR7 | 25 | 334 | VFGIELMEVDPIGHL | 161-175 |
| | | DR11 | 25 | 335 | GDNQIMPKAGLLIIV | 191-205 |
| | | DR11 | 25 | 336 | TSYVKVLHHMVKISG | 281-295 |
| | | DR13 | 19 | 337 | RKVAELVHFLLLKYRA | 111-126 |
| | | DR13 | 19 | 310 | FLLLKYRAREPVTKAE | 119-134 |
| MAGE-A4 | | A1 | 26 | 338 | EVDPASNTYJ | 169-177 |
| | | A2 | | 295 | YLEYRQVPV | |
| | | A2 | 44 | 339 | GVYDGREHTV | 230-239 |
| | | A24 | 20 | 340 | NYKRCFPVI | 143-151 |
| | | B37 | 3 | 341 | SESLKMIF | 156-163 |
| MAGE-A5 | | | | | | |
| MAGE-A6 | | A34 | 1 | 342 | MVKISGGPR | 290-298 |
| | | B35 | 20 | 343 | EVDPIGHVY | 168-176 |
| | | B37 | 3 | 302 | REPVTKAEML | 127-136 |
| | | Cw7 | 41 | 317 | EGDCAPEEK | 212-220 |
| | | Cw16 | 7 | 344 | ISGGPRISY | 293-301 |
| | | | | 327 | YLEYRQVPG | |
| | | DR13 | 19 | 318 | LLKYRAREPVTKAE | 121-134 |
| MAGE-A7 | | | | | | |
| MAGE-A8 | | A2 | | 345 | KVAELVRFL | |
| | | A2 | | 346 | GLMDVQIPT | |
| MAGE-A9 | | A2 | 44 | 347 | ALSVMGVYV | 223-231 |
| MAGE-A10 | | A2 | 44 | 348 | GLYDGMEHLI | 254-262 |
| | | A2 | | 327 | YLEYRQVPG | |
| | | A2 | | 349 | SLLKFLAKV | |
| | | B53 | 2 | 304 | DPARYEFLW | 290-298 |
| MAGE-A11 | | | | | | |
| MAGE-A12m | | A2g | 44 | 350 | FLWGPRALVE | 271-279 |
| | | A2 | | 295 | YLEYRQVPV | |
| | | Cw7 | 41 | 351 | VRIGHLYIL | 170-178 |
| | | Cw7 | 41 | 317 | EGDCAPEEK | 212-220 |
| | | DP4 | 75 | 352 | REPFTKAEMLGSVIR | 127-141 |
| | | | | 327 | YLEYRQVPG | |
| | | DR13 | 19 | 353 | AELVHFLLLKYRAR | 114-127 |
| MAGE-C1 | | A2 | 44 | 354 | ILFGISLREV | 959-968 |
| | | A2 | 44 | 355 | KVVEFLAML | 1083-1091 |
| | | DQ6 | 63 | 356 | SSALLSIFQSSPE | 137-149 |
| | | DQ6 | 63 | 357 | SFSYTLLSL | 450-458 |
| | | DR15 | 20 | 358 | VSSFFSYTL | 779-787 |
| MAGE-C2 | | A2 | 44 | 359 | LLFGLALIEV | 191-200 |
| | | A2 | 44 | 164 | ALKDVEERV | 336-344 |
| | | | | 360 | TLDEKVAELV | |
| | | | | 361 | KVLEFLAKL | |
| | | | | 362 | FLAKLNNTV | |
| | | | | 363 | VIWEVLNAV | |
| | | B44 | 21 | 364 | SESIKKKVL | 307-315 |
| | | B57 | 8 | 165 | ASSTLYLVF | 42-50 |
| | | DR15 | 20 | 365 | SSTLYLVFSPSSFST | 43-57 |
| MAGE-n | | A2 | | 366 | FLWGPRALA | |
| | | A2 | | 367 | QLVFGIEVV | |
| mucin | | | | 368 | PDTRPAPGSTAPPAHGVTSA | |
| NA88-A | | B13 | 6 | 369 | QGQHFLQKV | |
| NY-ESO-1 / LAGE-2 | | A2 | 44 | 167 | SLLMWITQC | 157-165 |
| | | A2 | 44 | 281 | MLMAQEALAFL | ORF2 |
| | | A2 | | 370 | SLLMWITQCFL | |
| | | A2 | | 371 | QLSLLMWIT | |
| | | A24 | | 372 | LLMWITQCF | |
| | | A24 | 20 | 373 | YLAMPFATPME | 91-101 |
| | | A31 | 5 | 374 | ASGPGGGAPR | 53-62 |
| | | A31 | 5 | 282 | LAAQERRVPR | ORF2 |
| | | | | | | (18-27) |
| | | A68 | 8 | 375 | TVSGNILTIR | 127-136 |
| | | B7 | 17 | 376 | APRGPHGGAASGL | 60-72 |
| | | B35 | 20 | 377 | MPFATPMEAEL | 94-104 |
| | | B49 | | 378 | KEFTVSGNILTI | 124-135 |
| | | B51 | 12 | 379 | MPFATPMEA | 94-102 |
| | | B52 | 5 | 380 | FATPMEAEL | 96-104 |
| | | C12 | 12 | 381 | FATPMEAELAR | 96-106 |
| | | Cw3 | 17 | 382 | LAMPFATPM | 92-100 |
| | | Cw6 | 18 | 383 | ARGPESRLL | 80-88 |
| | | DP4 | 75 | 285 | SLLMWITQCFLPVF | 157-170 |
| | | DP4 | 75 | 384 | LLEFYLAMPFATPMEAELARRSL | 87-111 |
| | | | | | AQ | |
| | | DR1 | 18 | 384 | LLEFYLAMPFATPMEAELARRSL | 87-111 |
| | | | | | AQ | |
| | | DR1 | 18 | 385 | EFYLAMPFATPM | 89-100 |
| | | DR1 | 18 | 386 | PGVLLKEFTVSGNILTIRLTAADH | 119-143 |
| | | | | | R | |
| | | DR2 | 25 | 387 | RLLEFYLAMPFA | 86-97 |
| | | DR3 | 21 | 286 | QGAMLAAQERRVPRAAEVPR | ORF2 |
| | | | | | | (14-33) |
| | | DR4 | 24 | 388 | PFATPMEAELARR | 95-107 |
| | | DR4 | 24 | 389 | PGVLLKEFTVSGNILTIRLT | 119-138 |
| | | DR4 | 24 | 390 | VLLKEFTVSG | 121-130 |
| | | DR4 | 24 | 287 | AADHRQLQLSISSCLQQL | 139-156 |
| | | DR4 | 24 | 384 | LLEFYLAMPFATPMEAELARRSL | 87-111 |
| | | | | | AQ | |
| | | DR52b | 25 | 391 | LKEFTVSGNILTIRL | 123-137 |
| | | DR7 | 25 | 386 | PGVLLKEFTVSGNILTIRLTAADH | 119-143 |
| | | | | | R | |
| | | DR7 | 25 | 384 | LLEFYLAMPFATPMEAELARRSL | 87-111 |
| | | | | | AQ | |
| | | DR8 | 4 | 392 | KEFTVSGNILT | 124-134 |
| | | DR9 | 3 | 393 | LLEFYLAMPFATPM | 87-100 |
| | | DR15 | 20 | 290 | AGATGGRGPRGAGA | 37-50 |
| Neutrophil granule | | | | 394 | VLQELNVTV | |
| proteases | | | | | | |
| OFA-iLR | | A2 | | 395 | ALCNTDSPL | |
| | | A2 | | 396 | LLAARAIVAI | |
| PTH-rP | | A2 | | 397 | TSTTSLELD | |
| | | A2 | | 398 | FLHHLIAEIH | |
| S2 | | A26 | | 399 | DLWKETVFT | |
| SAGE | | A24 | 20 | 400 | LYATVIHDI | 715-723 |
| Sp17 | | A1 | 26 | 401 | ILDSSEEDK | 103-111 |
| SSX-2 | | A2 | 44 | 402 | KASEKIFYV | 41-49 |
| | | A2 | | 403 | RLQGISPKI | |
| | | DP1 | 14 | 404 | EKIQKAFDDIAKYFSK | 19-34 |
| | | DR1 | 18 | 405 | FGRLQGISPKI | 101-111 |
| | | DR3 | 21 | 406 | WEKMKASEKIFYVYMKRK | 37-54 |
| | | DR4 | 24 | 407 | KIFYVYMKRKYEAMT | 45-59 |
| | | DR11 | 25 | 408 | KIFYVYMKRKYEAM | 45-58 |
| SSX-4 | | DP10 | 2 | 409 | INKTSGPKRGKHAWTHRLRE | 151-170 |
| | | DR3 | 21 | 410 | YFSKKEWEKMKSSEKIVYVY | 31-50 |
| | | DR8 | 4 | 411 | MKLNYEVMTKLGFKVTLPPF | 51-70 |
| | | DR8 | 4 | 412 | KHAWTHRLRERKQLVVYEEI | 161-180 |
| | | DR11 | 25 | 413 | LGFKVTLPPFMRSKRAADFH | 61-80 |
| | | DR15 | 20 | 414 | KSSEKIVYVYMKLNYEVMTK | 41-60 |
| | | DR52 | 41 | 412 | KHAWTHRLRERKQLVVYEEI | 161-180 |
| TAG | | A3 | | 415 | RLSNRLLLR | |
| TAG-1 | | A2 | 44 | 416 | SLGWLFLLL | 78-86 |
| | | B8 | 14 | 417 | LSRLSNRLL | 42-50 |
| TAG-2 | | B8 | 14 | 417 | LSRLSNRLL | 42-50 |
| | | A1, A2, | | 418 | LTYVSFRNL | |
| TPBG | | A3, B7 | | | | |
| | | A2 | | 419 | DLPAYVRNL | |
| | | A2 | | 420 | FLTGNQLAV | |
| | | A2 | | 421 | GAFEHLPSL | |
| | | A2 | | 422 | RLARLALVL | |
| | | Cw7 | | 423 | PLADLSPFA | |
| TRAG-3 | | A2 | | 424 | ILLRDAGLV | |
| | | DR1 | 18 | 425 | CEFHACWPAFTVLGE | 34-48 |
| | | DR4 | 24 | 425 | CEFHACWPAFTVLGE | 34-48 |
| | | DR7 | 25 | 425 | CEFHACWPAFTVLGE | 34-48 |
| TRP2-6b | | A2 | | 426 | ATTNILEHY | |
| TRP2-INT2g | | A68 | 8 | 427 | EVISCKLIKR | intron 2 |
| TTK | | A24 | | 428 | SYRNEIAYL | |
| XAGE-1b/GAGED2a | | A2 | 44 | 429 | RQKKIRIQL | 21-29 |
| | | DR4 | 24 | 430 | HLGSRQKKIRIQLRSQ | 17-32 |
| | | DR9 | 3 | 431 | CATWKVICKSCISQTPG | 33-49 |
| ART-4 | | A24 | | 432 | AFLRHAAL | |
| | | A24 | | 433 | DYPSLSATDI | |
| CDCA1/NUF2 | | A2 | | 434 | YMMPVNSEV | |
| | | A2 | | 435 | KLATAQFKI | |
| Cep55/c10or13 | | A24 | | 436 | VYVKGLLAKI | |
| CML28 (EXOSC5) | | A2 | | 437 | ALVDAGVPM | |
| DAM-6, -10 | | A2 | | 438 | FLWGPRAYA | |
| (MAGE-B1) | | | | | | |
| IMP-3 | | A2 | | 439 | RLLVPTQFV | |
| | | A2 | | 440 | NLSSAEVVV | |
| | | A24 | | 441 | KTVNELQNL | |
| OVA66 | | A2 | | 442 | FLPDHINIV | |
| OY-TES-1 | | A24 | | 443 | KTPFVSPLL | |
| PASD1 | | A2 | | 444 | QLLDGFMITL | |
| | | A2 | | 445 | YLVGNVCIL | |
| | | A2 | | 446 | ELSDSLGPV | |
| RHAMM/CD168 | | A2 | | 447 | ILSLELMKL | |
| | | A2 | | 448 | SLEENIVIL | |
| SART-1 | | A2 | | 449 | KGSGKMKTE | |
| | | A24 | | 450 | EYRGFTQDF | |
| SART-3 | | A2 | | 451 | RLAEYQAYI | |
| | | A2 | | 452 | LLQAEAPRL | |
| | | A3 | | 453 | WLEYYNLER | |
| | | A3 | | 454 | QIRPIFSNR | |
| | | A24 | | 455 | VYDYNCHVDL | |
| | | A24 | | 456 | AYIDFEMKI | |
| | | A26 | | 455 | VYDYNCHVDL | |
| CEA | gut carcinoma | A2 | 44 | 457 | YLSGANLNLG | 605-613 |
| | | A2 | 44 | 458 | IMIGVLVGV | 691-699 |
| | | A2 | 44 | 459 | GVLVGVALI | 694-702 |
| | | A2 | | 460 | VLYGPDAPTV | |
| | | A2 | | 461 | YLSGANLNV | |
| | | A2 | | 462 | ATVGIMIGV | |
| | | A3 | 22 | 463 | HLFGYSWYK | 61-69 |
| | | A24 | 20 | 464 | QYSWFVNGTF | 268-277 |
| | | A24 | 20 | 465 | TYACFVSNL | 652-660 |
| | | B27 | | 466 | HRWCIPWQRL | |
| | | DR3 | 21 | 467 | AYVCGIQNSVSANRS | 568-582 |
| | | DR4 | 24 | 468 | DTGFYTLHVIKSDLVNEEATGQF | 116-140 |
| | | | | | RV | |
| | | DR4 | 24 | 469 | YSWRINGIPQQHTQV | 625-639 |
| | | DR7 | 25 | 470 | TYYRPGVNLSLSC | 425-437 |
| | | DR7 | 25 | 471 | EIIYPNASLLIQN | 99-111 |
| | | DR9 | 3 | 472 | YACFVSNLATGRNNS | 653-667 |
| | | DR11 | 25 | 473 | LWWVNNQSLPVSP | 177-189 |
| | | | | | | and |
| | | | | | | 355-367 |
| | | DR13 | 19 | 473 | LWWVNNQSLPVSP | 177-189 |
| | | | | | | and |
| | | | | | | 355-367 |
| | | DR14 | 6 | 473 | LWWVNNQSLPVSP | 177-189 |
| | | | | | | and |
| | | | | | | 355-367 |
| | | DR14 | 6 | 471 | EIIYPNASLLIQN | 99-111 |
| | | DR14 | 6 | 474 | NSIVKSITVSASG | 666-678 |
| gp100 / Pmel17 | melanoma | A2 | 44 | 475 | KTWGQYWQV | 154-162 |
| | | A2 | 44 | 476 | (A)MLGTHTMEV | 177(8)-186 |
| | | A2 | | 477 | IMDQVPFSV | |
| | | A2 | 44 | 158 | ITDQVPFSV | 209-217 |
| | | A2 | 44 | 478 | YLEPGPVTA | 280-288 |
| | | A2 | 44 | 479 | LLDGTATLRL | 457-466 |
| | | A2 | 44 | 480 | VLYRYGSFSV | 476-485 |
| | | A2 | 44 | 481 | SLADTNSLAV | 570-579 |
| | | A2 | 44 | 482 | RLMKQDFSV | 619-627 |
| | | A2 | 44 | 483 | RLPRIFCSC | 639-647 |
| | | A3 | 22 | 484 | LIYRRRLMK | 614-622 |
| | | A3 | 22 | 485 | ALLAVGATK | 17-25 |
| | | A3 | 22 | 486 | IALNFPGSQK | 86-95 |
| | | A3 | 22 | 487 | RSYVPLAHR | 195-202 and |
| | | | | | | 191 or 192e |
| | | A3 | 22 | 488 | ALNFPGSQK | 87-95 |
| | | A11 | 13 | 488 | ALNFPGSQK | 87-95 |
| | | A24 | 20 | 489 | VYFFLPDHL | intron 4 |
| | | A32 | 8 | 490 | RTKQLYPEW | 40-42 |
| | | | | | | and |
| | | | | | | 47-52e |
| | | A68 | 8 | 491 | HTMEVTVYHR | 182-191 |
| | | B7 | 17 | 492 | SSPGCQPPA | 529-537 |
| | | B35 | 20 | 493 | VPLDCVLYRY | 471-480 |
| | | B35 | 20 | 494 | LPHSSSHWL | 630-638 |
| | | Cw8 | -c | 495 | SNDGPTLI | 71-78 |
| | | DQ6 | 63 | 496 | GRAMLGTHTMEVTVY | 175-189 |
| | | DR4 | 24 | 497 | WNRQLYPEWTEAQRLD | 44-59 |
| | | DR7 | 25 | 498 | TTEWVETTARELPIPEPE | 420-437 |
| | | DR7 | 25 | 499 | TGRAMLGTHTMEVTVYH | 174-190 |
| | | DR53 | 49 | 496 | GRAMLGTHTMEVTVY | 175-189 |
| mammaglobin-A | breast cancer | A2 | | 500 | FLNQTDETL | |
| | | A2 | | 501 | MQLIYDSSL | |
| | | A2 | | 502 | KLLMVLMLA | |
| | | A2 | | 503 | LIYDSSLCDL | |
| | | A3 | | 504 | AIDELKECF | |
| | | A3 | | 505 | TTNAIDELK | |
| | | A3 | 22 | 506 | PLLENVISK | 23-31 |
| | | A3 | | 502 | KLLMVLMLA | |
| Melan-A / MART-1 | melanoma | A2 | 44 | 160 | (E)AAGIGILTV | 26(27)-35 |
| | | A2 | 44 | 507 | ILTVILGVL | 32-40 |
| | | B35 | 20 | 160 | EAAGIGILTV | 26-35 |
| | | B45 | 2 | 508 | AEEAAGIGIL(T) | 24-33(34) |
| | | Cw7 | 41 | 509 | RNGYRALMDKS | 51-61 |
| | | DP5 | 3 | 510 | YTTAEEAAGIGILTVILGVLLLIGC | 21-50 |
| | | | | | WYCRR | |
| | | DQ6 | 63 | 511 | EEAAGIGILTVI | 25-36 |
| | | DR1 | 18 | 512 | AAGIGILTVILGVL | 27-40 |
| | | DR1 | 18 | 513 | APPAYEKLPSAEQF | 100-111 |
| | | DR3 | 21 | 511 | EEAAGIGILTVI | 25-36 |
| | | DR4 | 24 | 514 | RNGYRALMDKSLHVGTQCALTR | 51-73 |
| | | | | | R | |
| | | DR11 | 25 | 515 | MPREDAHFIYGYPKKGHGHS | 1-20 |
| | | DR52 | 41 | 516 | KNCEPVVPNAPPAYEKLSAE | 91-110 |
| MC1R | | A2 | | 517 | TILLGIFFL | |
| | | A3 | | 518 | FLALIICNA | |
| Mesothelin | | A2 | | 519 | KLLGPHVEGL | |
| | | A2 | | 520 | KLLGPHVLGV | |
| NGEP | | A2 | | 521 | GLFDEYLEMV | |
| NY-BR-1 | breast cancer | A2 | 44 | 522 | SLSKILDTV | 904-912 |
| | | A2 | | 523 | LLSHGAVIEV | |
| OA1 | melanoma | A24 | 20 | 524 | LYSACFWWL | 126-134 |
| PAP | prostate cancer | A2 | 44 | 525 | FLFLLFFWL | 18-26 |
| | | A2 | 44 | 526 | TLMSAMTNL | 112-120 |
| | | A2 | 44 | 527 | ALDVYNGLL | 299-307 |
| P Polypeptide | | A2 | | 528 | IMLCLIAAV | |
| PSA | prostate carcinoma | A2 | 44 | 529 | FLTPKKLQCV | 165-174 |
| | | A2 | 44 | 530 | VISNDVCAQV | 178-187 |
| RAB38 /NY-MEL-1 | melanoma | A2 | 44 | 531 | VLHWDPETV | 50-58 |
| TARP | | A2 | | 532 | FLRNFSLMV | |
| | | A2 | | 533 | FVFLRNFSL | |
| | | A2 | | 534 | FLRNFSLML | |
| TRP-1 / gp75 | melanoma | A31 | 5 | 535 | MSLQRQFLR | alt. ORF |
| | | DR4 | 24 | 536 | ISPNSVFSQWRVVCDSLEDYD | 277-297 |
| | | DR15 | 20 | 537 | SLPYWNFATG | 245-254 |
| | | DR17 | 21 | 538 | SQWRVVCDSLEDYDT | 284-298 |
| TRP-2 | melanoma | A1, A2 | | 539 | VYDFFVWLHY | |
| | | A2 | | 540 | SLDDYNHLV | |
| | | A2 | | 541 | FVWLHYYSV | |
| | | A2 | 44 | 542 | SVYDFFVWL | 180-188 |
| | | A2 | 44 | 543 | TLDSQVMSL | 360-368 |
| | | A31 | 5 | 544 | LLGPGRPYR | 197-205 |
| | | A33 | 5 | 544 | LLGPGRPYR | 197-205 |
| | | Cw8 | -c | 545 | ANDPIFVVL | 387-395 |
| | | DR3 | 21 | 546 | QCTEVRADTRPWSGP | 60-74 |
| | | DR15 | 20 | 547 | ALPYWNFATG | 241-250 |
| tyrosinase | melanoma | A1 | 26 | 548 | KCDICTDEY | 243-251 |
| | | A1 | | 549 | DSDPDSFQDY | |
| | | A1 | 26 | 550 | SSDYVIPIGTY | 146-156 |
| | | A2 | 44 | 551 | MLLAVLYCL | 1-9 |
| | | A2 | 44 | 552 | CLLWSFQTSA | 8-17 |
| | | A2 | 44 | 159 | YMDGTMSQV | 369-377 |
| | | A24 | 20 | 553 | AFLPWHRLF | 206-214 |
| | | A24 | 20 | 554 | IYMDGTADFSF | 368-373 and |
| | | | | | | 336-340e |
| | | A26 | 8 | 555 | QCSGNFMGF | 90-98 |
| | | B35 | 20 | 556 | TPRLPSSADVEF | 309-320 |
| | | B35 | 20 | 557 | LPSSADVEF | 312-320 |
| | | B38 | 5 | 558 | LHHAFVDSIF | 388-397 |
| | | B44 | 21 | 559 | SEIWRDIDFD | 192-200 |
| | | DR4 | 24 | 560 | QNILLSNAPLGPQFP | 56-70 |
| | | DR4 | 24 | 561 | SYLQDSDPDSFQD | 450-462 |
| | | DR15 | 20 | 562 | FLLHHAFVDSIFEQWLQRHRP | 386-406 |
| human chorionic | Differentiation | | | | | |
| gonadotropin | | | | | | |
| P501s (prostein) | Differentiation | Cw5 | | 563 | SACDVSVRVV | |
| | | Cw5 | | 564 | YTDFVGEGL | |
| ADAM17 | | A2 | | 565 | YLIELIDRV | |
| adipophilin | adipocytes, | A2 | 44 | 566 | SVASTITGV | 129-137 |
| | macrophages | | | | | |
| ADP-ribosylation | | A2 | | 567 | CITFQVWDV | |
| factor | | A2 | | 568 | FLPHFQALHV | |
| AIM-2 | ubiquitous (low level) | A1 | 26 | 569 | RSDSGQQARY | intron |
| ALDH1A1 | mucosa, keratinocytes | A2 | 44 | 570 | LLYKLADLI | 88-96 |
| ALK | | A2 | | 571 | SLAMLDLLHV | |
| ATIC (AICRT) | | A2 | | 572 | MVYDLYKTL | |
| | | A2 | | 573 | RLDFNLIRV | |
| BA46 (MFGE8) | | A2 | | 574 | GLQHWVPEL | |
| | | A2 | | 575 | NLFETPVEA | |
| BAP31 | | A2 | | 576 | KLDVGNAEV | |
| BAX-delta | | A2 | | 577 | YLLQGMIAAV | |
| | | A2 | | 578 | YLQGMIAAV | |
| Bcl-2 | | A2 | | 579 | PLFDFSWLSL | |
| | | A2 | | 580 | WLSLKTLLSL | |
| BCLX (L) | ubiquitous (low level) | A2 | 44 | 581 | YLNDHLEPWI | 173-182 |
| BING-4 | ubiquitous (low level) | A2 | 44 | 582 | CQWGRLWQL | ORF2 |
| BTBD2 | | A24 | | 583 | VFLPCDSWNL | |
| C19orf48 | | A2 | | 584 | CIPPDSLLFPA | |
| CA125 | | A2 | | 585 | YTLDRDSLYV | |
| Cadherin 3/P- | | A2 | | 586 | FIIENLKAA | |
| cadherin | | A2 | | 587 | FILPVLGAV | |
| CALCA | thyroid | A2 | 44 | 588 | VLLQAGSLHA | 16-25 |
| CLCA2 | | A2 | | 589 | LLGNCLPTV | |
| | | A2 | | 590 | SLQALKVTV | |
| CLP (coactosin-like | | A2 | | 591 | NLVRDDGSAV | |
| protein) | | A2 | 592 | | RLFAFVRFT | |
| | | A2 | 593 | | VVQNFAKEFV | |
| CD45 | proliferating cells, | A24 | 20 | 594 | KFLDALISL | 556-564 |
| | testis, multiple tissues | | | | | |
| | (low level) | | | | | |
| CD274 | multiple tissues (lung, | A2 | 44 | 595 | LLNAFTVTV | 15-23 |
| | heart, . . . ) and | | | | | |
| | induced by IFN-γ | | | | | |
| CDKN1A | | A2 | | 596 | FAWERVRGL | |
| | | A2 | | 597 | GLGLPKLYL | |
| | | A2 | | 598 | LMAGCIQEA | |
| Cdr2 | | A2 | | 599 | LLEEMFLTV | |
| CPSF | ubiquitous (low level) | A2 | 44 | 600 | KVHPVIWSL | 250-258 |
| | | A2 | 44 | 601 | LMLQNALTTM | 1360-1369 |
| c-MET | | A2 | | 602 | YVDPVITSI | |
| COA-1 (UBXN11) | | A2 | | 603 | FMTRKLWDL | |
| | | A2 | | 604 | RLLASLQDL | |
| Cox2 | | A2 | | 605 | ALYGDIDAV | |
| | | A3 | | 605 | ALYGDIDAV | |
| Cyclin I | | A2 | | 606 | LLDRFLATV | |
| cyclin B1 | | A2 | | 607 | ILIDWLVQV | |
| | | A2 | | 608 | AKYLMELTM | |
| | | A2 | | 609 | AGYLMELCC | |
| cyclin D1 | ubiquitous (low level) | A2 | 44 | 610 | LLGATCMFV | 101-109 |
| | | DR4 | 24 | 611 | NPPSMVAAGSVVAAV | 198-212 |
| cyclophilin B (Cyp- | | A2 | | 612 | VLEGMEVV | |
| B) | | A2 | | 613 | KLKHYGPGWV | |
| | | A24 | | 614 | DFMIQGGDF | |
| | | A24 | | 615 | KFHRVIKDF | |
| CYP1B1 | | A2 | | 616 | WLQYFPNPV | |
| DKK1 | testis, prostate, | A2 | 44 | 617 | ALGGHPLLGV | 20-29 |
| | mesenchymal stem | | | | | |
| | cells | | | | | |
| EGFR | | A2 | | 618 | ITDFGLAKL | |
| | | A2 | | 618 | ITDFGLAKL | |
| ENAH (hMena) | breast, prostate stroma | A2 | 44 | 619 | TMNGSKSPV | 502-510 |
| | and epithelium of | | | | | |
| | colon-rectum, pancreas, | | | | | |
| | endometrium | | | | | |
| EpCAM | epithelial cells | A2 | | 620 | YQLDPKFIV | |
| | | A2 | | 621 | GLKAGVIAV | |
| | | A2 | | 622 | ILYENNVITV | |
| | | A2 | | 623 | ILYENNVIV | |
| | | A24 | 20 | 624 | RYQLDPKFI | 173-181 |

EphA2

| | | | | | | |
|---|---|---|---|---|---|---|
| | | A2 | | 625 | VLLLVLAGV | |
| | | A2 | | 626 | TLADFDPRV | |
| | | A2 | | 627 | VLAGVGFFI | |
| | | A2 | | 628 | IMNDMPIYM | |
| EphA3 | many | DR11 | 25 | 629 | DVTFNIICKKCG | 356-367 |
| EZH2 | ubiquitous (low level) | A2 | 44 | 630 | FMVEDETVL | 120-128 |
| | | A2 | 44 | 631 | FINDEIFVEL | 165-174 |
| | | A24 | 20 | 632 | KYDCFLHPF | 291-299 |
| | | A24 | 20 | 633 | KYVGIEREM | 735-743 |
| FGF5 | brain, kidney | A3 | 22 | 634 | NTYASPRFKF | 172-176 |
| | | | | | | and |
| | | | | | | 217-220 |
| glypican-3 | placental and multiple | A2 | 44 | 635 | FVGEFFTDV | 144-152 |
| | tissues | A24 | 20 | 636 | EYILSLEEL | 298-306 |
| G250 / MN / CAIX | stomach, liver, | A2 | 44 | 637 | HLSTAFARV | 254-262 |
| | pancreas | | | | | |
| HBD | | A24 | | 638 | KYLKLSSSEL | |
| hCG-beta | | A2 | | 639 | TMTRVLQGV | |
| | | A2 | | 640 | GVNPVVSYAV | |
| | | A2 | | 641 | VLQVGLPAL | |
| Heparanase | | A2 | | 642 | PAFSYSFFV | |
| | | A2 | | 643 | LLLGPLGPL | |
| | | A2 | | 644 | KMLKSFLKA | |
| | | A2 | | 645 | ALPPPLMLL | |
| | | A2 | | 646 | WLSLLFKKL | |
| HER-2 / neu | ubiquitous (low level) | A2 | 44 | 647 | KIFGSLAFL | 369-377 |
| | | A2 | 44 | 648 | IISAVVGIL | 654-662 |
| | | A2 | 44 | 649 | ALCRWGLLL | 5-13 |
| | | A2 | 44 | 650 | ILHNGAYSL | 435-443 |
| | | A2 | 44 | 651 | RLLQETELV | 689-697 |
| | | A2 | 44 | 652 | VVLGVVFGI | 665-673 |
| | | A2 | 44 | 653 | YMIMVKCWMI | 952-961 |
| | | A2 | 44 | 654 | HLYQGCQVV | 48-56 |
| | | A2 | 44 | 655 | YLVPQQGFFC | 1023-1032 |
| | | A2 | 44 | 656 | PLQPEQLQV | 391-399 |
| | | A2 | 44 | 657 | TLEEITGYL | 402-410 |
| | | A2 | 44 | 658 | ALIHHNTHL | 466-474 |
| | | A2 | 44 | 659 | PLTSIISAV | 650-658 |
| | | A2 | | 660 | KLFGSLAFV | |
| | | A2 | | 661 | ITDFGLARL | |
| | | A2 | | 662 | KVFGSLAFV | |
| | | A2 | | 663 | AVVGILLVV | |
| | | A2 | | 664 | QLFEDNYAL | |
| | | A2 | | 665 | QIAKGMSYL | |
| | | A2 | | 666 | LIAHNQVRQV | |
| | | A3 | 22 | 667 | VLRENTSPK | 754-762 |
| | | A24 | 20 | 668 | TYLPTNASL | 63-71 |
| HIFPH3 | | A24 | | 669 | RYAMTVWYF | |
| HLA-DOB | B lymphocytes, | A2 | 44 | 670 | FLLGLIFLL | 232-240 |
| | monocytes, blood cells, | | | | | |
| | adrenal | | | | | |
| HM1.24 | | A2 | | 671 | LLLGIGILV | |
| HMW-MAA | | A2 | | 672 | LLQLGYSGRL | |
| | | A2 | | 673 | LLQLYSGRL | |
| Hepsin | kidney, liver, skin | A2 | 44 | 674 | SLLSGDWVL | 191-199 |
| | | A2 | 44 | 675 | GLQLGVQAV | 229-237 |
| | | A2 | 44 | 676 | PLTEYIQPV | 268-276 |
| HO-1 | | B8 | | 677 | APLLRWVL | |
| Hsp70 | | A2 | | 678 | LLLLDVAPL | |
| | | A2 | | 679 | LLDVAPLSL | |
| HST-2 (FGF-6) | | A31 | | 680 | YSWMDISCWI | |
| ICE | | B7 | | 681 | SPRWWPTCL | |
| IDO1 | lymph nodes, placenta, | A2 | 44 | 682 | ALLEIASCL | 199-207 |
| | and many cell types in | | | | | |
| | the course of | | | | | |
| | inflammatory response | | | | | |
| IEX-1 | | A11 | | 683 | APAGRPSASR | |
| | | A11 | | 684 | RSRRVLYPR | |
| | | A31 | | 684 | RSRRVLYPR | |
| | | A31 | | 683 | APAGRPSASR | |
| | | A33 | | 684 | RSRRVLYPR | |
| | | A33 | | 685 | APAGRPSAS | |
| | | A33 | | 686 | VLYPRVVRR | |
| IGF2B3 | ubiquitous (low level) | A2 | 44 | 440 | NLSSAEVVV | 515-523 |
| | | A3 | 44 | 439 | RLLVPTQFV | 199-207 |
| IL13Ralpha2 | | A2 | 44 | 687 | WLPFGFILI | 345-353 |
| | | A24 | | 688 | WYEGLDHAL | |
| integrin beta subunit | | A2 | | 689 | ALMEQQHYV | |
| Intestinal carboxyl | liver, intestine, kidney | B7 | 17 | 681 | SPRWWPTCL | alt. ORF |
| esterase | | | | | | |
| alpha-foetoprotein | liver | A2 | 44 | 690 | GVALQTMKQ | 542-550 |
| | | A2 | 44 | 691 | FMNKFIYEI | 158-166 |
| | | A2 | | 692 | GLSPNLNRFL | |
| | | A2 | | 693 | PLFQVPEPV | |
| | | A3 | | 694 | ILLWAARYD | |
| | | A24 | | 695 | EYSRRHPQL | |
| | | A24 | | 696 | AYTKKAPQL | |
| | | A24 | | 697 | EYYLQNAFL | |
| | | A24 | | 698 | KYIQESQAL | |
| | | A24 | | 699 | RSCGLFQKL | |
| | | DR13 | 19 | 700 | QLAVSVILRV | 364-373 |
| JARID1B | | A2 | | 701 | QLYALPCVL | |
| Keratin 18 | | A2 | | 702 | ALLNIKVKL | |
| Kallikrein 4 | prostate and ovarian | A2 | 44 | 703 | FLGYLILGV | 11-19 |
| | cancer | DP4 | 75 | 704 | SVSESDTIRSISIAS | 125-139 |
| | | DR4 | 24 | 705 | LLANGRMPTVLQCVN | 155-169 |
| | | DR7 | 25 | 706 | RMPTVLQCVNVSVVS | 160-174 |
| KIF20A | ubiquitous (low level) | A2 | 44 | 707 | LLSDDDVVV | 12-20 |
| | | A2 | 44 | 708 | AQPDTAPLPV | 284-293 |
| | | A2 | 44 | 709 | CIAEQYHTV | 809-817 |
| L10a | | A26 | | 710 | ETVELQISL | |
| | | A26 | | 711 | TLYEAVREV | |
| Lck | | A2 | | 712 | KLVERLGAA | |
| | | A2 | | 713 | DVWSFGILL | |
| | | A24 | | 714 | TFDYLRSVL | |
| | | A24 | | 715 | HYTNASDGL | |
| | | A24 | | 716 | DYLRSVLEDF | |
| Livin (ML-IAP) | | A2 | | 717 | QLCPICRAPV | |
| | | A2 | | 718 | RLASFYDWLP | |
| | | A2 | | 719 | SLGSPVLGL | |
| LRRC8A | | B7 | | 720 | GPRESRPPA | |
| Lengsin | eye lens and low level | A2 | 44 | 721 | FLPEFGISSA | 270-279 |
| | in multiple tissues | | | | | |
| M2BP | | A2 | | 722 | RIDITLSSV | |
| | | A24 | | 723 | GYCASLFAIL | |
| M-CSF | liver, kidney | B35 | 20 | 724 | LPAVVGLSPGEQEY | alt. ORF |
| MCSP | endothelial cells, | DR11 | 25 | 725 | VGQDVSVLFRVTGALQ | 693-708 |
| | chondrocytes, smooth | | | | | |
| | muscle cells | | | | | |
| mdm-2 | ubiquitous (brain, | A2 | 44 | 726 | VLFYLGQY | 53-60 |
| | muscle, lung) | A2 | | 727 | LLGDLFGV | |
| Meloe | ubiquitous (low level) | A2 | 44 | 728 | TLNDECWPA | 36-44 |
| | | DQ2 | 41 | 729 | ERISSTLNDECWPA | 31-44 |
| | | DQ6 | 63 | 405 | FGRLQGISPKI | 32-44 |
| | | DR1 | 18 | 730 | TSREQFLPSEGAA | 11-23 |
| | | DR11 | 25 | 731 | CPPWHPSERISSTL | 24-37 |
| Meloe-2 | | A2 | | 732 | RLPPKPPLA | |
| | | A2 | | 733 | RCPPKPPLA | |
| MG50 | | A2 | | 734 | TLKCDCEIL | |
| | | A2 | | 735 | RLGPTLMCL | |
| | | A2 | | 736 | LLLEAVPAV | |
| | | A2 | | 737 | WLPKILGEV | |
| | | A2 | | 738 | VLSVNVPDV | |
| | | A2 | | 739 | CMHLLLEAV | |
| Midkine | ubiquitous (low level) | A2 | 44 | 740 | ALLALTSAV | 13-21 |
| | | A2 | 44 | 741 | AQCQETIRV | 114-122 |
| | | DR4 | 24 | 742 | LTLLALLALTSAVAK | 12-23 |
| MMP-2 | ubiquitous | A2 | 44 | 743 | GLPPDVQRVH | 560-568 |
| MMP-7 | ubiquitous (low level) | A3 | 22 | 744 | SLFPNSPKWTSK | 96-107 |
| MPP-11 | | A2 | | 745 | QLLIKAVNL | |
| | | A2 | | 746 | STLCQVEPV | |
| MRP3 | | A24 | | 747 | AYVPQQAWI | |
| | | A24 | | 748 | VYSDADIFL | |
| | | A24 | | 749 | NYSVRYRPGL | |
| | | A24 | | 750 | LYAWEPSFL | |
| MUC1 | glandular epithelia | A2 | 44 | 751 | STAPPVHNV | 950-958 |
| | | A2 | 44 | 752 | LLLLTVLTV | 12-20 |
| | | A11 | | 753 | STAPPAHGV | |
| | | A68 | | 754 | DVTSAPDNK | |
| | | B7 | | 755 | VPGWGIALL | |
| | | B7 | | 756 | DPSTDYYQEL | |
| | | B44 | | 757 | TEAASRYNL | |
| | | DR3 | 21 | 758 | PGSTAPPAHGVT | repeated |
| | | | | | | region |
| MUC5AC | surface mucosal cells, | A24 | 20 | 759 | TCQPTCRSL | 716-724 |
| | respiratory tract, and | | | | | |
| | stomach epithelia | | | | | |
| Nucleophosmin | | A1 | | 760 | GCELKADKDY | |
| P15 | | A24 | | 761 | AYGLDFYIL | |
| p53 | ubiquitous (low level) | A2 | 44 | 762 | LLGRNSFEV | 264-272 |
| | | A2 | 44 | 763 | RMPEAAPPV | 65-73 |
| | | A2 | | 764 | LLPENNVLSPV | |
| | | A2 | | 765 | SLPPPGTRV | |
| | | A2 | | 257 | VVPCEPPEV | |
| | | A2 | | 766 | YLGSYGFRL | |
| | | A2 | | 767 | SMPPPGTRV | |
| | | A2 | | 768 | GLAPPQHLIRV | |
| | | A2 | | 769 | KLCPVQLWV | |
| | | A2 | | 770 | KTCPVQLWV | |
| | | A24 | | 771 | AIYKQSQHM | |
| | | B46 | 0.1 | 772 | SQKTYQGSY | 99-107 |
| | | Cw7 | | 773 | TRVLAMAIY | |
| | | DP5 | 3 | 774 | PGTRVRAMAIYKQ | 153-165 |
| | | DR14 | 6 | 775 | HLIRVEGNLRVE | 193-204 |
| PAK2 | | A2 | | 776 | KLAKPLSSL | |
| | | A2 | | 777 | VLLGMEGSV | |
| Papillomavirus | | A2 | | 778 | ALPSFQIPV | |
| binding | | | | | | |
| PAX3 | | A2 | | 779 | QLMAFNHLV | |
| PAX5 | hemopoietic system | A2 | 44 | 780 | TLPGYPPHV | 311-319 |
| PBF | ovary, pancreas, spleen, | B55 | 4 | 781 | CTACRWKKACQR | 499-510 |
| | liver | | | | | |
| PGK1 | | A2 | | 782 | IIGGGMAFT | |
| PRAME | testis, ovary, | A2 | 44 | 783 | VLDGLDVLL | 100-108 |
| | endometrium, adrenals | A2 | 44 | 784 | SLYSFPEPEA | 142-151 |
| | | A2 | 44 | 785 | ALYVDSLFFL | 300-309 |
| | | A2 | 44 | 786 | SLLQHLIGL | 425-433 |
| | | A24 | 20 | 787 | LYVDSLFFLC | 301-309 |
| | | B52 | | 788 | GQHLHLETF | |
| PRDI-BF1 | | A2 | | 789 | FGLFPRLCPV | |
| Preprocalcitonin | | A2 | | 588 | VLLQAGSLHA | |
| (ppCT) | | | | | | |
| Prostatic acid | | | | 527 | ALDVYNGLL | |
| phosphatase | | | | 525 | FLFLLFFWL | |
| | | | | 526 | TLMSAMTNL | |
| | | | | 790 | ILLWQPIPV | |
| | | | | 791 | YLPFRNCRP | |
| | | | | 791 | YLPFRNCRP | |
| PSMA | prostate, CNS, liver | A1 | | 792 | HSTNGVTRIY | |
| | | A2 | | 793 | VLAGGFFLL | |
| | | A24 | | 794 | LYSDPADYF | |
| | | A24 | 20 | 795 | NYARTEDFF | 178-186 |
| RAGE-1 | retina | A2 | 44 | 796 | LKLSGVVRL | 352-360 |
| | | A2 | 44 | 797 | PLPPARNGGLG | 32-40 |
| | | B7 | 17 | 798 | SPSSNRIRNT | 11-20 |
| Ran | | A2 | | 799 | YMFDVTSRV | |
| | | A2 | | 800 | IMFDVTSRV | |
| | | A33 | | 801 | HPLVFHTNR | |
| | | A33 | | 802 | IIMFDVTSR | |
| | heart, skeletal muscle, | A2 | 44 | 803 | LAALPHSCL | 5-13 |
| RGS5 | pericytes | A3 | 22 | 804 | GLASFKSFLK | 74-83 |
| | | B8 | | 805 | MAQKRIHAL | |
| Ribosomal protein | | A31 | | 806 | KNKRILMEH | |
| L19 | | | | | | |
| RhoC | ubiquitous (low level) | A3 | 22 | 807 | RAGLQVRKNK | 176-185 |
| RNF43 | | A2 | 44 | 808 | ALWPWLLMA(T) | 11-19(20) |
| | | A24 | 20 | 809 | NSQPVWLCL | 721-729 |
| RU2AS | testis, kidney, bladder | B7 | 17 | 810 | LPRWPPPQL | antisense |
| SART-2 | | A24 | | 811 | DYSARWNEI | |
| | | A24 | | 812 | AYDFLYNYL | |
| | | A24 | | 813 | SYTRLFLIL | |
| secemin 1 | ubiquitous | A2 | 44 | 814 | KMDAEHPEL | 196-204 |
| SH3GLB2 | | A2 | | 815 | FLTPLRNFL | |
| SOX4 | | Cw*14 | | | | |
| | | 02 | | | | |
| SOX10 | ubiquitous (low level) | A2 | 44 | 816 | AWISKPPGV | 332-340 |
| | | A2 | 44 | 817 | SAWISKPPGV | 331-340 |
| SPARC | | A24 | | 818 | MYIFPVHWQF | |
| | | A24 | | 819 | DYIGPCKYI | |
| STAT1-alpha/beta | | A2 | | 820 | KLQELNYNL | |
| STEAP1 | prostate | A2 | 44 | 821 | MIAVFLPIV | 292-300 |
| | | A2 | | 822 | FLYTLLREV | |
| | | A2 | | 823 | LLLGTIHAL | |
| | | A2 | 44 | 824 | HQQYFYKIPILVINK | 102-116 |
| survivin | ubiquitous | A2 | 44 | 825 | ELTLGEFLKL | 95-104 |
| | | A2 | | 826 | QMFFCFKEL | |
| | | A2 | | 827 | LMLGEFLKL | |
| | | A2 | | 828 | TLPPAWQPFL | |
| | | DR1 | 18 | 829 | TLGEFLKLDRERAKN | 97-111 |
| survivin-2B | | A24 | | 830 | AYACNTSTL | |
| Telomerase | testis, thymus, bone | A2 | 44 | 831 | ILAKFLHWL | 540-548 |
| | marrow, lymph nodes | A2 | 44 | 832 | RLVDDFLLV | 865-873 |
| | | A2 | | 833 | LLTSRLRFI | |
| | | A2 | | 834 | RLFFYRKSV | |
| | | A3 | | 835 | KLFGVLRLK | |
| | | DR7 | 25 | 836 | RPGLLGASVLGLDDI | 672-686 |
| | | DR11 | 25 | 837 | LTDLQPYMRQFVAHL | 766-780 |
| Tie2 | | A2 | | 838 | FLPATLTMV | |
| Topoisomerase II | | A2 | | 839 | FLYDDNQRV | |
| TRG | | B52 | | 840 | YQLCLTNIF | |
| | | B62 | | 840 | YQLCLTNIF | |
| TPBG | multiple tissues | A2 | 44 | 422 | RLARLALVL | 17-25 |
| | (including esophagus, | | | | | |
| | bladder) | | | | | |
| TYMS | | A2 | | 841 | LMALPPCHAL | |
| VEGF | ubiquitous (low level) | B27 | 7 | 842 | SRFGGAVVR | -i |
| VEGFR2/KDR | | A2 | | 843 | FLSTLTIDGV | |
| WHSC2 | | A26 | | 844 | ASLDSDPWV | |
| WNK2/ppMAPkkk | | A26 | | 845 | DLLSHAFFA | |
| WT1 | testis, ovary, bone | A1 | 26 | 846 | TSEKRPFMCAY | 317-327 |
| | marrow, spleen | A2 | | 847 | RMFPNAPYL | |
| | | A2 | | 848 | YMFPNAPYL | |
| | | A24 | 20 | 849 | CMTWNQMNL | 235-243 |
| | | A24 | | 850 | CYTWNQMNL | |
| | | A24 | | 851 | RWPSCQKKF | |
| | | DP5 | 3 | 852 | LSHLQMHSRKH | 337-347 |
| | | DP5 | 3 | 853 | KRYFKLSHLQMHSRKH | 332-347 |
| | | DR4 | 24 | 853 | KRYFKLSHLQMHSRKH | 332-347 |
| XBP1 | | A2 | | 854 | LLSGQPASA | |
| CD33 | | | | | | |
| CD123 | | | | | | |
| CD38 | | | | | | |
| gastrin-17 | | | | | | |
| guanylyl cyclase C | | | | | | |
| HNRPL | | A26 | | 855 | NVLHFFNAPL | |
| EHD2 | | A3 | | 856 | KLPNSVLGR | |
| 707-AP | | A2 | | 857 | RVAALARDAP | |
| mAb MF11-30 VH | | A2 | | 858 | LLVLLYSKL | |
| Replication protein A | | A2 | | 859 | YLMDTSGKV | |
| RU1 | | B51 | | 860 | VPYGSFKHV | |
| septin 2, Nedd5 | | A2 | | 861 | RLYPWGVVEV | |
| SGT1B | | B39 | | 862 | CHILLGNYC | |
| MOG | | | | 863 | MEVGWYRSPFSRVVHLYRNGK | 35-55 |
| Epstein Barr Virus | | A2 | | 152 | CLGGLLTMV | |
| | | A2 | | 153 | GLCTLVAML | |
| | | A2 | | 864 | FLYALALLL | |
| | | A2 | | 865 | YVLDHLIVV | |
| | | A3 | | 146 | RLRAEAQVK | |
| | | A11 | | 866 | AVFDRKSDAK | |
| | | B7 | | 867 | RPPIFIRLL | |
| | | A2 | | 868 | VLQWASLAV | |
| | | | | 869 | FMVFLQTHI | |
| | | | | 870 | FLQTHIFAEV | |
| | | | | 871 | SIVCYFMVFL | |
| Cytomegalovirus | | A1 | | 872 | YSEHPTFTSQY | |
| | | A1 | | 873 | VTEHDTLLY | |
| | | A2 | | 155 | NLVPMVATV | |
| | | A2 | | 874 | VLEETSVML | |
| | | A3 | | 875 | TTVYPPSSTAK | |
| | | A11 | | 876 | GPISGHVLK | |
| | | B7 | | 877 | TPRVTGGGAM | |
| | | B7 | | 878 | RPHERNGFTV | |
| Influenza virus | | A1 | | 879 | VSDGGPNLY | |
| | | A2 | | 880 | GILGFVFTL | |
| | | A2 | | 43 | TIHDIILECV | 29-38 |
| Human papilloma | | A2 | | 41 | YMLDLQPET | 11-19 |
| virus | | A2 | | 168 | YMLDLQPETT | 11-20 |

In some embodiments, the exogenous antigenic polypeptide comprises or consist of a neoantigen polypeptide provided in Tables 16 and 17, or a fragment or variant thereof, or an antibody molecule thereto. Table 17 provides non-limiting examples of HLA polypeptide alleles that may be incorporated into a wild-type or loadable exogenous antigen-presenting polypeptide described herein, for binding to the specified neoantigen polypeptide.

In other embodiments, the exogenous antigenic polypeptide is an exogenous antigenic polypeptide derived from any one of the antigens disclosed herein. For example, in some embodiments the exogenous antigenic polypeptide is an exogenous antigenic polypeptide from an antigen selected from the antigens disclosed in Tables 7-8 and 16-26 and Table B. In some embodiments, the exogenous antigenic polypeptide is an antigenic polypeptide from an antigen selected from the antigens disclosed in Table 16. In some embodiments, the exogenous antigenic polypeptide is an antigenic polypeptide from an antigen selected from the antigens disclosed in Table 17. In some embodiments, the exogenous antigenic polypeptide is an antigenic polypeptide from an antigen selected from the antigens disclosed in Table 18. In some embodiments, the exogenous antigenic polypeptide is an antigenic polypeptide from an antigen selected from the antigens disclosed in Table 19. In some embodiments, the exogenous antigenic polypeptide is an antigenic polypeptide from an antigen selected from the antigens disclosed in Table 20. In some embodiments, the exogenous antigenic polypeptide is an antigenic polypeptide from an antigen selected from the antigens disclosed in Table 21. In some embodiments, the exogenous antigenic polypeptide is an antigenic polypeptide from an antigen selected from the antigens disclosed in Table 22 In some embodiments, the exogenous antigenic polypeptide is an antigenic polypeptide from an antigen selected from the antigens disclosed in Table 23 In some embodiments, the exogenous antigenic polypeptide is an antigenic polypeptide from an antigen selected from the antigens disclosed in Table 24. In some embodiments, the exogenous antigenic polypeptide is an antigenic polypeptide from an antigen selected from the antigens disclosed in Table 25. In some embodiments, the exogenous antigenic polypeptide is an antigenic polypeptide from an antigen selected from the antigens disclosed in Table 26. In some embodiments, the exogenous antigenic polypeptide is an antigenic polypeptide from an antigen selected from the antigens disclosed in Table B.

In some embodiments, an engineered erythroid cell or enucleated cell of the present disclosure comprises one or more exogenous antigenic polypeptides, wherein the one or more exogenous antigenic polypeptides is an HPV antigen. In some embodiments, an engineered erythroid cell or enucleated cell of the present disclosure comprises one or more exogenous antigenic polypeptides, wherein the one or more exogenous antigenic polypeptides is an HPV-E7 antigen. In some embodiments, an engineered erythroid cell or enucleated cell of the present disclosure comprises one or more exogenous antigenic polypeptides, wherein the one or more exogenous antigenic polypeptides is an HPV-E6 antigen. In some embodiments, an engineered erythroid cell or enucleated cell of the present disclosure comprises two or more exogenous antigenic polypeptides, wherein the two or more exogenous antigenic polypeptides comprise an HPV-E6 antigen and an HPV-E7 antigen.

In certain embodiments, the exogenous antigenic polypeptides are presented on a wild-type or loadable exogenous antigen-presenting polypeptide, e.g., the exogenous antigenic polypeptide is bound to the wild-type or loadable exogenous antigen-presenting polypeptide.

In some embodiments, the exogenous antigenic polypeptide is 8 amino acids in length to 24 amino acids in length, for example 8, 9, 10, 11, 12, 13, 14, 15, 16, 17, 18, 19, 20, 21, 22, 23, or 24 amino acids in length. In further embodiments, a cleavable site is introduced into the exogenous antigenic polypeptide.

In some embodiments, the exogenous antigenic polypeptide is covalently attached to the wild-type or loadable exogenous antigen-presenting polypeptide. In some embodiments, the exogenous antigenic polypeptide is non-covalently attached to the wild-type or loadable exogenous antigen-presenting polypeptide. Further details of covalent attachments are described in the methods below.

In some embodiments, an engineered erythroid cell (e.g., engineered enucleated erythroid cell) or enucleated cell (e.g., modified enucleated cell) provide herein comprises two types of exogenous polypeptides: at least one (one, two, three, or more) wild-type or loadable exogenous antigen-presenting polypeptides, and at least one (one, two, three, or more) exogenous antigenic polypeptide (e.g. a first and/or a second exogenous antigenic polypeptide). In some embodiments, a portion of the exogenous antigenic polypeptide is capable of binding to the antigen-binding cleft of the wild-type or loadable exogenous antigen-presenting polypeptide. In some embodiments, the at least one exogenous antigenic polypeptide(s) comprises a transmembrane domain such as a Type I membrane protein transmembrane domain (e.g., a GPA transmembrane domain), or a Type II membrane protein transmembrane domain (e.g., a small integral membrane protein 1 (SMIM1) transmembrane domain), as either an N-terminal or C-terminal fusion, e.g., such that the portion of the antigenic polypeptide that is capable of binding to a wild-type or loadable exogenous antigen-presenting polypeptide described herein is present on the outer side of the surface of the engineered erythroid cell or enucleated cell. In some embodiments, the exogenous antigenic polypeptide comprises a transmembrane domain, a linker, and an amino acid sequence (e.g., an antigen) that is capable of binding to the antigen-binding cleft of a wild-type or loadable exogenous antigen-presenting polypeptide. In some embodiments, the linker is a GlySer linker. In some embodiments, the linker is from about 30 to about 100 amino acid residues in length. In other embodiments, the linker is between about 40 amino acid residues in length and 70 amino acids in length. In some embodiments, the linker is a cleavable linker (e.g., comprising an enzymatic cleavage site described herein). In some embodiments, the exogenous antigenic polypeptide may be covalently-linked to an exogenous antigen-presenting polypeptide as described herein.

In some embodiments, the loadable exogenous antigen-presenting polypeptide has a higher affinity for the exogenous antigenic polypeptide than for an exogenous displaceable polypeptide. In some embodiments, the exogenous antigenic polypeptide binds to the loadable exogenous antigen-presenting polypeptide with a K_{D} of from about 1 picomolar to about 100 nanomolar, from about 5 picomolar to about 100 nanomolar, from about 10 picomolar to about 100 nanomolar, from about 20 picomolar to about 100 nanomolar, from about 30 picomolar to about 100 nanomolar, from about 50 picomolar to about 100 nanomolar, from about 100 picomolar to about 100 nanomolar, from about 200 picomolar to about 100 nanomolar, from about 300 picomolar to about 100 nanomolar, from about 400 picomolar to about 100 nanomolar, from about 500 picomolar to about 100 nanomolar, from about 600 picomolar to about 100 nanomolar, from about 700 picomolar to about 100 nanomolar, from about 800 picomolar to about 100 nanomolar, from about 900 picomolar to about 100 nanomolar, from about 1 nanomolar to about 100 nanomolar, from about 2 nanomolar to about 100 nanomolar, from about 5 nanomolar to about 100 nanomolar, from about 10 nanomolar to about 100 nanomolar, from about 20 nanomolar to about 100 nanomolar, from about 30 nanomolar to about 100 nanomolar, from about 40 nanomolar to about 100 nanomolar, from about 50 nanomolar to about 100 nanomolar, from about 60 nanomolar to about 100 nanomolar, from about 70 nanomolar to about 100 nanomolar, from about 80 nanomolar to about 100 nanomolar, or from about 90 nanomolar to about 100 nanomolar.

Methods of detecting peptides that specifically bind to particular HLA polypeptide and methods of determining the affinity of the peptide for the HLA polypeptide are described herein.

Methods of Displacing A Displaceable Polypeptide from a Loadable Exogenous Antigen-Presenting Polypeptide

In some embodiments, the present disclosure provides an engineered erythroid cell (e.g., engineered enucleated erythroid cell) or enucleated cell (e.g., modified enucleated cell) comprising a loadable exogenous antigen-presenting polypeptide comprising a displaceable exogenous polypeptide. In some embodiments, the displaceable exogenous polypeptide is displaced from the wild-type or loadable exogenous antigen-presenting polypeptide, e.g., from the antigen-binding cleft of a HLA class I or class II polypeptide present in the exogenous antigen-presenting polypeptide, and a selected exogenous antigenic polypeptide can be bound to the wild-type or loadable exogenous antigen-presenting polypeptide.

In some embodiments, displacing the displaceable exogenous polypeptide from the wild-type or loadable exogenous antigen-presenting polypeptide and specifically-binding an exogenous antigenic polypeptide to the wild-type or loadable exogenous antigen-presenting polypeptide comprises contacting the engineered erythroid cell or enucleated cell in vitro with the exogenous antigenic polypeptide, wherein the wild-type or loadable exogenous antigen-presenting polypeptide has a higher affinity for the exogenous antigenic polypeptide than for an exogenous displaceable polypeptide. In some embodiments, the exogenous displaceable polypeptide binds to the wild-type or loadable exogenous antigen-presenting polypeptide with a K_{D} of from about 1 nM to about 100 μM. In some embodiments, the exogenous antigenic polypeptide binds to the wild-type or loadable exogenous antigen-presenting polypeptide with a K_{D} of from about 1 picomolar to about 100 nanomolar.

Multiple displaceable exogenous polypeptides are described above. In some embodiments, the displaceable exogenous polypeptide comprises an amino acid sequence provided in Table 6. In some embodiments, the displaceable exogenous polypeptide comprises or consists of the amino acid sequence ILKEPVHGV (SEQ ID NO: 138). In some embodiments, the displaceable exogenous polypeptide is IAKEPVHGV (SEQ ID NO: 139). In some embodiments, the displaceable exogenous polypeptide comprises or consists of the amino acid sequence ILKEPVHGA (SEQ ID NO: 140). In some embodiments, the displaceable exogenous polypeptide comprises or consists of the amino acid sequence IAKEPVHGA (SEQ ID NO: 141). In some embodiments, the displaceable exogenous polypeptide comprises or consists of the amino acid sequence GLKEPQIQV (SEQ ID NO: 142). In some embodiments, the displaceable exogenous polypeptide comprises or consists of the amino acid sequence NLVPMVATA (SEQ ID NO: 143). In some embodiments, the displaceable exogenous polypeptide comprises or consists of the amino acid sequence IRAAPPPLA (SEQ ID NO: 144).

In some embodiments, a wild-type or loadable exogenous antigen-presenting polypeptide described herein, or an engineered erythroid cell (e.g., engineered enucleated erythroid cell) or enucleated cell (e.g., modified enucleated cell) comprising the same is contacted with a dipeptide to accelerate the dissociation of a prebound exogenous displaceable polypeptide (see, e.g., Saini et al. (2015) Proc. Nat'l. Acad. Sci. USA 112: 202-7, incorporated in its entirety herein by reference). In some embodiments, a wild-type or loadable exogenous antigen-presenting polypeptide described herein or an engineered erythroid cell or enucleated cell comprising the same is concurrently contacted with a dipeptide and an exogenous antigenic polypeptide. In some embodiments, the engineered erythroid cell (e.g., engineered enucleated erythroid cell) or enucleated cell (e.g., modified enucleated cell) is sequentially contacted with a dipeptide and an exogenous antigenic polypeptide, in any order. In some embodiments, the loadable exogenous antigen-presenting polypeptide comprises an amino acid sequence derived from an HLA class I allele, and the dipeptide is selected from glycyl-leucine (GL), glycyl-methionine (GM), GG, glycyl-alanine (GA), glycyl-valine (GV), glycyl-phenylalanine (GF), leucyl-glycine (LG), glycyl-cyclohexylalanine (G-Cha), glycyl-homoleucine (GHLe), acetylated leucine, and glycyl-arginine (GR), or a combination thereof. In some embodiments, the loadable exogenous antigen-presenting polypeptide comprises an amino acid sequence derived from an allele selected from HLA-A:02:01, HLA-A1:01, HLA-A3:01, HLA-A24:02, HLA-A26:01, HLA-B7:02, HLA-B08:01, HLA-B27:05, HLA-B39:01, HLA-B40:01, HLA-B58:01, HLA-B15:01, and HLA-E01:01, and the dipeptide is selected from glycyl-leucine (GL), glycyl-methionine (GM), GG, glycyl-alanine (GA), glycyl-valine (GV), glycyl-phenylalanine (GF), leucyl-glycine (LG), glycyl-cyclohexylalanine (G-Cha), glycyl-homoleucine (GHLe), acetylated leucine, and glycyl-arginine (GR), or a combination thereof. In some embodiments, the loadable exogenous antigen-presenting polypeptide comprises HLA-B:27:05 and the dipeptide is GR or G-Cha.

Multiple assays for determining whether a dipeptide specifically binds to a particular ligand (e.g., an exogenous antigen-presenting polypeptide) are known in the art. For example, in some embodiments, a fluorescence anisotropy assay can be used to observe the binding kinetics and determine the peptide association and dissociation rates in a polypeptide-complex (see, e.g., Saini et al. (2015)). The thermal stability of the HLA class I-polypeptide complex can be measured by TDTF experiments as described in Saini et al. (2013) Mol. Immunol 54(3-4):386-96, incorporated in its entirety herein by reference. Furthermore, cell-surface peptide exchange can be determined by flow cytometry (see, e.g., Saini et al. (2015)).

In some embodiments, displacing the displaceable exogenous polypeptide from the loadable exogenous antigen-presenting polypeptide comprises enzymatically cleaving a linker disposed between the exogenous displaceable polypeptide sequence and the displaceable exogenous polypeptide sequence. Cleavage of the linker facilitates the release and removal of the exogenous displaceable polypeptide from the loadable exogenous antigen-presenting polypeptide. Thus, in some embodiments, the loadable exogenous antigen-presenting polypeptide and the exogenous displaceable polypeptide are connected via a linker, and the linker comprises an enzymatic cleavage site. Suitable enzymatic cleavage sites include, but are not limited to human rhinovirus (HRV) 3C protease cleavage site and tobacco etch virus (TEV) protease cleavage site. In some embodiments, the loadable exogenous antigen-presenting polypeptide or a cell comprising the same may be contacted with an enzyme specific for the enzymatic cleavage site. In some embodiments, the enzymatic cleavage site comprises the amino acid sequence LEVLFQGP (SEQ ID NO: 1). In some embodiments, the enzymatic cleavage site comprises the amino acid sequence ENLYFQG (SEQ ID NO: 2).

In some embodiments, enzymatic cleavage of the linker exposes a motif that may be used to conjugate a desired exogenous antigenic polypeptide to the linker, thereby facilitating loading (e.g., specific binding) of the exogenous antigenic polypeptide onto the loadable exogenous antigen-presenting polypeptide. Any motif(s) that are recognized by enzymes that catalyze the conjugation of polypeptides may be incorporated into either the linker and/or the desired exogenous antigenic polypeptide as long as they are compatible in order to facilitate the conjugation reaction. For instance, motifs recognized by enzymes such as sortase (e.g., GGG, GG, G and any one of LPTXG (SEQ ID NO: 3), IPKTG (SEQ ID NO: 881), MPXTG (SEQ ID NO: 882), LAETG (SEQ ID NO: 883), LPXAG (SEQ ID NO: 884), LPESG (SEQ ID NO: 885), LPELG (SEQ ID NO: 886), LPEVG (SEQ ID NO: 887) (see, e.g., Antos et al. (2016) Curr. Opin. Struct. Biol. 38:111-8), butelase 1 (e.g., C-terminal NHV and any one of N-terminal X₁X₂ amino acid sequence, wherein X1 is any amino acid and X2 is I, L, V, or C (see, e.g., Nguyen et al. 2016 Nat Protocols 11: 1977-88)), or SpyCatcher (AHIVMVDAYKPTK (SEQ ID NO: 4) or ATHIKFSKRD (SEQ ID NO: 5)) may be used.

In some embodiments, any sortase known in the art can be used to conjugate an exogenous antigenic polypeptide to an antigen-presenting polypeptide described herein. Sortases have been classified into 4 classes, designated A, B, C, and D, based on sequence alignment and phylogenetic analysis (see, e.g., Dramsi et al. (2005) Res. Microbiol. 156 (3): 289-97). The term “sortase A” is used herein to refer to a class A sortase, usually named SrtA in any particular bacterial species, e.g., SrtA from Staphylococcus aureus or S. pyogenes. Likewise “sortase B” is used herein to refer to a class B sortase, usually named SrtB in any particular bacterial species, e.g., SrtB from S. aureus. The present disclosure encompasses embodiments relating to any of the sortase classes known in the art (e.g., a sortase A from any bacterial species or strain, a sortase B from any bacterial species or strain, a class C sortase from any bacterial species or strain, and a class D sortase from any bacterial species or strain). In some embodiments, a sortase that utilizes a nucleophilic acceptor sequence having an N-terminal glycine, e.g., 1-5 N-terminal glycines, is used, such as SrtA from S. aureus. In some embodiments it is contemplated to use two or more sortases. In some embodiments the sortases may utilize different sortase recognition sequences and/or different nucleophilic acceptor sequences. For example, SrtA from S. pyogenes can utilize a nucleophilic acceptor sequence having one or more N-terminal alanines, e.g., 1-5 N-terminal alanines and/or may utilize a sortase recognition motif comprising LPXTA (SEQ ID NO: 37).

An exemplary wild type S. aureus SrtA sequence (Gene ID: 1125243, NCBI RefSeq Acc. No. NP_375640.1) is shown below in

SEQ ID NO: 888
MKKWTNRLMTIAGVVLILVAAYLFAKPHIDNYLHDKDKDEKIEQYDKNVK
EQASKDNKQQAKPQIPKDKSKVAGYIEIPDADIKEPVYPGPATPEQLNRG
VSFAEENESLDDQNISIAGHTFIDRPNYQFTNLKAAKKGSMVYFKVGNET
RKYKMTSIRDVKPTDVEVLDEQKGKDKQLTLITCDDYNEKTGVWEKRKIF
VATEVK.

One of ordinary skill in the art will appreciate that different subspecies, strains, and isolates may differ in sequence at positions that do not significantly affect activity. For example, another exemplary wild type S. aureus SrtA sequence (Gene ID: 3238307, NCBI RefSeq Acc. No. YP_187332.1; GenBank Acc. No. AAD48437) has a K residue at position 57 and a G residue at position 167, as shown below in

SEQ ID NO: 889
MKKWTNRLMTIAGVVLILVAAYLFAKPHIDNYLHDKDKDEKIEQYDKNVK
EQASKDKKQQAKPQIPKDKSKVAGYIEIPDADIKEPVYPGPATPEQLNRG
VSFAEENESLDDQNISIAGHTFIDRPNYQFTNLKAAKKGSMVYFKVGNET
RKYKMTSIRDVKPTDVGVLDEQKGKDKQLTLITCDDYNEKTGVWEKRKIF
VATEVK.

In some embodiments, a calcium-independent sortase A variant may be used as described herein and comprises at least three amino acid substitutions relative to a wild-type sortase A, wherein the amino acid substitutions comprise a) a K residue at position 105; b) a Q or A residue at position 108; and c) at least one amino acid substitution selected from the group consisting of i) a R residue at position 94; ii) a S residue at position 94; iii) a N residue at position 160; iv) a A residue at position 165; v) a E residue at position 190; and vi) a T residue at position 196. In some embodiments a calcium-independent sortase A variant comprises the following amino acid substitutions relative to a wild-type sortase A: a) a K residue at position 105; b) a Q or A residue at position 108; c) an S residue at position 94 or R residue at position 94; d) an N residue at position 160; e) an A residue at position 165, and a T residue at position 196. In some embodiments, a calcium-independent sortase A variant comprises the following amino acid substitutions relative to a wild-type sortase A: a) a K residue at position 105; b) a Q or A residue at position 108; c) a R or S residue at position 94; d) a N residue at position 160; e) a A residue at position 165; f) a E residue at position 190; and g) a T residue at position 196. In some embodiments, a sortase comprises the following sequence, in which amino acids at positions 94, 105, 108, 160, 165, 190, and 196 relative to a full length S. aureus SrtA sequence are shown in bold:

(SEQ ID NO: 890)
MQAKPQIPKDKSKVAGYIEIPDADIKEPVYPGPATREQLNRGVSFAEENE
SLDDQNISIAGHTFIDRPNYQFTNLKAAKKGSMVYFKVGNETRKYKMTSI
RNVKPTAVEVLDEQKGKDKQLTLITCDDYNEETGVWETRKIFVATEVK.

In some embodiments, a transamidase that has an altered substrate selectivity as compared with a naturally occurring sortase may be used. For example, variants of S. aureus sortase A that accept aromatic amino acids (e.g., phenylalanine), as well as amino acids with small side chains such as Ala, Asp, Ser, Pro, and Gly, at position 1 of the sortase recognition motif (instead of L) have been identified (Piotukh et al. (2011) J. Am. Chem. Soc. 133(44): 17536-9, the entire content of which is incorporated herein by reference). In some embodiments such a sortase is used in a composition or method of the invention. A sortase with an altered substrate selectivity with regard to the sortase recognition motif may be generated by engineering one or more mutations in the sortase, e.g., in a region of the protein that is involved in recognition and/or binding of the sortase recognition motif, e.g., the putative substrate recognition loop (e.g., the loop connecting strands P6 and P7 (β6/β7 loop) in SrtA (Va1161-Asp176). In some embodiments, a phage-display, yeast display, or other screen of a mutant sortase library randomized in the substrate recognition loop may be performed, and variants with altered substrate specificity may be identified.

In some embodiments the sortase is a sortase B (SrtB), e.g., a sortase B of S. aureus, Bacillus anthracis, or Listeria monocytogenes. Motifs recognized by sortases of the B class (SrtB) often fall within the consensus sequences NPXTX, e.g., NP[Q/K]-[T/sHN/G/s], such as NPQTN (SEQ ID NO: 891) or NPKTG (SEQ ID NO: 892), and can be adapted for use as described herein. For example, sortase B of S. aureus or B. anthracis cleaves the NPQTN (SEQ ID NO: 891) or NPKTG motif (SEQ ID NO: 892) of IsdC in the respective bacteria (see, e.g., Marraffini and Schneewind (2007) J. Bact. 189(17): 6425-36). Other recognition motifs found in putative substrates of class B sortases are NSKTA (SEQ ID NO: 893), NPQTG (SEQ ID NO: 894), NAKTN (SEQ ID NO: 895), and NPQSS (SEQ ID NO: 896). For example, SrtB from L. monocytogenes recognizes certain motifs lacking P at position 2 and/or lacking Q or K at position 3, such as NAKTN (SEQ ID NO: 895) and NPQSS (SEQ ID NO: 896).

In some embodiments, the sortase is a class C sortase. Class C sortases may utilize LPXTG (SEQ ID NO: 36) as a recognition motif.

In some embodiments, the sortase is a class D sortase. Sortases in this class are predicted to recognize motifs with a consensus sequence NA-[E/A/S/H]-TG (SEQ ID NO: 897). Class D sortases have been found, e.g., in Streptomyces spp., Corynebacterium spp., Tropheryma whipplei, Thermobifida fusca, and Bifidobacterium longhum. LPXTA (SEQ ID NO: 37) or LAXTG (SEQ ID NO: 898) may serve as a recognition sequence for class D sortases

Motifs recognizable by a sortase are well known in the art, and any such known motifs may be utilized. One exemplary motif recognizable by a sortase, particularly sortase A, is LPXTG (SEQ ID NO: 36), in which X can be any amino acid residue (naturally-occurring or non-naturally occurring), e.g., any of the 20 standard amino acids found most commonly in proteins found in living organisms. In some embodiments, the recognition motif is LPXTG (SEQ ID NO: 36) or LPXT, in which X is D, E, A, N, Q, K, or R. In some embodiments, X is selected from K, E, N, Q, A in an LPXTG (SEQ ID NO: 36) or LPXT motif, which are recognizable by a sortase A. In some embodiments, X is selected from K, S, E, L, A, N in an LPXTG (SEQ ID NO: 36) or LPXT motif, which are recognizable by a class C sortase. Exemplary sortase recognition motifs include, but are not limited to, LPKTG (SEQ ID NO: 899), LPITG (SEQ ID NO: 900), LPDTA (SEQ ID NO: 901), SPKTG (SEQ ID NO: 902), LAETG (SEQ ID NO: 883), LAATG (SEQ ID NO: 903), LAHTG (SEQ ID NO: 904), LASTG (SEQ ID NO: 905), LPLTG (SEQ ID NO: 906), LSRTG (SEQ ID NO: 907), LPETG (SEQ ID NO: 908), VPDTG (SEQ ID NO: 909), IPQTG (SEQ ID NO: 910), YPRRG (SEQ ID NO: 911), LPMTG (SEQ ID NO: 912), LAFTG (SEQ ID NO: 913), LPQTS (SEQ ID NO: 914), LPXT, LAXT, LPXA, LGXT, IPXT, NPXT, NPQS (SEQ ID NO: 915), LPST (SEQ ID NO: 916), NSKT (SEQ ID NO: 917), NPQT (SEQ ID NO: 918), NAKT (SEQ ID NO: 919), LPIT (SEQ ID NO: 920), LAET (SEQ ID NO: 921), LPXTX, LPKTG (SEQ ID NO: 899), LPATG (SEQ ID NO: 923), LPNTG (SEQ ID NO: 924), or LPETG (SEQ ID NO: 908); wherein each occurrence of X represents independently any amino acid residue. In some embodiments the motif comprises an ‘A’ rather than a ‘T’ at position 4, e.g., LPXAG (SEQ ID NO: 884), e.g., LPNAG (SEQ ID NO: 925), wherein each occurrence of X represents independently any amino acid residue). In some embodiments the motif comprises an ‘A’ rather than a G′ at position 5, e.g., LPXTA (SEQ ID NO: 37), e.g., LPNTA (SEQ ID NO: 926), wherein each occurrence of X represents independently any amino acid residue). In some embodiments the motif comprises a G′ rather than ‘P’ at position 2, e.g., LGXTG (SEQ ID NO: 927), e.g., LGATG (SEQ ID NO: 928), wherein each occurrence of X represents independently any amino acid residue). In some embodiments the motif comprises an ‘I’ rather than 1′ at position 1, e.g., IPXTG (SEQ ID NO: 929), e.g., IPNTG (SEQ ID NO: 930) or IPETG (SEQ ID NO: 931), wherein each occurrence of X represents independently any amino acid residue). The motifs may be present immediately adjacent to the enzymatic cleavage site or within a range of amino acids such that they are recognized by the conjugating enzyme.

In some embodiments, the enzymatic cleavage site is within 10 amino acids or less from a GGG motif in the linker, and is located between the GGG motif and the exogenous displaceable polypeptide. In some embodiments, the GGG motif is C terminal to the enzymatic cleavage site.

In some embodiments, the enzymatic cleavage site is within 10 amino acids or less from an LPTXG motif (SEQ ID NO: 3), and is located between the LPTXG motif (SEQ ID NO: 3) and the exogenous displaceable polypeptide. In some embodiments, the LPTXG motif (SEQ ID NO: 3) is C terminal to the enzymatic cleavage site.

In some embodiments, the enzymatic cleavage site is within 10 amino acids or less from an NHV motif, and is located between the NHV motif and the exogenous displaceable polypeptide. In some embodiments, the NHV motif is C terminal to the enzymatic cleavage site. In some embodiments, the enzymatic cleavage site is adjacent to a SpyTag sequence (AHIVMVDAYKPTK (SEQ ID NO: 4)), and is located between the SpyTag sequence and the exogenous displaceable polypeptide. In some embodiments, the SpyTag sequence is C terminal to the enzymatic cleavage site. In some embodiments, the enzymatic cleavage site is within 10 amino acids or less from the amino acid sequence AHIVMVDAYKPTK (SEQ ID NO: 4).

In some embodiments, the enzymatic cleavage site is adjacent to a KTag sequence (ATHIKFSKRD (SEQ ID NO: 5)), and is located between the KTag sequence and the exogenous displaceable polypeptide. In some embodiments, the KTag sequence is C terminal to the enzymatic cleavage site. In some embodiments, the enzymatic cleavage site is within 10 amino acids or less from the amino acid sequence ATHIKFSKRD (SEQ ID NO: 5).

### Methods of Conjugating an Exogenous Antigenic Polypeptide to a Wild-Type or Loadable Exogenous Antigen-Presenting Polypeptide

In some embodiments, the present disclosure provides an engineered erythroid cell (e.g., engineered enucleated erythroid cell) or enucleated cell (e.g., modified enucleated cell) comprising a wild-type or loadable exogenous antigen-presenting polypeptide on the cell surface, wherein the loadable exogenous antigen-presenting polypeptide comprises one or more amino acid substitutions which stabilize the loadable exogenous antigen-presenting polypeptide on the cell surface. In some embodiments, the loadable exogenous antigen-presenting polypeptide is stabilized on the cell surface in the absence of a polypeptide bound to the exogenous antigen-presenting polypeptide. In some embodiments, an exogenous antigenic polypeptide is bound to the wild-type or loadable exogenous antigen-presenting polypeptide. In other embodiments, the loadable exogenous antigen-presenting polypeptide comprises a displaceable exogenous polypeptide which is displaced from the loadable exogenous antigen-presenting polypeptide, and a selected exogenous antigenic polypeptide is bound to the loadable exogenous antigen-presenting polypeptide.

In some embodiments, specifically binding an exogenous antigenic polypeptide to the wild-type or loadable exogenous antigen-presenting polypeptide comprises conjugating the exogenous antigenic polypeptide to the wild-type or loadable exogenous antigen-presenting polypeptide. For example, in some embodiments, the exogenous antigenic polypeptide can be conjugated to the wild-type or loadable exogenous antigen-presenting polypeptide using click chemistry, as described in detail herein.

In some embodiments, coupling reagents can be used to couple an exogenous antigenic polypeptide to a wild-type or loadable exogenous antigen-presenting polypeptide. Click chemistry and other conjugation methods for functionalizing erythroid cells is described in International Patent Publication No. WO 2018/151829, the entire contents of which are incorporated herein by reference.

In some embodiments, an engineered erythroid cell (e.g., engineered enucleated erythroid cell) or enucleated cell (e.g., modified enucleated cell) described herein comprises many as, at least, more than, or about 5,000, 10,000, 50,000, 100,000, 200,000, 300,000, 400,000, 500,000 coupling reagents per cell. In some embodiments, the engineered erythroid cells (e.g., engineered enucleated erythroid cells) or enucleated cells (e.g., modified enucleated cells) are made by a method comprising a) coupling a first coupling reagent to a wild-type or loadable exogenous antigen-presenting polypeptide, thereby making a pharmaceutical preparation, product, or intermediate. In an embodiment, the method further comprises: b) contacting the wild-type or loadable exogenous antigen-presenting polypeptide with an exogenous antigenic polypeptide coupled to a second coupling reagent e.g., under conditions suitable for reaction of the first coupling reagent with the second coupling reagent. In some embodiments, two or more exogenous antigenic polypeptides are coupled to the wild-type or loadable exogenous antigen-presenting polypeptide (e.g., using click chemistry).

In some embodiments, the coupling reagent comprises an azide coupling reagent. In some embodiments, the azide coupling reagent comprises an azidoalkyl moiety, azidoaryl moiety, or an azidoheteroaryl moiety. Exemplary azide coupling reagents include 3-azidopropionic acid sulfo-NHS ester, azidoacetic acid NHS ester, azido-PEG-NHS ester, azidopropylamine, azido-PEG-amine, azido-PEG-maleimide, bis-sulfone-PEG-azide, or a derivative thereof. Coupling reagents may also comprise an alkene moiety, e.g., a transcycloalkene moiety, an oxanorbornadiene moiety, or a tetrazine moiety. Additional coupling reagents can be found in Click Chemistry Tools (available on the world wide web at clickchemistrytools.com) or Lahann, J. (ed.) (2009) Click Chemistry for Biotechnology and Materials Science, each of which is incorporated herein by reference in its entirety.

In some embodiments, the exogenous antigenic polypeptide is attached to the wild-type or loadable exogenous antigen-presenting polypeptide, via a covalent attachment to generate an engineered erythroid cell or enucleated cell comprising an engineered erythroid cell or enucleated cell presenting one or more exogenous antigenic polypeptides (e.g. a first exogenous antigenic polypeptide, or a first antigenic polypeptide and a second exogenous antigenic polypeptide). For example, the exogenous antigenic polypeptide may be derivatized and bound to the wild-type or loadable exogenous antigen-presenting polypeptide using a coupling compound containing an electrophilic group that will react with nucleophiles on the engineered erythroid cell or enucleated cell (e.g., nucleophiles present on the wild-type or loadable exogenous antigen-presenting polypeptide on the cell) to form the interbonded relationship. Representative of these electrophilic groups are αβ unsaturated carbonyls, alkyl halides and thiol reagents such as substituted maleimides. In addition, the coupling compound can be coupled to an antigenic polypeptide via one or more of the functional groups in the polypeptide such as amino, carboxyl and tryosine groups. For this purpose, coupling compounds should contain free carboxyl groups, free amino groups, aromatic amino groups, and other groups capable of reaction with enzyme functional groups. Highly charged antigenic polypeptides can also be prepared for immobilization on, e.g., an exogenous wild-type or loadable antigen-presenting polypeptide, through electrostatic bonding to generate a modified enucleated cell. Examples of these derivatives would include polylysyl and polyglutamyl enzymes.

The choice of the reactive group embodied in the derivative depends on the reactive conditions employed to couple the electrophile with the nucleophilic groups on the exogenous wild-type or loadable antigen-presenting polypeptide for immobilization. A controlling factor is the desire not to inactivate the coupling reagent prior to coupling of the exogenous antigenic polypeptide immobilized by the attachment to the exogenous wild-type or loadable antigen-presenting polypeptide. Such coupling immobilization reactions can proceed in a number of ways. Typically, a coupling reagent can be used to form a bridge between the exogenous antigenic polypeptide and the wild-type or loadable exogenous antigen-presenting polypeptide. In this case, the coupling reagent should possess a functional group such as a carboxyl group which can be caused to react with the exogenous antigenic polypeptide. One way of preparing the exogenous antigenic polypeptide for conjugation includes the utilization of carboxyl groups in the coupling reagent to form mixed anhydrides which react with the exogenous antigenic polypeptide, in which use is made of an activator which is capable of forming the mixed anhydride. Representative of such activators are isobutylchloroformate or other chloroformates which give a mixed anhydride with coupling reagents such as 5,5′-(dithiobis(2-nitrobenzoic acid) (DTNB), p-chloromercuribenzoate (CMB), or m-maleimidobenzoic acid (MBA). The mixed anhydride of the coupling reagent reacts with the exogenous antigenic polypeptide to yield the reactive derivative which in turn can react with nucleophilic groups on the engineered erythroid cell or enucleated cell (e.g., on an exogenous wild-type or loadable antigen-presenting polypeptide present on the cell surface) to immobilize the exogenous antigenic polypeptide.

Functional groups on an antigenic polypeptide, such as carboxyl groups can be activated with carbodiimides and the like activators. Subsequently, functional groups on the bridging reagent, such as amino groups, will react with the activated group on the exogenous antigenic polypeptide to form the reactive derivative. In addition, the coupling reagent should possess a second reactive group which will react with appropriate nucleophilic groups on the wild-type or loadable antigen-presenting polypeptide to form the bridge. Typical of such reactive groups are alkylating agents such as iodoacetic acid, αβ unsaturated carbonyl compounds, such as acrylic acid and the like, thiol reagents, such as mercurials, substituted maleimides and the like.

Alternatively, functional groups on the exogenous antigenic polypeptide can be activated so as to react directly with nucleophiles on, e.g., on a wild-type or loadable exogenous antigen-presenting polypeptide, to obviate the need for a bridge-forming compound. For this purpose, use is made of an activator such as Woodward's Reagent K or the like reagent which brings about the formation of carboxyl groups in the exogenous antigenic polypeptide into enol esters, as distinguished from mixed anhydrides. The enol ester derivatives of exogenous antigenic polypeptides subsequently react with nucleophilic groups on, e.g., a wild-type or loadable exogenous antigen-presenting polypeptide to effect immobilization of the exogenous antigenic polypeptide, thereby conjugating the exogenous antigenic polypeptide onto the wild-type or loadable antigen-presenting polypeptide.

In some embodiments, the exogenous antigenic polypeptide can be derivatized or activated as described above to yield an exogenous antigenic polypeptide bearing one or more reactive functional groups that can react with one or more amino acid residues located on the wild-type or loadable exogenous antigen-presenting polypeptide of the engineered erythroid cells (e.g., engineered enucleated erythroid cells) or enucleated cells (e.g., modified enucleated cells).

In some embodiments, the exogenous antigenic polypeptide comprises one or more (e.g., two, three, four, five, etc.) thiol-reactive groups that can react with one or more cysteine thiol groups located on the wild-type or loadable exogenous antigen-presenting polypeptide of the engineered erythroid cells (e.g., engineered enucleated erythroid cells) or enucleated cells (e.g., modified enucleated cells).

As used herein, a thiol-reactive functional group is a functional group that can readily react with the thiol group (e.g., a thiol group of a cysteine residue) to form a covalent bond. Any suitable thiol-reactive functional groups can be used.

In some embodiments, the thiol-reactive groups include, but are not limited to, (i) 2-cyanobenzothiazole (CBT) (see Ren et al. (2009) Agnew. Chem. Int. Ed. 48: 9658-62; Chen et al. (2018) ChemistryOpen 7: 256-61; Zhang and Liang (2018) Sci. China Chem. 61: 1-11); (ii) maleimide or maleimide derivatives (Ravasco et al. (2019) Chem. Eur. 1 25: 43-59) (e.g., N-aryl maleimide, beta amino maleimide, acetal containing maleimides, exocyclic maleimides, dialkylmaleimide, halogenated maleimide (e.g., bromomaleimide, dibromomaleimide, diiodomaleimide (Behrens et al. (2015) Mol. Pharm. 12(11): 3986-98)) or aryldithiomaleimide (e.g., dithiophenylmaleimide (Nunes et al. (2015) Chem. Commun. (Camb). 51(53): 10624-7))); (iii) arylpropionitriles (e.g., 3-arylpropiolonitrile (Koniev et al. (2014) Bioconjug. Chem. 25(2): 202-6) or 3,3′-arylene-dipropiolonitrile (Koniev et al. (2018) Medchemcomm. 9(5):827-30); (iv) sulfones (e.g., phenyloxadiazolyl sulfone (Patterson et al. (2014) Bioconjugate Chem. 25, 1402-7), benzothiazolyl sulfone (Toda et al. (2013) Agnew Chem. Int. Ed. Engl. 52(48): 12592-6), or bis-sulfone (Badescu et al. (2014) Bioconjug. Chem. 25(6): 1124-36); (v) allenamide (e.g., benzylallenamide, cyclohexylallenamide, naphthylallenamide, aminoethylallenamide, etc. (Abbas et al. (2014) Angew Chem. Int. Ed. Engl. 53(29): 7491-4)); (vi) dibromopyridazinedione (e.g., 4,5-dibromo-1,2-dihydro-pyridazine-3,6-dione (Bahou et al. (2018) Org. Biomol. Chem. 16(8): 1359-66)), (vii) disulfides (e.g., nitropyridyl disulfide (Sadowsky et al. (2017) Bioconjug. Chem. 28(8): 2086-98)), and (viii) haloacetamide (e.g., iodoacetamide, bromoacetamide or chloroacetamide (Free et al. (2006) Org. Biomol. Chem. 4(9): 1817-30)).

Table A lists exemplary exogenous antigenic polypeptides having a thiol-reactive funtional group and the resulting modified engineered erythroid cell or enucleated cell. P₁ represents the exogenous antigenic polypeptide; L is absent or represents a spacer connecting the reactive functional group and the peptide; P₂ represents the wild-type or loadable exogenous antigen-presenting polypeptide located on the surface of the engineered erythroid cells (e.g., engineered enucleated erythroid cells) or enucleated cells (e.g., modified enucleated cells). Cys-SH represents the N- or C-terminal cysteine of the wild-type or loadable exogenous antigen-presenting polypeptide.

In some embodiments, the cysteine of the wild-type or loadable exogenous antigen-presenting polypeptide located on the surface of the engineered erythroid cells (e.g., engineered enucleated erythroid cells) or enucleated cells (e.g., modified enucleated cells) is an N-terminal cysteine of the wild-type or loadable exogenous antigen-presenting polypeptide, e.g., wherein the wild-type or loadable exogenous antigen-presenting polypeptide comprises a Type I membrane protein transmembrane domain (e.g., GPA). In some embodiments, the N-terminal cysteine is located in a linker, e.g., a linker disposed adjacent to, e.g., N-terminal to, the β2M polypeptide. In some embodiments, the cysteine of the wild-type or loadable exogenous antigen-presenting polypeptide is a C-terminal cysteine of the wild-type or loadable exogenous antigen-presenting polypeptide, e.g., wherein the wild-type or loadable exogenous antigen-presenting polypeptide comprises a Type II membrane protein transmembrane domain (e.g., SMIM1). In some embodiments, the C-terminal cysteine is located in a linker, e.g., a linker disposed adjacent to, e.g., C-terminal to, the β2M polypeptide. For example, the linker may be any one of the linkers presented in Table 3, where the N-terminal or C-terminal amino acid residue is replaced with a cysteine.

In some embodiments, where the wild-type or loadable exogenous antigen-presenting polypeptide comprises an exogenous displaceable polypeptide, an enzymatic cleavage site may be disposed in the linker connecting the HLA polypeptide and the exogenous displaceable polypeptide, wherein the enzymatic cleavage of the linker exposes an N-terminal or C-terminal cysteine that may be used to conjugate a desired exogenous antigenic polypeptide to the linker, thereby facilitating loading (e.g., specific binding) of the exogenous antigenic polypeptide onto the wild-type or loadable exogenous antigen-presenting polypeptide.

TABLE A

| Reactive Group | |
|---|---|
| Cyanobenzothiazole (CBT) | 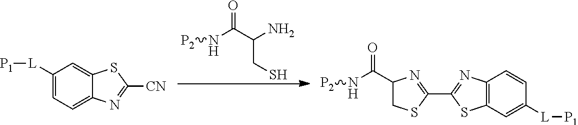 |
| Maleimide or maleimide derivative | 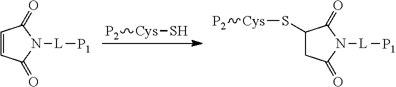 |
| | 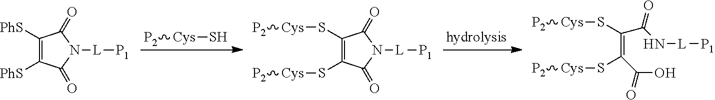 |
| | 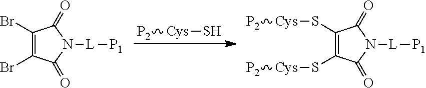 |
| Arylpropionitrile | 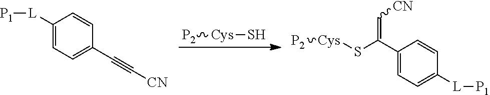 |
| Sulfone | 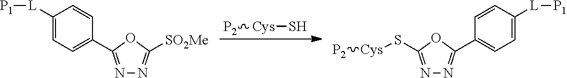 |
| | 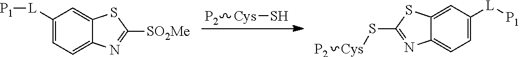 |
| Allenamide | 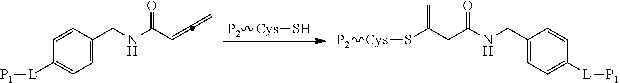 |
| Dibromopyridazinedione | 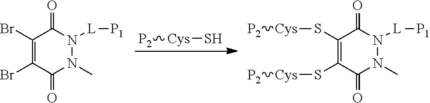 |
| Disulfide | 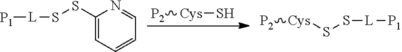 |
| Haloacetamide | 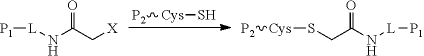 |
| | X = Br, Cl or I |

In some embodiments, the exogenous antigenic polypeptide having one or more (e.g., two, three, four, five, etc.) thiol-reactive groups is covalently linked to a N-terminal cysteine and/or a C-terminal cysteine of the wild-type or loadable exogenous antigen-presenting polypeptide. In some embodiments, the exogenous antigenic polypeptide derivative having one or more cyanobenzothiazole groups is covalently linked to a N-terminal cysteine of the wild-type or loadable exogenous antigen-presenting polypeptide. In some embodiments, the exogenous antigenic polypeptide having one or more maleimide or maleimide derivative groups is covalently linked to a N-terminal cysteine and/or a C-terminal cysteine of the wild-type or loadable exogenous antigen-presenting polypeptide. In some embodiments, the maleimide or maleimide derivative is a N-aryl maleimide. In some embodiments, the maleimide or maleimide derivative is a beta amino maleimide. In some embodiments, the maleimide or maleimide derivative is an acetal containing maleimide. In some embodiments, the maleimide or maleimide derivative is an exocyclic maleimide. In some embodiments, the maleimide or maleimide derivative is a dialkylmaleimide. In some embodiments, the maleimide or maleimide derivative is a halogenated maleimide e.g., bromomaleimide, dibromomaleimide, or diiodomaleimide. In some embodiments, the maleimide or maleimide derivative is an aryldithiomaleimide, e.g., dithiophenylmaleimide. In some embodiments, the exogenous antigenic polypeptide having one or more arylpropionitrile groups is covalently linked to a N-terminal cysteine and/or a C-terminal cysteine of the wild-type or loadable exogenous antigen-presenting polypeptide. In some embodiments, the arylpropionitrile is a 3-arylpropiolonitrile. In some embodiments, the arylpropionitrile is a 3,3′-arylene-dipropiolonitrile. In some embodiments, the exogenous antigenic polypeptide having one or more sulfone groups is covalently linked to a N-terminal cysteine and/or a C-terminal cysteine of the wild-type or loadable exogenous antigen-presenting polypeptide. In some embodiments, the sulfone is a phenyloxadiazolyl sulfone. In some embodiments, the sulfone is a benzothiazolyl sulfone. In some embodiments, the sulfone is a bis-sulfone. In some embodiments, the exogenous antigenic polypeptide having one or more allenamide groups is covalently linked to a N-terminal cysteine and/or a C-terminal cysteine of the wild-type or loadable exogenous antigen-presenting polypeptide. In some embodiments, the allenamide is a benzylallenamide. In some embodiments, the allenamide is a cyclohexylallenamide. In some embodiments, the allenamide is a naphthylallenamide. In some embodiments, the allenamide is a aminoethylallenamide. In some embodiments, the exogenous antigenic polypeptide having one or more dibromopyridazinedione groups is covalently linked to a N-terminal cysteine and/or a C-terminal cysteine of the wild-type or loadable exogenous antigen-presenting polypeptide. In some embodiments, the dibromopyridazinedione is a 4,5-dibromo-1,2-dihydro-pyridazine-3,6-dione. In some embodiments, the exogenous antigenic polypeptide having one or more disulfide groups is covalently linked to a N-terminal cysteine and/or a C-terminal cysteine of the wild-type or loadable exogenous antigen-presenting polypeptide. In some embodiments, the disulfide is a nitropyridyl disulfide. In some embodiments, the exogenous antigenic polypeptide having one or more haloacetamide groups is covalently linked to a N-terminal cysteine and/or a C-terminal cysteine of the wild-type or loadable exogenous antigen-presenting polypeptide. In some embodiments, the haloacetamide is a iodoacetamide. In some embodiments, the haloacetamide is a bromoacetamide. In some embodiments, the haloacetamide is a chloroacetamide.

In some embodiments, the exogenous antigenic polypeptide comprises one or more (e.g., two, three, four, etc.) diazirine groups (Yang et al. (2015) Chem. Sci. 6: 1011-7; Yang et al. (2016) Nat. Chem. Biol. 12(2): 70-2), which can react with one or more amino acid residues located in the wild-type or loadable exogenous antigen-presenting polypeptide of the engineered erythroid cells (e.g., engineered enucleated erythroid cells) or enucleated cells (e.g., modified enucleated cells). In some embodiments, the one or more amino acid residues are located in the antigen-binding cleft of the wild-type or loadable exogenous antigen-presenting polypeptide. In some embodiments, the modified exogenous antigenic polypeptide bearing one or more diazarine groups is reacted with the wild-type or loadable exogenous antigen-presenting polypeptide under UV irradiation to covalently link the exogenous antigenic polypeptide to the wild-type or loadable exogenous antigen-presenting polypeptide.

In some embodiments, the exogenous antigenic polypeptide bearing a reactive functional group (e.g., a thiol-reactive group or a diazarine group) described above further comprises a spacer between the polypeptide and the reactive groups (e.g., thiol-reactive groups or diazirine groups). Any suitable spacer that covalently links the exogenous antigenic polypeptide and reactive functional group can be used. Exemplary spacers include, but are not limited to, PEG spacers, diamines (e.g., ethylene diamine), an amino acid, and peptide (e.g., a peptide having 1 to 40 amino acid residues).

In some embodiments, the engineered erythroid cell (e.g., engineered enucleated erythroid cell) or enucleated cell (e.g., modified enucleated cell) presents one or more exogenous antigenic polypeptides, wherein the one or more exogenous antigenic polypeptides are enzymatically conjugated to the wild-type or loadable exogenous antigen-presenting polypeptide.

In specific embodiments, the exogenous antigenic polypeptide can be conjugated to the wild-type or loadable exogenous antigen-presenting polypeptide by various chemical and enzymatic means, including but not limited to, chemical conjugation with bifunctional cross-linking agents such as, e.g., an NHS ester-maleimide heterobifunctional crosslinker to connect a primary amine group with a reduced thiol group.

Exogenous antigenic polypeptides may also be conjugated to a wild-type or loadable exogenous antigen presenting polypeptide on an engineered erythroid cell or enucleated cell provided herein using a sortase described herein (e.g., a sortase A). For example, a first exogenous polypeptide (e.g., a wild-type or loadable exogenous antigen presenting polypeptide or an exogenous antigenic polypeptide(s)) comprises or is engineered to include either an acceptor sequence (e.g., LPXTG (SEQ ID NO: 36) or LPXTA (SEQ ID NO: 37)), and the second exogenous polypeptide (e.g., a wild-type or loadable exogenous antigen presenting polypeptide or an exogenous antigenic polypeptide(s)) comprises or is engineered to include an N-terminal donor sequence (e.g., G, GG, GGG, A, AA, and AAA).

When contacted with a suitable sortase (e.g., a Streptococcus aureus sortase A or a S. pyogenes sortase A) a transpeptidation reaction ocurrs such that both the wild-type or loadable exogenous antigen presenting polypeptide and the exogenous antigenic polypeptide(s) are conjugated (see, e.g., Swee et al. (2013) Proc. Nat'l. Acad. Sci. USA 110(4): 1428-33, incorporated herein by reference). In some embodiments, the N-terminus of the exogenous wild-type or loadable exogenous antigen presenting polypeptide comprises an N-terminal donor sequence G, GG, GGG, A, AA, or AAA. In some embodiments, N-terminal donor sequence (e.g., GG, GGG) of the wild-type or loadable exogenous antigen presenting polypeptide is conjugated to an exogenous antigenic polypeptide containing the acceptor sequence LPXTG (SEQ ID NO: 36) or LPXTA (SEQ ID NO: 37), via a sortase-mediated reaction (e.g., a sortase A-mediated reaction). Additional acceptor sequences and donor sequences that can be used for sortase-mediated conjugation reactions and methods of utilizing sortagging are described in Antos et al. (2016) Curr Opin Struct Biol. 38: 111-8, the contents of which are hereby incorporated herein by reference.

Exogenous antigenic polypeptide(s) can be conjugated to a wild-type or loadable exogenous antigen presenting polypeptide on an engineered erythroid cell (e.g., engineered enucleated erythroid cell) or enucleated cell (e.g., modified enucleated cell) using a butelase 1, e.g., Clitoria ternatea butelase 1 (UniProtKB Accession No. A0A060D9Z7), as described e.g., in Nguyen et al. (2016). For example, a first exogenous polypeptide (e.g., the wild-type or loadable exogenous antigen presenting polypeptide or the exogenous antigenic polypeptide(s)) comprises or is engineered to include a C-terminal butelase-1 tripeptide recognition sequence Asx-His-Val (wherein Asx is Asp or Asn). The second exogenous polypeptide (e.g., the wild-type or loadable exogenous antigen presenting polypeptide or the exogenous antigenic polypeptide(s)) is engineered to include an N-terminal X₁X₂, wherein X1 is any amino acid and X₂ is I, L, V, or C. When contacted with butelase 1, both exogenous polypeptides (e.g., the wild-type or loadable exogenous antigen presenting polypeptide and the exogenous antigenic polypeptide(s)) are conjugated by the enzyme (see, e.g., Nguyen et al. (2016)).

Alternatively, exogenous polypeptides can be conjugated onto the engineered erythroid cells (e.g., engineered enucleated erythroid cells) or enucleated cells (e.g., modified enucleated cells) described herein, or exogenous polypeptides can be conjugated to one another, using a catalytic bond-forming polypeptide (e.g., a SpyTag/SpyCatcher system). For example, the engineered erythroid cells or enucleated cells provided herein can be engineered to include an exogenous polypeptide comprising either a SpyTag or a SpyCatcher polypeptide (e.g., on the extracellular portion of the exogenous polypeptide). Alternatively, an exogenous polypeptide described herein (e.g., a wild-type or loadable exogenous antigen presenting polypeptide or an exogenous antigenic polypeptide(s)) can be engineered to include either a SpyTag or SpyCatcher polypeptide. For example, in some embodiments, a wild-type or loadable exogenous antigen presenting polypeptide comprises an N-terminal SpyCatcher polyepeptide and an exogenous antigenic polypeptide comprises a SpyTag polypeptide. Upon contacting of the SpyTag and SpyCatcher polypeptides, a covalent bond can be formed (see, e.g., Zakeri et al. (2012) Proc. Nat'l. Acad. Sci. U.S.A. 109: E690-7.

Exogenous polypeptides provided herein (e.g., a wild-type or loadable exogenous antigen presenting polypeptide or an exogenous antigenic polypeptide(s)) can be conjugated onto engineered erythroid cells or enucleated cells described herein, and/or the exogenous polypeptides (e.g., a wild-type or loadable exogenous antigen presenting polypeptide or an exogenous antigenic polypeptide(s)) can be conjugated to one another, using combination methods (e.g., an enzymatic conjugation in combination with click chemistry). For example, a sortase-mediated conjugation can be used to attach a click-chemistry handles (e.g., an azide or an alkyne) onto a cell or an exogenous polypeptide. Subsequently, click chemistry (e.g., a cyclo-addition reaction) can be used to conjugate an additional exogenous polypeptide onto the cell, or onto an exogenous polypeptide (e.g., an exogenous antigenic polypeptide to a wild-type or loadable exogenous antigen presenting polypeptide); see, e.g., Neves et al. (2013) Bioconjugate Chemistry 24(6): 934-41. Sortase-mediated modification of proteins and cells is described in International Publication Nos. WO 2014/183066 and WO 2014/183071, both of which are incorporated by reference in their entireties herein.

The engineered erythroid cells (e.g., engineered enucleated erythroid cells) or enucleated cells (e.g., modified enucleated cells), described herein can also be produced using one or more of the coupling reagents, as described herein, for e.g., click chemistry, to attach an exogenous polypeptide (e.g., a wild-type or loadable exogenous antigen-presenting polypeptide) to the surface of the cell. In some embodiments, coupling reagents, for e.g., transglutaminase-mediated conjugation and fucosylation-mediated conjugation can be used to couple an exogenous antigenic polypeptide to a wild-type or loadable exogenous antigen-presenting polypeptide, wherein thereafter the complex of the wild-type or loadable exogenous antigen-presenting polypeptide bound to an exogenous antigenic polypeptide, can be attached to the surface of the cell, using one of the coupling reagents described herein, for e.g., click chemistry.

In some embodiments, the methods of conjugating an exogenous antigenic polypeptide to a loadable exogenous antigen-presenting polypeptide, as described herein, can be performed after a displaceable exogenous polypeptide that was previously bound to the loadable exogenous antigen-presenting polypeptide has been displaced from the loadable exogenous antigen-presenting polypeptide (e.g., using a method described herein).

In some embodiments, the methods of conjugating an exogenous antigenic polypeptide to a wild-type or loadable exogenous antigen-presenting polypeptide, as described herein, can be performed using a wild-type or loadable exogenous antigen-presenting polypeptide, wherein a displaceable exogenous polypeptide was not previously bound to the wild-type or loadable exogenous antigen-presenting polypeptide.

While some exemplary methods for conjugating an exogenous antigenic polypeptide to the wild-type or loadable exogenous antigen-presenting polypeptide are provided herein, these are exemplary and not meant to limit the scope of the present disclosure. Additional suitable methods for conjugating an exogenous antigenic polypeptide to a wild-type or loadable exogenous antigen-presenting polypeptide will be apparent to those of skill in the art.

### Exogenous Costimulatory Polypeptides

In some embodiments, an engineered erythroid cell or enucleated cell provided herein includes an exogenous costimulatory polypeptide. In some embodiments, the exogenous costimulatory polypeptide is capable of specifically binding to a cognate costimulatory molecule on a T cell (e.g., an HLA molecule, B and T lymphocyte attenuator (CD272) and a Toll ligand receptor), thereby providing a signal which mediates a T cell response, including, but not limited to, proliferation, activation, differentiation, and the like. Exogenous costimulatory polypeptides may also include antibodies that specifically bind costimulatory molecules present on a T cell. Such antibody preferably binds and acts as an agonist to the costimulatory molecule on the T cell.

In some embodiments, the desired response is cell death, e.g., of an infected cell. In some embodiments, the costimulatory polypeptides trigger multiple T cell activation pathways to induce an immune response. In some embodiments, an engineered erythroid cell or enucleated cell described herein includes, inter alia, one or more exogenous costimulatory polypeptides capable of promoting T cell proliferation. In some embodiments, the one or more (e.g., 2, 3, 4, or 5 or more) costimulatory polypeptides comprise an activating polypeptide of Table 9, below, or a T-cell activating variant (e.g., fragment) thereof. In some embodiments, one or more (e.g., 2, 3, 4, or 5 or more) costimulatory polypeptides comprise an antibody molecule (e.g. agonizing antibody) that binds a target receptor of Table 9 or a T-cell activating variant (e.g., fragment) thereof. In some embodiments, the costimulatory polypeptides comprise different T cell activation ligands, e.g., one or more activating polypeptides of Table 9, in any combination thereof, to stimulate T cells. In some embodiments, the engineered erythroid cell or enucleated cell comprises at least one exogenous costimulatory polypeptide on the cell surface comprising either 4-1BBL, OX40L, and CD40L, or fragments or variants thereof. In some embodiments, these exogenous costimulatory polypeptides are capable of signaling through complementary activation pathways. The exogenous costimulatory polypeptides can be derived from endogenous T cell activation ligands or from antibody molecules to the target receptors.

TABLE 9
Costimulatory Polypeptides

| Activating Polypeptide (Ligand) | Target Receptor |
|---|---|
| B7-H2 (e.g., Accession Number | ICOS, CD28 (e.g., Accession |
| NP_056074.1) | Number NP_006130.1) |
| B7-1 (e.g., Accession Number | CD28 (e.g., Accession Number |
| NP_005182.1) | NP_006130.1) |
| B7-2 (e.g., Accession Number | CD28 (e.g., Accession Number |
| AAA86473) | NP_006130.1) |
| CD70 (e.g., Accession Number | CD27 (e.g., Accession Number |
| NP_001243.1) | NP_001233.1) |
| LIGHT (e.g., Accession Number | HVEM (e.g., Accession Number |
| NP_003 798.2) | AAQ89238.1) |
| HVEM (e.g., Accession Number | LIGHT (e.g., Accession Number |
| AAQ89238.1) | NP_003798.2) |
| CD40L (e.g., Accession Number | CD40 (e.g., Accession Number |
| BAA06599.1) | NP_001241.1) |
| 4-1BBL (e.g., Accession Number | 4-1BB (e.g., Accession |
| NP_003802.1) | NP_001552.2) |
| OX40L (e.g., Accession Number | OX40 (e.g., Accession Number |
| NP_003317.1) | NP_003318.1) |
| TL1A (e.g., Accession Number | DR3 (e.g., Accession Number |
| NP_005109.2) | NP_683866.1) |
| GITRL (e.g., Accession Number | GITR (e.g., Accession Number |
| NP_005083.2) | NP_004186.1) |
| CD30L (e.g., Accession Number | CD30 (e.g., Accession Number |
| NP_001235.1), | NP_001234.3) |
| TIM4 (e.g., Accession Number | TIM1 (e.g., Accession Number |
| NP_612388.2) | NP_036338.2) |
| SLAM (e.g., Accession Number | SLAM (e.g., Accession Number |
| AAK77968.1) | AAK77968.1) |
| CD48 (e.g., Accession Number | CD2 (e.g., Accession Number |
| CAG33293.1) | NP_001315538.1) |
| CD58 (e.g., Accession Number | CD2 (e.g., Accession Number |
| CAG33220.1) | NP_001315538.1) |
| CD155 (e.g., Accession Number | CD226 (e.g., Accession Number |
| NP_001129240.1) | NP_006557.2) |
| CD112 (e.g., Accession Number | CD226 (e.g., Accession Number |
| NP_001036189.1) | NP_006557.2) |
| CD137L (e.g., Accession Number | CD137 (e.g., Accession |
| NP_003802.1) | NP_001552.2) |

In some embodiments, the exogenous costimulatory polypeptide comprises an N-terminal truncated 4-1BBL. In some embodiments, the exogenous costimulatory polypeptide comprises a full length 4-1BBL.

In some embodiments, the one or more exogenous costimulatory polypeptides comprises an activating cytokine, interferon or TNF family member, e.g., IFNα, IL2, IL6 or any combination thereof. In some embodiments, the one or more exogenous costimulatory polypeptides comprises one or more activating cytokine, interferon or TNF family member, and further comprises one or more activating polypeptide or ligand (e.g., of Table 9) or a T-cell activating variant (e.g., fragment) thereof, or one or more antibody molecules (e.g. agonizing antibody) that binds a target costimulatory T cell receptor (e.g., of Table 9) or a T-cell activating variant (e.g., fragment) thereof.

In certain embodiments, the disclosure features engineered erythroid cells (e.g., engineered enucleated erythroid cells) or enucleated cells (e.g., modified enucleated cells) that can be used to specifically induce proliferation of a T cell expressing a known co-stimulatory molecule. T cell that are contacted (e.g., in vivo or in vitro) with an engineered erythroid cell or enucleated cell presenting (e.g. comprising on the cell surface) an exogenous costimulatory polypeptide that specifically binds to a co-stimulatory molecule expressed by the T-cell can be expanded, stimulated and/or induced to proliferate such that a large numbers of specific T cells can be readily produced. The engineered erythroid cell or enucleated cell can expand the T cell specifically in that only T cells expressing the particular costimulatory molecule are expanded. Thus, where the T cell to be expanded is present in a mixture of cells, only the T cell of interest will be induced to proliferate and expand. The T cell can be further purified using a wide variety of cell separation and purification techniques, such as those known in the art and/or described elsewhere herein.

As would be appreciated by the skilled artisan, based upon the disclosure provided herein, the T cell of interest need not be identified or isolated prior to expansion using the engineered erythroid cell or enucleated cell because only T cell(s) expressing the cognate costimulatory molecule will be induced to expand.

In some embodiments, an engineered erythroid cell (e.g., engineered enucleated erythroid cell) or enucleated cell (e.g., modified enucleated cell) targets multiple T cell activating pathways in combination (e.g., as described in Table 9, above), e.g., using exogenous costimulatory polypeptides comprising ligands or antibody molecules, or both, co-expressed (or co-presented) on an engineered erythroid cell or enucleated cell.

In some embodiments, the at least one exogenous costimulatory polypeptide comprises either 4-1BBL, LIGHT, CD80, CD86, CD70, IL-7, IL-12, OX40L, GITRL, TIM4, SLAM, CD48, CD58, CD83, CD155, CD112, IL-15Ra fused to IL-15, IL-2, IL-21, a ligand for ICAM-1, a ligand for LFA-1, a functional fragment thereof, and combinations thereof. In some embodiments, the at least one exogenous costimulatory polypeptide is an agonist antibody to a cognate costimulatory ligand receptor. For example, in certain embodiments, the costimulatory polypeptide comprises an antibody (e.g., an agonist antibody) to 4-1BB (e.g., urelumab (Bristol-Myers Squibb), utomilumab (Pfizer), ATOR-1017 (Alligator Bioscience), AGEN2373 (Agenus), CTX-471 (Compass Therapeutics), ADG106 (Adagene), and LVGN6051 (Lyvgen Biopharma)), LIGHT receptor (HVEM), CD80 receptor, CD86 receptor, OX40 (e.g., MEDI6469 (Medimmune), ivuxolimab (Pfizer), GSK3174998 (GlaxoSmithKline), BMS-986178 (Bristol-Myers Squibb), vonlerizumab (Genentech), KHK4083 (Kirin Pharma), tavolimab (Medimmune), INCAGN1949 (Agenus), GBR 830 (Ichnos), MEDI6383 (Medimmune), IBI101 (Innovent), BGB-A445 (BeiGene), and INBRX-106 (Inhibrx)), ICOS (e.g., GSK3359609 (GlaxoSmithKline), MEDI-570 (Medimmune), vopratelimab (Jounce Therapeutics), KY1044 (Kymab), GITR, TIM4 receptor (TIM1), SLAM receptor, CD28 (e.g., lulizumab (Bristol-Myers Squibb), TABO8 (TheraMab), FR104 (OSE Immunotherapeutics), TGN1412 (Tegenero)), CD40 (e.g., dacetuzumab (Genentech), APX005 (Apexigen), iscalimab (Novartis), selicrelumab (Hoffmann-La Roche), BI 655064 (Boehringer), bleselumab (Astellas), lucatumumab (Novartis), RG7876 (Genentech), FFP104 (Fast Forward Pharma), mitazalimab (Alligator Bioscience), Chi Lob 7/4 (Cancer Research UK), 2141-V11 (Bristol-Myers Squibb), SEA-CD40 (Seattle Genetics), CDX-1140 (Celldex), NG-350A (PsiOxus), XmAb 5485 (Xencor), PG120 (PanGenetics), teneliximab (Bristol-Myers Squibb), SBT-040 (Opi Vi), ravagalimab (Abbvie)), CD48 receptor (CD2), CD58 receptor (CD2), CD83 receptor, CD155 receptor (CD226), CD112 receptor (CD226), IL-2 receptor (CD25, CD122, CD132), IL-21 receptor, ICAM, and combinations thereof, or an antigen-binding fragment therof (e.g., an scFV). In some embodiments, the at least one exogenous costimulatory polypeptides comprises an anti CD3 antibody, an anti-CD38 antibody, and combinations thereof, or an antigen-binding fragment therof.

In some embodiments, the engineered erythroid cell or enucleated cell presents, e.g. comprises on the cell surface, at least two, at least 3, at least 4, or at least 5 exogenous costimulatory polypeptides. In some embodiments, the exogenous costimulatory polypeptides are fused to each other, for example IL-21 fused to IL-2.

In some embodiments, the one or more exogenous costimulatory polypeptides include or are fused to a transmembrane domain. In some embodiments, the transmembrane domain is selected from a sequence set forth in Table 2. In some embodiments, the one or more exogenous costimulatory polypeptides include or are fused to a leader sequence. In some embodiments, the leader sequence is selected from a sequence set forth in Table 1.

### Exogenous Co-inhibitory Polypeptides

In some embodiments, an engineered erythroid cell or enucleated cell provided herein includes an exogenous co-inhibitory polypeptide. An exogenous co-inhibitory polypeptide is any polypeptide that suppresses a T cell, including inhibition of T cell activity, inhibition of T cell proliferation, anergizing of a T cell, or induction of apoptosis of a T cell. In some embodiments, an exogenous co-inhibitory polypeptide is capable of specifically binds to a cognate coinhibitory molecule on a T cell. In some embodiments, the exogeonous co-inhibitory polypeptide ligand is an inhibitory polypeptide shown in Table 10.

In some embodiments, the exogenous co-inhibitory polypeptide comprises an agonist (e.g. an antibody) that specifically binds a coinhibitory receptor on a T cell. In some embodiments, the agonist is an antibody that binds a receptor selected from the group consisting of: PD1, CTLA4, TIM3, CEACAM (e.g., CEACAM-1, CEACAM-3 and/or CEACAM-5), LAG3, VISTA, BTLA, TIGIT, LAIR1, CD160, and 2B4. In other embodiments, the agonist is an antibody that binds a target receptor on a T cell shown in Table 10.

TABLE 10
Coinhibitory Polypeptides

| Inhibitory Polypeptide | Target Receptor |
|---|---|
| B7-1 | CTLA4, B7H1 |
| B7-2 | CTLA4 |
| B7DC | PD1 |
| B7H1 | PD1, B7-1 |
| HVEM | CD160, BTLA |
| COLLAGEN | LAIR1 |
| GALECTIN9 | TIM3 |
| CD48, TIM4 | TIM4R |
| CD48 | 2B4 |
| CD155, CD112, CD113 | TIGIT |
| PDL1 | PD1 |
| | LAG3 |

In some embodiments, an exogenous co-inhibitory polypeptide comprises an antibody (or an antigen-binding fragment therof (e.g., an scFv)) that blocks binding of a costimulatory polypeptide to its cognate costimulatory receptor. In various embodiments, the exogenous co-inhibitory polypeptide comprises an antibody (or an antigen-binding fragment therof) that blocks binding of 4-1BBL, LIGHT, CD80, CD86, CD70, OX40L, GITRL, TIM4, SLAM, CD48, CD58, CD83, CD155, CD112, IL-15Rα fused to IL-15, IL-2, IL-21, ICAM, a ligand for LFA-1, an anti CD3 antibody or an anti CD28 antibody, to its receptor.

In some embodiments, the exogenous co-inhibitory polypeptide comprises IL-35, IL-10, or VSIG-3, or a functional fragment thereof. In some embodiments, the exogenous co-inhibitory polypeptide comprises VSIG-3, or a functional fragment thereof.

In some embodiments, an engineered erythroid cellor enucleated cell targets multiple T cell inhibitory pathways in combination (e.g., as described in Table 10, above), e.g., using exogenous co-inhibitory polypeptides comprising ligands or antibody molecules, or both, co-expressed on the engineered erythroid cell or enucleated cell.

In some embodiments, the engineered erythroid cell or enucleated cell presents, e.g. comprises on the cell surface, at least two, at least 3, at least 4, or at least 5 exogenous co-inhibitory polypeptides.

In some embodiments, the one or more exogenous co-inhibitory polypeptides include or are fused to a transmembrane domain. In some embodiments, the transmembrane domain is selected from a sequence set forth in Table 2. In some embodiments, the one or more exogenous co-inhibitory polypeptides include or are fused to a leader sequence. In some embodiments, the leader sequence is selected from a sequence set forth in Table 1.

### T-Cell Activation Signals

For efficient induction of T-cell proliferation, activation and expansion, several signals need to be transmitted from the engineered erythroid cell (e.g., engineered enucleated erythroid cell) or enucleated cell (e.g., modified enucleated cell) to naïve T cells. These signals are commonly referred to as Signal 1, Signal 2 and Signal 3, and are described below. In some embodiments, Signal 1 comprises a loadable exogenous antigen-presenting polypeptide on the cell surface, wherein the loadable exogenous antigen-presenting polypeptide comprises one or more amino acid substitutions which stabilize the loadable exogenous antigen-presenting polypeptide on the cell surface, further comprising an exogenous antigenic polypeptide bound to the loadable exogenous antigen-presenting polypeptide. In some embodiments, Signal 1 comprises a wild-type exogenous antigen-presenting polypeptide. In some embodiments, the engineered erythroid cells or enucleated cells described herein comprise one or more exogenous polypeptides comprising Signal 1, one or more exogenous polypeptides comprising Signal 2, and/or one or more exogenous polypeptides comprising Signal 3, in any combination as set forth below. In some embodiments, in addition to Signal 1, Signal 2 and Signal 3, the engineered erythroid cells or enucleated cells described herein further comprise one or more exogenous polypeptides comprising one or more cell adhesion molecules to further facilitate the interaction between T-cells and the engineered erythroid cells or enucleated cells. It is to be understood that when an engineered erythroid cell or enucleated cell comprises the one or more exogenous polypeptides comprising Signal 1 and/or the one or more exogenous polypeptides comprising Signal 2 and/or the one or more exogenous polypeptides comprising Signal 3 (and optionally the one or more polypeptides comprising a cell adhesion molecules), the exogenous polypeptides comprising Signal 1 and/or Signal 2 and/or Signal 3 and/or a cell adhesion molecule are all comprised on the same engineered erythroid cells or enucleated cells.

The engineered erythroid cells or enucleated cells described herein offer numerous advantages over the use of spherical nanoparticles, such as rigid, bead-based APCs. For example, the membrane of an engineered erythroid cell or enucleated cell as described herein is much more dynamic and fluid than the outer surface of a nanoparticle, which is rigid and immobile, and therefore limits the movement of the polypeptides on its surface. The fluidity of the engineered erythroid cell or enucleated cell membrane allows for greater molecular mobility and more efficient molecular reorganization, and is advantageous for immunological synapse formation and T cell stimulation. In some embodiments, the engineered erythroid cells or enucleated cells described herein comprising one or more exogenous polypeptides comprising Signal 1, one or more exogenous polypeptides comprising Signal 2, and/or one or more exogenous polypeptides comprising Signal 3, in any combination as set forth below, on the surface of the cells, provide a more controlled stimulation of T-cells, thereby allowing for the propagation of T-cells with a specific phenotype and activity. In some embodiments, by engineering the engineered erythroid cells or enucleated cells to comprise Signal 1 and/or Signal 2 and/or Signal 3 on the surface of the cell, the engineered erythroid cells or enucleated cells provide optimal control over the signals provided to T-cells.

The engineered erythroid cells or enucleated cells described herein, e.g., comprising the one or more exogenous polypeptides comprising Signal 1 and/or the one or more exogenous polypeptides comprising Signal 2 and/or the one or more exogenous polypeptides comprising Signal 3 (and optionally the one or more polypeptides comprising a cell adhesion molecules), can be used to activate an antigen-specific T cell population, by contacting the T cell population with the engineered enucleated erythroid cell, thereby activating the antigen-specific T cell population. In some embodiments, the engineered enucleated erythroid cell described herein can be contacted with multiple antigen-specific T cell populations to allow the activation of the multiple antigen-specific T cell populations. In some embodiments, the engineered enucleated erythroid cell comprising one or more exogenous antigenic polypeptides can be contacted with one or more antigen-specific T cell populations, to allow the activation of the one or more antigen-specific T cell populations. In some embodiments, the engineered enucleated erythroid cell comprising two or more exogenous antigenic polypeptides can be contacted with two or more antigen-specific T cell populations, to allow the activation of the two or more antigen-specific T cell populations. In some embodiments, the engineered enucleated erythroid cell comprising three or more exogenous antigenic polypeptides can be contacted with three or more antigen-specific T cell populations, to allow the activation of the three or more antigen-specific T cell populations. In some embodiments, the engineered enucleated erythroid cell comprising four or more exogenous antigenic polypeptides can be contacted with four or more antigen-specific T cell populations, to allow the activation of the four or more antigen-specific T cell populations. In some embodiments, the engineered enucleated erythroid cell comprising five or more exogenous antigenic polypeptides can be contacted with five or more antigen-specific T cell populations, to allow the activation of the five or more antigen-specific T cell populations. In some embodiments, the one, two, three, four, five or more exogenous antigenic polypeptides may be present on the same engineered enucleated erythroid cell, or they may be present on distinct engineered enucleated erythroid cells.

The one or more exogenous antigenic polypeptides may comprise any antigenic polypeptide described herein. In some embodiments, the one or more exogenous antigenic polypeptides comprises an HPV-E6 antigen. In some embodiments, the one or more exogenous antigenic polypeptides comprises an HPV-E7 antigen. In some embodiments, the two or more exogenous antigenic polypeptides comprise an HPV-E6 antigen and an HPV-E7.

Signal 1—Antigen Recognition

T cell activation occurs after a T cell receptor (TCR) recognizes a specific peptide antigen presented on an exogenous antigen-binding polypeptide of an engineered erythroid cell or enucleated cell as described herein. Generally, exogenous antigenic polypeptides presented on an exogenous antigen-binding polypeptides comprising a HLA class II polypeptide are recognized by the TCR in conjunction with the CD4 T cell co-receptor. Exogenous antigenic polypeptides presented on an exogenous antigen-binding polypeptides comprising a HLA class I polypeptide are recognized by the TCR in conjunction with a CD8 T cell co-receptor. Ligation of the TCR by a peptide-HLA complex leads to transduction of the signals necessary for activation of the T cell.

In some embodiments, Signal 1 comprises one or more exogenous polypeptides comprising a wild-type or loadable exogenous antigen-presenting polypeptide. In some embodiments, Signal 1 comprises a loadable exogenous antigen-presenting polypeptide on the cell surface, wherein the loadable exogenous antigen-presenting polypeptide comprises one or more amino acid substitutions which stabilize the loadable exogenous antigen-presenting polypeptide on the cell surface, further comprising an exogenous antigenic polypeptide bound to the loadable exogenous antigen-presenting polypeptide. In some embodiments, Signal 1 comprises a wild-type exogenous antigen-presenting polypeptide comprising a exogenous antigenic polypeptide bound thereto. In some embodiments, the wild-type or loadable exogenous antigen-presenting polypeptide comprises an HLA class I polypeptide, an HLA class I single chain fusion, an HLA class II polypeptide, or an HLA class II single chain fusion, as provided above. In some embodiments, the HLA class I polypeptide is selected from a HLA-A, a HLA-B, a HLA-C, a HLA-E, and a HLA-G polypeptide (or fragments thereof). In some embodiments, the HLA class II polypeptide is selected from a HLA-DPα, a HLA-DPβ, a HLA-DMA, a HLA-DMB, a HLA DOA, a HLA DOB, a HLA DQα, a HLA DQβ, a HLA DRα, and a HLA DRβ, or fragments thereof, and combinations thereof.

Signal 2—Co-Stimulation

To become fully activated, T cells require a second signal in addition to TCR-mediated antigen recognition. This second signal (i.e., Signal 2), or co-stimulation, is important for proper T cell activation. In some embodiments, Signal 2 comprises one or more exogenous costimulatory polypeptides provided herein. In some embodiments, the one or more exogenous costimulatory polypeptides comprise 4-1BBL, LIGHT, anti CD28, CD80, CD86, CD70, OX40L, GITRL, TIM4, SLAM, CD48, CD58, CD83, CD155, CD112, IL-15, IL-15Ra fused to IL-15, IL-21, ICAM-1, a ligand for LFA-1, anti CD3 antibody, fragments thereof, and any combination thereof. In some embodiments, Signal 2 comprises one or more exogenous costimulatory polypeptides comprising 4-1BBL, CD80, CD86, CD83, CD70, LIGHT, HVEM,CD40L, OX40L, TL1A, GITRL, CD3OL, and fragments thereof.

Signal 3—Cytokines

To induce more efficient expansion and specific differentiation of T cells, a third signal (Signal 3) can be used. Therefore, an engineered erythroid cell or enucleated cell provided herein can further include an exogenous polypeptide comprising Signal 3 (e.g., a cytokine, a co-inhibitory polypeptide, or fragments thereof). In some embodiments, Signal 3 comprises one or more exogenous polypeptides comprising one or more cytokines. In some embodiments, Signal 3 comprises one or more exogenous polypeptides selected from the group consisting of IL-2, IL-15, IL-7, IL-12, IL-18, IL-21, IL-4, IL-6, IL-23, IL-27, IL-17, IL-10, TGF-beta, IFN-gamma, IL-1 beta, GM-CSF, IL-15, IL-15Rα fused to IL-15, and IL-25, or fragments thereof.

In addition to immunostimulatory cytokines, immunoinhibitory cytokines are capable of dampening the immune response or can lead to tolerance. Accordingly, in some embodiments, Signal 3 comprises one or more exogenous co-inhibitory polypeptides. In some embodiments, the one or more exogenous co-inhibitory polypeptide comprise IL-35, IL-10, VSIG-3, or a LAG3 agonist, and fragments thereof.

### Cell Adhesion Molecules

In some embodiments, in addition to Signal 1, Signal 2 and/or Signal 3, the engineered erythroid cells or enucleated cells described herein further comprise at the cell surface one or more exogenous polypeptides comprising a cell adhesion molecule. Cell adhesion molecules further facilitate the integration between T-cells and the engineered erythroid cells or enucleated cells. In some embodiments, the engineered erythroid cells or enucleated cell comprise an exogenous polypeptide comprising a cell adhesion molecule that mediates or facilitates the formation of the immunological synapse. In some embodiments, the exogenous polypeptide comprises one or more cell adhesion molecules selected from ICAM4/LW, CD36, CD58/LFA3, CD47, VLA4, BCAM/Lu, CD44, CD99/MIC2, ICAM1, JAM1, CD147, fragments thereof, or any combination thereof.

In some embodiments, the engineered erythroid cell or enucleated cell comprises at the cell surface an exogenous polypeptide comprising Signal 1, an exogenous polypeptide comprising Signal 2, and one or more exogenous polypeptides comprising a cell adhesion molecule. In some embodiments, the engineered erythroid cell or enucleated cell comprises at the cell surface an exogenous polypeptide comprising Signal 1, an exogenous polypeptide comprising Signal 2, an exogenous polypeptide comprising Signal 3, and one or more exogenous polypeptides comprising a cell adhesion molecule.

In some embodiments, the engineered erythroid cell or enucleated cell comprises at the cell surface more than one exogenous polypeptide comprising more than one Signal 1, an exogenous polypeptide comprising Signal 2, and one or more exogenous polypeptides comprising cell adhesion molecules. In some embodiments, the engineered erythroid cell or enucleated cell comprises at the cell surface more than one exogenous polypeptide comprising more than one Signal 1, an exogenous polypeptide comprising Signal 2, an exogenous polypeptide comprising Signal 3, and one or more exogenous polypeptides comprising cell adhesion molecules.

In some embodiments, the engineered erythroid cell or enucleated cell comprises at the cell surface an exogenous polypeptide comprising Signal 1, more than one exogenous polypeptide comprising more than one Signal 2, and one or more exogenous polypeptides comprising a cell adhesion molecule. In some embodiments, the engineered erythroid cell or enucleated cell comprises at the cell surface an exogenous polypeptide comprising Signal 1, more than one exogenous polypeptide comprising more than one Signal 2, an exogenous polypeptide comprising Signal 3, and one or more exogenous polypeptides comprising a cell adhesion molecule.

In some embodiments, the engineered erythroid cell or enucleated cell comprises at the cell surface an exogenous polypeptide comprising Signal 1, an exogenous polypeptide comprising Signal 2, more than one exogenous polypeptide comprising more than one Signal 3, and one or more exogenous polypeptides comprising a cell adhesion molecule.

In some embodiments, the engineered erythroid cell or enucleated cell comprises at the cell surface more than one exogenous polypeptide comprising more than one Signal 1, an exogenous polypeptide comprising Signal 2, more than one exogenous polypeptide comprising more than one Signal 3, and one or more exogenous polypeptides comprising a cell adhesion molecule.

In some embodiments, the engineered erythroid cell or enucleated cell comprises at the cell surface more than one exogenous polypeptide comprising more than one Signal 1, more than one exogenous polypeptide comprising more than one Signal 2, an exogenous polypeptide comprising Signal 3, and one or more exogenous polypeptides comprising a cell adhesion molecule.

In some embodiments, the engineered erythroid cell or enucleated cell comprises at the cell surface more than one exogenous polypeptide comprising more than one Signal 1, more than one exogenous polypeptide comprising more than one Signal 2, more than one exogenous polypeptide comprising more than one Signal 3, and one or more exogenous polypeptides comprising a cell adhesion molecule.

In some embodiments, the one or more exogenous polypeptides comprising Signal 1, the one or more exogenous polypeptides comprising Signal 2, the one or more exogenous polypeptides comprising Signal 3, and the one or more exogenous polypeptides comprising a cell adhesion molecule are selected from the exogenous polypeptides shown in Table 11.

TABLE 11
Cell Adhesion Molecules

| | |
|---|---|
| Signal 1 | A wild-type or loadable exogenous antigen- |
| | presenting polypeptide on the cell surface. |
| Signal 2 | 4-1BBL; CD80; CD86; CD83; CD70; |
| | LIGHT; HVEM; CD40L; OX40L; TL1A; |
| | GITRL; and CD30L |
| Signal 3 | IL-2; IL-15; IL-7; IL-12; IL-18; IL-21; IL-4; |
| | IL-6; IL-23; IL-27; IL-17; IL-10; TGF-beta; |
| | IFN-gamma; IL-1 beta; GM-CSF; and IL-25 |
| Adhesion Molecules | ICAM4/LW; CD36; CD58/LFA3; CD47; |
| | VLA4; BCAM/Lu; CD44; CD99/MIC2; |
| | ICAM1; JAM1 and CD147 |

### Immunological Synapse

As described herein, the engineered erythroid cells or enucleated cells of the present disclosure provide numerous advantages over the use of spherical nanoparticles, such as rigid, bead-based engineered erythroid cells or enucleated cells. Molecular mobility (e.g. movement of ligands in the cell membrane) and molecular clustering are important features of immunological synapse formation. The membrane of engineered erythroid cell or enucleated cell described herein is much more dynamic and fluid than the outer surface of a nanoparticle, and thus allows a much more efficient molecular reorganization and MEW clustering during the formation of an immunological synapse, or in mediating trogocytosis. Further, in contrast to the small size of the nanoparticles, the engineered erythroid cells or enucleated cells described herein a greater surface area for the formation of functional micron-scaled clusters in an immunological synapse. In some embodiments, the engineered erythroid cells or enucleated cells as described herein are engineered to form an immunological synapse, wherein the immunological synapse facilitates T cell activation.

An immunological synapse (or immune synapse, or IS) is the interface between an antigen-presenting cell and a lymphocyte such as a TB cell or an NK cell. An immunological synapse can consist of molecules involved in T cell activation, which compose typical patterns, called activation clusters. According to the most well studied model, the immune synapse is also known as the supramolecular activation cluster (SMAC) (Monks et al. (1998) Nature 395(6697): 82-6; incorporated in their entirety herein by reference), which is composed of concentric rings (central, peripheral or distal regions) each containing segregated clusters of proteins. Molecules in the immunological synapse include wild-type or loadable exogenous antigen-presenting polypeptides, adhesion molecules, co-stimulatory molecules, and co-inhibitory molecules.

The immunological synapse is a dynamic structure formed after T cell receptors cluster together in microclusters that eventually move towards the immunological synapse center. The spatial and temporal changes of these molecules at the interface of T lymphocyte and APC regulate the structure of the immune synapse and T lymphocyte immune response. In general, efficient CD4+ and CD8+ T cell activation is associated with the formation of a functional immunological synapse (Kaizuka et al. (2007) Proc. Natl. Acad. Sci. U.S.A. 104: 20296-301, incorporated by reference in its entirety herein).

In some embodiments, the disclosure features an engineered erythroid cell or enucleated cell that can form an immunological synapse between the engineered erythroid cell or enucleated cell and an immune cell such as a T cell, B cell or an NK cell. In some embodiments, the engineered erythroid cell or enucleated cell provided herein has the ability to assemble more than one exogenous antigen-presenting polypeptide in the immunological synapse.

The initial interaction at the immunological synapse occurs between the lymphocyte function-associated antigen-1 (LFA-1) present in the peripheral-SMAC of a T-cell, and integrin adhesion molecules (such as ICAM-1 or ICAM-2) on an APC. When bound to an APC, the T-cell can then extend pseudopodia and scan the surface of target cell to find a specific peptide-MHC complex. The process of formation begins when the T-cell receptor (TCR) binds to the peptide-MHC complex on the APC and initiates signaling activation through formation of microclusters/lipid rafts (Varma et al. (2006) Immunity 25(1): 117-27; incorporated in their entirety herein by reference).

Accordingly, in some embodiments, the engineered erythroid cells or enucleated cells of the present disclosure comprise one or more exogenous cell adhesion polypeptides to mediate or facilitate the formation of the immunological synapse. In some embodiments, the one or more cell adhesion molecule is selected from the group consisting of ICAM4/LW, CD36, CD58/LFA3, CD47, VLA4, BCAM/Lu, CD44, CD99/MIC2, ICAM1, JAM1, and CD147, fragments thereof, or any combination thereof.

It is an advantage of the engineered erythroid cells or enucleated cells described herein have a fluid cell membrane that provides dynamic molecular movement and thus allows efficient molecular reorganization and MHC clustering, which is required for T cell stimulation. Signaling is initiated and sustained in TCR microclusters that are formed continuously in the periphery of the immunological synapse and transported to the center to form the central SMAC. During the formation of the central SMAC the microclusters can move independently of each other, and can fuse to form larger clusters with continuous movements. A threshold MHCI cluster density is required to sustain active immune signaling (Anikeeva et al. (2012) PLoS One 7(8): e41466; Bullock et al. (2000) J. Immunol. 164(5): 2354-61; Bullock et al. (2003) J. Immunol. 170(4): 1822-9; Jiang et al. (2011) Immunity 34(1): 13-23; each of which is incorporated in its entirety herein by reference). Accordingly, in some embodiments, an engineered erythroid cell or enucleated cell provided herein can mediate the clustering of exogenous antigen-presenting polypeptides at a density that is effective to form a functional immunological synapse and to activate immune signaling.

Another consequence of the molecular reorganization in immune synapse formation is the intercellular transfer of APC membrane proteins to the T cell. T cells acquire MHC class I and class II glycoproteins from APCs, together with co-stimulatory molecules and membrane patches, by a mechanism referred to as trogocytosis. As described herein, the membrane of an engineered erythroid cell or enucleated cell provided herein allows efficient molecular reorganization and MHC clustering due to its fluidity. In some embodiments, an engineered erythroid cell or enucleated cell described herein allows or mediates the molecular reorganization in immune synapse formation such that trogocytosis occurs.

The sizes of the immunological synapse can be determined by numerous methods known in the art, including microscopy, such as total internal reflection fluorescence microscopy (TIRFM) (Varma et al. (2006); Dustin et al. (2002) Science 298(5594):785-9; each of which are incorporated in their entirety herein by reference). In some embodiments, an engineered erythroid cell or enucleated cell described herein can form an immunological synapse of an average diameter between about 0.5 μm and 5.0 μm. In some embodiments, an engineered erythroid cell or enucleated cell described herein can form an immunological synapse of an average diameter of at least about 0.5 μm. In some embodiments, an engineered erythroid cell or enucleated cell described herein can form a functional immunological synapse of an average diameter between about 0.5 μm and 4.5 μm, between about 0.5 μm and 4.0 μm, between about 0.5 μm and 3.5 μm, between about 0.5 μm and 3.0 μm, between about 0.5 μm and 2.5 μm, between about 0.5 μm and 2.0 μm, between about 0.5 μm and 1.5 μm, between about 0.5 μm and 1.0 μm, between about 1.0 μm and 5.0 μm, between about 1.0 μm and 4.5 μm, between about 1.0 μm and 4.0 μm, between about 1.0 μm and 3.5 μm, between about 1.0 μm and 3.0 μm, between about 1.0 μm and 2.5 μm, between about 1.0 μm and 2.0 μm, between about 1.0 μm and 1.5 μm, between about 1.5 μm and 5.0 μm, between about 1.5 μm and 4.5 μm, between about 1.5 μm and 4.0 μm, between about 1.5 μm and 3.5 μm, between about 1.5 μm and 3.0 μm, between about 1.5 μm and 2.5 μm, between about 1.5 μm and 2.0 μm, between about 2.0 μm and 5.0 μm, between about 2.0 μm and 4.5 μm, between about 2.0 μm and 4.0 μm, between about 2.0 μm and 3.5 μm, between about 2.0 μm and 3.0 μm, between about 2.0 μm and 2.5 μm, between about 2.0 μm and 5.0 μm, between about 2.5 μm and 4.5 μm, between about 2.5 μm and 4.0 μm, between about 2.5 μm and 3.5 μm, between about 2.5 μm and 3.0 μm, between about 3.0 μm and 5.0 μm, between about 3.0 μm and 4.5 μm, between about 3.0 μm and 4.0 μm, between about 3.0 μm and 3.5 μm, between about 3.5 μm and 5.0 μm, between about 3.5 μm and 4.5 μm, between about 3.5 μm and 4.0 μm, between about 4.0 μm and 5.0 μm, between about 4.0 μm and 4.5 μm, between about 4.5 μm and 5.0 μm. In some embodiments, the engineered erythroid cell or enucleated cell described herein can form a functional immunological synapse of an average diameter of at least 0.5 μm, 0.6 μm, 0.7 μm, 0.8 μm, 0.9 μm, 1.0 μm, 1.5 μm, 2.0 μm, 2.5 μm, 3μm, 3.5 μm, 4.0 μm or 5 μm.

As described herein, an advantage of the engineered erythroid cells or enucleated cells of the present disclosure is the fluidity of the engineered erythroid cell or enucleated cell membrane that allows efficient molecular reorganization. Specific signaling pathways lead to polarization of the T-cell by orienting its centrosome toward the site of the immunological synapse. The accumulation and polarization of actin is triggered by TCR/CD3 interactions with integrins and small GTPases. These interactions promote actin polymerization, and as actin is accumulated and reorganized, it promotes clustering of the TCRs and integrins. These highly dynamic contacts are characterized by continuous cytoskeletal remodeling events, which not only structure the interface but also exert a considerable amount of mechanical forces, which influence information transfer both into and out of the immune cell (Basu et al. (2017) Trends Cell Biol. 27(4): 241-54; Hivroz et al. (2016) Front Immunol. 7: 46; each of which are incorporated in their entirety herein by reference). The adhesive forces of tensile strengths between the TCRs and integrins at the site of immunological synapse can be determined by, e.g., atomic force microscopy, biomembrane force probe (BFP) technique, traction force microscopy, etc. (see, e.g., Hivroz et al. (2016)).

In some embodiments, tensile strength is a measure of the adhesive forces between the T cell receptor and the molecules of the immunological synapse, e.g., peptide-MHC complex, formed by the engineered erythroid cell or enucleated cell. In some embodiments, an engineered erythroid cell or enucleated cell is capable of forming an immunological synapse with a tensile strength sufficient to activate an immune cell. In some embodiments, an engineered erythroid cell or enucleated cell of the present disclosure can form a synapse with a tensile strength of between about 1 pN and 30,000 pN. In some embodiments, an engineered erythroid cell or enucleated cell of the present disclosure can form a synapse with a tensile strength of between about 1 pN and 20,000 pN, between about 1 pN and 10,000 pN, between about 1 pN and 9,000 pN, between about 1 pN and 8,000 pN, between about 1 pN and 7,000 pN, between about 1 pN and 6,000 pN, between about 1 pN and 5,000 pN, between about 1 pN and 4,000 pN, between about 1 pN and 3,000 pN, between about 1 pN and 2,000 pN, between about 1 pN and 1,000 pN, between about 1,000 pN and 30,000 pN, between about 1,000 pN and 20,000 pN, between about 1,000 pN and 10,000 pN, between about 1,000 pN and 9,000 pN, between about 1,000 pN and 8,000 pN, between about 1,000 pN and 7,000 pN, between about 1,000 pN and 6,000 pN, between about 1,000 pN and 5,000 pN, between about 1,000 pN and 4,000 pN, between about 1,000 pN and 3,000 pN, between about 1,000 pN and 2,000 pN. In some embodiments, the optimum mechanical force between the peptide-MHC complex (i.e., the exogenous-antigenic polypeptide-exogenous antigen-presenting polypeptide complex) and the TCR at the immunological synapse is at least 1 pN, 1.5 pN, 2.0 pN, 3.0 pN, 4.0 pN, 5.0 pN, 6.0 pN, 7.0 pN, 8.0 pN, 9.0 pN, 10 pN, 20 pN, 30 pN, 40 pN, 50 pN, 60 pN, 70 pN, 80 pN, 90 pN, 100 pN, 500 pN, 1,000 pN, 2,000 pN, 3,000 pN, 4,000 pN, 5,000 pN, 6,000 pN, 7,000 pN, 8,000 pN, 9,000 pN, 10,000 pN, 11,000 pN, 12,000 pN, 13,000 pN, 14,000 pN, 15,000 pN, or 20,000 pN. In some embodiments, an engineered erythroid cell or enucleated cell as described herein can trigger mechanical forces between the peptide-MHC complex and the TCR at the immunological synapse, to activate an immune cell.

### Treg Costimulatory and Coinhibitory Polypeptides

In certain embodiments, the engineered erythroid cells or enucleated cells further comprise an exogenous Treg costimulatory polypeptide (e.g., to expand regulatory T-cells (Tregs) cells). In some embodiments, the Treg costimulatory polypeptides expand Treg cells by stimulating at least one of three signals involved in Treg cell development. Signal 1 involves TCR, and can be stimulated with antibodies, such as anti-CD3 antibodies, or with antigens that signals through TCR. Signal 2 can be mediated by several different molecules, including immune co-stimulatory molecules such as CD80 and 4-1BBL. Signal 3 is transduced via cytokines, such as IL-2, or TGFβ. In some embodiments, the Treg costimulatory polypeptides stimulate one of these signals. In another embodiment, the Treg costimulatory polypeptides stimulate two of these signals. In yet another embodiment, the Treg costimulatory polypeptides stimulate three of these signals.

Signal 1

In some embodiments, the exogenous costimulatory polypeptide comprises an antigen useful as Treg costimulatory polypeptides for stimulating Signal 1, including antigens associated with a target disease or condition. In some embodiments, the exogenous Treg costimulatory polypeptide comprises an autoantigen, insulin (particularly suitable for treating type 1 diabetes), collagen (particularly suitable for treating rheumatoid arthritis), myelin basic protein (particularly suitable for treating multiple sclerosis) or MHC (for treating and preventing foreign graft rejection). The antigens may be administered as part of a conjugate. Optionally, the antigen is provided as part of an MHC/antigen complex. In this embodiment, the MHC and antigen can independently be foreign or syngeneic. For example donor MHC and an allogenic or syngeneic antigen can be used.

Signal 2

In some embodiments, the exogenous Treg costimulatory polypeptide for stimulating Signal 2 include a member of the B7 and TNF families, for example B7 and CD28 family members, shown below in Table 12, and TNF family members shown in Table 13, or a fragment thereof.

TABLE 12
Treg Costimulatory Polypeptides: B7 and CD28 Family Members

| LIGAND | RECEPTOR |
|---|---|
| B7.1 (CD80) | CD28, CTLA-4 (CD 152) |
| B7.2 (CD86) | CD28, CTLA-4 |
| ICOSL (B7h, B7-H2, B7RP-1, | ICOS (AILIM) |
| GL5O, LICOS) | |
| PD-L1 (B7-H1) | PD-1 |
| PD-L2 (B7-DC) | PD-1 |
| B7-H3 | Unknown |
| B7-H4 (B7x; B7S1) | Unknown (BTLA?) |
| Unknown (HVEM*) | BTLA |
| ICOSL (B7h, B7-H2, B7RP-1, | ICOS (AILIM) |

TABLE 13
Treg Costimulatory Polypeptides: TNF Family Members

| LIGAND | RECEPTOR |
|---|---|
| OX40L | OX40 (CD134) |
| 4-1BBL | 4-1BB (CD137) |
| CD40L (CD154) | CD40 |
| CD27L (CD70) | CD27 |
| CD30L | CD30 |
| LIGHT | HVEM, LTβR, DcR3 |
| GITRL | GITR |
| BAFF (BLyS) ** | BAFF-R, TACI, BCMA |
| APRIL ** | TACI, BCMA |
| VEGI/TL1A | DR3 |
| TNF alpha (mutants) | TNFR2 |

Signal 3

In some embodiments, the exogenous Treg costimulatory polypeptide for stimulating Signal 3 includes a cytokine or growth factors that stimulates Signal 3, such as IL-2, IL-4, and TGF-β (including TGF-β1, TGF-β2 and TGF-β3), or a fragment thereof. IL-2 and IL-4 moieties useful in immunotherapeutic methods are known in the art. See, e.g., Earle et al. (2005), supra; Thorton et al. (2004) J. Immunol. 172: 6519-23; Thorton et al. (2004) Eur. J. Immunol. 34: 366-76. In some embodiments, the mature portion of the cytokine is used.

In some embodiments, the Treg costimulatory polypeptide comprises a CD25-specific IL-2, or a fragment thereof. In some embodiments, the Treg costimulatory polypeptide comprises TNFR2-specific TNF, or a fragment thereof. In some embodiments, the Treg costimulatory polypeptide comprises an anti-DR3 agonist (VEGUTL1A specific), or a fragment thereof. In some embodiments, the Treg costimulatory peptide comprises 4-1BBL, or a fragment thereof. In some embodiments, the Treg costimulatory peptide comprises TGFbeta, or a fragment thereof.

In other embodiments, the engineered erythroid cell or enucleated cell comprises an exogenous Treg co-inhibitory polypeptide capable of inhibiting Treg cells. In certain embodiments, Treg inhibition is useful in the treatment of cancer, for example, by targeting chemokines that are involved in Treg trafficking. In some embodiments, the exogenous Treg co-inhibitory polypeptide can target any of the receptors listed in Tables 10 or 11, for example, anti-OX40, anti-GITR or anti-CTLA4, or TLR ligands.

In some embodiments, the exogenous Treg co-stimulatory polypeptides or exogenous Treg co-inhibitory polypeptide, or an active fragment thereof, can be linked or expressed as a fusion protein with a binding pair member for use as described herein. An exemplary binding pair is biotin and streptavidin (SA) or avidin.

In some embodiments, the exogenous Treg costimulatory polypeptides, or an active fragment thereof, is part of a fusion protein, comprising a Treg costimulatory polypeptide and a binding pair member, such as CSA. Fusion proteins can be made by any of a number of different methods known in the art. For example, one or more of the component polypeptides of the fusion proteins can be chemically synthesized or can be generated using well known recombinant nucleic acid technology.

In some embodiments, the engineered erythroid cell or enucleated cell presents, e.g. comprises on the cell surface, at least two, at least 3, at least 4, or at least 5 exogenous Treg co-stimulatory polypeptides.

In some embodiments, the engineered erythroid cell or enucleated cell presents, e.g. comprises on the cell surface, at least two, at least 3, at least 4, or at least 5 exogenous Treg inhibitory polypeptides.

In some embodiments, the one or more Treg co-stimulatory or co-inhibitory polypeptides include or are fused to a transmembrane domain. In some embodiments, the transmembrane domain is selected from a sequence set forth in Table 2. In some embodiments, the one or more Treg co-stimulatory or co-inhibitory polypeptides include or are fused to a leader sequence. In some embodiments, the leader sequence is selected from a sequence set forth in Table 1.

### Cytokines/Chemokines

Additionally, the engineered erythroid cells or enucleated cells provided herein further comprise at least one exogenous polypeptide comprising a cytokine, at least one exogenous polypeptide comprising a chemokine, or both.

Exemplary cytokines include a hematopoietic growth factor, an interleukin, an interferon, an immunoglobulin superfamily molecule, and a tumor necrosis factor family molecule, granulocyte macrophage colony stimulating factor (GM-CSF), tumor necrosis factor alpha (TNFα), tumor necrosis factor beta (TNFβ), macrophage colony stimulating factor (M-CSF), interleukin-1 (IL-1), interleukin-2 (IL-2), interleukin-4 (IL-4), interleukin-5 (IL-5), interleukin-6 (IL-6), interleukin-7 (IL-7), interleukin-10 (IL-10), interleukin-12 (IL-12), interleukin-15 (IL-15), interleukin-21 (IL-21), interleukin-35 (IL-35), interferon alpha (IFN-α), interferon beta (IFN-β), interferon gamma (IFN-γ), and IGIF, and fragments thereof.

Exemplary chemokines, include an alpha-chemokine or a beta-chemokine, including, but not limited to, a C5a, interleukin-8 (IL-8), monocyte chemotactic protein lalpha (MIP1α), monocyte chemotactic protein 1 beta (MIP1β), monocyte chemoattractant protein 1 (MCP-1), monocyte chemoattractant protein 3 (MCP-3), platelet activating factor (PAFR), N-formyl-methionyl-leucyl-[³H]phenylalanine (FMLPR), leukotriene B4 (LTB4R), gastrin releasing peptide (GRP), RANTES, eotaxin, lymphotactin, IP10, 1-309, ENA78, GCP-2, NAP-2, and/or MGSA/gro.

In some embodiments, the exogenous polypeptide comprising a cytokine on the engineered erythroid cell or enucleated cell serves as a polypeptide for stimulating Signal 3. It will be understood that in some embodiments, the engineered erythroid cells or enucleated cells of the disclosure can expand and/or activate T cells by stimulating all three signals involved in T cell development. Signal 1 involves TCR, and can be stimulated with antigens that signal through TCR. Signal 2 can be mediated by several different molecules, including any immune co-stimulatory molecules described herein, such as 4-1BBL. Signal 3 can be transduced via cytokines, such as IL-15. Without being bound by theory, it is thought that the presence of signal 3, for example from a third exogenous polypeptide on the engineered erythroid cell or enucleated cell, in addition to signals 1 and 2, from a first and second exogenous polypeptide, respectively, e.g., an antigen and costimulatory polypeptide as described herein, increases the capacity of the engineered erythroid cells or enucleated cells to boost the memory T cell population and thereby provide longer efficacy, e.g., efficacy against a relapse of a tumor or re-challenge with an infectious agent. In some embodiments, the polypeptide for stimulating Signal 3 is an exogenous polypeptide comprising IL-15, or a fragment thereof. In some embodiments, the engineered erythroid cell or enucleated cell comprises a third exogenous polypeptide that stimulates Signal 3 (e.g., IL-15 or a fragment thereof).

In some embodiments, an engineered erythroid cell or enucleated cell , comprises one or more (e.g., 2, 3, 4, 5, or more) exogenous polypeptides comprising a cytokine receptor subunits from Table 14 or cytokine-binding variants or fragments thereof. In some embodiments, an engineered erythroid cell or enucleated cell comprises two or three (e.g., all) cytokine receptor subunits from a single row of Table 14 or cytokine-binding variants or functional fragments thereof. The exogenous polypeptide comprising a cytokine receptor can be present on the surface of the engineered erythroid cell or enucleated cell. The expressed receptors typically have the wild type human receptor sequence or a variant or fragment thereof that is able to bind and sequester its target ligand. In some embodiments, two or more exogenous polypeptides comprising a cytokine receptor subunit are linked to each other, e.g., as a fusion protein.

In some embodiments, the one or more exogenous polypeptides comprising a cytokine, a chemokine, or a cytokine receptor subunit include or are fused to a transmembrane domain. In some embodiments, the transmembrane domain is selected from a sequence set forth in Table 14. In some embodiments, the one or more exogenous polyepeptides comprising a cytokine, a chemokine, or a cytokine receptor subunit include or are fused to a leader sequence. In some embodiments, the leader sequence is selected from a sequence set forth in Table 14.

In some embodiments, the erythroid cell further comprises a targeting moiety, e.g., an address moiety or targeting moiety described in International Publication No. WO 2007/030708, e.g., at pages 34-45 therein, which application is herein incorporated by reference in its entirety.

TABLE 14
Cytokines and Receptors

| Name | Cytokine Receptor(s)(Da) and Form |
|---|---|
| Interleukins | |
| IL-1-like | |
| IL-1α | CD121a, CDw121b |
| IL-1β | CD121a, CDw121b |
| IL-1RA | CD121a |
| IL-18 | IL-18Rα, β |
| Common g chain (CD132) | |
| IL-2 | CD25, 122, 132 |
| IL-4 | CD124, 213a13, 132 |
| IL-7 | CD127, 132 |
| IL-9 | IL-9R, CD132 |
| IL-13 | CD213a1, 213a2, |
| IL-15 | IL-15Ra, CD122, 132 |
| IL-21 | IL21R |
| Common b chain (CD131) | |
| IL-3 | CD123, CDw131 |
| IL-5 | CDw125, 131 |
| Also related | |
| GM-CSF | CD116, CDw131 |
| IL-6-like | |
| IL-6 | CD126, 130 |
| IL-11 | IL-11Ra, CD130 |
| Also related | |
| G-CSF | CD114 |
| IL-12 | CD212 |
| IL-35 | IL35R |
| LIF | LIFR, CD130 |
| OSM | OSMR, CD130 |
| IL-10-like | |
| IL-10 | CDw210 |
| IL-20 | IL-20Rα, β |
| Others | |
| IL-14 | IL-14R |
| IL-16 | CD4 |
| IL-17 | CDw217 |
| Interferons | |
| IFN-α | CD118 |
| IFN-β | CD118 |
| IFN-γ | CDw119 |
| TNF | |
| CD154 | CD40 |
| LT-β | LTβR |
| TNF-α | CD120a, b |
| TNF-β (LT-α) | CD120a, b |
| 4-1BBL | CD137 (4-1BB) |
| APRIL | BCMA, TACI |
| CD70 | CD27 |
| CD153 | CD30 |
| CD178 | CD95 (Fas) |
| GITRL | GITR |
| LIGHT | LTbR, HVEM |
| OX40L | OX40 |
| TALL-1 | BCMA, TACI |
| TRAIL | TRAILR1-4 |
| TWEAK | Apo3 |
| TRANCE | RANK, OPG |
| TGF-β | |
| TGF-β1 | TGF-βR1 |
| TGF-β2 | TGF-βR2 |
| TGF-β3 | TGF-βR3 |
| Miscellaneous hematopoietins | |
| Epo | EpoR |
| Tpo | TpoR |
| Flt-3L | Flt-3 |
| SCF | CD117 |
| M-CSF | CD115 |
| MSP | CDw136 |

### Engineered Erythroid Cells Including Wild-Type or Loadable Exogenous Antigen-Presenting Polypeptides

The present disclosure features engineered erythroid cells (e.g., engineered enucleated erythroid cells) or enucleated cells (e.g., modified enucleated cells) that include a wild-type or loadable antigen-presenting polypeptide and additional polypeptide(s) of interest, as described herein (e.g., an exogenous antigenic polypeptide or an exogenous displaceable polypeptide). In some embodiments, an enucleated cell is a cell that lacks a nucleus (e.g., due to a differentiation process such as erythropoiesis). In some embodiments, an enucleated cell is incapable of expressing a polypeptide. In some embodiments, an enucleated cell is an erythrocyte, a reticulocyte, or a platelet. Engineered erythroid cells (e.g., engineered enucleated erythroid cells) or enucleated cells (e.g., modified enucleated cells) may be advantageously used for the treatment of, for example, cancer, autoimmune diseases, or infectious diseases.

In some aspects, the present disclosure provides an engineered erythroid cell or enucleated cell, engineered to activate T cells, wherein the cell presents, e.g. comprises on the cell surface, an exogenous loadable antigen-presenting polypeptide comprising one or more amino acid substitutions which stabilize the loadable exogenous antigen-presenting polypeptide on the cell surface.

In some embodiments, the wild-type or loadable antigen-presenting polypeptide is bound to an exogenous antigenic polypeptide disclosed in Tables 7-8 or an exogenous displaceable polypeptide disclosed herein.

In some embodiments, the engineered erythroid cell (e.g., engineered enucleated erythroid cell) or enucleated cell (e.g., modified enucleated cell) comprises a first exogenous polypeptide. In some embodiments, the engineered erythroid cell (e.g., engineered enucleated erythroid cell) or enucleated cell (e.g., modified enucleated cell) further comprises a second, different, exogenous polypeptide. The engineered erythroid cell (e.g., engineered enucleated erythroid cell) or enucleated cell (e.g., modified enucleated cell) optionally further comprises second and third, different, exogenous polypeptides. The engineered erythroid cell (e.g., engineered enucleated erythroid cell) or enucleated cell (e.g., modified enucleated cell) optionally further comprises second, third and fourth, different, exogenous polypeptides. The engineered erythroid cell (e.g., engineered enucleated erythroid cell) or enucleated cell (e.g., modified enucleated cell) optionally further comprises second, third, fourth and fifth, different, exogenous polypeptides. In some embodiments, the engineered erythroid cell (e.g., engineered enucleated erythroid cell) or enucleated cell (e.g., modified enucleated cell) optionally further comprises 1, 2, 3, 4, 5, 6, 7, 8, 9, 10, 11, 12, 13, 14, 15, 16, 17, 18, 19, 20 or more different, exogenous polypeptides. In some embodiments, the engineered erythroid cell (e.g., engineered enucleated erythroid cell) or enucleated cell (e.g., modified enucleated cell) optionally further comprises between 1-100, 1-200 different, exogenous polypeptides.

An exogenous antigenic polypeptide can be presented by the wild-type or loadable exogenous antigen-presenting polypeptide, i.e., the loadable exogenous antigen-presenting polypeptide is loaded with or bound to the exogenous antigen polypeptide. Thus, in some aspects, the present disclosure provides an engineered erythroid cell or enucleated cell that presents (e.g., comprises on the cell surface) a loaded exogenous antigen-presenting polypeptide including an exogenous antigenic polypeptide. In other embodiments, the loadable exogenous antigen-presenting polypeptide is bound to an exogenous displaceable polypeptide, as described herein, which can be displaced and replaced by an exogenous antigenic polypeptide.

In other aspects, the present disclosure provides an engineered erythroid cell (e.g., engineered enucleated erythroid cell) or enucleated cell (e.g., modified enucleated cell), wherein the cell includes a wild-type or loadable exogenous antigen-presenting polypeptide, with or without an exogenous displaceable polypeptide, and also comprises one or more of an exogenous antigenic polypeptide, an exogenous coinhibitory polypeptide, an exogenous costimulatory polypeptide, and/or an exogenous polypeptide comprising a cytokine, a chemokine or a cytokine receptor subunit.

### Circulation Time

In some embodiments, the engineered erythroid cell or enucleated cell (e.g., enucleated erythroid cell) of the present disclosure resides in circulation after administration to a subject for at least about 1 day to about 240 days (e.g., for at least about 1 day, 2 days, 3 days, 4 day, 5 days, 6 days, 7 days, 8 days, 9 days, 10 days, 11 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, 121 days, 122 days, 123 days, 124 days, 125 days, 126 days, 127 days, 128 days, 129 days, 130 days, 131 days, 132 days, 133 days, 134 days, 135 days, 136 days, 137 days, 138 days, 139 days, 140 days, 141 days, 142 days, 143 days, 144 days, 145 days, 146 days, 147 days, 148 days, 149 days, 150 days, 151 days, 152 days, 153 days, 154 days, 155 days, 156 days, 157 days, 158 days, 159 days, 160 days, 161 days, 162 days, 163 days, 164 days, 165 days, 166 days, 167 days, 168 days, 169 days, 170 days, 171 days, 172 days, 173 days, 174 days, 175 days, 176 days, 177 days, 178 days, 179 days, 180 days, 181 days, 182 days, 183 days, 184 days, 185 days, 186 days, 187 days, 188 days, 189 days, 190 days, 191 days, 192 days, 193 days, 194 days, 195 days, 196 days, 197 days, 198 days, 199 days, 200 days, 201 days, 202 days, 203 days, 204 days, 205 days, 206 days, 207 days, 208 days, 209 days, 210 days, 211 days, 212 days, 213 days, 214 days, 215 days, 216 days, 217 days, 218 days, 219 days, 220 days, 221 days, 222 days, 223 days, 224 days, 225 days, 226 days, 227 days, 228 days, 229 days, 230 days, 231 days, 232 days, 233 days, 234 days, 235 days, 236 days, 237 days, 238 days, 239 days, or 240 days.

### Copy Number

In some embodiments, one or more exogenous polypeptides (e.g., exogenous antigenic polypeptide, exogenous antigen-presenting polypeptides, exogenous costimulatory polypeptides, and exogenous coinhibitory polypeptides) have an abundance ratio of about 1:1, from about 2:1 to 1:2, from about 5:1 to 1:5, from about 10:1 to 1:10, from about 20:1 to 1:20, from about 50:1 to 1:50, from about 100:1 to 1:100 by weight or by copy number.

In some embodiments, the engineered erythroid cell (e.g., engineered enucleated erythroid cell) or enucleated cell (e.g., modified enucleated cell) comprises at least at least 10 copies, 100 copies, 1,000 copies, 5,000 copies 10,000 copies, 25,000 copies, 50,000 copies, or 100,000 copies of each of a first exogenous polypeptide and a second exogenous polypeptide. In some embodiments, the copy number of a first exogenous polypeptide is no more than 10%, 20%, 30%, 40%, 50%, 60%, 70%, 80%, or 90% greater, or no more than 2, 5, 10, 20, 50, 100, 200, 500, or 1000 times greater than the copy number of a second exogenous polypeptide. In some embodiments, the copy number of a second exogenous polypeptide is no more than 10%, 20%, 30%, 40%, 50%, 60%, 70%, 80%, or 90% greater, or no more than 2, 5, 10, 20, 50, 100, 200, 500, or 1000 times greater than the copy number of a first exogenous polypeptide.

In some embodiments, the first exogenous polypeptide comprises between about 50,000 to about 600,000 copies of a first exogenous polypeptide, for example about 50,000, 60,000, 60,000, 80,000, 90,000, 100,000, 110,000, 120,000, 130,000, 140,000, 150,000, 155,000, 160,000, 165,000, 170,000, 175,000, 180,000, 185,000, 190,000, 195,000, 200,000, 205,000, 210,000, 215,000, 220,000, 225,000, 230,000, 235,000, 240,000, 245,000, 250,000, 255,000, 260,000, 265,000, 270,000, 275,000, 280,000, 285,000, 290,000, 295,000, 300,000, 305,000, 310,000, 315,000, 320,000, 325,000, 330,000, 335,000, 340,000, 345,000, 350,000, 355,000, 360,000, 365,000, 370,000, 375,000, 380,000, 385,000, 390,000, 395,000, 400,000, 450,000, 500,000, 550,000, 600,000 copies of a first exogenous polypeptide. In some embodiments, the engineered erythroid cell or enucleated cell comprises between about 50,000-600,000, between about 100,000-600,000, between about 100,000-500,000, between about 100,000-400,000, between about 100,000-150,000, between about 150,000-300,000, or between 150,000-200,000 copies of a first exogenous polypeptide. In some embodiments, the engineered erythroid cell or enucleated cell comprises at least about 75,000 copies, about 100,000 copies, about 125,000 copies, about 150,000 copies, about 175,000 copies, about 200,000 copies, about 250,000 copies, about 300,000 copies about 400,000, or about 500,000 copies of a first exogenous polypeptide. In some embodiments, the engineered erythroid cell or enucleated cell comprises between about 50,000 to about 600,000 copies of a second exogenous polypeptide, for example about 50,000, 60,000, 60,000, 80,000, 90,000, 100,000, 110,000, 120,000, 130,000, 140,000, 150,000, 155,000, 160,000, 165,000, 170,000, 175,000, 180,000, 185,000, 190,000, 195,000, 200,000, 205,000, 210,000, 215,000, 220,000, 225,000, 230,000, 235,000, 240,000, 245,000, 250,000, 255,000, 260,000, 265,000, 270,000, 275,000, 280,000, 285,000, 290,000, 295,000, 300,000, 305,000, 310,000, 315,000, 320,000, 325,000, 330,000, 335,000, 340,000, 345,000, 350,000, 355,000, 360,000, 365,000, 370,000, 375,000, 380,000, 385,000, 390,000, 395,000, 400,000, 450,000, 500,000, 550,000, 600,000 copies of the second polypeptide. In some embodiments, the engineered erythroid cell comprises between about 50,000-600,000, between about 100,000-600,000, between about 100,000-500,000, between about 100,000-400,000, between about 100,000-150,000, between about 150,000-300,000, or between 150,000-200,000 copies of a second exogenous polypeptide. In some embodiments, the engineered erythroid cell comprises at least about 75,000 copies about 100,000 copies, about 125,000 copies, about 150,000 copies, about 175,000 copies, about 200,000 copies, about 250,000 copies, about 300,000 copies about 400,000, or about 500,000 copies of a second exogenous polypeptide.

### Populations of Engineered Erythroid Cells

In one aspect, the disclosure features populations of the engineered erythroid cells or enucleated cells described herein e.g., a plurality or population of the engineered enucleated erythroid cells. The terms “plurality” and “population” are used interchangeably herein. In some embodiments, a population of engineered erythroid cells or enucleated cells may comprise predominantly enucleated cells (e.g., greater than 70%), predominantly nucleated cells (e.g., greater than 70%), or any mixture of enucleated and nucleated cells. In some embodiments, a population of engineered erythroid cells or enucleated cells may comprise reticulocytes, erythrocytes, or a mixture of reticulocytes and erythrocytes. In some embodiments, a population of engineered erythroid cells or enucleated cells may predominantly comprise reticulocytes. In some embodiments, a population of engineered erythroid cells or enucleated cells may predominantly comprise erythrocytes (e.g., immature or mature erythrocytes).

In some embodiments, a population of engineered erythroid cells consists essentially of enucleated cells. In some embodiments, a population of engineered erythroid cells comprises predominantly or substantially enucleated cells. For example, in some embodiments, a population of engineered erythroid cells comprises at least about 70% or more enucleated cells. In some embodiments, the population provided herein comprises at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99, or about 100% enucleated cells. In some embodiments, the population provided herein comprises greater than about 70% enucleated cells. In some embodiments, the population of engineered erythroid cells comprises greater than about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% enucleated cells. In some embodiments, the population of engineered erythroid cells comprises between about 80% and about 100% enucleated cells, for example between about 80% and about 95%, about 80% and about 90%, about 80% and about 85%, about 85% and about 100%, about 85% and about 95%, about 85% and about 90%, about 90% and about 100%, about 90% and about 95%, or about 95% and about 100% of enucleated cells.

In some embodiments, the population of engineered erythroid cells comprises less than about 30% nucleated cells. For example, in embodiments, the population of engineered erythroid cells comprises less than about 1%, about 2%, about 3%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or less than about 30% nucleated cells. In some embodiments, the population of engineered erythroid cells comprises less than about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, or about 19%, about 21%, about 22%, about 23%, about 24%, about 25%, about 26%, about 27%, about 28%, about 29%, or about 30% nucleated cells. In some embodiments, the population of engineered erythroid cells comprises between 0% and 30% nucleated cells. In some embodiments, the populations of engineered erythroid cells comprise between about 0% and 20% nucleated cells, for example between about 0% and 19%, between about 0% and 15%, between about 0% and 10%, between about 0% and 5%, between about 0% and 4%, between about 0% and 3%, between about 0% and 2% nucleated cells, or between about 5% and 20%, between about 10% and 20%, or between about 15% and 20% nucleated cells.

In some embodiments, the disclosure features a population of the engineered erythroid cells as described herein, wherein the population of engineered erythroid cells comprises less than 30% nucleated cells and at least 70% enucleated cells, or comprises less than 20% nucleated cells and at least 80% enucleated cells, or comprises less than 15% nucleated cells and at least 85% nucleated cells, or comprises less than 10% nucleated cells and at least 90% enucleated cells, or comprises less than 5% nucleated cells and at least 95% enucleated cells. In some embodiments, the disclosure features populations of the engineered erythroid cells as described herein, wherein the population of engineered erythroid cells comprises about 0% nucleated cells and about 100% enucleated cells, about 1% nucleated cells and about 99% enucleated cells, about 2% nucleated cells and about 98% enucleated cells, about 3% nucleated cells and about 97% enucleated cells, about 4% nucleated cells and about 96% enucleated cells, about 5% nucleated cells and about 95% enucleated cells, about 6% nucleated cells and about 94% enucleated cells, about 7% nucleated cells and about 93% enucleated cells, about 8% nucleated cells and about 92% enucleated cells, about 9% nucleated cells and about 91% enucleated cells, about 10% nucleated cells and about 90% enucleated cells, about 11% nucleated cells and about 89% enucleated cells, about 12% nucleated cells and about 88% enucleated cells, about 13% nucleated cells and about 87% enucleated cells, about 14% nucleated cells and about 86% enucleated cells, about 85% nucleated cells and about 85% enucleated cells, about 16% nucleated cells and about 84% enucleated cells, about 17% nucleated cells and about 83% enucleated cells, about 18% nucleated cells and about 82% enucleated cells, about 19% nucleated cells and about 81% enucleated cells, or about 20% nucleated cells and about 80% enucleated cells.

In other embodiments, the engineered erythroid cell population comprises predominantly or substantially nucleated cells. In some embodiments, the engineered erythroid cell population consists essentially of nucleated cells. In various embodiments, the nucleated cells in the engineered erythroid cell population are erythroid precursor cells. In some embodiments, the erythroid precursor cells are selected from the group consisting of pluripotent hematopoietic stem cells (HSCs), multipotent myeloid progenitor cells, CFU-S cells, BFU-E cells, CFU-E cells, pronormoblasts, basophilic normoblasts, polychromatophilic normoblasts and orthochromatophilic normoblasts.

In certain embodiments, the population of engineered erythroid cells comprises at least about 10%, at least about 20%, at least about 30%, at least about 40%, at least about 50%, at least about 60%, at least about 70%, at least about 75%, at least about 80%, at least about 85%, at least about 90%, at least about 95%, at least about 98%, at least about 99% or 100% nucleated cells.

It will be understood that during the preparation of the engineered erythroid cells or enucleated cells of the as described herein, some fraction of cells may not include an exogenous polypeptide (e.g., due to lack of expression or transduction or conjugation with an exogenous nucleic acid). Accordingly, in some embodiments, a population of engineered erythroid cells or enucleated cells provided herein comprises a mixture of engineered erythroid cells and unmodified erythroid cells, or a mixture of modified enucleated cells and unmodified enucleate cells, i.e., some fraction of cells in the population will not include (e.g., express) an exogenous polypeptide. For example, a population of engineered erythroid cells or enucleated cells can comprise, in various embodiments, at least about 10%, 20%, 30%, 40%, 50%, 60%, 70%, 80%, 90%, 91%, 92%, 93%, 94%, 95%, 96%, 97%, 98% or 99% erythroid cells or enucleated cells that include an exogenous polypeptide, wherein the remaining erythroid cells or enucleated cells in the population are do not include an exogenous polypeptide. In some embodiments, a single unit dose of engineered erythroid cells (e.g., engineered enucleated erythroid cells) or enucleated cells comprises at least about 10%, 20%, 30%, 40%, 50%, 60%, 65%, 70%, 75%, 80%, 85%, 90%, 91%, 92%, 93%, 94%, 95%, 96%, 97%, 98% or 99% erythroid cells or enucleated cells including an exogenous polypeptide, wherein the remaining erythroid cells or enucleated cells in the dose do not include an exogenous polypeptide.

### II. Methods of Making Engineered Erythroid Cells or Enucleated Cells

Various methods of making engineered erythroid cells (e.g., engineered enucleated erythroid cells) or enucleated cells (e.g., modified enucleated cells) are contemplated by the present disclosure. In some aspects, the present disclosure features a method of making an engineered erythroid cell or enucleated cell, wherein the cell comprises an engineered erythroid cell or enucleated cell that includes an exogenous immunogenic polypeptide and, in some embodiments, an exogenous antigenic polypeptide.

In some embodiments, the description provides a method of producing the engineered erythroid cell or enucleated cell comprising a loadable exogenous antigen-presenting polypeptide on the cell surface, the method comprising introducing an exogenous nucleic acid encoding the loadable exogenous antigen-presenting polypeptide into a nucleated erythroid precursor cell, wherein the loadable exogenous antigen-presenting polypeptide comprises one or more amino acid substitutions which stabilize the loadable exogenous antigen-presenting polypeptide on the cell surface; culturing the nucleated erythroid precursor cell under conditions suitable for enucleation and production of the loadable exogenous antigen-presenting polypeptide, thereby making an engineered enucleated erythroid cell comprising a loadable exogenous antigen-presenting polypeptide on the cell surface, thereby making the engineered erythroid cell (e.g., engineered enucleated erythroid cell) or enucleated cell (e.g., modified enucleated cell). In some embodiments, the method further comprises introducing at least one (e.g., one, two, or three) exogenous nucleic acid encoding at least one (e.g., one, two, or three) exogenous antigenic polypeptide into the erythroid precursor cell. In some embodiments, the at least one exogenous antigenic polypeptide comprises a transmembrane domain and optionally a linker. In some embodiments, the method further comprises introducing an exogenous nucleic acid encoding an exogenous displaceable polypeptide into the erythroid precursor cell.

The present disclosure further provides a method of contacting the engineered erythroid cell (e.g., engineered enucleated erythroid cell) or enucleated cell (e.g., modified enucleated cell) with at least one exogenous antigenic polypeptide, wherein the at least one exogenous antigenic polypeptide specifically binds to the wild-type or loadable exogenous antigen-presenting polypeptide which is present on the cell surface of the engineered enucleated erythroid cell. In some embodiments, the exogenous antigenic polypeptide is conjugated (e.g., covalently) to the wild-type or loadable exogenous antigen-presenting polypeptide.

In some embodiments, the method comprises contacting the engineered erythroid cell (e.g., engineered enucleated erythroid cell) or enucleated cell (e.g., modified enucleated cell) with multiple different (e.g., two, three, four, five or more) exogenous antigenic polypeptides, wherein the multiple different (e.g., two, three, four, five or more) exogenous antigenic polypeptides specifically bind to the wild-type or loadable exogenous antigen-presenting polypeptide which is present on the cell surface of the engineered enucleated erythroid cell. In some embodiments, the enucleated erythroid cell (e.g., engineered enucleated erythroid cell) or enucleated cell (e.g., modified enucleated cell) is contacted with two or more exogenous antigenic polypeptides, wherein the two or more exogenous antigenic polypeptides specifically bind to the wild-type or loadable exogenous antigen-presenting polypeptide. In some embodiments, the enucleated erythroid cell (e.g., engineered enucleated erythroid cell) or enucleated cell (e.g., modified enucleated cell) is contacted with three or more exogenous antigenic polypeptides, wherein the three or more exogenous antigenic polypeptides specifically bind to the wild-type or loadable exogenous antigen-presenting polypeptide. In some embodiments, the enucleated erythroid cell (e.g., engineered enucleated erythroid cell) or enucleated cell (e.g., modified enucleated cell) is contacted with four or more exogenous antigenic polypeptides, wherein the four or more exogenous antigenic polypeptides specifically bind to the wild-type or loadable exogenous antigen-presenting polypeptide. In some embodiments, the enucleated erythroid cell (e.g., engineered enucleated erythroid cell) or enucleated cell (e.g., modified enucleated cell) is contacted with five or more exogenous antigenic polypeptides, wherein the five or more exogenous antigenic polypeptides specifically bind to the wild-type or loadable exogenous antigen-presenting polypeptide. In some embodiments, the wild-type or loadable exogenous antigen-presenting polypeptide comprises an MEW class I polypeptide. In some embodiments, the wild-type or loadable exogenous antigen-presenting polypeptide comprises an MEW class II polypeptide.

In some embodiments, the exogenous nucleic acid further encodes an exogenous displaceable polypeptide. In other embodiments, the exogenous nucleic acid further encodes a linker. In other embodiments, the exogenous nucleic acid further encodes a transmembrane domain. In some embodiments, the exogenous wild-type or loadable antigen-presenting polypeptide, transmembrane domain, linker and exogenous displaceable polypeptide are comprised in a single chain fusion protein. In some embodiments, the single chain fusion protein comprises a linker disposed between the wild-type or loadable exogenous antigen-presenting polypeptide and the exogenous displaceable polypeptide.

In some embodiments, the methods described herein further comprise displacing the displaceable exogenous polypeptide from the loadable exogenous antigen-presenting polypeptide by contacting the engineered enucleated erythroid cell in vitro with an exogenous antigenic polypeptide, wherein the loadable exogenous antigen-presenting polypeptide has a higher affinity for the exogenous antigenic polypeptide than for an exogenous displaceable polypeptide.

In some embodiments, the exogenous nucleic acid comprises DNA or RNA. In some embodiments, the introducing step comprises viral transduction. In some embodiments, the introducing step comprises electroporation. In some embodiments, the introducing step comprises utilizing one or more of: liposome mediated transfer, adenovirus, adeno-associated virus, herpes virus, a retroviral based vector, lipofection, and a lentiviral vector.

In some embodiments, the engineered erythroid cell (e.g., engineered enucleated erythroid cell) or enucleated cell (e.g., modified enucleated cell) further comprises an additional exogenous polypeptide, include, for example, an exogenous coinhibitory polypeptide, a costimulatory polypeptide, a cytokine, or a membrane anchor polypeptide.

Methods of making an engineered erythroid cell (e.g., engineered enucleated erythroid cell) or enucleated cell (e.g., modified enucleated cell) are described herein, however it is to be understood that these methods are non-limiting.

The processes of making the engineered erythroid cells and enucleated cells are described in more detail below.

### Methods of Manufacturing Enucleated Erythroid Cells

Methods of manufacturing enucleated erythroid cells comprising an exogenous agent (e.g., a polypeptide) are described, e.g., in International Application Publication Nos. WO2015/073587 and WO2015/153102, each of which is incorporated by reference in its entirety.

In some embodiments, hematopoietic progenitor cells, e.g., CD34⁺ hematopoietic progenitor cells (e.g., human (e.g., adult human) or mouse cells), are contacted with a nucleic acid or nucleic acids encoding one or more exogenous polypeptides, and the cells are allowed to expand and differentiate in culture. In some embodiments, the CD34⁺ cells are immortalized, e.g., comprise a human papilloma virus (HPV; e.g., HPV type 16) E6 and/or E7 genes. In some embodiments, the immortalized CD34⁺ hematopoietic progenitor cell is a BEL-A cell line cell (see Trakarnasanga et al. (2017) Nat. Commun. 8: 14750). Additional immortalized CD34⁺ hematopoietic progenitor cells are described in U.S. Pat. Nos. 9,951,350, and 8,975,072. In some embodiments, an immortalized CD34⁺ hematopoietic progenitor cell is contacted with a nucleic acid or nucleic acids encoding one or more exogenous polypeptides, and the cells are allowed to expand and differentiate in culture.

In some embodiments, the erythroid cells described herein are made by a method comprising contacting a nucleated erythroid cell (e.g., an erythroid precursor cell) with an exogenous nucleic acid. In some embodiments, the exogenous nucleic acid is codon-optimized. For instance, the exogenous nucleic acid may comprise one or more codons that differ from the wild-type codons in a way that does not change the amino acid encoded by that codon, but that increases translation of the nucleic acid, e.g., by using a codon preferred by the host cell, e.g., a mammalian cell, e.g., an erythroid cell.

The exogenous nucleic acid may be, e.g., DNA or RNA (e.g., mRNA). A number of viruses may be used as gene transfer vehicles including retroviruses, Moloney murine leukemia virus (MMLV), adenovirus, adeno-associated virus (AAV), herpes simplex virus (HSV), lentiviruses such as human immunodeficiency virus 1 (HIV 1), and spumaviruses such as foamy viruses, for example.

In some embodiments, the exogenous nucleic acid is operatively linked to a constitutive promoter. In some embodiments, a constitutive promoter is used to drive expression of the targeting moiety.

In some embodiments, the exogenous nucleic acid is operatively linked to an inducible or repressible promoter, e.g., to drive expression of the exogenous polypeptide. For instance, the promoter may be doxycycline-inducible, e.g., a P-TRE3GS promoter or active fragment or variant thereof. Examples of inducible promoters include, but are not limited, to a metallothionine-inducible promoter, a glucocorticoid-inducible promoter, a progesterone-inducible promoter, and a tetracycline-inducible promoter (which may also be doxycycline-inducible). In some embodiments, the inducer is added to culture media comprising cells that comprise the inducible promoter, e.g., at a specific stage of cell differentiation. In some embodiments, the inducer (e.g., doxycycline) is added at an amount of about 1-5, 2-4, or 3 μg/mL. In some embodiments, a repressor is withdrawn from to culture media comprising cells that comprise the repressible promoter, e.g., at a specific stage of cell differentiation. In some embodiments, the inducer is added, or the repressor is withdrawn, during maturation phase, e.g., between days 1-10, 2-9, 3-8, 4-6, or about day 5 of maturation phase. In some embodiments, the inducer is present, or the repressor is absent, between day 1, 2, 3, 4, 5, 6, 7,8, 9 or 10 of maturation and enucleation. In some embodiments, the inducer is present, or the repressor is absent, for at least 1, 2, 3, 4, 5, 6, 7, 8, 9, or 10 days. In some embodiments, the inducer is present, or the repressor is absent, from maturation day 5 to the end of differentiation. In some embodiments, the inducer is present, or the repressor is absent at maturation day 9. In some embodiments, the inducer is added, or the repressor is withdrawn, when the population of erythroid cells comprises a plurality of normoblasts (e.g., basophilic, polychromatic, or orthochromatic normoblasts or a combination thereof), e.g., when 10-20%, 20-30%, 30-40%, 40-50%, 50-60%, 60-70%, or 70-80% of the cells in the population are normoblasts. In some embodiments, the inducer is added, or the repressor is withdrawn, when the population of erythroid cells comprises a plurality of pro-erythroblasts, e.g., when 10-20%, 20-30%, 30-40%, 40-50%, 50-60%, 60-70%, or 70-80% of the cells in the population are pro-erythroblasts. In some embodiments, the inducer is added, or the repressor is withdrawn, when the population of erythroid cells comprises a plurality of erythroblasts at terminal differentiation e.g., when 10-20%, 20-30%, 30-40%, 40-50%, 50-60%, 60-70%, or 70-80% of the cells in the population are erythroblasts at terminal differentiation. In some embodiments, the erythroid cell or population of erythroid cells comprises an additional exogenous protein, e.g., a transactivator, e.g., a Tet-inducible transactivator (e.g., a Tet-on-3G transactivator).

In some embodiments, the inducer is added, or the repressor is withdrawn, when the population of erythroid cells comprises one or more of (e.g., all of) endogenous GPA, band 3, or alpha4 integrin. In some embodiments, the inducer is added, or the repressor is withdrawn, during a time when about 84-100%, 85-100%, 90-100%, or 95-100% of the cells in the population are GPA-positive (e.g., when the population first reaches that level); during a time when 50-100%, 60-100%, 70-100%, 80-100%, 90-100%, 95-100%, or 98-100% of the cells in the population are band 3-positive (e.g., when the population first reaches that level); and/or during a time when about 70-100%, 80-90%, or about 85% of the cells in the population are alpha4 integrin-positive (e.g., when the population first reaches that level).

GPA, band 3, and alpha4 integrin can be detected, e.g., by a flow cytometry assay, e.g., a flow cytometry assay of Example 10 of International Application Publication No. WO2018/009838, incorporated herein by reference.

In some embodiments, the cells are produced using conjugation, e.g., sortagging or sortase-mediated conjugation, e.g., as described in International Application Publication Nos. WO2014/183071 or WO2014/183066, each of which is incorporated by reference in its entirety. In some embodiments, the cells are made by a method that does not comprise sortase-mediated conjunction.

In some embodiments, the cells are made by a method that does not comprise hypotonic loading. In some embodiments, the cells are made by a method that does not comprise a hypotonic dialysis step. In some embodiments, the cells are made by a method that does not comprise controlled cell deformation.

In some embodiments, the erythroid cells are expanded at least 1000, 2000, 5000, 10,000, 20,000, 50,000, or 100,000 fold (and optionally up to 100,000, 200,000, or 500,000 fold). The number of cells is measured, in some embodiments, using an automated cell counter. In some embodiments, the population of erythroid cells comprises at least about 30%, 35%, 40%, 45%, 50%, 55%, 60%, 65%, 70%, 75%, 80%, 85%, 90%, 95%, 96% or 98% (and optionally up to about 80, 90, or 100%) enucleated erythroid cells. In some embodiments, the population of erythroid cells comprises 70%-100%, 75%-100%, 80%-100%, 85%-100%, or 90%-100% enucleated cells. In some embodiments, the population of erythroid cells contains less than 1% live nucleated cells, e.g., contains no detectable live nucleated cells. Enucleation is measured, in some embodiments, by FACS using a nuclear stain. In some embodiments, at least about 30%, 35%, 40%, 45%, 50%, 55%, 60%, 65%, 70%, 75%, 80%, 85%, 90%, 95%, 96% or 98% (and optionally up to about 70, 80, 90, or 100%) of erythroid cells in the population comprise one or more (e.g., 2, 3, 4 or more) of the exogenous polypeptides. Level of the polypeptides is measured, in some embodiments, by erythroid cells using labeled antibodies against the polypeptides. In some embodiments, at least about 30%, 35%, 40%, 45%, 50%, 55%, 60%, 65%, 70%, 75%, 80%, 85%, 90%, 95%, 96% or 98% (and optionally up to about 70, 80, 90, or 100%) of erythroid cells in the population are enucleated and comprise one or more (e.g., 2, 3, 4, or more) of the exogenous polypeptides. In some embodiments, the population of erythroid cells comprises about 1×10⁹-2×10⁹, 2×10⁹-5×10⁹, 5×10⁹-1×10¹⁰, 1×10¹⁰, 2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

### Physical Characteristics of Engineered Erythroid Cells

In some embodiments, the engineered erythroid cells (e.g., engineered enucleated erythroid cells) or enucleated cells (e.g., modified enucleated cells) described herein have one or more (e.g., 2, 3, 4, or more) physical characteristics described herein, e.g., osmotic fragility, cell size, hemoglobin concentration, or phosphatidylserine content. While not wishing to be bound by theory, in some embodiments, an engineered erythroid cell, (e.g., an engineered enucleated erythroid cell) or an enucleated cell (e.g., modified enucleated cell), that includes an exogenous polypeptide described herein has physical characteristics that resemble a wild-type, untreated erythroid cell or enucleated cell. In contrast, a hypotonically-loaded erythroid cell may sometimes display aberrant physical characteristics such as increased osmotic fragility, altered cell size, reduced hemoglobin concentration, or increased phosphatidylserine levels on the outer leaflet of the cell membrane.

Osmotic Fragility

In some embodiments, the engineered erythroid cell or enucleated cell exhibits substantially the same osmotic membrane fragility as an isolated, uncultured erythroid cell that does not comprise an exogenous polypeptide. In some embodiments, the population of engineered erythroid cells or enucleated cells has an osmotic fragility of less than 50% cell lysis at 0.3%, 0.35%, 0.4%, 0.45%, or 0.5% NaCl. Osmotic fragility can be assayed using the method of Example 59 of WO2015/073587, which is herein incorporated by reference in its entirety.

Cell Size

In some embodiments, the engineered erythroid cell (e.g., engineered enucleated erythroid cell) or enucleated cell (e.g., modified enucleated cell) has approximately the diameter or volume as a wild-type, untreated enucleated erythroid cell.

In some embodiments, the population of engineered erythroid cells (e.g., engineered enucleated erythroid cells) or enucleated cells (e.g., modified enucleated cells) has an average diameter of about 4, 5, 6, 7, or 8 microns, and optionally the standard deviation of the population is less than 1, 2, or 3 microns. In some embodiments, the one or more engineered erythroid cell (e.g., engineered enucleated erythroid cell) or enucleated cell (e.g., modified enucleated cell) has a diameter of about 4-8, 5-7, or about 6 microns. In some embodiments, the diameter of the erythroid cell is less than about 1 micron, larger than about 20 microns, between about 1 micron and about 20 microns, between about 2 microns and about 20 microns, between about 3 microns and about 20 microns, between about 4 microns and about 20 microns, between about 5 microns and about 20 microns, between about 6 microns and about 20 microns, between about 5 microns and about 15 microns or between about 10 microns and about 30 microns. Cell diameter is measured, in some embodiments, using an Advia 120 hematology system.

In some embodiment the volume of the mean corpuscular volume of the erythroid cells is greater than 10 fL, 20 fL, 30 fL, 40 fL, 50 fL, 60 fL, 70 fL, 80 fL, 90 fL, 100 fL, 110 fL, 120 fL, 130 fL, 140 fL, 150 fL, or greater than 150 fL. In some embodiments, the mean corpuscular volume of the erythroid cells is less than 30 fL, 40 fL, 50 fL, 60 fL, 70 fL, 80 fL, 90 fL, 100 fL, 110 fL, 120 fL, 130 fL, 140 fL, 150 fL, 160 fL, 170 fL, 180 fL, 190 fL, 200 fL, or less than 200 fL. In some embodiments, the mean corpuscular volume of the erythroid cells is between 80-100, 100-200, 200-300, 300-400, or 400-500 femtoliters (fL). In some embodiments, a population of engineered erythroid cells (e.g., engineered enucleated erythroid cells) or enucleated cells (e.g., modified enucleated cells) has a mean corpuscular volume set out in this paragraph and the standard deviation of the population is less than 50, 40, 30, 20, 10, 5, or 2 fL. The mean corpuscular volume is measured, in some embodiments, using a hematological analysis instrument, e.g., a Coulter counter.

Hemoglobin Concentration

In some embodiments, the engineered erythroid cell (e.g., engineered enucleated erythroid cell) or enucleated cell (e.g., modified enucleated cell) has a hemoglobin content similar to a wild-type, untreated enucleated erythroid cell or enucleated cell. In some embodiments, the engineered erythroid cells (e.g., engineered enucleated erythroid cells) or enucleated cells (e.g., modified enucleated cells) comprise at least about 20, 22, 24, 26, 28, or 30 pg, and optionally up to about 30 pg, of total hemoglobin. Hemoglobin levels are determined, in some embodiments, using the Drabkin's reagent method of Example 33 of International Application Publication No. WO2015/073587, which is herein incorporated by reference in its entirety.

Phosphatidylserine Content

In some embodiments, the engineered erythroid cells (e.g., engineered enucleated erythroid cells) or enucleated cells (e.g., modified enucleated cells), has approximately the same phosphatidylserine content on the outer leaflet of its cell membrane as a wild-type, untreated erythroid cell or enucleated cell. Phosphatidylserine is predominantly on the inner leaflet of the cell membrane of wild-type, untreated erythroid cells, and hypotonic loading can cause the phosphatidylserine to distribute to the outer leaflet where it can trigger an immune response. In some embodiments, the population of engineered erythroid cells (e.g., engineered enucleated erythroid cells) or enucleated cells (e.g., modified enucleated cells) comprises less than about 30, 25, 20, 15, 10, 9, 8, 6, 5, 4, 3, 2, or 1% of cells that are positive for annexin V staining. Phosphatidylserine exposure is assessed, in some embodiments, by staining for annexin-V-FITC, which binds preferentially to PS, and measuring FITC fluorescence by flow cytometry, e.g., using the method of Example 54 of International Application Publication Nos. WO2015/073587, which is herein incorporated by reference in its entirety.

Other Characteristics

In some embodiments, an engineered erythroid cell (e.g., engineered enucleated erythroid cell) or an enucleated cell, or a population of engineered erythroid cells or enucleated cells comprises one or more of (e.g., all of) endogenous GPA (C235a), transferrin receptor (CD71), Band 3 (CD233), or integrin alpha4 (C49d). These proteins can be measured, e.g., as described in Example 10 of International Application Publication No. WO2018/009838, which is herein incorporated by reference in its entirety. The percentage of GPA-positive cells and Band 3-positive cells typically increases during maturation of an erythroid cell, and the percentage of integrin alpha4-positive typically remains high throughout maturation.

In some embodiments, the population of engineered erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% GPA+(i.e., CD235a⁺) cells. In some embodiments, the population of engineered erythroid cells or enucleated cells comprises between about 50% and about 100% (e.g., from about 60% and about 100%, from about 65% and about 100%, from about 70% and about 100%, from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) GPA⁺ cells. The presence of GPA is detected, in some embodiments, using FACS.

In some embodiments, the population of engineered erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD71⁺ cells. In some embodiments, the population of engineered erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD71⁺ cells. The presence of CD71 (transferrin receptor) is detected, in some embodiments, using FACS.

In some embodiments, the population of engineered erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD233³⁰ cells. In some embodiments, the population of engineered erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD233⁺ cells. The presence of CD233 (Band 3) is detected, in some embodiments, using FACS.

In some embodiments, the population of engineered erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 8′7%, 88%, 89%, 90%, 91%, 92%, 93%, 94%, 95%, 96%, 97%, 98%, 99%, or 100% CD47⁺ cells. In some embodiments, the population of engineered erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD47⁺ cells. The presence of CD47 (integrin associate protein) is detected, in some embodiments, using FACS.

In some embodiments, the population of engineered erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD36⁺ (CD36-negative) cells. In some embodiments, the population of engineered erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD36⁻ (CD36-negative) cells. The presence of CD36 is detected, in some embodiments, using FACS.

In some embodiments, the population of engineered erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD34⁻ (CD34-negative) cells. In some embodiments, the population of engineered erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD34⁻ (CD34-negative) cells. The presence of CD34 is detected, in some embodiments, using FACS.

In some embodiments, the population of engineered erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD235a⁺/CD47⁺/CD233⁺ cells. In some embodiments, the population of engineered erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD235a⁺/CD47⁺/CD233⁺ cells.

In some embodiments, the population of engineered erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD235a⁺/CD47⁺/CD233⁺/CD34⁻/CD36⁻ cells. In some embodiments, the population of engineered erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD235a⁺/CD47⁺/CD233⁺/CD34⁻/CD36⁻0 cells.

In some embodiments, a population of engineered erythroid cells or enucleated cells comprising erythroid cells comprises less than about 10%, 9%, 8%, 7%, 6%, 5%, 4%, 3%, 2%, or 1% echinocytes. In some embodiments, a population of engineered erythroid cells or enucleated cells comprising comprises less than about 10%, 9%, 8%, 7%, 6%, 5%, 4%, 3%, 2%, or 1% pyrenocytes.

### Universal Donor Erythroid Cells

In some embodiments, erythroid cells or enucleated cells described herein are autologous and/or allogeneic to the subject to which the cells will be administered. For example, erythroid cells allogeneic to the subject include one or more of blood type specific erythroid cells (e.g., the cells can be of the same blood type as the subject) or one or more universal donor erythroid cells. In some embodiments, the enucleated erythroid cells described herein have reduced immunogenicity compared to a reference cell, e.g., have lowered levels of one or more blood group antigens.

Where allogeneic cells are used for transfusion, a compatible ABO blood group can be chosen to prevent an acute intravascular hemolytic transfusion reaction. The ABO blood types are defined based on the presence or absence of the blood type antigens A and B, monosaccharide carbohydrate structures that are found at the termini of oligosaccharide chains associated with glycoproteins and glycolipids on the surface of the erythrocytes (reviewed in Liu et al. (2007) Nat. Biotech. 25: 454-64). Because group 0 erythrocytes contain neither A nor B antigens, they can be safely transfused into recipients of any ABO blood group, e.g., group A, B, AB, or O recipients. Group O erythrocytes are considered universal and may be used in all blood transfusions. Thus, in some embodiments, an erythroid cell described herein is type O. In contrast, group A erythroid cells may be given to group A and AB recipients, group B erythroid cells may be given to group B and AB recipients, and group AB erythroid cells may be given to AB recipients.

In some instances, it may be beneficial to convert a non-group O erythroid cell to a universal blood type. Enzymatic removal of the immunodominant monosaccharides on the surface of group A and group B erythrocytes may be used to generate a population of group O-like erythroid cells (See, e.g., Liu et al. (2007)). Group B erythroid cells may be converted using an a-galactosidase from green coffee beans. Alternatively or in addition, α-N-acetylgalactosaminidase and a-galactosidase enzymatic activities from E. meningosepticum bacteria may be used to respectively remove the immunodominant A and B antigens (Liu et al. (2007)), if present on the erythroid cells. In one example, packed erythroid cells isolated as described herein, are incubated in 200 mM glycine (pH 6.8) and 3 mM NaCl in the presence of either a-N-acetylgalactosaminidase and a-galactosidase (about 300 μg/ml packed erythroid cells) for 60 min at 26° C. After treatment, the erythroid cells are washed by 3-4 rinses in saline with centrifugation and ABO-typed according to standard blood banking techniques.

While the ABO blood group system is the most important in transfusion and transplantation, in some embodiments it can be useful to match other blood groups between the erythroid cells to be administered and the recipient, or to select or make erythroid cells that are universal for one or more other (e.g., minor) blood groups. A second blood group is the Rh system, wherein an individual can be Rh+or Rh-. Thus, in some embodiments, an erythroid cell described herein is Rh-. In some embodiments, the erythroid cell is Type O and Rh-.

In some embodiments, an erythroid cell described herein is negative for one or more minor blood group antigens, e.g., Le(a-b-) (for Lewis antigen system), Fy(a-b-) (for Duffy system), Jk(a-b-) (for Kidd system), M-N- (for MNS system), K-k- (for Kell system), Lu(a-b-) (for Lutheran system), and H-antigen negative (Bombay phenotype), or any combination thereof. In some embodiments, the erythroid cell is also Type O and/or Rh-. Minor blood groups are described, e.g., in Agarwal et al. (2013) Blood Res. 48(1): 51-4 and Mitra et al. (2014) Indian J Anaesth. 58(5): 524-8, each of which is incorporated herein by reference in its entirety.

### Isolating Erythrocytes

Mature erythrocytes may be isolated using various methods such as, for example, a cell washer, a continuous flow cell separator, density gradient separation, fluorescence-activated cell sorting (FACS), Miltenyi immunomagnetic depletion (MACS), or a combination of these methods (See, e.g., van der Berg et al. (1987) Clin. Chem. 33: 1081-2; Bar-Zvi et al. (1987) J. Biol. Chem. 262: 17719-23; Goodman et al. (2007) Exp. Biol. Med. 232: 1470-6).

Erythrocytes may be isolated from whole blood by simple centrifugation (see, e.g., van der Berg et al. (1987)). For example, EDTA-anticoagulated whole blood may be centrifuged at 800×g for 10 min at 4° C. The platelet-rich plasma and buffy coat are removed and the red blood cells are washed three times with isotonic saline solution (NaCl, 9 g/L).

Alternatively, erythrocytes may be isolated using density gradient centrifugation with various separation mediums such as, for example, Ficoll, Hypaque, Histopaque, Percoll, Sigmacell, or combinations thereof. For example, a volume of Histopaque-1077 is layered on top of an equal volume of Histopaque-1119. EDTA-anticoagulated whole blood diluted 1:1 in an equal volume of isotonic saline solution (NaCl, 9 g/L) is layered on top of the Histopaque and the sample is centrifuged at 700×g for 30 min at room temperature. Under these conditions, granulocytes migrate to the 1077/1119 interface, lymphocytes, other mononuclear cells and platelets remain at the plasma/1077 interface, and the red blood cells are pelleted. The red blood cells are washed twice with isotonic saline solution.

Alternatively, erythrocytes may be isolated by centrifugation using a Percoll step gradient (See, e.g., Bar-Zvi et al. (1987)). For example, fresh blood is mixed with an anticoagulant solution containing 75 mM sodium citrate and 38 mM citric acid and the cells washed briefly in HEPES-buffered saline. Leukocytes and platelets are removed by adsorption with a mixture of α-cellulose and Sigmacell (1:1). The erythrocytes are further isolated from reticulocytes and residual white blood cells by centrifugation through a 45/75% Percoll step gradient for 10 min at 2500 rpm in a Sorvall SS34 rotor. The erythrocytes are recovered in the pellet while reticulocytes band at the 45/75% interface and the remaining white blood cells band at the 0/45% interface. The Percoll is removed from the erythrocytes by several washes in Hepes-buffered saline. Other materials that may be used to generate density gradients for isolation of erythrocytes include OPTIPREP, a 60% solution of iodixanol in water (from Axis-Shield, Dundee, Scotland).

Erythrocytes may be separated from reticulocytes, for example, using flow cytometry (See, e.g., Goodman et al. (2007)). In this instance, whole blood is centrifuged (550×g, 20 min, 25° C.) to separate cells from plasma. The cell pellet is resuspended in phosphate buffered saline solution and further fractionated on Ficoll-Paque (1.077 density), for example, by centrifugation (400×g, 30 min, 25° C.) to separate the erythrocytes from the white blood cells. The resulting cell pellet is resuspended in RPMI supplemented with 10% fetal bovine serum and sorted on a FACS instrument such as, for example, a Becton Dickinson FACSCalibur (BD Biosciences, Franklin Lakes, N.J., USA) based on size and granularity.

Erythrocytes may be isolated by immunomagnetic depletion (See, e.g., Goodman et al. (2007)). In this instance, magnetic beads with cell-type specific antibodies are used to eliminate non-erythrocytes. For example, erythrocytes are isolated from the majority of other blood components using a density gradient as described herein followed by immunomagnetic depletion of any residual reticulocytes. The cells are pre-treated with human antibody serum for 20 min at 25° C. and then treated with antibodies against reticulocyte specific antigens such as, for example, CD71 and CD36. The antibodies may be directly attached to magnetic beads or conjugated to PE, for example, to which magnetic beads with anti-PE antibody will react. The antibody-magnetic bead complex is able to selectively extract residual reticulocytes, for example, from the erythrocyte population.

Erythrocytes may also be isolated using apheresis. The process of apheresis involves removal of whole blood from a subject or donor, separation of blood components using centrifugation or cell sorting, withdrawal of one or more of the separated portions, and transfusion of remaining components back into the subject or donor. A number of instruments are currently in use for this purpose such as for example the Amicus and Alyx instruments from Baxter (Deerfield, Ill., USA), the Trima Accel instrument from Gambro BCT (Lakewood, Colo., USA), and the MCS+9000 instrument from Haemonetics (Braintree, Mass., USA). Additional purification methods may be necessary to achieve the appropriate degree of cell purity.

Reticulocytes are immature red blood cells and compose approximately 1% of the red blood cells in the human body. Reticulocytes develop and mature in the bone marrow. Once released into circulation, reticulocytes rapidly undergo terminal differentiation to mature erythrocytes. Like mature erythrocytes, reticulocytes do not have a cell nucleus. Unlike mature erythrocytes, reticulocytes maintain the ability to perform protein synthesis. In some embodiments, the engineered erythroid cell comprises an enucleated reticulocyte.

Reticulocytes of varying age may be isolated from peripheral blood based on the differences in cell density as the reticulocytes mature. Reticulocytes may be isolated from peripheral blood using differential centrifugation through various density gradients. For example, Percoll gradients may be used to isolate reticulocytes (See, e.g., Noble el al., Blood 74:475-481 (1989)). Sterile isotonic Percoll solutions of density 1.096 and 1.058 g/ml are made by diluting Percoll (Sigma-Aldrich, Saint Louis, Mo., USA) to a final concentration of 10 mM triethanolamine, 117 mM NaCl, 5 mM glucose, and 1.5 mg/ml bovine serum albumin (BSA). These solutions have an osmolarity between 295 and 310 mOsm. Five milliliters, for example, of the first Percoll solution (density 1.096) is added to a sterile 15 ml conical centrifuge tube. Two milliliters, for example, of the second Percoll solution (density 1.058) is layered over the higher density first Percoll solution. Two to four milliliters of whole blood are layered on top of the tube. The tube is centrifuged at 250xg for 30 min in a refrigerated centrifuge with swing-out tube holders. Reticulocytes and some white cells migrate to the interface between the two Percoll layers. The cells at the interface are transferred to a new tube and washed twice with phosphate buffered saline (PBS) with 5 mM glucose, 0.03 mM sodium azide and 1 mg/ml BSA. Residual white blood cells are removed by chromatography in PBS over a size exclusion column.

Alternatively, reticulocytes may be isolated by positive selection using an immunomagnetic separation approach (See, e.g., Brun et al. (1990) Blood 76: 2397-403). This approach takes advantage of the large number of transferrin receptors that are expressed on the surface of reticulocytes relative to erythrocytes prior to maturation. Magnetic beads coated with an antibody to the transferrin receptor may be used to selectively isolate reticulocytes from a mixed blood cell population. Antibodies to the transferrin receptor of a variety of mammalian species, including human, are available from commercial sources (e.g., Affinity BioReagents, Golden, Colo., USA; Sigma-Aldrich, Saint Louis, Mo., USA). The transferrin antibody may be directly linked to the magnetic beads. Alternatively, the transferrin antibody may be indirectly linked to the magnetic beads via a secondary antibody. For example, mouse monoclonal antibody 10D2 (Affinity BioReagents, Golden, Colo., USA) against human transferrin may be mixed with immunomagnetic beads coated with a sheep anti-mouse immunoglobulin G (Dynal/Invitrogen, Carlsbad, Calif., USA). The immunomagnetic beads are then incubated with a leukocyte-depleted red blood cell fraction. The beads and red blood cells are incubated at 22° C. with gentle mixing for 60-90 min followed by isolation of the beads with attached reticulocytes using a magnetic field. The isolated reticulocytes may be removed from the magnetic beads using, for example, DETACHaBEAD solution (from Invitrogen, Carlsbad, Calif., USA). Alternatively, reticulocytes may be isolated from in vitro growth and maturation of CD34+ hematopoietic stem cells using the methods described herein.

Terminally-differentiated enucleated erythrocytes can be separated from other cells based on their DNA content. In a non-limiting example, cells are first labeled with a vital DNA dye, such as Hoechst 33342 (Invitrogen Corp.). Hoechst 33342 is a cell-permeant nuclear counterstain that emits blue fluorescence when bound to double-stranded DNA. Undifferentiated precursor cells, macrophages or other nucleated cells in the culture are stained by Hoechst 33342, while enucleated erythrocytes are Hoechst-negative. The Hoechst-positive cells can be separated from enucleated erythrocytes by using fluorescence activated cell sorters or other cell sorting techniques. The Hoechst dye can be removed from the isolated erythrocytes by dialysis or other suitable methods.

Vehicles for Polypeptides Described Herein

While in many embodiments herein, the one or more (e.g., two or more) exogenous polypeptides are situated on or in an erythroid cell (e.g., engineered enucleated erythroid cell) or enucleated cell (e.g., modified enucleated cell), it is understood that any polypeptide or combination of exogenous polypeptides described herein can also be situated on or in another vehicle. The vehicle can comprise, e.g., a cell, an erythroid cell, a corpuscle, a nanoparticle, a micelle, a liposome, or an exosome. For instance, in some aspects, the present disclosure provides a vehicle (e.g., a cell, an erythroid cell, a corpuscle, a nanoparticle, a micelle, a liposome, or an exosome) comprising, e.g., on its surface, one or more agents described herein. In some embodiments, the one or more agents comprise an agent selected from the exogenous polypeptides described herein, or a fragment or variant thereof. In some embodiments, the vehicle comprises two or more agents described herein, e.g., any pair of agents described herein.

In some embodiments, the vehicle comprises an engineered erythroid cell (e.g. an engineered enucleated erythroid cell) or an enucleated cell.

### Heterogeneous Populations of Cells

While in many embodiments herein, the one or more (e.g., two or more) exogenous polypeptides are situated on or in a single cell, it is understood that any polypeptide or combination of polypeptides described herein can also be situated on a plurality of cells. For instance, in some aspects, the disclosure provides a plurality of erythroid cells or enucleated cells, wherein a first cell of the plurality comprises a first exogenous polypeptide (e.g., comprising a first exogenous antigenic polypeptide, exogenous antigen-presenting polypeptide, exogenous costimulatory polypeptide, exogenous coinhibitory polypeptide, cytokine, and exogenous Treg costimulatory polypeptide, or a combination thereof) and a second cell of the plurality comprises a second exogenous polypeptide (e.g., comprising a second exogenous antigenic polypeptide, exogenous antigen-presenting polypeptide, exogenous costimulatory polypeptide, exogenous coinhibitory polypeptide, cytokine, and exogenous Treg costimulatory polypeptide, or a combination thereof). In some embodiments, the plurality of cells comprises two or more polypeptides described herein, e.g., any pair of polypeptides described herein. In some embodiments, less than 90%, 80%, 70%, 60%, 50%, 40%, 30%, 20%, 10%, 5%, 2%, or 1% of the cells in the plurality comprise both the first exogenous polypeptide and the second exogenous polypeptide.

Cells Encapsulated in a Membrane

In some embodiments, engineered erythroid cells (e.g. engineered enucleated erythroid cells) or enucleated cells (e.g., modified enucleated cells), or other vehicles described herein are encapsulated in a membrane, e.g., semi-permeable membrane. In some embodiments, the membrane comprises a polysaccharide, e.g., an anionic polysaccharide alginate. In some embodiments, the semipermeable membrane does not allow cells to pass through, but allows passage of small molecules or macromolecules, e.g., metabolites, proteins, or DNA. In some embodiments, the membrane is one described in Lienert et al. (2014) Nat. Rev. Mol. Cell Biol. 15: 95-107, incorporated herein by reference in its entirety.

### Erythrocyte Precursor Cells

Provided herein are engineered erythrocyte precursor cells, and methods of making the engineered erythrocyte precursor cells.

Pluripotent stem cells can give rise to erythrocytes by the process of erythropoiesis. The stem cell looks like a small lymphocyte and lacks the functional capabilities of the erythrocyte. The stem cells have the capacity of infinite division, something the mature cells lack. Some of the daughter cells arising from the stem cell acquire erythroid characters over generations and time. Most of the erythroid cells in the bone marrow have a distinct morphology but commitment to erythroid maturation is seen even in cells that have not acquired morphological features distinctive of the erythroid lineage. These cells are recognized by the type of colonies they form in vitro. Two such cells are recognized. Burst-forming unit erythroid (BFU-E) arise from the stem cell and gives rise to colony-forming unit erythroid (CFU-E). CFU-E gives rise to pronormoblast, the most immature of erythroid cells with a distinct morphology. BFU-E and CFU-E form a very small fraction of bone marrow cells. Morphologically five erythroid precursors are identifiable in the bone marrow stained with Romanovsky stains. The five stages from the most immature to the most mature are the proerythroblast, the basophilic normoblast (early erythroblast), polychromatophilic normoblast (intermediate erythroblast), orthochromatophilic normoblast (late erythroblast) and reticulocyte. BFU-E (burst forming unit-erythroid), CFU-E (erythroid colony-forming unit), pronormoblast (proerythroblast), basophilic normoblast, polychromatophilic normoblast and orthochromatophilic normoblast are lineage restricted.

Table 15 below summarizes the morphological features of erythrocyte precursor cells.

TABLE 15

| Cell | Nucleus |
|---|---|
| Hematopoietic stem cell (HSC) | Yes |
| CMP (Common myeloid progenitor) | Yes |
| CFU-S (spleen colony forming cell; | Yes; Can differentiate into |
| myeloid precursor cell) | erythrocytes, platelets, |
| | macrophages. |
| BFU-E (burst forming unit-erythroid) | Yes |
| CFU-E (erythroid colony-forming | Yes |
| unit) | |
| Pronormoblast (proerythroblast) | Yes; fine chromatin, many nucleoli |
| Basophilic Normoblast | Yes; granular chromatin, no |
| | nucleoli |
| Polychromatophilic Normoblast | Yes; chromatin is visibly clumped |
| | with dark staining areas |
| Orthochromatophilic normoblast | Yes; featureless nucleus with dense |
| | chromatin |

Normal human erythrocytes express CD36, an adhesion molecule of monocytes, platelets, and endothelial cells (van Schravendijk et al. (1992) Blood 80(8): 2105-14). Accordingly, in some embodiments, an anti-CD36 antibody can be used to identify human erythrocytes.

Any type of cell known in the art that is capable of differentiating into an erythrocyte, i.e., any erythrocyte precursor cell, can be modified in accordance with the methods described herein to produce engineered erythrocyte precursor cells. In certain embodiments, the erythrocyte precursor cells modified in accordance with the methods described herein are cells that are in the process of differentiating into an erythrocyte, i.e., the cells are of a type known to exist during mammalian erythropoiesis. For example, the cells may be pluripotent hematopoietic stem cells (HSCs) or CD34+ cells, multipotent myeloid progenitor cells, CFU-S cells, BFU-E cells, CFU-E cells, pronormoblasts (proerythroblast), basophilic normoblasts, polychromatophilic normoblasts, or orthochromatophilic normoblasts. The modified erythrocyte precursor cells provided herein can be differentiated into engineered enucleated erythroid cells (e.g., reticulocytes or erythrocytes) in vitro using methods known in the art, i.e., using molecules known to promote erythropoiesis, e.g., SCF, Erythropoietin, IL-3, and/or GM-CSF, described herein below. Alternatively, the modified erythrocyte precursor cells are provided in a composition as described herein, and are capable of differentiating into erythrocytes upon administration to a subject in vivo.

### Culturing

Sources for generating engineered erythroid cells described herein include circulating erythroid cells. A suitable cell source may be isolated from a subject as described herein from subject-derived hematopoietic or erythroid precursor cells, derived from immortalized erythroid cell lines, or derived from induced pluripotent stem cells, optionally cultured and differentiated. Methods for generating erythrocytes using cell culture techniques are well known in the art, e.g., Giarratana et al. (2011) Blood 118: 5071-9, Huang et al. (2014), and Kurita et al. (2013) PLOS One 8: e59890. Protocols vary according to growth factors, starting cell lines, culture period, and morphological traits by which the resulting cells are characterized. Culture systems have also been established for blood production that may substitute for donor transfusions (Fibach et al. (1989) Blood 73: 100-3).

Provided herein are culturing methods for erythroid cells and engineered erythroid cells. Erythroid cells can be cultured from hematopoietic progenitor cells, including, for example, CD34+ hematopoietic progenitor cells (Giarratana et al. (2011)), induced pluripotent stem cells (Kurita et al. (2013)), and embryonic stem cells (Hirose et al. (2013) Stem Cell Reports 1: 499-508). Cocktails of growth and differentiation factors that are suitable to expand and differentiate progenitor cells are known in the art. Examples of suitable expansion and differentiation factors include, but are not limited to, stem cell factor (SCF), an interleukin (IL) such as IL-1, IL-2, IL-3, IL-4, IL-5, IL-6, IL-7, IL-8, IL-9, IL-11, IL-12, CSF, G-CSF, thrombopoietin (TPO), GM-CSF, erythropoietin (EPO), Flt3, Flt2, PIXY 321, and leukemia inhibitory factor (LIF).

Erythroid cells can be cultured from hematopoietic progenitors, such as CD34⁺ cells, by contacting the progenitor cells with defined factors in a multi-step culture process. For example, in some embodiments, erythroid cells can be cultured from hematopoietic progenitors in a three-step process, outlined below.

The first step may comprise contacting the cells in culture with stem cell factor (SCF) at 1-1000 ng/mL, erythropoietin (EPO) at 1-100 U/mL, and interleukin-3 (IL-3) at 0.1-100 ng/mL.

The first step optionally comprises contacting the cells in culture with a ligand that binds and activates a nuclear hormone receptor, such as e.g., the glucocorticoid receptor, the estrogen receptor, the progesterone receptor, the androgen receptor, or the pregnane x receptor. The ligands for these receptors include, for example, a corticosteroid, such as, e.g., dexamethasone at 10 nM-100 μM or hydrocortisone at 10 nM-100 μM; an estrogen, such as, e.g., beta-estradiol at 10 nM-100 μM; a progestogen, such as, e.g., progesterone at 10 nM-100 μM, hydroxyprogesterone at 10 nM-100 μM, 5a-dihydroprogesterone at 10 nM-100 μM, 11-deoxycorticosterone at 10 nM-100 μM, or a synthetic progestin, such as, e.g., chlormadinone acetate at 10 nM-100 μM; an androgen, such as, e.g., testosterone at 10 nM-100 μM, dihydrotestosterone at 10 nM-100 μM or androstenedione at 10 nM-100 μM; or a pregnane x receptor ligand, such as, e.g., rifampicin at 10 nM-100 μM, hyperforin at 10 nM-100 St. John's Wort (hypericin) at 10 nM-100 μM, or vitamin E-like molecules, such as, e.g., tocopherol at 10 nM-100. The first step may also optionally comprise contacting the cells in culture with an insulin-like molecule, such as, e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The first step further may optionally comprise contacting the cells in culture with transferrin at 0.1-5 mg/mL.

The first step may optionally comprise contacting the cells in culture with one or more interleukins (IL) or growth factors such as, e.g., IL-1, IL-2, IL-4, IL-5, IL-6, IL-7, IL-8, IL-9, IL-11, IL-12, granulocyte colony-stimulating factor (G-CSF), macrophage colony-stimulating factor (M-CSF), granulocyte-macrophage colony-stimulating factor (GM-CSF), thrombopoietin, fibroblast growth factor (FGF), platelet-derived growth factor (PDGF), transforming growth factor beta (TGF-B), tumor necrosis factor alpha (TNF-A), megakaryocyte growth and development factor (MGDF), leukemia inhibitory factor (LIF), and Flt3 ligand. Each interleukin or growth factor may typically be supplied at a concentration of 0.1-100 ng/mL. The first step may also optionally comprise contacting the cells in culture with serum proteins or non-protein molecules such as, e.g., fetal bovine serum (1-20%), human plasma (1-20%), plasmanate (1-20%), human serum (1-20%), albumin (0.1-100 mg/mL), or heparin (0.1-10 U/mL).

The second step may comprise contacting the cells in culture with stem cell factor (SCF) at 1-1000 ng/mL and erythropoietin (EPO) at 1-100 U/mL. The second step may also optionally comprise contacting the cells in culture with an insulin-like molecule, such as e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The second step may further optionally comprise contacting the cells in culture with transferrin at 0.1-5 mg/mL. The second may also optionally comprise contacting the cells in culture with serum proteins or non-protein molecules such as, e.g., fetal bovine serum (1-20%), human plasma (1-20%), plasmanate (1-20%), human serum (1-20%), albumin (0.1-100 mg/mL), or heparin (0.1-10 U/mL).

The third step may comprise contacting the cells in culture with erythropoietin (EPO) at 1-100 U/mL. The third step may optionally comprise contacting the cells in culture with stem cell factor (SCF) at 1-1000 ng/mL. The third step may further optionally comprise contacting the cells in culture with an insulin-like molecule, such as e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The third step may also optionally comprise contacting the cells in culture with transferrin at 0.1-5 mg/mL. The third step may also optionally comprise contacting the cells in culture with serum proteins or non-protein molecules such as, e.g., fetal bovine serum (1-20%), human plasma (1-20%), plasmanate (1-20%), human serum (1-20%), albumin (0.1-100 mg/mL), or heparin (0.1-10 U/mL).

In some embodiments, methods of expansion and differentiation of the engineered erythroid cells presenting one or more exogenous polypeptides, do not include culturing the engineered erythroid cells in a medium comprising a myeloproliferative receptor (mpl) ligand.

The culture process may optionally comprise contacting cells by a method known in the art with a molecule, e.g., a DNA molecule, an RNA molecule, a mRNA, an siRNA, a microRNA, a lncRNA, a shRNA, a hormone, or a small molecule, that activates or knocks down one or more genes. Target genes can include, for example, genes that encode a transcription factor, a growth factor, or a growth factor receptor, including but not limited to, e.g., GATA1, GATA2, CMyc, hTERT, p53, EPO, SCF, insulin, EPO-R, SCF-R, transferrin-R, insulin-R.

In some embodiments, CD34+ cells are placed in a culture containing varying amounts of IMDM, FBS, glutamine, BSA, holotransferrin, insulin, dexamethasone, .beta.-estradiol, IL-3, SCF, and erythropoietin, in three separate differentiation stages for a total of 22 days.

In some embodiments, CD34+ cells are placed in a culture containing varying amounts of IMDM, FBS, glutamine, BSA, holotransferrin, insulin, dexamethasone, β-estradiol, IL-3, SCF, and thrombopoietin, in three separate differentiation stages for a total of 14 days.

In some embodiments, CD34+cells are placed in a culture containing varying amounts of IMDM, FBS, glutamine, BSA, holotransferrin, insulin, dexamethasone, β-estradiol, IL-3, SCF, and GCSF, in three separate differentiation stages for a total of 15 days.

In some embodiments, the erythroid cells are expanded at least 100, 1000, 2000, 5000, 10,000, 20,000, 50,000, or 100,000 fold (and optionally up to 100,000, 200,000, or 500,000 fold). Number of cells is measured, in some embodiments, using an automated cell counter.

In some embodiments, it may be desirable during culturing to only partially differentiate the erythroid precursor cells, e.g., hematopoietic stem cells, in vitro, allowing further differentiation, e.g., differentiation into reticulocytes or fully mature erythrocytes, to occur upon introduction to a subject in vivo (see, e.g., Neildez-Nguyen et al. (2002) Nature Biotech. 20: 467-72). It will be understood that, in various embodiments, as described herein, maturation and/or differentiation in vitro may be arrested at any stage desired. For example, isolated CD34+ hematopoietic stem cells may be expanded in vitro as described elsewhere herein, e.g., in medium containing various factors, including, for example, interleukin 3, Flt3 ligand, stem cell factor, thrombopoietin, erythropoietin, transferrin, and insulin growth factor, to reach a desired stage of differentiation. The resulting engineered erythroid cells may be characterized by the surface expression of CD36 and GPA, and other characteristics specific to the particular desired cell type, and may be transfused into a subject where terminal differentiation to mature erythrocytes is allowed to occur.

In some embodiments, engineered erythroid cells are partially expanded from erythroid precursor cells to any stage of maturation prior to but not including enucleation, and thus remain nucleated cells, e.g., erythrocyte precursor cells. In certain embodiments, the resulting cells are nucleated and erythroid lineage restricted. In certain embodiments, the resulting cells are selected from multipotent myeloid progenitor cells, CFU-S cells, BFU-E cells, CFU-E cells, pronormoblasts (proerythroblast), basophilic normoblasts, polychromatophilic normoblasts and orthochromatophilic normoblasts. The final differentiation steps, including enucleation, occur only after administration of the engineered erythroid cell to a subject, that is, in such embodiments, the enucleation step occurs in vivo. In other embodiments, engineered erythroid cells are expanded and differentiated in vitro through the stage of enucleation to become, e.g., reticulocytes. In such embodiments where the engineered erythroid cells are differentiated to the stage of reticuloyctes, the final differentiation step to become erythrocytes occurs only after administration of the engineered erythroid cell to a subject, that is, the terminal differentiation step occurs in vivo. In other embodiments, engineered erythroid cells are expanded and differentiated in vitro through the terminal differentiation stage to become erythrocytes.

It will be further recognized that in some embodiments, the engineered erythroid cells may be expanded and differentiated from erythroid precursor cells, e.g., hematopoietic stem cells, to become hematopoietic cells of different lineage, such as, for example, to become platelets. Methods for maturing and differentiating hematopoietic cells of various lineages, such as platelets, are well known in the art to the skilled artisan. Such engineered platelets expressing exogenous polypeptides as described herein are considered to be encompassed by the present disclosure.

In some embodiments, an enucleated cell provided herein is a platelet. Methods of manufacturing platelets in vitro are known in the art (see, e.g., Wang and Zheng (2016) Springerplus 5(1): 787, and U.S. Pat. No. 9,574,178). Methods of manufacturing platelets including an exogenous polypeptide are described, e.g., in International Patent Application Publication Nos. WO2015/073587 and WO2015/153102, each of which is incorporated by reference in its entirety. Platelet production is in part regulated by signaling mechanisms induced by interaction between thrombopoietin (TPO) and its cellular receptor TPOR/MPUc-MPL. In addition, multiple cytokines (e.g., stem cell factor (SCF), IL-3, IL-6, IL-11, leukemia inhibiting factor (LIF), G-CSF, GM-CSF, M-CSF, erythropoietin (EPO), kit ligand, and interferon) have been shown to possess thrombocytopoietic activity.

In some embodiments, platelets are generated from hematopoietic progenitor cells, such as CD34+ hematopoietic stem cells, induced pluripotent stem cells or embryonic stem cells. In some embodiments, platelets are produced by contacting the progenitor cells with defined factors in a multi-step culture process. In some embodiments, the multi-step culture process comprises: culturing a population of hematopoietic progenitor cells under conditions suitable to produce a population of megakaryocyte progenitor cells, and culturing the population of megakaryocyte progenitor cells under conditions suitable to produce platelets. Cocktails of growth and differentiation factors that are suitable to expand and differentiate progenitor cells and produce platelets are known in the art. Examples of suitable expansion and differentiation factors include, but are not limited to, stem cell factor (SCF), Flt-3/Flk-2 ligand (FL), TPO, IL-11, IL-3, IL-6, and IL-9. For instance, in some embodiments, platelets may be produced by seeding CD34⁺ HSCs in a serum-free medium at 2-4 ×10⁴ cells/mL, and refreshing the medium on culture day 4 by adding an equal volume of media. On culture day 6, cells are counted and analyzed: 1.5 ×10⁵ cells are washed and placed in 1 mL of the same medium supplemented with a cytokine cocktail comprising TPO (30 ng/mL), SCF (1 ng/mL), IL-6 (7.5 ng/mL), and IL-9 (13.5 ng/mL) to induce megakaryocyte differentiation. At culture day 10, from about one quarter to about half of the suspension culture is replaced with fresh media. The cells are cultured in a humidified atmosphere (10% CO₂) at 39° C. for the first 6 culture days, and at 37° C. for the last 8 culture days. Viable nucleated cells are counted with a hemocytometer following trypan blue staining. The differentiation state of platelets in culture can be assessed by flow cytometry or quantitative PCR as described in Examples 44 and 45 of in International Patent Application Publication No. WO2015/073587, incorporated herein by reference.

### Expression of Exogenous Polypeptides

In some embodiments, the engineered erythroid cells described herein are generated by contacting a suitable isolated cell, e.g., a nucleated erythroid cell, an erythroid precursor cell, or a nucleated platelet precursor cell, with an exogenous nucleic acid encoding a polypeptide of the disclosure (e.g., exogenous antigen-presenting polypeptides, exogenous antigenic polypeptides, exogenous coinhibitory polypeptides, cytokines, and exogenous Treg costimulatory polypeptides, or a combination thereof).

In some embodiments, the exogenous polypeptide is encoded by a DNA, which is contacted with a nucleated erythroid precursor cell or a nucleated platelet precursor cell. In some embodiments, the exogenous polypeptide is encoded by an RNA (e.g., an mRNA), which is contacted with a nucleated erythroid cell, an erythroid precursor cell, a nucleated platelet precursor cell.

In some embodiments, the exogenous polypeptide is contacted with a platelet, a nucleated erythroid cell, an erythroid precursor cell, a nucleated platelet precursor cell, a reticulocyte, or an erythrocyte.

In some embodiments, the exogenous polypeptide comprises an epitope tag sequence, which may be one, or a combination of, an HA-tag, Green fluorescent protein tag, Myc-tag, chitin binding protein, maltose binding protein, glutathione-S-transferase, poly(His)tag, thioredoxin, poly(NANP), FLAG-tag, V5-tag, AviTag, Calmodulin-tag, polyglutamate-tag, E-tag, S-tag, SBP-tag, Softag-1, Softag-3, Strep-tag, TC-tag, VSV-tag, Xpress-tag, Isopeptag, SpyTag, biotin carboxyl carrier protein, Nus-tag, Fc-tag, or Ty-tag. In some embodiments, the exogenous nucleic acid encoding an exogenous polypeptide comprises the 3′ end of a gene sequence for the exogenous polypeptide that is fused to an epitope tag sequence (e.g., at the N-terminus or C-terminus), of which may be one, or a combination of:, an; an HA-tag, gGreen fluorescent protein tag, Myc-tag, chitin binding protein, maltose binding protein, glutathione-S-transferase, poly(His)tag, thioredoxin, poly(NANP), FLAG-tag, V5-tag, AviTag, Calmodulin-tag, polyglutamate-tag, E-tag, S-tag, SBP-tag, Softag-1, Softag-3, Strep-tag, TC-tag, VSV-tag, Xpress-tag, Isopeptag, SpyTag, biotin carboxyl carrier protein, Nus-tag, Fc-tag, or Ty-tag. In some embodiments, the exogenous polypeptide comprises an epitope tag, such as an HA epitope tag(YPYDVPDYA (SEQ ID NO:27)), a cMyc tag (EQKLISEEDL (SEQ ID NO:28)), or a Flag tag (DYKDDDDK (SEQ ID NO:29)). The epitope tag may be used for the easy detection and quantification of expression using antibodies against the epitope tag by flow cytometry, western blot, or immunoprecipitation.

An exogenous polypeptide may be expressed from a transgene introduced into an erythroid cell by electroporation, chemical or polymeric transfection, viral transduction, mechanical membrane disruption, or other method; an exogenous polypeptide that is expressed from mRNA that is introduced into a cell by electroporation, chemical or polymeric transfection, viral transduction, mechanical membrane disruption, or other method; an exogenous polypeptide that is over-expressed from the native locus by the introduction of an external factor, e.g., a transcriptional activator, transcriptional repressor, or secretory pathway enhancer; and/or a polypeptide that is synthesized, extracted, or produced from a production cell or other external system and incorporated into the erythroid cell.

In certain embodiments, the introducing step comprises viral transduction. In some embodiments, the introducing step comprises electroporation. In other embodiments, the introducing step comprises utilizing one or more of liposome mediated transfer, adenovirus, adeno-associated virus, herpes virus, a retroviral based vector, lipofection, and a lentiviral vector.

In some embodiments, the introducing step comprises introducing the first exogenous nucleic acid encoding the first exogenous polypeptide by transfection of a lentiviral vector.

Exogenous nucleic acids (e.g., comprising DNA or RNA) encoding an exogenous polypeptide (e.g., exogenous antigen-presenting polypeptides, exogenous costimulatory polypeptides, exogenous coinhibitory polypeptides, cytokines, and exogenous Treg costimulatory polypeptides) can be introduced by transfection of single or multiple copies of genes, transduction with a virus, or electroporation. Methods for expression of exogenous proteins in mammalian cells are well known in the art. For example, expression of exogenous factor IX in hematopoietic cells is induced by viral transduction of CD34⁺ progenitor cells, see Chang et al. (2006) Nat. Biotechnol. 24: 1017-21.

In some embodiments, the DNA or RNA is codon optimized.

In some embodiments, the two or more polypeptides are encoded in a single nucleic acid, e.g. a single vector. In some embodiments, the single vector has a separate promoter for each gene, has two proteins that are initially transcribed into a single polypeptide having a protease cleavage site in the middle, so that subsequent proteolytic processing yields two proteins, or any other suitable configuration. In some embodiments, when there are more than one polypeptides (e.g. two or more) the polypeptides may be encoded in a single nucleic acid, e.g. a single vector. When exogenous immunogenic polypeptides, exogenous antigen-presenting polypeptides, exogenous coinhibitory polypeptides, cytokines, and exogenous Treg costimulatory polypeptides, or a combination thereof are encoded by the same exogenous nucleic acid (e.g., a vector), there are multiple possible sub-strategies useful for co-expression of the polypeptides. In some embodiments, the single exogenous nucleic acid (e.g., vector) has a separate promoter for each gene encoding an exogenous nucleic acid. In some embodiments, the exogenous nucleic acid encodes the two (or more) exogenous polypeptides whereby a “self-cleaving” 2A element is disposed between the cistrons encoding the exogenous polypeptides. The 2A element is believed to function by making the ribosome skip the synthesis of a peptide bond at the C-terminus of a 2A element, leading to separation between the end of the 2A sequence and the next polypeptide downstream (see, e.g., Holst et al. (2008) Nat. Immunol. 6:658-66).

For dual expression via two promoters, the MSCV promoter may be used as a first promoter and the EF1 promoter as a second promoter, although the disclosure is not to be limited by these two exemplary promoters. Another strategy is to express both two or more exogenous polypeptides by inserting an internal ribosome entry site (IRES) between the two genes encoding the polypeptides. Still another strategy is to express two or more exogenous polypeptides as direct peptide fusions separated by a linker.

In some embodiments, the two or more polypeptides are encoded by two or more exogenous nucleic acids, e.g., each vector encodes one of the exogenous polypeptides.

For dual expression via two promoters, the MSCV promoter may be used as a first promoter and the EF1 promoter as a second promoter, although the disclosure is not to be limited by these two exemplary promoters. Another strategy is to express both two or more exogenous polypeptides by inserting an internal ribosome entry site (IRES) between the two genes encoding the polypeptides. Still another strategy is to express two or more exogenous polypeptides as direct peptide fusions separated by a linker.

In some embodiments, the two or more polypeptides are encoded by two or more exogenous nucleic acids, e.g., each vector encodes one of the exogenous polypeptides.

In certain embodiments, the lentiviral vector is used which comprises a promoter selected from the group consisting of beta-globin promoter, murine stem cell virus (MSCV) promoter, Gibbon ape leukemia virus (GALV) promoter, human elongation factor lalpha (EFlalpha) promoter, CAG CMV immediate early enhancer and the chicken beta-actin (CAG), and human phosphoglycerate kinase 1 (PGK) promoter.

In some embodiments, the two or more polypeptides are encoded in two or more nucleic acids, e.g., each vector encodes one of the polypeptides.

Nucleic acids such as DNA expression vectors or mRNA for producing the exogenous polypeptides may be introduced into progenitor cells (e.g., an erythroid cell progenitor or a platelet progenitor and the like) that are suitable to produce the exogenous polypeptides described herein. The progenitor cells can be isolated from an original source or obtained from expanded progenitor cell population via routine recombinant technology as provided herein. In some instances, the expression vectors can be designed such that they can incorporate into the genome of cells by homologous or non-homologous recombination by methods known in the art.

In some embodiments, hematopoietic progenitor cells, e.g., CD34⁺ hematopoietic stem cells, are contacted with a nucleic acid or nucleic acids encoding one or more exogenous polypeptides, and the cells are allowed to expand and differentiate in culture.

In some instances, e.g., for an engineered erythroid cell (e.g., engineered enucleated erythroid cell) or enucleated cell (e.g., modified enucleated cell) comprising one or more exogenous polypeptides (exogenous immunogenic polypeptides, exogenous antigen-presenting polypeptides, exogenous coinhibitory polypeptides, cytokines, and exogenous Treg costimulatory polypeptides, or a combination thereof), a nucleic acid encoding a polypeptide that can selectively target and cut the genome, for example a CRISPR/Cas9, transcriptional activator-like effector nuclease (TALEN), or zinc finger nuclease, is used to direct the insertion of the exogenous nucleic acid of the expression vector encoding the exogenous polypeptide to a particular genomic location, for example the CR1 locus (1q32.2), the hemoglobin locus (11p15.4).

In some embodiments, one or more exogenous polypeptides (e.g., exogenous immunogenic polypeptides, exogenous antigen-presenting polypeptides, exogenous coinhibitory polypeptides, cytokines, and exogenous Treg costimulatory polypeptides, or a combination thereof) may be cloned into plasmid constructs for transfection. Methods for transferring expression vectors into cells that are suitable to produce the engineered erythroid cells (e.g., engineered enucleated erythroid cells) or enucleated cells (e.g., modified enucleated cells) described herein include, but are not limited to, viral mediated gene transfer, liposome mediated transfer, transformation, gene guns, transfection and transduction, e.g., viral mediated gene transfer such as the use of vectors based on DNA viruses such as adenovirus, adenoassociated virus and herpes virus, as well as retroviral based vectors. Examples of modes of gene transfer include e.g., naked DNA, CaPO₄ precipitation, DEAE dextran, electroporation, protoplast fusion, lipofection, and cell microinjection.

In some embodiments, recombinant DNA encoding each exogenous polypeptide may be cloned into a lentiviral vector plasmid for integration into erythroid cells. In some embodiments, the lentiviral vector comprises DNA encoding a single exogenous polypeptide for integration into erythroid cells. In other embodiments, the lentiviral vector comprises two, three, four or more exogenous polypeptides as described herein for integration into erythroid cells. In some embodiments, recombinant DNA encoding the one or more exogenous polypeptides may be cloned into a plasmid DNA construct encoding a selectable trait, such as an antibiotic resistance gene. In some embodiments, recombinant DNA encoding the exogenous polypeptides may be cloned into a plasmid construct that is adapted to stably express each recombinant protein in the erythroid cells.

In some embodiments, the lentiviral system may be employed where the transfer vector with exogenous polypeptides sequences (e.g., one, two, three, four or more exogenous polypeptide sequences), an envelope vector, and/or one or more packaging vectors are each transfected into host cells for virus production. In some embodiments, the lentiviral vectors may be transfected into host cells by any of calcium phosphate precipitation transfection, lipid-based transfection, or electroporation, and incubated overnight. For embodiments where the exogenous polypeptide sequence may be accompanied by a fluorescence reporter, inspection of the host cells for florescence may be checked after overnight incubation. The culture medium of the host cells comprising virus particles may be harvested 2 or 3 times every 8-12 hours and centrifuged to sediment detached cells and debris. The culture medium may then be used directly, frozen or concentrated as needed.

A progenitor cell subject to transfer of an exogenous nucleic acid that encodes an exogenous polypeptide can be cultured under suitable conditions allowing for differentiation and enucleation, e.g., the in vitro culturing process described herein.

Isolated erythroid precursor cells (e.g., a CD34⁺ hematopoietic stem cells) may be transfected with mRNA encoding one or more exogenous polypeptides (e.g., exogenous immunogenic polypeptides, exogenous antigen-presenting polypeptides, exogenous coinhibitory polypeptides, cytokines, and exogenous Treg costimulatory polypeptides, or a combination thereof) to generate an engineered erythroid cell (e.g., engineered enucleated erythroid cell) or enucleated cell (e.g., modified enucleated cell). Messenger RNA may be derived from in vitro transcription of a cDNA plasmid construct containing the coding sequence corresponding to the one or more exogenous polypeptides. For example, the cDNA sequence corresponding to the exogenous polypeptide may be inserted into a cloning vector containing a promoter sequence compatible with specific RNA polymerases. For example, the cloning vector ZAP EXPRESS pBK-CMV (Stratagene, La Jolla, Calif., USA) contains T3 and T7 promoter sequence compatible with T3 and T7 RNA polymerase, respectively. For in vitro transcription of sense mRNA, the plasmid is linearized at a restriction site downstream of the stop codon(s) corresponding to the end of the coding sequence of the exogenous polypeptide. The mRNA is transcribed from the linear DNA template using a commercially available kit such as, for example, the RNAMAXX High Yield Transcription Kit (from Stratagene, La Jolla, Calif, USA). In some instances, it may be desirable to generate 5′-m7GpppG-capped mRNA. As such, transcription of a linearized cDNA template may be carried out using, for example, the mMESSAGE mMACHINE High Yield Capped RNA Transcription Kit from Ambion (Austin, Tex., USA). Transcription may be carried out in a reaction volume of 20-100 μl at 37° C. for 30 min to 4 h. The transcribed mRNA is purified from the reaction mix by a brief treatment with DNase Ito eliminate the linearized DNA template followed by precipitation in 70% ethanol in the presence of lithium chloride, sodium acetate or ammonium acetate. The integrity of the transcribed mRNA may be assessed using electrophoresis with an agarose-formaldehyde gel or commercially available Novex pre-cast TBE gels (e.g., Novex, Invitrogen, Carlsbad, Calif., USA).

Messenger RNA encoding the one or more exogenous polypeptides (e.g., exogenous antigenic polypeptides, exogenous antigen-presenting polypeptides, exogenous coinhibitory polypeptides, cytokines, and exogenous Treg costimulatory polypeptides, or a combination thereof) may be introduced into erythroid precursor cells (e.g., a CD34⁺ hematopoietic stem cell) using a variety of approaches including, for example, lipofection and electroporation (van Tandeloo et al. (2001) Blood 98: 49-56). For lipofection, for example, 5μg of in vitro transcribed mRNA in Opti-MEM (Invitrogen, Carlsbad, Calif, USA) is incubated for 5-15 min at a 1:4 ratio with the cationic lipid DMRIE-C(Invitrogen). Alternatively, a variety of other cationic lipids or cationic polymers may be used to transfect cells with mRNA including, for example, DOTAP, various forms of polyethylenimine, and polyL-lysine (Sigma-Aldrich, Saint Louis, Mo., USA), and Superfect (Qiagen, Inc., Valencia, Calif, USA; See, e.g., Bettinger et al. (2001) Nucleic Acids Res. 29: 3882-91). The resulting mRNA/lipid complexes are incubated with cells (1-2×10⁶ cells/ml) for 2 h at 37° C., washed and returned to culture. For electroporation, for example, about 5 to 20×10⁶ cells in 500 μl of Opti-MEM (Invitrogen, Carlsbad, Calif., USA) are mixed with about 20 μg of in vitro transcribed mRNA and electroporated in a 0.4-cm cuvette using, for example, and Easyject Plus device (EquiBio, Kent, United Kingdom). In some instances, it may be necessary to test various voltages, capacitances and electroporation volumes to determine the useful conditions for transfection of a particular mRNA into a . In general, the electroporation parameters required to efficiently transfect cells with mRNA appear to be less detrimental to cells than those required for electroporation of DNA (van Tandeloo et al. (2001)).

Alternatively, mRNA may be transfected into a erythroid precursor cells (e.g., a CD34⁺ cell) using a peptide-mediated RNA delivery strategy (see, e.g., Bettinger et al. (2001)). For example, the cationic lipid polyethylenimine 2 kDA (Sigma-Aldrich, Saint Louis, Mo., USA) may be combined with the melittin peptide (Alta Biosciences, Birmingham, UK) to increase the efficiency of mRNA transfection, particularly in post-mitotic primary cells. The mellitin peptide may be conjugated to the PEI using a disulfide cross-linker such as, for example, the hetero-bifunctional cross-linker succinimidyl 3-(2-pyridyldithio) propionate. In vitro transcribed mRNA is preincubated for 5 to 15 min with the mellitin-PEI to form an RNA/peptide/lipid complex. This complex is then added to cells in serum-free culture medium for 2 to 4 h at 37° C. in a 5% CO₂ humidified environment and then removed and the transfected cells allowed to continue growing in culture.

In some embodiments, the engineered erythroid cell (e.g., engineered enucleated erythroid cell) or enucleated cell (e.g., modified enucleated cell) is generated by contacting a suitable isolated erythroid precursor cell or a platelet precursor cell with an exogenous nucleic acid encoding one or more exogenous polypeptides (e.g.,, exogenous antigen-presenting polypeptides, exogenous coinhibitory polypeptides, cytokines, and exogenous Treg costimulatory polypeptides, or a combination thereof). In some embodiments, the exogenous polypeptide is encoded by a DNA, which is contacted with a nucleated erythroid precursor cell or a nucleated platelet precursor cell. In some embodiments, the exogenous polypeptide is encoded by an RNA, which is contacted with a platelet, a nucleated erythroid cell, or a nucleated platelet precursor cell.

The one or more exogenous polypeptides (e.g., exogenous antigen-presenting polypeptides, exogenous coinhibitory polypeptides, cytokines, and exogenous Treg costimulatory polypeptides, or a combination thereof) may be genetically introduced into erythroid precursor cells, platelet precursor, or nucleated erythroid cells prior to terminal differentiation using a variety of DNA techniques, including transient or stable transfections and gene therapy approaches. The exogenous polypeptides may be expressed on the surface and/or in the cytoplasm of erythroid cell or platelet.

Viral gene transfer may be used to transfect the cells with DNA encoding one or more exogenous polypeptides (e.g., exogenous antigen-presenting polypeptides, exogenous coinhibitory polypeptides, cytokines, and exogenous Treg costimulatory polypeptides, or a combination thereof). A number of viruses may be used as gene transfer vehicles including Moloney murine leukemia virus (MMLV), adenovirus, adeno-associated virus (AAV), herpes simplex virus (HSV), lentiviruses such as human immunodeficiency virus 1 (HIV 1), and spumaviruses such as foamy viruses, for example (See, e.g., Osten et al. (2007) Handb. Exp. Pharmacol. 178: 177-202). Retroviruses, for example, efficiently transduce mammalian cells including human cells and integrate into chromosomes, conferring stable gene transfer.

One or more exogenous polypeptides (e.g., exogenous antigen-presenting polypeptides, exogenous coinhibitory polypeptides, cytokines, and exogenous Treg costimulatory polypeptides, or a combination thereof) may be transfected into an erythroid precursor cell, a platelet precursor, or a nucleated erythroid cell, expressed and subsequently retained and exhibited in an engineered erythroid cell (e.g., an engineered enucleated erythroid cell) or an enucleated cell (e.g., modified enucleated cell), as described herein. A suitable vector is the Moloney murine leukemia virus (MMLV) vector backbone (Malik et al. (1998) Blood 91: 2664-71). For example, a DNA construct containing the cDNA encoding an exogenous polypeptide can be generated in the MMLV vector backbone using standard molecular biology techniques. The construct is transfected into a packaging cell line such as, for example, PA317 cells and the viral supernatant is used to transfect producer cells such as, for example, PG13 cells. The PG13 viral supernatant is incubated with an erythroid precursor cell, a platelet precursor, or a nucleated erythroid cell that has been isolated and cultured or has been freshly isolated as described herein. The expression of the exogenous polypeptide may be monitored using FACS analysis (fluorescence-activated cell sorting), for example, with a fluorescently labeled antibody directed against the exogenous polypeptide, if it is located on the surface of the engineered erythroid cell (e.g., engineered enucleated erythroid cell) or enucleated cell (e.g., modified enucleated cell). Similar methods may be used to express an exogenous polypeptide that is located in the inside of the engineered erythroid cell (e.g., engineered enucleated erythroid cell) or enucleated cell (e.g., modified enucleated cell).

Optionally, a fluorescent tracking molecule such as, for example, green fluorescent protein (GFP) may be transfected using a viral-based approach (Tao et al. (2007) Stem Cells 25: 670-8). Ecotopic retroviral vectors containing DNA encoding the enhanced green fluorescent protein (EGFP) or a red fluorescent protein (e.g., DsRed-Express) are packaged using a packaging cell such as, for example, the Phoenix-Eco cell line (distributed by Orbigen, San Diego, Calif.). Packaging cell lines stably express viral proteins needed for proper viral packaging including, for example, gag, pol, and env. Supernatants from the Phoenix-Eco cells into which viral particles have been shed are used to transduce e.g., erythroid precursor cells, platelet precursors, or a nucleated erythroid cells. In some instances, transduction may be performed on a specially coated surface such as, for example, fragments of recombinant fibronectin to improve the efficiency of retroviral mediated gene transfer (e.g., RetroNectin, Takara Bio USA, Madison, Wis.). Cells are incubated in RetroNectin-coated plates with retroviral Phoenix-Eco supernatants plus suitable co-factors. Transduction may be repeated the next day. In this instance, the percentage of cells expressing EGFP or DsRed-Express may be assessed by FACS. Other reporter genes that may be used to assess transduction efficiency include, for example, beta-galactosidase, chloramphenicol acetyltransferase, and luciferase as well as low-affinity nerve growth factor receptor (LNGFR), and the human cell surface CD24 antigen (Bierhuizen et al. (1999)Leukemia 13: 605-13).

Nonviral vectors may be used to introduce genetic material into suitable erythroid cells, platelets or precursors thereof to generate engineered erythroid cells (e.g., engineered enucleated erythroid cells) or enucleated cells (e.g., modified enucleated cells). Nonviral-mediated gene transfer differs from viral-mediated gene transfer in that the plasmid vectors contain no proteins, are less toxic and easier to scale up, and have no host cell preferences. The “naked DNA” of plasmid vectors is by itself inefficient in delivering genetic material encoding a polypeptide to a cell and therefore is combined with a gene delivery method that enables entry into cells. A number of delivery methods may be used to transfer nonviral vectors into suitable erythroid cells, platelets or precursors thereof including chemical and physical methods.

A nonviral vector encoding one or more exogenous polypeptides (e.g., exogenous antigen-presenting polypeptides, exogenous coinhibitory polypeptides, cytokines, and exogenous Treg costimulatory polypeptides, or a combination thereof) may be introduced into suitable erythroid cells, platelets or precursors thereof using synthetic macromolecules such as cationic lipids and polymers (Papapetrou et al. (2005) Gene Therapy 12: S118-30). Cationic liposomes, for example form complexes with DNA through charge interactions. The positively charged DNA/lipid complexes bind to the negative cell surface and are taken up by the cell by endocytosis. This approach may be used, for example, to transfect hematopoietic cells (See, e.g., Keller et al. (1999) Gene Therapy 6: 931-8). For erythroid cells, platelets or precursors thereof the plasmid DNA (approximately 0.5 μg in 25-100 μL of a serum free medium, such as, for example, OptiMEM (Invitrogen, Carlsbad, Calif.)) is mixed with a cationic liposome (approximately 4 μg in 25 μL of serum free medium) such as the commercially available transfection reagent Lipofectamine™ (Invitrogen, Carlsbad, Calif.) and allowed to incubate for at least 20 min to form complexes. The DNA/liposome complex is added to suitable erythroid cells, platelets or precursors thereof and allowed to incubate for 5-24 h, after which time transgene expression of the polypeptide may be assayed. Alternatively, other commercially available liposome transfection agents may be used (e.g., In vivo GeneSHUTTLE, Qbiogene, Carlsbad, Calif.).

Optionally, a cationic polymer such as, for example, polyethylenimine (PEI) may be used to efficiently transfect erythroid cell progenitor cells, for example hematopoietic and umbilical cord blood-derived CD34+ cells (See, e.g., Shin et al. (2005) Biochim. Biophys. Acta 1725: 377-84). Human CD34+ cells are isolated from human umbilical cord blood and cultured in Iscove's modified Dulbecco's medium supplemented with 200 ng/ml stem cell factor and 20% heat-inactivated fetal bovine serum. Plasmid DNA encoding the exogenous polypeptide is incubated with branched or linear PEIs varying in size from 0.8 K to 750 K (Sigma Aldrich, Saint Louis, Mo., USA; Fermetas, Hanover, Md., USA). PEI is prepared as a stock solution at 4.2 mg/ml distilled water and slightly acidified to pH 5.0 using HC1. The DNA may be combined with the PEI for 30 min at room temperature at various nitrogen/phosphate ratios based on the calculation that 1μg of DNA contains 3 nmol phosphate and 1 μl of PEI stock solution contains 10 nmol amine nitrogen. The isolated CD34+cells are seeded with the DNA/cationic complex, centrifuged at 280×g for 5 min and incubated in culture medium for 4 or more h until gene expression of the polypeptide is assessed.

A plasmid vector may be introduced into suitable erythroid cells, platelets or precursors thereof using a physical method such as particle-mediated transfection, “gene gun”, biolistics, or particle bombardment technology (Papapetrou et al. (2005)). In this instance, DNA encoding the polypeptide is absorbed onto gold particles and administered to cells by a particle gun. This approach may be used, for example, to transfect erythroid precursor cells, e.g., hematopoietic stem cells derived from umbilical cord blood (See, e.g., Verma et al. (1998) Gene Therapy 5: 692-9). As such, umbilical cord blood is isolated and diluted three fold in phosphate buffered saline. CD34+ cells are purified using an anti-CD34 monoclonal antibody in combination with magnetic microbeads coated with a secondary antibody and a magnetic isolation system (e.g., Miltenyi MiniMac System, Auburn, Calif., USA). The CD34+ enriched cells may be cultured as described herein. For transfection, plasmid DNA encoding the polypeptide is precipitated onto a particle, for example gold beads, by treatment with calcium chloride and spermidine. Following washing of the DNA-coated beads with ethanol, the beads may be delivered into the cultured cells using, for example, a Biolistic PDS-1000/He System (Bio-Rad, Hercules, Calif., USA). A reporter gene such as, for example, beta-galactosidase, chloramphenicol acetyltransferase, luciferase, or green fluorescent protein may be used to assess efficiency of transfection.

Optionally, electroporation methods may be used to introduce a plasmid vector into suitable erythroid cells, platelets or precursors thereof. Electroporation creates transient pores in the cell membrane, allowing for the introduction of various molecules into the cells including, for example, DNA and RNA as well as antibodies and drugs. As such, CD34+ cells are isolated and cultured as described herein. Immediately prior to electroporation, the cells are isolated by centrifugation for 10 min at 250xg at room temperature and resuspended at 0.2-10×10⁶ viable cells/ml in an electroporation buffer such as, for example, X-VIVO™ 10 media supplemented with 1.0% human serum albumin (HSA). The plasmid DNA (1-50 μg) is added to an appropriate electroporation cuvette along with 500 μl of cell suspension. Electroporation may be done using, for example, an ECM 600 electroporator (Genetronics, San Diego, Calif, USA) with voltages ranging from 200 V to 280 V and pulse lengths ranging from 25 to 70 milliseconds. A number of alternative electroporation instruments are commercially available and may be used for this purpose (e.g., Gene Pulser XCELL, BioRad, Hercules, Calif.; Cellject Duo, Thermo Science, Milford, Mass.). Alternatively, efficient electroporation of isolated CD34+cells may be performed using the following parameters: 4 mm cuvette, 1600 μF, 550 V/cm, and 10 μg of DNA per 500 μl of cells at 1×10⁵ cells/ml (Oldak et al. (2002) Acta Biochimica Polonica 49: 625-32).

Nucleofection, a form of electroporation, may also be used to transfect suitable erythroid cells, platelets or precursors thereof In this instance, transfection is performed using electrical parameters in cell-type specific solutions that enable DNA (or other reagents) to be directly transported to the nucleus thus reducing the risk of possible degradation in the cytoplasm. For example, a Human CD34 CELL NUCLEOFECTOR Kit (from Amaxa Inc.) may be used to transfect suitable erythroid cells, platelets or precursors thereof. In this instance, 1-5×10⁶ cells in Human CD34 Cell NUCLEOFECTOR Solution are mixed with 1-5 μg of DNA and transfected in the NUCLEOFECTOR instrument using preprogrammed settings as determined by the manufacturer.

Erythroid cells, platelets or precursors thereof may be non-virally transfected with a conventional expression vector which is unable to self-replicate in mammalian cells unless it is integrated in the genome. Alternatively, erythroid cells, platelets or precursors thereof may be transfected with an episomal vector which may persist in the host nucleus as autonomously replicating genetic units without integration into chromosomes (Papapetrou et al. (2005)). These vectors exploit genetic elements derived from viruses that are normally extrachromosomally replicating in cells upon latent infection such as, for example, EBV, human polyomavirus BK, bovine papilloma virus-1 (BPV-1), herpes simplex virus-1 (HSV) and Simian virus 40 (SV40). Mammalian artificial chromosomes may also be used for nonviral gene transfer (Vanderbyl et al. (2005) Exp. Hematol. 33: 1470-6).

Exogenous nucleic acids encoding one or more exogenous polypeptides (e.g., exogenous antigen-presenting polypeptides, exogenous coinhibitory polypeptides, cytokines, and exogenous Treg costimulatory polypeptides, or a combination thereof) may be assembled into expression vectors by standard molecular biology methods known in the art, e.g., restriction digestion, overlap-extension PCR, and Gibson assembly.

Exogenous nucleic acids may comprise a gene encoding one or more exogenous polypeptides (e.g., exogenous antigen-presenting polypeptides, exogenous coinhibitory polypeptides, cytokines, and exogenous Treg costimulatory polypeptides, or a combination thereof) that are not normally expressed on the cell surface, e.g., of an erythroid cell, fused to a gene that encodes an endogenous or native membrane protein, such that the exogenous polypeptide is expressed on the cell surface. For example, a exogenous gene encoding an exogenous antigenic polypeptide can be cloned at the N terminus following the leader sequence of a type 1 membrane protein, at the C terminus of a type 2 membrane protein, or upstream of the GPI attachment site of a GPI-linked membrane protein.

Standard cloning methods can be used to introduce flexible amino acid linkers between two fused genes. For example, the flexible linker is a poly-glycine poly-serine linker such as [Gly4Ser]3 (SEQ ID NO:31) commonly used in generating single-chain antibody fragments from full-length antibodies (Antibody Engineering: Methods & Protocols, Lo 2004), or Ala-Gly-Ser-Thr polypeptides such as those used to generate single-chain Arc repressors (Robinson & Sauer, Proc. Nat'l. Acad. Sci. USA 1998). In some embodiments, the flexible linker provides the polypeptide with more flexibility and steric freedom than the equivalent construct without the flexible linker.

An epitope tag may be placed between two fused genes, such as, e.g., a nucleic acid sequence encoding an HA epitope tag-amino acids YPYDVPDYA (SEQ ID NO:32), a CMyc tag-amino acids EQKLISEEDL (SEQ ID NO:33), or a Flag tag-amino acids DYKDDDDK (SEQ ID NO:34). The epitope tag may be used for the facile detection and quantification of expression using antibodies against the epitope tag by flow cytometry, western blot, or immunoprecipitation.

In some embodiments, the engineered erythroid cell (e.g., engineered enucleated erythroid cell) or enucleated cell (e.g., modified enucleated cell) comprises one or more exogenous polypeptides (e.g., exogenous antigen-presenting polypeptides, exogenous coinhibitory polypeptides, cytokines, and exogenous Treg costimulatory polypeptides, or a combination thereof) and at least one other heterologous polypeptide. The at least one other heterologous polypeptide can be a fluorescent protein. The fluorescent protein can be used as a reporter to assess transduction efficiency. In some embodiments, the fluorescent protein is used as a reporter to assess expression levels of the exogenous polypeptide if both are made from the same transcript. In some embodiments, the at least one other polypeptide is heterologous and provides a function, such as, e.g., multiple antigens, multiple capture targets, enzyme cascade. In some embodiments, the recombinant nucleic acid comprises a gene encoding an antigenic polypeptide and a second gene, wherein the second gene is separated from the gene encoding the antigenic polypeptide by a viral-derived T2A sequence (gagggcagaggaagtcttctaacatgcggtgacgtggaggsgsstcccggccct (SEQ ID NO:35)) that is post-translationally cleaved into two mature proteins.

In some embodiments, the exogenous nucleic acid encoding an exogenous antigen-presenting polypeptide comprises a gene sequence for an HLA cell surface protein that is fused to the 3′ end of the sequence for Kell and amplified using PCR. In some embodiments, the exogenous nucleic acid encoding an exogenous antigen-presenting polypeptide comprises a gene sequence for an HLA cell surface protein that is fused to a poly-glycine/serine linker, followed by the 3′ end of the sequence for Kell, and amplified using PCR. In some embodiments, the exogenous nucleic acid encoding an exogenous antigen-presenting polypeptide comprises the 3′ end of a gene sequence for an HLA cell surface protein that is fused to an epitope tag sequence, of which may be one, or a combination of, an; HA-tag, Green fluorescent protein tag, Myc-tag, chitin binding protein, maltose binding protein, glutathione-S-transferase, poly(His)tag, thioredoxin, poly(NANP), FLAG-tag, V5-tag, AviTag, Calmodulin-tag, polyglutamate-tag, E-tag, S-tag, SBP-tag, Softag-1, Softag-3, Strep-tag, TC-tag, VSV-tag, Xpress-tag, Isopeptag, SpyTag, biotin carboxyl carrier protein, Nus-tag, Fc-tag, or Ty-tag. The entire construct is fused to the 3′ end of the sequence for Kell and then amplified using PCR. The exogenous gene constructs encoding the various exogenous antigen-presenting polypeptides are, for example, subsequently loaded into a lentiviral vector and used to transduce a cell population.

In some embodiments, a population of erythroid cells is incubated with lentiviral vectors comprising exogenous nucleic acid encoding one or more exogenous polypeptides (e.g., exogenous antigen-presenting polypeptides, exogenous costimulatory polypeptides, exogenous coinhibitory polypeptides, cytokines, and exogenous Treg costimulatory polypeptides), specific plasmids of which may include; pLKO.1 puro, PLK0.1—TRC cloning vector, pSico, FUGW, pLVTHM, pLJM1, pLion11, pMD2.G, pCMV-VSV-G, pCI-VSVG, pCMV-dR8.2 dvpr, psPAX2, pRSV-Rev, and pMDLg/pRRE to generate an engineered erythroid cell (e.g., engineered enucleated erythroid cell) or enucleated cell (e.g., modified enucleated cell). The vectors may be administered at 10, 100, 1,000, 10,000 pfu and incubated for 12 hrs.

Erythroid cells described herein can also be produced using coupling reagents to link an exogenous polypeptide to a cell (e.g., using click chemistry as described in detail above). In some embodiments, a first exogenous polypeptide is coupled to the cell (e.g., using click chemistry) and a second exogenous polypeptide comprises a polypeptide expressed from an exogenous nucleic acid.

Methods of manufacturing enucleated erythroid cells comprising (e.g., expressing) an exogenous polypeptides are described, e.g., in WO2015/073587 and WO2015/153102, each of which is incorporated by reference in its entirety.

### III. Methods of Using Engineered Erythroid Cells or Enucleated Cells

The present disclosure contemplates various methods of using the engineered erythroid cells (e.g., engineered enucleated erythroid cells) or enucleated cells (e.g., modified enucleated cells) including a wild-type or loadable exogenous antigen-presenting polypeptide, as described herein.

As would be understood by one skilled in the art, based upon the disclosure provided herein, the dose and timing of administration of the engineered erythroid cells or enucleated cells can be specifically tailored for each application described herein. In essence, the engineered erythroid cells or enucleated cells of the disclosure, and the methods disclosed herein, provide an almost limitless number of variations and the disclosure is not limited in any way to any particular combination or approach. The skilled artisan, armed with the teachings provided herein and the knowledge available in the art, can readily determine the desired approach for each particular subject, disease indication, or target immune cell population.

In some aspects, the disclosure provides a method of treating a subject in need of an altered immune response, the method comprising determining an HLA status of the subject, selecting an engineered erythroid cell or enucleated cell comprising a loadable exogenous antigen-presenting polypeptide on the cell surface, wherein the antigen-presenting polypeptide is immunologically compatible with the subject, and wherein the loadable exogenous antigen-presenting polypeptide comprises one or more amino acid substitutions which stabilize the loadable exogenous antigen-presenting polypeptide on the cell surface, contacting the engineered enucleated erythroid cell with an exogenous antigenic polypeptide to the loadable exogenous antigen-presenting polypeptide; and administering the engineered erythroid cell or enucleated cell to the subject, thereby treating the subject. In some embodiments, the loadable exogenous antigen-presenting polypeptide is stabilized in the absence of a polypeptide bound to the exogenous antigen-presenting polypeptide.

In some aspects, the disclosure provides a method of treating a subject in need of an altered immune response, the method comprising determining an HLA status of the subject, selecting an engineered erythroid cell or enucleated cell comprising a wild-type exogenous antigen-presenting polypeptide on the cell surface, wherein the antigen-presenting polypeptide is immunologically compatible with the subject, contacting the engineered enucleated erythroid cell with an exogenous antigenic polypeptide to the wild-type exogenous antigen-presenting polypeptide; and administering the engineered erythroid cell or enucleated cell to the subject, thereby treating the subject.

In some embodiments, the method comprises conjugating an exogenous antigenic polypeptide to the wild-type or loadable exogenous antigen-presenting polypeptide.

In some embodiments, a displaceable exogenous polypeptide is bound to the loadable exogenous antigen-presenting polypeptide. In some embodiments, the displaceable exogenous polypeptide is displaced from the loadable exogenous antigen-presenting polypeptide with the exogenous antigenic polypeptide prior to administering the engineered enucleated erythroid cell to the subject. In some embodiments, the method further comprises conjugating the exogenous antigenic polypeptide to the loadable exogenous antigen-presenting polypeptide

In some embodiments, the method comprises selecting an exogenous antigenic polypeptide. In some embodiments, the subject has or is at risk of developing a cancer. In some embodiments, the subject has or is at risk of developing an autoimmune disease. In some embodiments, the subject has or is at risk of developing an infectious disease.

In other aspects, the disclosure provides a method of making an engineered enucleated erythroid cell comprising an antigen-loaded wild-type or loadable exogenous antigen-presenting polypeptide, the method comprising obtaining an engineered enucleated erythroid cell comprising a loadable exogenous antigen-presenting polypeptide on the cell surface, wherein the antigen-presenting polypeptide is immunologically compatible with the subject, and wherein the loadable exogenous antigen-presenting polypeptide comprises one or more amino acid substitutions which stabilize the loadable exogenous antigen-presenting polypeptide on the cell surface, and contacting the engineered enucleated erythroid cell with an exogenous antigenic polypeptide to the loadable exogenous antigen-presenting polypeptide; thereby preparing an engineered enucleated erythroid cell comprising an antigen-loaded exogenous antigen-presenting polypeptide. In some embodiments, the loadable exogenous antigen-presenting polypeptide is stabilized in the absence of a polypeptide bound to the exogenous antigen-presenting polypeptide.

In some embodiments, the method comprises conjugating the exogenous antigenic polypeptide to the loadable exogenous antigen-presenting polypeptide. In other embodiments, the method further comprising selecting an exogenous antigenic polypeptide suitable for administration to the subject.

In some embodiments, a displaceable exogenous polypeptide is bound to the loadable exogenous antigen-presenting polypeptide

In some embodiments, the method comprises, displacing the displaceable exogenous polypeptide from the loadable exogenous antigen-presenting polypeptide with the exogenous antigenic polypeptide.

### Treatment of Conditions That Would Benefit From Modulation of T Cell Response

Methods of administering engineered erythroid cells comprising (e.g., presenting) exogenous polypeptides are described, e.g., in WO2015/073587 and WO2015/153102, each of which is incorporated by reference in its entirety.

In some embodiments, the engineered erythroid cells or enucleated cells described herein are administered to a subject, e.g., a mammal, e.g., a human. Exemplary mammals that can be treated include without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like). The methods described herein are applicable to both human therapy and veterinary applications. In some embodiments, the engineered erythroid cell is an enucleated cell. In some embodiments, the erythroid cell is a nucleated cell.

In some embodiments, the erythroid cells are administered to a subject every 1, 2, 3, 4, 5, or 6 months.

In some embodiments, a dose of erythroid cells comprises about 1×10⁹-2×10⁹, 2×10⁹-5×10⁹, 5×10⁹-1×10¹⁰, 1×10¹⁰-2×10¹⁰, 2×10¹⁰-5×10¹¹, 5×10¹¹-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

In some embodiments, the erythroid cells are administered to a subject in a dosing regimen (dose and periodicity of administration) sufficient to maintain function of the administered erythroid cells in the bloodstream of the subject over a period of 2 weeks to a year, e.g., one month to one year or longer, e.g., at least 2 weeks, 4 weeks, 6 weeks, 8 weeks, 3 months, 6 months, a year, 2 years.

In some embodiments, the engineered erythroid cells or enucleated cells as described herein are administered to a subject in two doses or more (e.g. 2, 3, 4 or more doses). In further embodiments, the engineered erythroid cells or enucleated cells are administered to a subject in two doses or more, wherein the second dose is administered at a time after the first dose when T-cell proliferation is determined to be at a peak. In some embodiments, the engineered erythroid cells or enucleated cells of the disclosure are administered in a first dose, wherein the first dose stimulates T cell proliferation and activation. Following the first dose, the engineered erythroid cells or enucleated cells of the disclosure are administered in a second dose, when T cells are activated and proliferation is at its peak, to stimulate T cell expansion. Without being bound by theory, administering the engineered erythroid cells or enucleated cells of the disclosure in two doses or more, increases the capacity of the engineered erythroid cells or enucleated cells to boost the memory T cell population and thereby provide longer efficacy, e.g., efficacy against a relapse of a tumor or re-challenge with an infectious agent.

Peak T-cell proliferation can be determined using methods known to the skilled artisan. For example, peak T-cell proliferation can be determined by ³H-thymidine incorporation by proliferating T-cells, or by labelling proliferating T-cells with the fluorescent dye 5,6-carboxyfluorescein diacetate succinimidyl ester (CF SE).

In some aspects, the present disclosure provides a method of treating a disease or condition described herein, comprising administering to a subject in need thereof a composition described herein that includes an engineered erythroid cell or enucleated cell described herein. In some embodiments, the disease or condition is a cancer. In some embodiments, the disease or condition is an autoimmune disease. In some embodiments, the disease or condition is an autoimmune disease associated with or triggered by an infectious agent. In some embodiments, the disease or condition is an infectious disease.

In some aspects, the disclosure provides a use of an engineered erythroid cell or enucleated cell described herein for treating a disease or condition described herein, e.g., cancer, an autoimmune disease, such as an autoimmune disease triggered by an infectious agent, or an infectious disease. In some aspects, the disclosure provides a use of an engineered erythroid cell or enucleated cell described herein for manufacture of a medicament for treating a disease or condition described herein, e.g., cancer, an autoimmune disease, such as an autoimmune disease triggered by an infectious agent, or an infectious disease.

In some embodiments, the engineered erythroid cell or enucleated cell comprises a first exogenous polypeptide comprising an wild-type or loadable exogenous antigen-presenting polypeptide and a second exogenous polypeptide comprising an exogenous antigenic polypeptide, wherein the exogenous antigenic polypeptide comprises or consists of a tumor antigen, an antigen relating to an autoimmune disorder or condition (e..g., an autoimmune disease triggered by an infectious agent), or an antigen relating to an infectious disease or pathogen. In some embodiments, the wild-type or loadable exogenous antigen-presenting polypeptide is bound (e.g., covalently or non-covalently attached) to the exogenous antigenic polypeptide. In some embodiments, the wild-type or loadable exogenous antigen-presenting polypeptide is not bound (e.g., covalently or non-covalently attached) to the exogenous antigenic polypeptide (e.g., are two independent polypeptides). In some embodiments, the wild-type or loadable exogenous antigen-presenting polypeptide and the second exogenous polypeptide are part of a single chain fusion polypeptide. In some embodiments, the engineered erythroid cell or enucleated cell further comprises a third exogenous polypeptide (e.g., an exogenous polypeptide comprising at least one costimulatory polypeptide, at least one coinhibitory polypeptide, or at least one Treg expansion polypeptide as disclosed herein). In some embodiments, the engineered erythroid cell or enucleated cell further comprises a fourth exogenous polypeptide (e.g., an exogenous polypeptide comprising at least one costimulatory polypeptide, at least one coinhibitory polypeptide, or at least one Treg expansion polypeptide as disclosed herein). In some embodiments, the engineered erythroid cell or enucleated cell further comprises a fifth exogenous polypeptide (e.g., an exogenous polypeptide comprising at least one costimulatory polypeptide, at least one coinhibitory polypeptide, or at least one Treg expansion polypeptide as disclosed herein).

In some embodiments of the foregoing methods, a subject can be administered two different populations of engineered erythroid cells or enucleated cells. For example, a first population of engineered erythroid cells or enucleated cells can include an exogenous polypeptide comprising at least one costimulatory polypeptide, at least one coinhibitory polypeptide, or at least one Treg expansion polypeptide, and a second population of engineered erythroid cells or enucleated cells can include the wild-type or loadable exogenous antigen-presenting polypeptide and the exogenous antigenic polypeptide. In such embodiments, the two distinct populations of engineered erythroid cells or enucleated cells can be administered to a subject, e.g., sequentially or simultaneously.

In some embodiments, the effect of a first and a second exogenous polypeptide on an engineered erythroid cell or enucleated cell, or a first, a second, and a third exogenous polypeptide on an engineered erythroid cell or enucleated cell administered to a subject is synergistic. The term “synergistic” or “synergy” means a more than additive effect of a combination of two or more agents (e.g., polypeptides that are part of an engineered erythroid cell) compared to their individual effects. In certain embodiments, synergistic activity is a more-than-additive effect of administration to a subject of an engineered erythroid cell or enucleated cell comprising a first polypeptide and a second polypeptide, as compared to the effect of administration of two distinct engineered erythroid cells or enucleated cells (e.g., a first engineered erythroid cell or enucleated cell comprising the first polypeptide and a second engineered erythroid cell or enucleated cell comprising the second polypeptide). In some embodiments, synergistic activity is present when a first agent produces a detectable level of an output X, a second agent produces a detectable level of the output X, and the first and second agents together produce a more-than-additive level of the output X.

In some embodiments, at least one engineered erythroid cell or enucleated cell is administered to a subject (e.g., a mammal, e.g., a human) wherein the at least one engineered erythroid cell or enucleated cell comprises a Signal 1 on the cell surface, e.g., comprises a wild-type or loadable exogenous antigen-presenting polypeptide, wherein the wild-type or loadable exogenous antigen-presenting polypeptide comprises a bound exogenous antigenic polypeptide. In some embodiments, the at least one engineered erythroid cell or enucleated cell further comprises a Signal 2 and/or Signal 3 on the cell surface, e.g., further comprises an exogenous polypeptide comprising a Signal 2 polypeptide selected from the Signal 2 polypeptides set forth in Table 11 (e.g., 4-1BBL) and/or an exogenous polypeptide comprising a Signal 3 polypeptide selected from the Signal 3 polypeptides set forth in Table 11 (e.g., IL-12). In some embodiments, the at least one engineered erythroid cell or enucleated cell further comprises an exogenous polypeptide comprising 4-1BBL (Signal 2) and/or an exogenous polypeptide comprising IL-12 (Signal 3).

In some embodiments, two or more (e.g., two, three, four, or more) different engineered erythroid cells or enucleated cells (or populations thereof) are administered to a subject (e.g., a mammal, e.g., a human), wherein the first engineered erythroid cell or enucleated cell (or population thereof) comprises a Signal 1 on the cell surface (e.g., comprises a wild-type or loadable exogenous antigen-presenting polypeptide that is bound to an exogenous antigenic polypeptide), and the second engineered erythroid cell or enucleated cell (or population thereof) comprises a Signal 2 and/or a Signal 3 on the cell surface. For example, in some embodiments, the second engineered erythroid cell or enucleated cell (or population thereof) comprises an exogenous polypeptide comprising a Signal 2 polypeptide selected from the Signal 2 polypeptides set forth in Table 11 (e.g., 4-1BBL) and/or an exogenous polypeptide comprising a Signal 3 polypeptide selected from the Signal 3 polypeptides set forth in Table 11 (e.g., IL-12). In some embodiments, the second engineered erythroid cell or enucleated cell (or population thereof) comprises an exogenous polypeptide comprising 4-1BBL (Signal 2) and/or an exogenous polypeptide comprising IL-12 (Signal 3).

In some embodiments, three or more different engineered erythroid cells or enucleated cells (or populations thereof) are administered to a subject (e.g., a mammal, e.g., a human) wherein the first the engineered erythroid cell or enucleated cell comprises a Signal 1 on the cell surface (e.g., comprises a wild-type or loadable exogenous antigen-presenting polypeptide that is bound to an exogenous antigenic polypeptide), the second engineered erythroid cell or enucleated cell (or population thereof) comprises a Signal 2 on the cell surface (e.g., comprises an exogenous polypeptide comprising a Signal 2 polypeptide selected from the Signal 2 polypeptides set forth in Table 11 (e.g., 4-1BBL)), and the third engineered erythroid cell or enucleated cell (or population thereof) comprises a Signal 3 on the cell surface (e.g., comprises an exogenous polypeptide comprising a Signal 3 polypeptide selected from the Signal 3 polypeptides set forth in Table 11 (e.g., IL-12)).

In some embodiments, the Signal 1, Signal 2 or Signal 3 polypeptides are selected from the polypeptides set forth in Table 11.

In some embodiments, the two, three or more different engineered erythroid cells or enucleated cells (or populations thereof) are administered to a subject sequentially. In some embodiments, the two, three or more different engineered erythroid cells or enucleated cells (or populations thereof) are administered to a subject simultaneously.

Cancer

The engineered erythroid cells or enucleated cells provided herein may be used for the treatment of a cancer in a subject in need thereof. Accordingly, in some aspects, the disclosure provides a method of treating a subject having cancer, comprising administering to the subject an effective number or amount of the engineered erythroid cells or enucleated cells described herein to the subject, thereby treating the cancer.

In some embodiments, provided herein are methods of treating a cancer in a subject in need thereof, wherein the method comprises administering to a subject engineered erythroid cells or enucleated cells (or a pharmaceutical composition comprising the cells) , wherein the engineered erythroid cells or enucleated cells comprise a first exogenous polypeptide comprising an wild-type or loadable exogenous antigen-presenting polypeptide and a second exogenous polypeptide comprising an exogenous antigenic polypeptide, wherein the exogenous antigenic polypeptide comprises or consists of a tumor antigen, thereby treating the cancer. In some embodiments, provided herein are methods of treating a cancer in a subject in need thereof, wherein the method comprises administering to a subject engineered erythroid cells or enucleated cells (or a pharmaceutical composition comprising the cells), wherein the engineered erythroid cells or enucleated cells comprise a first exogenous polypeptide comprising an wild-type or loadable exogenous antigen-presenting polypeptide and a second exogenous polypeptide comprising an exogenous antigenic polypeptide, wherein the exogenous antigenic polypeptide comprises or consists of an amino acid sequence set forth in Tables 7 or 8, thereby treating the cancer. In some embodiments, provided herein are methods of treating a cancer in a subject in need thereof, wherein the method comprises administering to a subject engineered erythroid cells or enucleated cells (or a pharmaceutical composition comprising the cells), wherein the engineered erythroid cells or enucleated cells comprise a first exogenous polypeptide comprising an wild-type or loadable exogenous antigen-presenting polypeptide and a second exogenous polypeptide comprising an exogenous antigenic polypeptide, wherein the exogenous antigenic polypeptide comprises or consists a neoantigen polypeptide set forth in Tables 16 and 17, thereby treating the cancer. In some embodiments, the wild-type or loadable exogenous antigen-presenting polypeptide is bound (e.g., covalently or non-covalently attached) to the exogenous antigenic polypeptide. In some embodiments, the wild-type or loadable exogenous antigen-presenting polypeptide is not bound (e.g., covalently or non-covalently attached) to the exogenous antigenic polypeptide (e.g., are two independent polypeptides). In some embodiments, the engineered erythroid cell or enucleated cell further comprises an additional exogenous polypeptide, wherein the additional exogenous polypeptide provided herein (e.g., an exogenous polypeptide comprising at least one costimulatory polypeptide).

In some embodiments, the engineered erythroid cell or enucleated cell comprises a first exogenous polypeptide comprising a wild-type or loadable exogenous antigen-presenting polypeptide, and the second exogenous polypeptide comprises an exogenous antigenic polypeptide (e.g., comprising a tumor-associated antigen). In some embodiments, the engineered erythroid cell or enucleated cell comprises a first exogenous polypeptide comprising a wild-type or loadable exogenous antigen-presenting polypeptide, a second exogenous polypeptide comprising a first exogenous antigenic polypeptide (e.g., comprising a first tumor-associated antigen), and a third exogenous polypeptide comprises a second exogenous antigenic polypeptide (e.g., comprising a second tumor-associated antigen). The two exogenous antigenic polypeptides antigens may be antigens derived from two different proteins, or they may be derived from the same protein. In some embodiments, the first exogenous antigenic polypeptide or the second exogenous antigenic polypeptide comprises an HPV-E6 antigen. In some embodiments, the first exogenous antigenic polypeptide or the second exogenous antigenic polypeptide comprises an HPV-E7 antigen. In some embodiments, the first exogenous antigenic polypeptide comprises an HPV-E6 antigen and the second exogenous antigenic polypeptide comprises an HPV-E7 antigen.

It is contemplated in some embodiments that an engineered erythroid cell or enucleated cell that expands and activates antigen specific CD8+ T cells in the tumor (e.g., an engineered erythroid cell or enucleated cell comprising an exogenous antigenic polypeptide bound to an exogenous antigen-presenting polypeptide comprising an HLA class I polypeptide) can be administered with an engineered erythroid cell or enucleated cell that activates CD4+ T cells (e.g., an engineered erythroid cell or enucleated cell comprising an exogenous antigenic polypeptide bound to an exogenous antigen-presenting polypeptide comprising an HLA class II polypeptide), which activates both antigen-specific and naive CD8+ T-cells in the tumor and lymph nodes, thereby potentiating a robust anti-tumor response. In some embodiments, an engineered erythroid cell or enucleated cell that expands and activates antigen specific CD8+ T cells in the tumor can be administered with an engineered erythroid cell or enucleated cell that activates CD4+ T cells, which activates both antigen-specific and naïve CD8+ T-cells in the tumor and lymph nodes, thereby synergistically increasing the robustness of the immune response.

In some embodiments, a first engineered erythroid cell or enucleated cell that comprises a first wild-type or loadable exogenous antigen-presenting polypeptide (e.g., comprising an HLA class I or HLA class II polypeptide) and a second exogenous polypeptide comprising an exogenous antigenic polypeptide (e.g., comprising a first tumor-associated antigen) as a single chain fusion polypeptide can be administered to a subject together with a second engineered erythroid cell or enucleated cell that comprises a second wild-type or loadable exogenous antigen-presenting polypeptide (e.g., comprising an HLA class I or HLA class II polypeptide) and a second exogenous polypeptide comprising an exogenous antigenic polypeptide (e.g., comprising a second tumor-associated antigen). In some embodiments, the first engineered erythroid cell or enucleated cell comprises a first wild-type or loadable exogenous antigen-presenting polypeptide comprising an HLA class I polypeptide, and the second engineered erythroid cell or enucleated cell comprises a second wild-type or loadable exogenous antigen-presenting polypeptide comprising an HLA class II polypeptide. In some embodiments, the first engineered erythroid cell or enucleated cell and/or the second engineered erythroid cell or enucleated cell comprises an additional exogenous polypeptide (e.g., an exogenous costimulatory polypeptide provided herein). Without being bound by theory, it is believed that administration of cells that mediate MHC I and MHC II tumor antigen presentation (via antigen-presenting polypeptides comprising HLA class I and HLA class II polypeptides, respectively), combined with potent co-stimulation, has the potential to generate sustained tumor-specific killing.

As will be appreciated by the skilled artisan, in some embodiments the subject may be treated with engineered erythroid cells or enucleated cells comprising multiple, e.g., two, three, four, five or more, different exogenous antigenic polypeptides. In some embodiments, the multiple exogenous antigenic polypeptides are present on the same engineered enucleated erythroid cell. In some embodiments the multiple exogenous antigenic polypeptides are present on two or more distinct engineered enucleated erythroid cells, wherein a combination of the distinct engineered enucleated erythroid cells are administered to the subject to treat the disorder. The two, three, four, five or more exogenous antigenic polypeptides may be each from different proteins, or two or more of the exogenous antigenic polypeptides may be derived from the same protein (e.g., the same tumor antigen or the same neoantigen).

It is encompassed by the present disclosure that the exogenous antigenic polypeptide may include any tumor antigen, or antigenic-portion thereof, known in the art, including, without limitation, any one or more of the exogenous antigenic polypeptides, or antigenic-portions thereof, listed in Tables 7 and 8. Non-limiting, exemplary specific cancers that can be treated using these exogenous antigenic polypeptides are also described Tables 7-8.

Multiple cancers may be treated using the methods described herein. The present disclosure is not limited to a certain type of cancer, but rather any cancer is contemplated as being treated by the engineered erythroid cells or enucleated cells described herein. In certain embodiments, the cancer includes, but is not limited to, acute lymphoblastic leukemia (ALL), ACUTE myeloid leukemia (AML), anal cancer, bile duct cancer, bladder cancer, bone cancer, bowel cancer, brain tumors, breast cancer, cancer of unknown primary, cancer spread to bone, cancer spread to brain, cancer spread to liver, cancer spread to lung, carcinoid, cervical cancer, choriocarcinoma, chronic lymphocytic leukemia (CLL), chronic myeloid leukemia (CML), colon cancer, colorectal cancer, endometrial cancer, eye cancer, gallbladder cancer, gastric cancer, gestational trophoblastic tumors (GTT), hairy cell leukaemia, head and neck cancer, Hodgkin lymphoma, kidney cancer, laryngeal cancer, leukaemia, liver cancer, lung cancer, lymphoma, melanoma skin cancer, mesothelioma, men's cancer, molar pregnancy, mouth and oropharyngeal cancer, myeloma, nasal and sinus cancers, nasopharyngeal cancer, non Hodgkin lymphoma (NHL), oesophageal cancer, ovarian cancer, pancreatic cancer, penile cancer, prostate cancer, rare cancers, rectal cancer, salivary gland cancer, secondary cancers, skin cancer (non melanoma), soft tissue sarcoma, stomach cancer, testicular cancer, thyroid cancer, unknown primary cancer, uterine cancer, vaginal cancer, and vulval cancer.

In certain embodiments, the cancer is a leukemia, e.g. AML or ALL. In other embodiments, the cancer is a hepatic cell carcinoma. In still other embodiments, the cancer is selected from a cervical cancer, head and neck cancer, lymphomas, and kidney clear cell carcinoma.

A neoantigen is a class of tumor antigens that arises from a tumor-specific mutation(s) which alters the amino acid sequence of genome encoded proteins. A neoantigen can include a polypeptide sequence or a nucleotide sequence. A mutation can include a frameshift or non-frameshift indel (insertion or deletion), missense or nonsense substitution, splice site alteration, genomic rearrangement or gene fusion, or any genomic or expression alteration giving rise to a neoORF. A mutation can also include a splice variant. Post-translational modifications specific to a tumor cell can include aberrant phosphorylation. Post-translational modifications specific to a tumor cell can also include a proteasome-generated spliced antigen (see, e.g., Liepe et al. (2016) Science 354(6310): 354-8). A tumor neoantigen is a neoantigen present in a subject's tumor cell or tissue but not in the subject's corresponding normal cell or tissue.

Recent analyses of The Cancer Genome Atlas (TCGA) datasets have linked the genomic landscape of tumors with tumor immunity, implicating neoantigen load in driving T cell responses (Brown et al. (2014) Genome Res. 24(5): 743-50) and identifying somatic mutations associated with immune infiltrates (Rutledge et al. (2013) Clin Cancer Res. 19(18): 4951-60). Rooney et al. (2015) 160(1-2): 48-61) suggest that neoantigens and viruses are likely to drive cytolytic activity, and reveal known and novel mutations that enable tumors to resist immune attack.

In some embodiments, the exogenous antigenic polypeptide included in an engineered erythroid cell or enucleated cell described herein comprises a neoantigen identified from a cancer cell in a subject. In some embodiments, the neoantigen is a shared neoantigen. Methods of identifying neoantigens are known in the art and described, e.g., in U.S. Pat. No. 10,055,540, incorporated by reference in its entirety herein. Neoantigenic polypeptides and shared neoantigenic polypeptides are described, for example, in International Patent Publication No. WO 2016/187508; U.S. Publication No. 20180055922; Schumacher and Hacohen et al. (2016) Curr. Opin. Immunol. 41: 98-103; Gubin et al. (2014) Nature 515(7528): 577-81; Schumacher and Schreiber (2015) Science 348(6230): 69-74, Ott et al. (2017) Nature 547(7662): 217-21; each of which are incorporated by reference in their entireties herein.

Accordingly, in some embodiments, the exogenous antigenic polypeptide comprises or consists of a neoantigen polypeptide. In some embodiments, the exogenous antigenic polypeptide comprises or consists of a neoantigen polypeptide set forth in The Comprehensive Tumor-Specific Neoantigen Database (TSNAdb v1.0); available at biopharm.zju.edu.cnitsnadb and described in Wu et al. (2018) Genomics Proteomics Bioinformatics 16: 276-82. In some embodiments, the exogenous antigenic polypeptide is a neoantigen polypeptide set forth in U.S. Pat. No. 10,055,540, incorporated by reference in its entirety herein. In some embodiments, the exogenous antigenic polypeptide comprises a neoantigen polypeptide listed in Table 16.

TABLE 16
Neoantigen Polypeptides

| HLA class I | | SEQ ID |
|---|---|---|
| allele | Neoantigen Polypeptide | NO: |
| HLA-A*01:01 | YEMFNDKSF | 932 |
| HLA-A*02:01 | HROEIFSHDFJ | 933 |
| HLA-B*07:02 | FJIEJFOESS | 934 |
| HLA-B*08:01 | NEIOREIREI | 935 |
| HLA-C*01:03 | JFKSIFEMMSJDSSU | 936 |
| HLA-C*01:04 | KNFLENFIESOFI | 937 |

In some embodiments, the exogenous antigenic polypeptide comprises or consists of a neoantigen polypeptide listed in Table 17. Non-limiting examples of HLA polypeptide alleles which may bind to the neoantigen polypeptides are also described and may be used to design desirable wild-type or loadable antigen-presenting polypeptides for use with the specified neoantigen polypeptides.

TABLE 17
Neoantigen Polypeptides

| | | | SEQ |
|---|---|---|---|
| Genetic | | Neoantigen | ID |
| Mutation | Allele(s) | polypeptide | NO: |
| BRAF V600E | HLA-A*3:01; HLA-A*11:01 | KIGDFGLATEK | 938 |
| BRAF V600E | HLA-B*57:01 | FGLATEKSRW | 939 |
| BRAF V600E | HLA-C*12:03; HLA-C*03:03 | ATEKSRWSG | 940 |
| BRAF V600E | HLA-A*24:02; HLA-C*12:03; | DFGLATEK | 941 |
| | HLA-C*07:02 | | |
| BRAF V600E | HLA-C*03:03; HLA-C*12:03 | EKSRWSGSH | 942 |
| BRAF V600E | HLA-C*03:03; HLA-C*12:03; | FGLATEKSR | 248 |
| | HLA-C*07:02 | | |
| BRAF V600E | HLA-C*12:03; HLA-C*03:03; | GDFGLATEK | 249 |
| | HLA-A*11:01 | | |
| BRAF V600E | HLA-A*31:01 | GDFGLATEKSR | 943 |
| BRAF V600E | HLA-C*12:03; HLA-C*03:03 | GLATEKSRW | 944 |
| BRAF V600E | HLA-C*12:03; HLA-C*05:01; | IGDFGLATE | 945 |
| | HLA-C*08:02 | | |
| BRAF V600E | HLA-B*08:01; HLA-B*57:01 | LATEKSRW | 946 |
| BRAF V600E | HLA-C*03:03; HLA-C*12:03; | LATEKSRWS | 246 |
| | HLA-C*07:01 | | |
| BRAF V600E | HLA-B*18:01; HLA-C*12:03; | TEKSRWSG | 947 |
| | HLA-C*03:03 | | |
| BRAF V600M | HLA-C*12:03; HLA-C*03:03; | ATMKSRWSG | 948 |
| | HLA-A*30:01; HLA-C*15:02 | | |
| BRAF V600M | HLA-A*24:02 | DFGLATMK | 949 |
| BRAF V600M | HLA-C*12:03; HLA-C*07:02; | DFGLATMKS | 950 |
| | HLA-C*03:03 | | |
| BRAF V600M | HLA-A*02:01 | FGLATMKS | 951 |
| BRAF V600M | HLA-C*03:03; HLA-C*12:03; | FGLATMKSR | 952 |
| | HLA-C*07:02 | | |
| BRAF V600M | HLA-C*12:03; HLA-C*03:03; | GDFGLATMK | 953 |
| | HLA-A*11:01 | | |
| BRAF V600M | HLA-A*31:01 | GDFGLATMKSR | 954 |
| BRAF V600M | HLA-C*12:03; HLA-C*03:03 | GLATMKSRW | 955 |
| BRAF V600M | HLA-C*12:03; HLA-C*05:01; | IGDFGLATM | 956 |
| | HLA-C*08:02; HLA-C*16:01; | | |
| | HLA-C*07:02; HLA-C*03:03; | | |
| | HLA-C*03:04; HLA-B*35:01 | | |
| BRAF V600M | HLA-B*08:01 | LATMKSRW | 957 |
| BRAF V600M | HLA-C*03:03; HLA-C*12:03; | LATMKSRWS | 958 |
| | HLA-C*07:01 | | |
| BRAF V600M | HLA-C*12:03; HLA-C*03:03 | MKSRWSGSH | 959 |
| BRAF V600M | HLA-B*08:01 | TMKSRWSG | 960 |
| BRAF V600M | HLA-C*12:03; HLA-C*03:03 | TMKSRWSGS | 961 |
| BRAF V600M | HLA-B*15:01; HLA-B*08:01 | TMKSRWSGSH | 962 |
| EGFR A289V | HLA-C*12:03; HLA-C*03:03 | EGKYSFGVT | 963 |
| EGFR A289V | HLA-A*68:01; HLA-A*11:01 | EGKYSFGVTCVK | 964 |
| EGFR A289V | HLA-A*68:01; HLA-A*11:01 | EGKYSFGVTCVKK | 965 |
| EGFR A289V | HLA-C*03:03; HLA-C*12:03; | FGVTCVKKC | 966 |
| | HLA-C*07:02 | | |
| EGFR A289V | HLA-A*31:01 | FGVTCVKKCPR | 967 |
| EGFR A289V | HLA-C*12:03; HLA-C*03:03; | GKYSFGVTC | 968 |
| | HLA-C*07:02 | | |
| EGFR A289V | HLA-A*11:01 | GKYSFGVTCVK | 969 |
| EGFR A289V | HLA-A*68:01; HLA-A*11:01 | GKYSFGVTCVKK | 970 |
| EGFR A289V | HLA-C*12:03; HLA-C*03:03 | GVTCVKKCP | 971 |
| EGFR A289V | HLA-A*68:01; HLA-A*31:01 | GVTCVKKCPR | 972 |
| EGFR A289V | HLA-C*12:03; HLA-C*03:03; | KYSFGVTCV | 973 |
| | HLA-C*07:02 | | |
| EGFR A289V | HLA-A*03:01; HLA-A*68:01; | KYSFGVTCVK | 974 |
| | HLA-A*11:01; HLA-A*30:01; | | |
| | HLA-A*31:01 | | |
| EGFR A289V | HLA-A*68:01; HLA-A*11:01 | KYSFGVTCVKK | 975 |
| EGFR A289V | HLA-A*31:01; HLA-A*68:01 | KYSFGVTCVKKCPR | 976 |
| EGFR A289V | HLA-B*18:01; HLA-A*24:02 | PEGKYSFG | 977 |
| EGFR A289V | HLA-C*12:03 | PEGKYSFGV | 978 |
| EGFR A289V | HLA-A*68:01; HLA-A*11:01 | PEGKYSFGVTCVK | 979 |
| EGFR A289V | HLA-A*68:01; HLA-A*11:01 | PEGKYSFGVTCVKK | 980 |
| EGFR A289V | HLA-C*12:03; HLA-C*07:02; | SFGVTCVKK | 981 |
| | HLA-C*03:03 | | |
| EGFR A289V | HLA-A*31:01 | SFGVTCVKKCPR | 982 |
| EGFR A289V | HLA-A*31:01; HLA-B*08:01 | TCVKKCPR | 983 |
| EGFR A289V | HLA-A*31:01; HLA-C*03:03; | VTCVKKCPR | 984 |
| | HLA-C*12:03; HLA-A*68:01 | | |
| EGFR A289V | HLA-A*68:01; HLA-C*12:03; | YSFGVTCVK | 985 |
| | HLA-A*11:01; HLA-C*07:01; | | |
| | HLA-C*15:02; HLA-C*03:03; | | |
| | HLA-A*03:01; HLA-A*30:01; | | |
| | HLA-C*07:02 | | |
| EGFR A289V | HLA-A*68:01; HLA-A*11:01; | YSFGVTCVKK | 986 |
| | HLA-A*03:01 | | |
| EGFR A289V | HLA-A*68:01; HLA-A*31:01 | YSFGVTCVKKCPR | 987 |
| EGFR G598V | HLA-C*03:03; HLA-C*12:03; | AVVMGENNT | 988 |
| | HLA-C*05:01 | | |
| EGFR G598V | HLA-B*15:01 | AVVMGENNTL | 989 |
| EGFR G598V | HLA-C*12:03; HLA-C*03:03; | CPAVVMGEN | 990 |
| | HLA-B*35:01 | | |
| EGFR G598V | HLA-C*12:03; HLA-C*15:02; | CVKTCPAVV | 991 |
| | HLA-C*05:01; HLA-C*03:03 | | |
| EGFR G598V | HLA-B*15:01 | CVKTCPAVVM | 992 |
| EGFR G598V | HLA-C*12:03; HLA-C*03:03 | HCVKTCPAV | 993 |
| EGFR G598V | HLA-C*12:03; HLA-C*07:02; | KTCPAVVMG | 994 |
| | HLA-C*03:03; HLA-A*30:01 | | |
| EGFR G598V | HLA-C*12:03; HLA-C*03:03; | PAVVMGENN | 995 |
| | HLA-C*15:02 | | |
| EGFR G598V | HLA-C*12:03 | TCPAVVMGE | 996 |
| EGFR G598V | HLA-C*03:03; HLA-C*12:03; | VKTCPAVVM | 997 |
| | HLA-C*07:02 | | |
| EGFR G598V | HLA-C*03:04; HLA-C*12:03; | VVMGENNTL | 998 |
| | HLA-C*03:03; HLA-C*16:01; | | |
| | HLA-C*07:02; HLA-C*15:02; | | |
| | HLA-C*06:02; HLA-C*07:01 | | |
| EGFR L858R | HLA-A*24:02 | DFGRAKLL | 999 |
| EGFR L858R | HLA-C*12:03; HLA-C*03:03 | DFGRAKLLG | 1000 |
| EGFR L858R | HLA-B*08:01 | FGRAKLLG | 1001 |
| EGFR L858R | HLA-C*12:03; HLA-A*30:01 | FGRAKLLGA | 1002 |
| EGFR L858R | HLA-C*12:03; HLA-C*03:03; | GRAKLLGAE | 1003 |
| | HLA-C*07:02 | | |
| EGFR L858R | HLA-C*12:03; HLA-A*31:01; | HVKITDFGR | 1004 |
| | HLA-A*68:01; HLA-C*07:02; | | |
| | HLA-A*30:01; HLA-C*03:03 | | |
| EGFR L858R | HLA-C*12:03; HLA-C*05:01; | ITDFGRAKL | 1005 |
| | HLA-C*08:02; HLA-C*16:01; | | |
| | HLA-C*03:03 | | |
| EGFR L858R | HLA-C*12:03; HLA-C*03:03; | KITDFGRAK | 1006 |
| | HLA-A*30:01; HLA-A*11:01; | | |
| | HLA-A*03:01; HLA-C*07:02; | | |
| | HLA-A*31:01 | | |
| EGFR L858R | HLA-C*12:03; HLA-C*03:03; | RAKLLGAEE | 1007 |
| | HLA-C*15:02 | | |
| EGFR L858R | HLA-C*03:03; HLA-C*12:03; | TDFGRAKLL | 1008 |
| | HLA-C*07:01 | | |
| EGFR L858R | HLA-C*12:03; HLA-C*03:03 | VKITDFGRA | 1009 |
| ERBB2 S310F | HLA-A*24:02 | DVGFCTLV | 1010 |
| ERBB2 S310F | HLA-C*12:03; HLA-C*05:01 | DVGFCTLVC | 1011 |
| ERBB2 S310F | HLA-C*03:03; HLA-C*12:03; | FCTLVCPLH | 1012 |
| | HLA-C*08:02; HLA-C*07:02; | | |
| ERBB2 S310F | HLA-C*03:03; HLA-C*12:03; | GFCTLVCPL | 1013 |
| | HLA-C*07:02 | | |
| ERBB2 S310F | HLA-C*12:03; HLA-C*03:03; | LSTDVGFCT | 1014 |
| | HLA-C*15:02 | | |
| ERBB2 S310F | HLA-C*07:02; HLA-C*03:03; | NYLSTDVGF | 1015 |
| | HLA-C*12:03; HLA-A*24:02 | | |
| ERBB2 S310F | HLA-A*02:01 | NYLSTDVGFCTLV | 1016 |
| ERBB2 S310F | HLA-A*02:01 | NYLSTDVGFCTLVC | 1017 |
| ERBB2 S310F | HLA-C*12:03; HLA-C*05:01; | STDVGFCTL | 1018 |
| | HLA-C*16:01; HLA-C*15:02; | | |
| | HLA-C*03:04; HLA-C*03:03; | | |
| | HLA-A*32:01 | | |
| ERBB2 S310F | HLA-A*01:01 | STDVGFCTLV | 1019 |
| ERBB2 S310F | HLA-C*12:03; HLA-C*03:03 | TDVGFCTLV | 1020 |
| ERBB2 S310F | HLA-C*12:03; HLA-C*03:03; | VGFCTLVCP | 1021 |
| | HLA-C*07:02 | | |
| ERBB2 S310F | HLA-B*40:01 | VGFCTLVCPL | 1022 |
| ERBB2 S310F | HLA-C*03:03; HLA-C*12:03; | YLSTDVGFC | 1023 |
| | HLA-C*05:01; HLA-A*02:01 | | |
| ERBB2 S310F | HLA-A*02:01 | YLSTDVGFCT | 1024 |
| ERBB2 S310F | HLA-A*02:01 | YLSTDVGFCTLV | 1025 |
| ERBB2 S310F | HLA-A*02:01 | YLSTDVGFCTLVC | 1026 |
| ERBB2 S310F | HLA-A*02:01 | YLSTDVGFCTLVCP | 1027 |
| FBXW7 R465C | HLA-C*12:03; HLA-A*31:01 | CCMHLHEKR | 1028 |
| FBXW7 R465C | HLA-A*31:01 | CMHLHEKR | 1029 |
| FBXW7 R465C | HLA-C*03:03; HLA-C*12:03; | GHTSTVCCM | 1030 |
| | HLA-C*07:02 | | |
| FBXW7 R465C | HLA-A*11:01; HLA-A*68:01 | GHTSTVCCMHLHEK | 1031 |
| FBXW7 R465C | HLA-C*12:03; HLA-C*03:03; | HTSTVCCMH | 1032 |
| | HLA-C*15:02; HLA-A*68:01; | | |
| | HLA-C*05:01; HLA-C*07:02; | | |
| FBXW7 R465C | HLA-A*11:01; HLA-A*68:01 | HTSTVCCMHLHEK | 1033 |
| FBXW7 R465C | HLA-A*68:01; HLA-A*31:01 | HTSTVCCMHLHEKR | 1034 |
| FBXW7 R465C | HLA-C*12:03; HLA-C*03:03 | LYGHTSTVC | 1035 |
| FBXW7 R465C | HLA-B*35:01 | LYGHTSTVCCM | 1036 |
| FBXW7 R465C | HLA-C*12:03; HLA-C*03:03; | STVCCMHLH | 1037 |
| | HLA-C*15:02 | | |
| FBXW7 R465C | HLA-A*11:01 | STVCCMHLHEK | 1038 |
| FBXW7 R465C | HLA-A*31:01; HLA-A*68:01; | STVCCMHLHEKR | 1039 |
| | HLA-A*11:01 | | |
| FBXW7 R465C | HLA-C*03:03; HLA-C*12:03; | TSTVCCMHL | 1040 |
| | HLA-C*15:02; HLA-C*07:01; | | |
| | HLA-C*07:02 | | |
| FBXW7 R465C | HLA-A*11:01 | TSTVCCMHLHEK | 1041 |
| FBXW7 R465C | HLA-A*31:01; HLA-A*68:01 | TSTVCCMHLHEKR | 1042 |
| FBXW7 R465C | HLA-C*12:03; HLA-C*03:03 | TVCCMHLHE | 1043 |
| FBXW7 R465C | HLA-A*11:01; HLA-A*68:01; | TVCCMHLHEK | 1044 |
| | HLA-A*03:01; HLA-C*12:03; | | |
| | HLA-C*03:03 | | |
| FBXW7 R465C | HLA-A*31:01 | VCCMHLHEKR | 1045 |
| FBXW7 R465C | HLA-C*03:03; HLA-C*12:03; | YGHTSTVCC | 1046 |
| | HLA-C*07:02 | | |
| FBXW7 R465C | HLA-B*15:01 | YGHTSTVCCM | 1047 |
| FGFR3 S249C | HLA-C*12:03; HLA-C*03:03 | CPHRPILQA | 1048 |
| FGFR3 S249C | HLA-C*12:03; HLA-C*03:03; | DVLERCPHR | 1049 |
| | HLA-C*07:02; HLA-A*68:01; | | |
| | HLA-A*31:01 | | |
| FGFR3 S249C | HLA-A*02:01 | DVLERCPHRPI | 1050 |
| FGFR3 S249C | HLA-C*07:02; HLA-C*06:02; | ERCPHRPIL | 1051 |
| | HLA-C*07:01; HLA-C*03:03; | | |
| | HLA-C*12:03 | | |
| FGFR3 S249C | HLA-C*03:03; HLA-C*12:03 | LDVLERCPH | 1052 |
| FGFR3 S249C | HLA-C*12:03; HLA-B*40:01; | LERCPHRPI | 1053 |
| | HLA-C*03:03; HLA-B*44:02 | | |
| FGFR3 S249C | HLA-B*40:01 | LERCPHRPIL | 1054 |
| FGFR3 S249C | HLA-C*12:03 | RCPHRPILQ | 1055 |
| FGFR3 S249C | HLA-B*07:02 | RCPHRPILQA | 1056 |
| FGFR3 S249C | HLA-C*12:03; HLA-C*05:01; | TLDVLERCP | 1057 |
| | HLA-C*07:02; HLA-C*07:01 | | |
| FGFR3 S249C | HLA-C*12:03; HLA-C*03:03 | VLERCPHRP | 1058 |
| FGFR3 S249C | HLA-C*12:03; HLA-C*03:03; | YTLDVLERC | 1059 |
| | HLA-C*15:02 | | |
| FGFR3 S249C | HLA-A*68:01 | YTLDVLERCPHR | 1060 |
| FLT3 D835Y | HLA-C*12:03; HLA-C*03:03; | ARYIMSDSN | 1061 |
| | HLA-C*07:01; HLA-C*07:02 | | |
| FLT3 D835Y | HLA-B*15:01; HLA-B*27:05; | ARYIMSDSNY | 1062 |
| | HLA-A*29:02 | | |
| FLT3 D835Y | HLA-A*02:01 | ARYIMSDSNYV | 1063 |
| FLT3 D835Y | HLA-C*12:03; HLA-C*03:03; | CDFGLARYI | 1064 |
| | HLA-C*07:01; HLA-B*13:02; | | |
| | HLA-C*15:02 | | |
| FLT3 D835Y | HLA-B*15:01 | CDFGLARYIM | 1065 |
| FLT3 D835Y | HLA-A*24:02 | DFGLARYI | 1066 |
| FLT3 D835Y | HLA-C*12:03; HLA-C*07:02; | DFGLARYM | 1067 |
| | HLA-C*03:03; HLA-B*18:01; | | |
| FLT3 D835Y | HLA-B*08:01 | FGLARYIM | 1068 |
| FLT3 D835Y | HLA-C*03:03; HLA-C*12:03; | FGLARYIMS | 1069 |
| | HLA-C*07:02 | | |
| FLT3 D835Y | HLA-B*15:01 | FGLARYIMSD | 1070 |
| FLT3 D835Y | HLA-A*02:01 | FGLARYIMSDSNYV | 1071 |
| FLT3 D835Y | HLA-A*02:01 | GLARYIMS | 1072 |
| FLT3 D835Y | HLA-C*03:03; HLA-C*12:03 | GLARYIMSD | 1073 |
| FLT3 D835Y | HLA-A*02:01 | GLARYIMSDSNYV | 1074 |
| FLT3 D835Y | HLA-C*12:03; HLA-C*05:01; | ICDFGLARY | 1075 |
| | HLA-C*08:02; HLA-A*01:01 | | |
| FLT3 D835Y | HLA-A*02:01 | IMSDSNYV | 1076 |
| FLT3 D835Y | HLA-C*12:03; HLA-C*03:03 | LARYIMSDS | 1077 |
| FLT3 D835Y | HLA-A*02:01 | LARYIMSDSNYV | 1078 |
| FLT3 D835Y | HLA-C*12:03; HLA-C*03:03; | RYIMSDSNY | 1079 |
| | HLA-A*29:02; HLA-C*07:02 | | |
| FLT3 D835Y | HLA-A*02:01; HLA-A*24:02; | RYIMSDSNYV | 1080 |
| | HLA-B*15:01 | | |
| FLT3 D835Y | HLA-A*02:01; HLA-C*15:02; | YIMSDSNYV | 1081 |
| | HLA-C*12:03; HLA-C*03:04; | | |
| | HLA-C*02:02; HLA-C*16:01; | | |
| | HLA-C*06:02; HLA-C*07:01; | | |
| | HLA-C*03:03 | | |
| GNA11 Q209L | HLA-A*24:02 | DVGGLRSE | 1082 |
| GNA11 Q209L | HLA-C*12:03; HLA-A*68:01; | DVGGLRSER | 1083 |
| | HLA-C*03:03; HLA-C*07:02 | | |
| GNA11 Q209L | HLA-A*68:01; HLA-A*31:01 | DVGGLRSERR | 1084 |
| GNA11 Q209L | HLA-A*11:01 | DVGGLRSERRK | 1085 |
| GNA11 Q209L | HLA-C*06:02; HLA-C*07:01; | FRMVDVGGL | 1086 |
| | HLA-C*07:02; HLA-B*27:05; | | |
| | HLA-C*03:03; HLA-C*12:03; | | |
| | HLA-C*08:02 | | |
| GNA11 Q209L | HLA-A*68:01; HLA-B*27:05; | FRMVDVGGLR | 1087 |
| | HLA-A*31:01 | | |
| GNA11 Q209L | HLA-A*31:01; HLA-B*27:05 | FRMVDVGGLRSER | 1088 |
| GNA11 Q209L | HLA-A*31:01; HLA-B*27:05 | FRMVDVGGLRSWRR | 1089 |
| GNA11 Q209L | HLA-C*03:03; HLA-C*12:03 | GGLRSERRK | 1090 |
| | HLA-C*07:02 | | |
| GNA11 Q209L | HLA-C*06:02; HLA-C*07:01; | LRSERRKWI | 1092 |
| | HLA-C*12:03; HLA-C*03:03; | | |
| | HLA-C*07:02; HLA-B*27:05 | | |
| GNA11 Q209L | HLA-C*05:01; HLA-C*12:03; | MVDVGGLRS | 1093 |
| | HLA-C*07:01; HLA-C*08:02 | | |
| GNA11 Q209L | HLA-B*15:01 | MVDVGGLRS | 1094 |
| GNA11 Q209L | HLA-A*31:01; HLA-C*12:03; | RMVDVGGLR | 1095 |
| | HLA-C*03:03; HLA-A*68:01; | | |
| | HLA-A*03:01 | | |
| GNA11 Q209L | HLA-A*31:01 | RMVDVGGLRSER | 1096 |
| GNA11 Q209L | HLA-A*31:01 | RMVDVGGLRSERR | 1097 |
| GNA11 Q209L | HLA-A*03:01 | RMVDVGGLRSERRK | 1098 |
| GNA11 Q209L | HLA-C*12:03; HLA-C*03:03; | VDVGGLRSE | 1099 |
| | HLA-C*07:02 | | |
| GNA11 Q209L | HLA-A*11:01; HLA-A*31:01 | VDVGGLRSERR | 1100 |
| GNA11 Q209L | HLA-C*12:03; HLA-C*03:03; | VGGLRSERR | 1101 |
| | HLA-C*07:02; HLA-C*07:01 | | |
| GNAQ Q209P | HLA-C*12:03; HLA-C*03:03; | DVGGPRSER | 1102 |
| | HLA-A*68:01; HLA-C*07:02; | | |
| | HLA-C*07:01 | | |
| GNAQ Q209P | HLA-A*68:01 | DVGGPRSERR | 1103 |
| GNAQ Q209P | HLA-A*11:01 | DVGGPRSERRK | 1104 |
| GNAQ Q209P | HLA-C*07:01; HLA-C*06:02; | FRMVDVGGP | 1105 |
| | HLA-C*12:03; HLA-C*07:02; | | |
| | HLA-C*03:03 | | |
| GNAQ Q209P | HLA-B*27:05; HLA-A*68:01; | FRMVDVGGPR | 1106 |
| | HLA-A*31:01 | | |
| GNAQ Q209P | HLA-B*27:05 | FRMVDVGGPRS | 1107 |
| GNAQ Q209P | HLA-B*27:05 | FRMVDVGGPRSE | 1108 |
| GNAQ Q209P | HLA-A*31:01; HLA-B*27:05 | FRMVDVGGPRSER | 1109 |
| GNAQ Q209P | HLA-A*31:01; HLA-B*27:05 | FRMVDVGGPRSERR | 1110 |
| GNAQ Q209P | HLA-C*12:03; HLA-C*07:02 | GGPRSERRK | 1111 |
| GNAQ Q209P | HLA-C*12:03; HLA-B*07:02 | GPRSERRKW | 1112 |
| GNAQ Q209P | HLA-B*07:02 | GPRSERRKWI | 1113 |
| GNAQ Q209P | HLA-C*12:03; HLA-C*05:01; | MVDVGGPRS | 1114 |
| | HLA-C*07:01; HLA-C*08:02 | | |
| GNAQ Q209P | HLA-B*15:01 | MVDVGGPRSE | 1115 |
| GNAQ Q209P | HLA-C*06:02; HLA-C*07:01; | PRSERRKWI | 1116 |
| | HLA-C*12:03; HLA-C*03:03; | | |
| | HLA-C*07:02 | | |
| GNAQ Q209P | HLA-A*31:01; HLA-C*12:03; | RMVDVGGPR | 1117 |
| | HLA-C*03:03 | | |
| GNAQ Q209P | HLA-A*31:01 | RMVDVGGPRSER | 1118 |
| GNAQ Q209P | HLA-A*31:01 | RMVDVGGPRSERR | 1119 |
| GNAQ Q209P | HLA-C*03:03; HLA-C*12:03; | VDVGGPRSE | 1120 |
| | HLA-C*07:02 | | |
| GNAQ Q209P | HLA-A*11:01; HLA-A*31:01 | VDVGGPRSERR | 1121 |
| GNAQ Q209P | HLA-C*12:03; HLA-C*07:02; | VGGPRSERR | 1122 |
| | HLA-C*03:03; HLA-C*07:01 | | |
| H3F3A K27M | HLA-A*02:01 | RMSAPSTGGV | 1123 |
| HRAS Q61R | HLA-C*12:03; HLA-C*07:02; | AGREEYSAM | 1124 |
| | HLA-C*16:01; HLA-C*03:03 | | |
| HRAS Q61R | HLA-A*31:01 | AGREEYSAMR | 1125 |
| HRAS Q61R | HLA-C*12:03; HLA-C*03:03; | DILDTAGRE | 1126 |
| | HLA-C*15:02 | | |
| HRAS Q61R | HLA-A*29:02 | DILDTAGREEY | 1127 |
| HRAS Q61R | HLA-B*18:01 | DTAGREEY | 1128 |
| HRAS Q61R | HLA-C*03:03; HLA-C*12:03; | DTAGREEYS | 1129 |
| | HLA-C*07:01 | | |
| HRAS Q61R | HLA-A*26:01 | DTAGREEYSAM | 1130 |
| HRAS Q61R | HLA-A*68:01; HLA-A*26:01 | DTAGREEYSAMR | 1131 |
| HRAS Q61R | HLA-A*26:01 | DTAGREEYSAMRD | 1132 |
| HRAS Q61R | HLA-B*18:01 | EEYSAMRD | 1133 |
| HRAS Q61R | HLA-C*07:01; HLA-C*12:03; | GREEYSAMR | 1134 |
| | HLA-C*07:02; HLA-C*05:01; | | |
| | HLA-C*03:03 | | |
| HRAS Q61R | HLA*B*27:05 | GREEYSAMRD | 1135 |
| HRAS Q61R | HLA-C*12:03; HLA-C*05:01; | ILDTAGREE | 1136 |
| | HLA-C*08:02; HLA-C*07:01; | | |
| | HLA-C*03:03 | | |
| HRAS Q61R | HLA-B*15:01; HLA-A*01:01; | ILDTAGREEY | 230 |
| | HLA-A*29:02 | | |
| HRAS Q61R | HLA-A*68:01 | ILDTAGREEYSAMR | 1137 |
| HRAS Q61R | HLA-C*12:03; HLA-C*03:03; | LDILDTAGR | 1138 |
| | HLA-A*68:01 | | |
| HRAS Q61R | HLA-C*03:03; HLA-C*12:03; | LDTAGREEY | 1139 |
| | HLA-B*35:01 | | |
| HRAS Q61R | HLA-A*68:01 | LDTAGREEYSAMR | 1140 |
| HRAS Q61R | HLA-C*12:03; HLA-C*03:03; | REEYSAMRD | 1141 |
| | HLA-C*05:01 | | |
| HRAS Q61R | HLA-A*02:01 | TAGREEYS | 1142 |
| HRAS Q61R | HLA-C*12:03; HLA-C*03:03 | TAGREEYSA | 1143 |
| IDH1 R132H | HLA-C*12:03; HLA-C*03:03; | GHHAYGDQY | 1144 |
| | HLA-A*29:02; HLA-C*07:02 | | |
| IRH1 R132H | HLA-A*31:01; HLA-A*68:01 | GHHAYGDQYR | 1145 |
| IDH1 R132H | HLA-C*03:03; HLA-C*12:03; | HHAYGDQYR | 1146 |
| | HLA-A*68:01; HLA-C*07:02 | | |
| IDH1 R132H | HLA-C*03:03; HLA-C*12:03; | IGHHAYGDQ | 1147 |
| | HLA-C*07:02 | | |
| IDH1 R132H | HLA-A*29:02; HLA-B*18:01 | IGHHAYGDQY | 1148 |
| IDH1 R132H | HLA-C*12:03; HLA-C*03:03; | IIGHHAYGD | 1149 |
| | HLA-C*15:02 | | |
| IDH1 R132H | HLA-A*29:02 | IIGHHAYGDQY | 1150 |
| IDH1 R132H | HLA-A*31:01 | IIGHHAYGDQYR | 1151 |
| IDH1 R132H | HLA-A*29:02 | IIIGHHAY | 1152 |
| IDH1 R132H | HLA-C*03:03; HLA-C*12:03; | IIIGHHAYG | 1153 |
| | HLA-C*15:02 | | |
| IDH1 R132H | HLA-A*29:02 | IIIGHHAYGDQY | 1154 |
| IDH1 R132H | HLA-A*31:01; HLA-A*68:01 | IIIGHHAYGDQYR | 1155 |
| IDH1 R132H | HLA-C*12:03; HLA-C*03:03 | KPIIIGHHA | 1156 |
| IDH1 R132H | HLA-B*15:01; HLA-B*35:01; | KPIIIGHHAY | 1157 |
| | HLA-A*29:02; HLA-B*07:02 | | |
| IDH1 R132H | HLA-A*02:01 | KPIIIGHHAYG | 1158 |
| IDH1 R132H | HLA-A*29:02 | KPIIIGHHAYGDQY | 1159 |
| IDH1 R132H | HLA-C*12:03; HLA-A*29:02; | PIIIGHHAY | 1160 |
| | HLA-C*03:03; HLA-B*35:01 | | |
| IDH1 R132H | HLA-A*02:01 | PIIIGHHAYGD | 1161 |
| IDH1 R132H | HLA-A*29:02 | PIIIGHHAYGDQY | 1162 |
| IDH1 R132H | HLA-A*31:01 | PIIIGHHAYGDQYR | 1163 |
| IDH1 R132H | HLA-C*12:03; HLA-C*07:02 | VKPIIIGHH | 1164 |
| IDH1 R132H | HLA-A*29:02 | VKPIIIGHHAY | 1165 |
| IDH1 R132H | HLA-C*12:03; HLA-C*03:03; | WVKPIIIGH | 1166 |
| | HLA-C*07:02 | | |
| IDH1 R132H | HLA-A*29:02 | WVKPIIIGHHAY | 1167 |
| JAK1 K860Nfs*16 | HLA-C*12:03; HLA-C*03:03; | DIVSEKNQQ | 1168 |
| | HLA-C*07:02 | | |
| JAK1 K860Nfs*16 | HLA-A*11:01 | DIVSEKNQQ | 1169 |
| JAK1 K860Nfs*16 | HLA-C*12:03; HLA-C*05:01 | EKNQQLKWT | 1170 |
| JAK1 K860Nfs*16 | HLA-C*03:03; HLA-C*12:03; | IVSEKNQQL | 1171 |
| | HLA-C*05:01; HLA-C*16:01; | | |
| | HLA-C*07:01; HLA-C*07:02; | | |
| | HLA-C*15:02; HLA-C*06:02; | | |
| | HLA-C*03:04 | | |
| JAK1 K860Nfs*16 | HLA-A*68:01; HLA-A*03:01; | IVSEKNQQLK | 1172 |
| | HLA-A*11:01 | | |
| JAK1 K860Nfs*16 | HLA-B*44:02 | VSEKNQQLKW | 1173 |
| JAK1 K860Nfs*16 | HLA-B*44:02 | SEKNQQLKWT | 1174 |
| JAK1 K860Nfs*16 | HLA-C*12:03; HLA-C*07:02; | KNQQLKWTP | 1175 |
| JAK1 K860Nfs*16 | HLA-C*12:03; HLA-C*07:02 | KWTPHILKS | 1176 |
| | HLA-C*03:03 | | |
| JAK1 K860Nfs*16 | HLA-A*30:01 | KWTPHILKSA | 1177 |
| JAK1 K860Nfs*16 | HLA-C*12:03; HLA-C*03:03 | LKWTPHILK | 1178 |
| JAK1 K860Nfs*16 | HLA-C*12:03; HLA-C*05:01 | NPDIVSEKN | 1179 |
| JAK1 K860Nfs*16 | HLA-B*08:01 | NPDIVSEKNQQ | 1180 |
| JAK1 K860Nfs*16 | HLA-C*12:03; HLA-C*03:03 | NQQLKWTPH | 1181 |
| JAK1 K860Nfs*16 | HLA-C*12:03; HLA-C*03:03 | PDIVSEKNQ | 1182 |
| JAK1 K860Nfs*16 | HLA-C*12:03; HLA-C*03:03 | QLKWTPHIL | 1183 |
| JAK1 K860Nfs*16 | HLA-A*03:01; HLA-A*11:01 | QLKWTPHILK | 1184 |
| JAK1 K860Nfs*16 | HLA-A*31:01 | QLKWTPHILKS | 1185 |
| JAK1 K860Nfs*16 | HLA-C*12:03; HLA-C*03:03; | QQLKWTPHI | 1186 |
| | HLA-B*13:02; HLA-A*02:01 | | |
| JAK1 K860Nfs*16 | HLA-B*15:01 | QQLKWTPHIL | 1187 |
| JAK1 K860Nfs*16 | HLA-A*11:01; HLA-A*03:01 | QQLKWTPHILK | 1188 |
| JAK1 K860Nfs*16 | HLA-B*18:01 | SEKNQQLK | 1189 |
| JAK1 K860Nfs*16 | HLA-C*12:03; HLA-B*44:02; | SEKNQQLKW | 1190 |
| | HLA-B*44:03 | | |
| JAK1 K860Nfs*16 | HLA-B*07:02 | TPHILKSA | 1191 |
| JAK1 K860Nfs*16 | HLA-C*12:03; HLA-C*03:03; | TPHILKSAS | 1192 |
| | HLA-B*35:01 | | |
| JAK1 K860Nfs*16 | HLA-C*07:01; HLA-C*12:03; | VSEKNQQLK | 1193 |
| | HLA-C*05:01; HLA-C*15:02; | | |
| | HLA-C*03:03; HLA-A*11:01 | | |
| JAK1 K860Nfs*16 | HLA-C*12:03; HLA-C*15:02 | WTPHILKSA | 1194 |
| KRAS G12C | HLA-C*03:03; HLA-C*12:03 | ACGVGKSAL | 1195 |
| KRAS G12C | HLA-C*03:03; HLA-C*12:03 | CGVGKSALT | 1196 |
| KRAS G12C | HLA-C*12:03; HLA-C*03:03; | GACGVGKSA | 1197 |
| | HLA-C*15:02 | | |
| KRAS G12C | HLA-A*24:02 | GVGKSALT | 1198 |
| KRAS G12C | HLA-C*12:03; HLA-C*03:03; | KLVVVGACG | 1199 |
| | HLA-C*05:01 | | |
| KRAS G12C | HLA-A*02:01 | KLVVVGACGV | 1200 |
| KRAS G12C | HLA-A*03:01 | KLVVVGACGVGK | 1201 |
| KRAS G12C | HLA-C*12:03; HLA-C*15:02; | LVVVGACGV | 1202 |
| | HLA-C*03:03 | | |
| KRAS G12C | HLA-A*11:01 | LVVVGACGVGK | 1203 |
| KRAS G12C | HLA-C*12:03; HLA-C*03:03; | VGACGVGKS | 1204 |
| | HLA-C*07:02 | | |
| KRAS G12C | HLA-C*12:03; HLA-A*11:01; | VVGACGVGK | 1205 |
| | HLA-C*03:03; HLA-A*03:01; | | |
| | HLA-C*05:01 | | |
| KRAS G12C | HLA-C*12:03; HLA-C*03:03; | VVVGACGVG | 1206 |
| | HLA-C*07:02 | | |
| KRAS G12C | HLA-A*03:01; HLA-A*11:01; | VVVGACGVGK | 1207 |
| | HLA-A*68:01 | | |
| KRAS G12C | HLA-C*03:03; HLA-C*12:03; | YKLVVVGAC | 1208 |
| | HLA-C*07:02 | | |
| KRAS G12D | HLA-C*03:03; HLA-C*12:03 | ADGVGKSAL | 1209 |
| KRAS G12D | HLA-C*03:03; HLA-C*12:03 | DGVGKSALT | 1210 |
| KRAS G12D | HLA-C*12:03; HLA-C*15:01; | GADGVGKSA | 1211 |
| | HLA-C*08:02; HLA-C*15:02 | | |
| KRAS G12D | HLA-A*24:02 | GVGKSALT | 1198 |
| KRAS G12D | HLA-C*12:03; HLA-C*03:03; | KLVVVGADG | 1212 |
| | HLA-C*05:01 | | |
| KRAS G12D | HLA-A*02:01 | KLVVVGADGV | 1213 |
| KRAS G12D | HLA-C*12:03; HLA-C*15:02; | LVVVGADGV | 1214 |
| | HLA-C*03:03 | | |
| KRAS G12D | HLA-A*11:01 | LVVVGADGVGK | 1215 |
| KRAS G12D | HLA-C*03:03; HLA-C*12:03; | VGADGVGKS | 1216 |
| | HLA-C*07:02 | | |
| KRAS G12D | HLA-C*12:03; HLA-C*03:03; | VVGADGVGK | 1217 |
| | HLA-A*11:01; HLA-C*15:01 | | |
| KRAS G12D | HLA-C*03:03; HLA-C*12:03; | VVVGADGVG | 1218 |
| | HLA-C*07:02; HLA-C*15:02 | | |
| KRAS G12D | HLA-A*11:01; HLA-A*68:01; | VVVGADGVGK | 1219 |
| | HLA-A*03:01 | | |
| KRAS G12D | HLA-C*03:03; HLA-C*12:03; | YKLVVVGAD | 1220 |
| | HLA-C*07:02 | | |
| KRAS G12V | HLA-C*03:03; HLA-C*12:03; | AVGVGKSAL | 1221 |
| | HLA-B*07:02 | | |
| KRAS G12V | HLA-C*12:03; HLA-C*03:03; | GAVGVGKSA | 1222 |
| | HLA-C*15:02 | | |
| KRAS G12V | HLA-A*02:01 | KLVVVGAV | 1223 |
| KRAS G12V | HLA-A*24:02 | GVGKSALT | 1198 |
| KRAS G12V | HLA-C*12:03; HLA-C*03:03; | KLVVVGAVG | 1224 |
| | HLA-C*05:01 | | |
| KRAS G12V | HLA-A*02:01 | KLVVVGAVGV | 219 |
| KRAS G12V | HLA-A*03:01 | KLVVVGAVGVGK | 1225 |
| KRAS G12V | HLA-C*12:03; HLA-C*15:02; | LVVVGAVGV | 220 |
| | HLA-C*03:03 | | |
| KRAS G12V | HLA-B*15:01 | LVVVGAVGVG | 1226 |
| KRAS G12V | HLA-A*11:01 | LVVVGAVGVGK | 1227 |
| KRAS G12V | HLA-C*12:03; HLA-C*03:03; | VGAVGVGKS | 1228 |
| | HLA-C*07:02 | | |
| KRAS G12V | HLA-A*03:03; HLA-C*12:03 | VGVGKSALT | 1229 |
| KRAS G12V | HLA-C*12:03; HLA-A*11:01; | VVGAVGVGK | 1230 |
| | HLA-C*03:01; HLA-C*05:01; | | |
| | HLA-C*03:03 | | |
| KRAS G12V | HLA-C*12:03; HLA-C*03:03; | VVVGAVGVG | 221 |
| | HLA-C*07:02 | | |
| KRAS G12V | HLA-A*11:01; HLA-A*03:01; | VVVGAVGVGK | 1231 |
| | HLA-A*68:01 | | |
| KRAS G12V | HLA-C*12:03; HLA-C*03:03 | YKLVVVGAV | 1232 |
| KRAS G13D | HLA-C*12:03; HLA-C*05:01; | AGDVGKSAL | 1233 |
| | HLA-C*08:02; HLA-C*03:03; | | |
| | HLA-C*07:02 | | |
| KRAS G13D | HLA-A*24:02 | DVGKSALT | 1234 |
| KRAS G13D | HLA-C*12:03; HLA-C*15:02; | DVGKSALTI | 1235 |
| | HLA-C*03:03 | | |
| KRAS G13D | HLA-A*24:02; HLA-A*02:01 | GAGDVGKS | 1236 |
| KRAS G13D | HLA-C*12:03; HLA-C*03:03; | GAGDVGKSA | 1237 |
| | HLA-C*15:02; HLA-C*15:01 | | |
| KRAS G13D | HLA-C*03:03; HLA-C*12:03 | GDVGKSALT | 1238 |
| KRAS G13D | HLA-C*12:03; HLA-C*03:03; | KLVVVGAGD | 1239 |
| | HLA-C*05:01 | | |
| KRAS G13D | HLA-C*12:03; HLA-C*03:03; | LVVVGAGDV | 1240 |
| | HLA-C*15:02 | | |
| KRAS G13D | HLA-A*11:01 | LVVVGAGDVGK | 1241 |
| KRAS G13D | HLA-C*03:03; HLA-C*12:03; | VGAGDVGKS | 1242 |
| | HLA-C*07:02 | | |
| KRAS G13D | HLA-C*12:03; HLA-C*03:03; | VVGAGDVGK | 1243 |
| | HLA-A*11:01; HLA-C*05:01; | | |
| | HLA-C*07:02 | | |
| KRAS G13D | HLA-C*12:03; HLA-C*03:03; | VVVGAGDVG | 1244 |
| | HLA-C*15:02 | | |
| KRAS G13D | HLA-A*11:01; HLA-A*68:01; | VVVGAGDVGK | 1245 |
| | HLA-A*03:01 | | |
| NPM1 W288Cfs*12 | HLA-B*40:01; HLA-B*44:03; | QEAIWDLCL | 1246 |
| | HLA-C*03:03; HLA-C*07:02; | | |
| | HLA-C*12:03 | | |
| NPM1 W288Cfs*12 | HLA-C*03:03; HLA-C*12:03 | EAIWDLCLA | 1247 |
| NPM1 W288Cfs*12 | HLA-B*15:01 | QEAIWDLCLA | 1248 |
| NPM1 W288Cfs*12 | HLA-C*03:03; HLA-C*05:01; | DQEAIWDLC | 1249 |
| | HLA-C*12:03 | | |
| NPM1 W288Cfs*12 | HLA-B*15:01 | DQEAIWDLCL | 1250 |
| NPM1 W288Cfs*12 | HLA-A*02:01 | EAIWDLCLAV | 1251 |
| NPM1 W288Cfs*12 | HLA-C*12:03; HLA-A*02:01 | AIWDLCLAV | 1252 |
| NPM1 W288Cfs*12 | HLA-C*05:01; HLA-C*12:03 | IWDLCLAVE | 1253 |
| NPM1 W288Cfs*12 | HLA-A*02:01; HLA-B*15:01 | AIWDLCLAVE | 1254 |
| NPM1 W288Cfs*12 | HLA-C*03:03; HLA-C*12:03 | WDLCLAVEE | 1255 |
| NPM1 W288Cfs*12 | HLA-A*02:01 | WDLCLAVEEV | 1256 |
| NPM1 W288Cfs*12 | HLA-C*12:03; HLA-A*02:01; | AIQDLCLAV | 1257 |
| | HLA-C*15:02 | | |
| NPM1 W288Cfs*12 | HLA-B*15:01 | AIQDLCLAVE | 1258 |
| NPM1 W288Cfs*12 | HLA-C*05:01; HLA-C*12:03; | AVEEVSLRK | 1259 |
| | HLA-A*11:01; HLA-A*03:01; | | |
| | HLA-C*15:02; HLA-A*30:01 | | |
| NPM1 W288Cfs*12 | HLA-A*02:01 | CLAVEEVS | 1260 |
| NPM1 W288Cfs*12 | HLA-C*03:03; HLA-C*12:03; | CLAVEEVSL | 1261 |
| | HLA-C*07:02 | | |
| NPM1 W288Cfs*12 | HLA-A*68:01 | CLAVEEVSLR | 1262 |
| NPM1 W288Cfs*12 | HLA-A*11:01; HLA-A*03:01 | CLAVEEVSLRK | 1263 |
| NPM1 W288Cfs*12 | HLA-C*12:03 | DLCLAVEEV | 1264 |
| NPM1 W288Cfs*12 | HLA-B*15:01 | DLCLAVEEVS | 1265 |
| NPM1 W288Cfs*12 | HLA-C*12:03; HLA-C-05:01; | DQEAIQDLC | 1266 |
| | HLA-C*03:03 | | |
| NPM1 W288Cfs*12 | HLA-B*15:01 | DQEAIQDLCL | 1267 |
| NPM1 W288Cfs*12 | HLA-C*12:03; HLA-C*03:03; | EAIQDLCLA | 1268 |
| | HLA-C*15:02 | | |
| NPM1 W288Cfs*12 | HLA-C*12:03; HLA-C*05:01; | IQDLCLAVE | 1269 |
| | HLA-C*08:02 | | |
| NPM1 W288Cfs*12 | HLA-B*15:01 | IQDLCLAVEE | 1270 |
| NPM1 W288Cfs*12 | HLA-C*12:03; HLA-C*03:03; | LAVEEVSLR | 1271 |
| | HLA-A*68:01; HLA-C*05:01; | | |
| | HLA-A*31:01; | | |
| NPM1 W288Cfs*12 | HLA-A*11:01; HLA-A*68:01 | LAVEEVSLRK | 1272 |
| NPM1 W288Cfs*12 | HLA-C*12:03; HLA-C*03:03 | LCLAVEEVS | 1273 |
| NPM1 W288Cfs*12 | HLA-B*15:01; HLA-B*08:01 | LCLAVEEVSL | 1274 |
| NPM1 W288Cfs*12 | HLA-C*03:03; HLA-C*12:03 | QDLCLAVEE | 1275 |
| NPM1 W288Cfs*12 | HLA-B*18:01 | QEAIQDLC | 1276 |
| NPM1 W288Cfs*12 | HLA-C*03:03; HLA-B*40:01; | QEAIQDLCL | 1277 |
| | HLA-C*12:03; HLA-C*07:02 | | |
| NPM1 W288Cfs*12 | HLA-A*02:01 | QEAIQDLCLAV | 1278 |
| NRAS Q61K | HLA-C*12:03; HLA-C*03:03; | AGKEEYSAM | 1279 |
| | HLA-C*07:02 | | |
| NRAS Q61K | HLA-A*31:01 | AGKEEYSAMR | 1280 |
| NRAS Q61K | HLA-C*12:03; HLA-C*03:03; | DILDTAGKE | 1281 |
| | HLA-C*15:02 | | |
| NRAS Q61K | HLA-A*29:02 | DILDTAGKEEY | 1282 |
| NRAS Q61K | HLA-B*18:01 | DTAGKEEY | 1283 |
| NRAS Q61K | HLA-C*03:03; HLA-C*12:03 | DTAGKEEYS | 1284 |
| NRAS Q61K | HLA-A*26:01 | DTAGKEEYSAM | 1285 |
| NRAS Q61K | HLA-A*68:01; HLA-A*26:01 | DTAGKEEYSAMR | 1286 |
| NRAS Q61K | HLA-A*26:01 | DTAGKEEYSAMRD | 1287 |
| NRAS Q61K | HLA-B*18:01 | EEYSAMRD | 1133 |
| NRAS Q61K | HLA-C*12:03; HLA-C*05:01; | GKEEYSAMR | 1288 |
| | HLA-C*03:03 | | |
| NRAS Q61K | HLA-C*12:03; HLA-C*05:01; | ILDTAGKEE | 1289 |
| | HLA-C*08:02 | | |
| NRAS Q61K | HLA-B*15:01; HLA-A*01:01; | ILDTAGKEEY | 1290 |
| | HLA-A*29:02 | | |
| NRAS Q61K | HLA-A*68:01 | ILDTAGKEEYSAMR | 1291 |
| NRAS Q61K | HLA-C*12:03; HLA-C*05:01; | KEEYSAMRD | 1292 |
| | HLA-C*03:03 | | |
| NRAS Q61K | HLA-C*12:03; HLA-C*03:03 | LDILDTAGK | 1293 |
| NRAS Q61K | HLA-C*03:03; HLA-C*12:03; | LDTAGKEEY | 1294 |
| | HLA-B*35:01 | | |
| NRAS Q61K | HLA-A*68:01 | LDTAGKEEYSAMR | 1295 |
| NRAS Q61K | HLA-A*02:01 | TAGKEEYS | 1296 |
| NRAS Q61K | HLA-C*12:03; HLA-C*03:03 | TAGKEEYSA | 1297 |
| NRAS Q61R | HLA-C*12:03; HLA-C*07:02; | AGREEYSAM | 1124 |
| | HLA-C*16:01; HLA-C*03:03 | | |
| NRAS Q61R | HLA-A*31:01 | AGREEYSAMR | 1125 |
| NRAS Q61R | HLA-C*12:03; HLA-C*03:03; | DILDTAGRE | 1126 |
| | HLA-C*15:02 | | |
| NRAS Q61R | HLA-A*29:02 | DILDTAGREEY | 1127 |
| NRAS Q61R | HLA-B*18:01 | DTAGREEY | 1128 |
| NRAS Q61R | HLA-C*03:03; HLA-C*12:03; | DTAGREEYS | 1129 |
| | HLA-C*07:01 | | |
| NRAS Q61R | HLA-A*26:01 | DTAGREEYSAM | 1130 |
| NRAS Q61R | HLA-A*68:01; HLA-A*26:01 | DTAGREEYSAMR | 1131 |
| NRAS Q61R | HLA-A*26:01 | DTAGREEYSAMRD | 1132 |
| NRAS Q61R | HLA-B*18:01 | EEYSAMRD | 1133 |
| NRAS Q61R | HLA-C*07:01; HLA-C*12:03; | GREEYSAMR | 1134 |
| | HLA-C*07:02; HLA-C*05:01; | | |
| | HLA-C*03:03 | | |
| NRAS Q61R | HLA-B*27:05 | GREEYSAMRD | 1135 |
| NRAS Q61R | HLA-C*12:03; HLA-C*05:01; | ILDTAGREE | 1136 |
| | HLA-C*08:02; HLA-C*07:01; | | |
| | HLA-C*03:03 | | |
| NRAS Q61R | HLA-B*15:01; HLA-A*01:01; | ILDTAGREEY | 230 |
| | HLA-A*29:02 | | |
| NRAS Q61R | HLA-A*68:01 | ILDTAGREEYSAMR | 1137 |
| NRAS Q61R | HLA-C*12:03; HLA-C*03:03; | LDILDTAGR | 1138 |
| | HLA-A*68:01 | | |
| NRAS Q61R | HLA-C*03:03; HLA-C*12:03; | LDTAGREEY | 1139 |
| | HLA-B*35:01 | | |
| NRAS Q61R | HLA-A*68:01 | LDTAGREEYSAMR | 1140 |
| NRAS Q61R | HLA-C*12:03; HLA-C*03:03; | REEYSAMRD | 1141 |
| | HLA-C*05:01 | | |
| NRAS Q61R | HLA-A*02:01 | TAGREEYS | 1142 |
| NRAS Q61R | HLA-C*12:03; HLA-C*03:03 | TAGREEYSA | 1143 |
| P53 R175H | HLA-C*03:03; HLA-C*12:03; | EVVRHCPHH | 1298 |
| | HLA-C*07:02 | | |
| P53 R175H | HLA-A*31:01; HLA-A*68:01 | EVVRHCPHHER | 1299 |
| P53 R175H | HLA-C*12:03; HLA-C*07:02 | HCPHHERCS | 1300 |
| P53 R175H | HLA-C*12:03; HLA-C*07:02 | HMTEVVRHC | 1301 |
| P53 R175H | HLA-A*31:01; HLA-A*68:01 | HMTEVVRHCPHHER | 1302 |
| P53 R175H | HLA-C*12:03; HLA-C*15:02; | MTEVVRHCP | 1303 |
| | HLA-C*03:03 | | |
| P53 R175H | HLA-A*31:01; HLA-A*68:01 | MTEVVRHCPHHER | 1304 |
| P53 R175H | HLA-A*31:01 | MTEVVRHCPHHERC | 1305 |
| P53 R175H | HLA-C*12:03; HLA-C*03:03 | QHMTEVVRH | 1306 |
| P53 R175H | HLA-A*31:01 | RHCPHHER | 1307 |
| P53 R175H | HLA-C*12:03; HLA-C*07:02; | RHCPHHERC | 1308 |
| | HLA-C*03:03 | | |
| P53 R175H | HLA-B*18:01 | TEVVRHCP | 1309 |
| P53 R175H | HLA-C*03:03; HLA-C*12:03; | TEVVRHCPH | 1310 |
| | HLA-C*07:02 | | |
| P53 R175H | HLA-A*31:01; HLA-A*68:01 | TEVVRHCPHHER | 1311 |
| P53 R175H | HLA-C*07:01; HLA-C*12:03; | VRHCPHHER | 1312 |
| | HLA-C*03:03; HLA-C*07:02; | | |
| | HLA-B*27:05; HLA-A*31:01 | | |
| P53 R175H | HLA-C*12:03 | VVRHCPHHE | 1313 |
| P53 R175H | HLA-A*31:01; HLA-A*68:01 | VVRHCPHHER | 1314 |
| P53 R248Q | HLA-A*24:02 | CMGGMNQR | 1315 |
| P53 R248Q | HLA-C*12:03; HLA-C*03:03; | CMGGMNQRP | 1316 |
| | HLA-C*07:02 | | |
| P53 R248Q | HLA-C*07:02; HLA-C*12:03; | GGMNQRPIL | 1317 |
| | HLA-C 03:30 | | |
| P53 R248Q | HLA-A*02:01 | GGMNQRPILTI | 1318 |
| P53 R248Q | HLA-C*12:03; HLA-C*07:01; | GMNQRPILT | 1319 |
| | HLA-C*05:01; HLA-C*06:02 | | |
| P53 R248Q | HLA-B*15:01 | GMNQRPILTI | 1320 |
| P53 R248Q | HLA-A*02:01 | GMNQRPILTII | 1321 |
| P53 E248Q | HLA-A*24:02 | MGGMNQRP | 1322 |
| P53 R248Q | HLA-C*03:03; HLA-C*12:03; | MGGMNQRPI | 1323 |
| | HLA-C*07:02 | | |
| P53 R248Q | HLA-B*08:01 | MGGMNQRPIL | 1324 |
| P53 R248Q | HLA-C*12:03; HLA-C*03:03; | MNQRPILTI | 1325 |
| | HLA-A*32:01 | | |
| P53 R248Q | HLA-C*12:03 | NQRPILTII | 1326 |
| P53 R248Q | HLA-B*15:01 | NQRPILTIIT | 1327 |
| P53 R248Q | HLA-C*12:03; HLA-C*07:02 | QRPILTIIT | 1328 |
| P53 R248Q | HLA-C*12:03; HLA-C*03:03; | SCMGGMNQR | 1329 |
| | HLA-C*07:02; HLA-A*31:01 | | |
| P53 R248Q | HLA-C*12:03; HLA-C*15:02; | SSCMGGMNQ | 1330 |
| | HLA-C*03:03; HLA-C*07:01 | | |
| P53 R248Q | HLA-A*11:01; HLA-A*68:01; | SSCMGGMNQR | 1331 |
| | HLA-A*31:01 | | |
| P53 R248Q | HLA-A*11:01 | SSCMGGMNQRP | 1332 |
| P53 R273C | HLA-C*12:03; HLA-C*03:03 | CVCACPGRD | 1333 |
| P53 R273C | HLA-C*03:03; HLA-C*12:03 | EVCVCACPG | 1334 |
| P53 R273C | HLA-A*68:01; HLA-A*31:01 | EVCVCACPGR | 1335 |
| P53 R273C | HLA-B*18:01 | FEVCVCAC | 1336 |
| P53 R273C | HLA-C*03:03; HLA-C*12:03; | FEVCVCACP | 1337 |
| | HLA-C*07:02 | | |
| P53 R273C | HLA-C*12:03; HLA-C*07:01; | GRNSFEVCV | 1338 |
| | HLA-C*06:03; HLA-C*07:02 | | |
| P53 R273C | HLA-C*12:03 | LGRNSFEVC | 1339 |
| P53 R273C | HLA-C*12:03; HLA-C*07:01; | NSFEVCVCA | 1340 |
| | HLA-C*15:02; HLA-C*03:03; | | |
| | HLA-C*07:02; HLA-C*06:02 | | |
| P53 R273C | HLA-B*15:01 | NSFEVCVCAC | 1341 |
| P53 R273C | HLA-A*68:01 | NSFEVCVCACPGR | 1342 |
| P53 R273C | HLA-C*03:03; HLA-C*12:03; | RNSFEVCVC | 1343 |
| | HLA-C*15:02; HLA-C*07:03; | | |
| P53 R273C | HLA-A*68:01 | RNSFEVCVCACPGR | 1344 |
| P53 R273C | HLA-C*12:03; HLA-C*03:03; | SFEVCVCAC | 1345 |
| | HLA-C*07:02 | | |
| P53 R273C | HLA-C*12:03; HLA-C*03:03; | VCVCACPGR | 1346 |
| | HLA-C*07:02 | | |
| P53 R273H | HLA-C*03:03; HLA-C*12:03 | EVHVCACPG | 1347 |
| P53 R273H | HLA-A*68:01 | EVHVCACPGR | 1348 |
| P53 R273H | HLA-B*18:01 | FEVHVCAC | 1349 |
| P53 R273H | HLA-C*03:03; HLA-C*12:03; | FEVHVCACP | 1350 |
| | HLA-C*07:02; HLA-C*05:01 | | |
| P53 R273H | HLA-C*12:03; HLA-C*07:01; | GRNSFEVHV | 1351 |
| | HLA-C*06:02; HLA-C*07:02 | | |
| P53 R273H | HLA-C*12:03; HLA-C*03:03; | HVCACPGRD | 1352 |
| | HLA-C*07:02 | | |
| P53 R273H | HLA-C*12:03 | LGRNSFEVH | 1353 |
| P53 R273H | HLA-C*12:03; HLA-C*07:01; | NSFEVHVCA | 1354 |
| | HLA-C*15:02; HLA-C*03:03; | | |
| | HLA-C*06:02 | | |
| P53 R273H | HLA-B*15:01 | NSFEVHVCAC | 1355 |
| P53 R273H | HLA-A*68:01 | NSFEVHVCACPGR | 1356 |
| P53 R273H | HLA-C*12:03; HLA-C*03:03; | RNSFEVHVC | 1357 |
| | HLA-C*15:02 | | |
| P53 R273H | HLA-A*68:01 | RNSFEVHVCACPGR | 1358 |
| P53 R273H | HLA-C*12:03; HLA-C*03:03; | SFEVHVCAC | 1359 |
| | HLA-C*07:02 | | |
| P53 R273H | HLA-C*12:03; HLA-C*03:03; | VHVCACPGR | 1360 |
| | HLA-C*07:02 | | |
| PIK3CA E542K | HLA-C*03:03; HLA-C*12:03 | DPLSKITEQ | 1361 |
| PIK3CA E542K | HLA-A*11:01 | DPLSKITEQEK | 1362 |
| PIK3CA E542K | HLA-A*02:01 | ISTRDPLS | 1363 |
| PIK3CA E542K | HLA-C*15:02; HLA-C*12:03; | ISTRDPLSK | 1364 |
| | HLA-C*03:03; HLA-A*11:01; | | |
| | HLA-A*30:01; HLA-A*03:01; | | |
| | HLA-C*08:02 | | |
| PIK3CA E542K | HLA-B*08:01 | KITEQEKD | 1365 |
| PIK3CA E542K | HLA-C*12:03; HLA-C*03:03; | KITEQEKDF | 1366 |
| | HLA-C*07:02 | | |
| PIK3CA E542K | HLA-C*12:03; HLA-A*30:01; | LSKITEQEK | 1367 |
| | HLA-C*03:03; HLA-C*15:02 | | |
| PIK3CA E542K | HLA-A*31:01 | LSKITEQEKDF | 1368 |
| PIK3CA E542K | HLA-C*03:03; HLA-C*12:03; | PLSKITEQE | 1369 |
| | HLA-C*05:01 | | |
| PIK3CA E542K | HLA-C*12:03 | RDPLSKITE | 1370 |
| PIK3CA E542K | HLA-C*12:03; HLA-C*03:03 | SKITEQEKD | 1371 |
| PIK3CA E542K | HLA-A*30:01 | STRDPLSK | 1372 |
| PIK3CA E542K | HLA-C*12:03; HLA-A*30:01; | STRDPLSKI | 1373 |
| | HLA-C*15:02 | | |
| PIK3CA E542K | HLA-A*31:01 | STRDPLSKITE | 1374 |
| PIK3CA E542K | HLA-C*12:03; HLA-C*07:01; | TRDPLSKIT | 1375 |
| | HLA-C*06:02; HLA-C*07:02 | | |
| PIK3CA E542K | HLA-B*08:01 | TRDPLSKITEQ | 1376 |
| PIK3CA E545K | HLA-A*03:01 | STRDPLSEITK | 1377 |
| PIK3CA E545K | HLA-C*03:03; HLA-C*12:03 | DPLSEITKQ | 1378 |
| PIK3CA E545K | HLA-A*11:01 | DPLSEITKQEK | 1379 |
| PIK3CA E545K | HLA-B*08:01 | EITKQEKD | 1380 |
| PIK3CA E545K | HLA-C*03:03; HLA-C*12:03 | EITKQEKDF | 1381 |
| PIK3CA E545K | HLA-C*12:03; HLA-C*03:03; | ITKQEKDFL | 1382 |
| | HLA-A*30:01 | | |
| PIK3CA E545K | HLA-A*31:01 | ITKQEKDFLWS | 1383 |
| PIK3CA E545K | HLA-A*24:02 | KQEKDFLW | 1384 |
| PIK3CA E545K | HLA-3*12:03; HLA-C*05:01 | KQEKDFLWS | 1385 |
| PIK3CA E545K | HLA-C*12:03; HLA-C*05:01; | LSEITKQEK | 1386 |
| | HLA-C*15:02; HLA-C*03:03; | | |
| | HLA-C*07:01 | | |
| PIK3CA E545K | HLA-B*44:03 | LSEITKQEKDFLW | 1387 |
| PIK3CA E545K | HLA-C*12:03; HLA-A*03:03; | PLSEITKQE | 1388 |
| | HLA-C*05:01; HLA-C*07:02 | | |
| PIK3CA E545K | HLA-B*18:01; HLA-A*02:01 | QEKDFLWS | 1389 |
| PIK3CA E545K | HLA-C*12:03; HLA-C*07:02 | RDPLSEITK | 1390 |
| PIK3CA E545K | HLA-B*18:01 | SEITKQEK | 1391 |
| PIK3CA E545K | HLA-C*12:03; HLA-C*03:03 | SEITKQEKD | 1392 |
| PIK3CA E545K | HLA-B*44:02; HLA-B*15:01; | SEITKQEKDF | 1393 |
| | HLA-B*44:03 | | |
| PIK3CA E545K | HLA-B*44:02; HLA-B*44:03 | SEITKQEKDFLW | 1394 |
| PIK3CA E545K | HLA-B*44:03; HLA-B*44:02 | SEITKQEKDFLWS | 1395 |
| PIK3CA E545K | HLA-B*08:01 | TKQEKDFL | 1396 |
| PIK3CA E545K | HLA-C*12:03; HLA-C*03:03 | TKQEKDFLW | 1397 |
| PIK3CA H1047R | HLA-C*03:03; HLA-C*12:03; | ARHGGWTTK | 1398 |
| | HLA-C*07:01; HLA-B*27:05; | | |
| | HLA-C*07:02; HLA-A*30:01 | | |
| PIK3CA H1047R | HLA-B*27:05; HLA-C*07:01; | ARHGGWTTKM | 1399 |
| | HLA-C*07:02 | | |
| PIK3CA H1047R | HLA-C*12:03; HLA-C*15:02 | DARHGGWTT | 1400 |
| PIK3CA H1047R | HLA-C*12:03; HLA-C*03:03; | FMKQMNDAR | 1401 |
| | HLA-A*31:01; HLA-A*68:01; | | |
| | HLA-C*07:02 | | |
| PIK3CA H1047R | HLA-A*31:01 | FMKQMNDARHG | 1402 |
| PIK3CA H1047R | HLA-A*24:02 | HGGWTTKM | 1403 |
| PIK3CA H1047R | HLA-C*12:03; HLA-B*15:01 | KQMNDARHG | 1404 |
| PIK3CA H1047R | HLA-A*03:01 | KQMNDARHGGWTTK | 1405 |
| PIK3CA H1047R | HLA-C*12:03;HLA-C*03:03 | MKQMNDARH | 1406 |
| PIK3CA H1047R | HLA-B*15:01 | MKQMNDARHG | 1407 |
| PIK3CA H1047R | HLA-C*12:03; HLA-C*05:01; | MNDARHGGW | 1408 |
| | HLA-C*08:02; HLA-C*07:01 | | |
| PIK3CA H1047R | HLA-C*12:03; HLA-C*03:03 | NDARHGGWT | 1409 |
| PIK3CA H1047R | HLA-C*11:01 | NDARHGGWTTK | 1410 |
| PIK3CA H1047R | HLA-C*12:03; HLA-C*07:01 | QMNDARHGG | 1411 |
| PIK3CA H1047R | HLA-B*44:02 | QMNDARHGGW | 1412 |
| PIK3CA H1047R | HLA-A*02:01 | QMNDARHGGWT | 1413 |
| PIK3CA H1047R | HLA-A*24:02; HLA-B*27:05 | RHGGWTTK | 1414 |
| PIK3CA H1047R | HLA-C*12:03; HLA-C*03:03; | RHGGWTTKM | 1415 |
| | HLA-C*07:02 | | |
| PPP2R1A P179R | HLA-C*12:03; HLA-C*05:01; | CSDDTRMVR | 1416 |
| | HLA-C*07:01; HLA-C*15:02; | | |
| | HLA-A*68:01; HLA-A*31:01 | | |
| PPP2R1A P179R | HLA-A*31:01; HLA-A*68:01 | CSDDTRMVRR | 1417 |
| PPP2R1A P179R | HLA-C*12:03; HLA-C*03:03; | DDTRMVRRA | 1418 |
| | HLA-C*07:02 | | |
| PPP2R1A P179R | HLA-C*12:03; HLA-A*30:01 | DTRMVRRAA | 1419 |
| PPP2R1A P179R | HLA-B*08:01; HLA-B*15:01 | DTRMVRRAAA | 1420 |
| PPP2R1A P179R | HLA-A*31:01 | DTRMVRRAAAS | 1421 |
| PPP2R1A P179R | HLA-C*12:03; HLA-C*03:03; | LCSDDTRMV | 1422 |
| | HLA-C*07:01; HLA-C*06:02 | | |
| PPP2R1A P179R | HLA-A*31:01 | LCSDDTRMVR | 1423 |
| PPP2R1A P179R | HLA-C*03:03; HLA-C*07:02; | NLCSDDTRM | 1424 |
| | HLA-C*12:03 | | |
| PPP2R1A P179R | HLA-A*02:01 | NLCSDDTRMV | 1425 |
| PPP2R1A P179R | HLA-B*08:01 | RMVRRAAA | 1426 |
| PPP2R1A P179R | HLA-C*03:03; HLA-C*12:03 | RMVRRAAAS | 1427 |
| PPP2R1A P179R | HLA-C*03:03; HLA-C*12:03; | RNLCSDDTR | 1428 |
| | HLA-A*31:01; HLA-C*07:02 | | |
| PPP2R1A P179R | HLA-A*31:01 | RNLCSDDTRMVRR | 1429 |
| PPP2R1A P179R | HLA-C*12:03; HLA-C*05:01; | SDDTRMVRR | 1430 |
| | HLA-C*08:02 | | |
| PPPSR1A P179R | HLA-B*08:01 | TRMVRRAA | 1431 |
| PPP2R1A P179R | HLA-C*12:03; HLA-B*08:01; | TRMVRRAAA | 1432 |
| | HLA-C*07:01; HLA-C*06:02; | | |
| | HLA-C*03:03; HLA-B*14:02; | | |
| | HLA-C*07:02 | | |
| PTEN R130G | HLA-C*12:03; HLA-C*03:03; | AGKGGTGVM | 1433 |
| | HLA-C*07:02 | | |
| PTEN R130G | HLA-A*31:01 | AGKGGTGVMIC | 1434 |
| PTEN R130G | HLA-C*03:03; HLA-C*12:03 | CKAGKGGTG | 1435 |
| PTEN R130G | HLA-B*07:02 | CKAGKGGTGV | 1436 |
| PTEN R130G | HLA-C*12:03; HLA-A*03:03; | GGTGVMICA | 1437 |
| | HLA-C*07:02 | | |
| PTEN R130G | HLA-B*15:01; HLA-A*29:02 | GGTGVMICAY | 1438 |
| PTEN R130G | HLA-C*12:03; HLA-C*03:03 | GKGGTGVMI | 1439 |
| PTEN R130G | HLA-A*24:02 | GTGVMICA | 1440 |
| PTEN R130G | HLA-C*12:03; HLA-C*03:03; | GTGVMICAY | 1441 |
| | HLA-A*01:01; HLA-B*15:01; | | |
| | HLA-A*29:02 | | |
| PTEN R130G | HLA-C*12:03; HLA-C*03:03 | HCKAGKGGT | 1442 |
| PTEN R130G | HLA-A*31:01 | HCKAGKGGTGV | 1443 |
| PTEN R130G | HLA-C*12:03; HLA-C*03:03 | IHCKAGKGG | 1444 |
| PTEN R130G | HLA-C*12:03; HLA-C*15:02; | KAGKGGTGV | 1445 |
| | HLA-C*05:01; HLA-C*03:03 | | |
| PTEN R130G | HLA-A*24:02 | KGGTGVMI | 1446 |
| PTEN R130G | HLA-C*12:03; HLA-C*03:03; | KGGTGVMIC | 1447 |
| | HLA-C*07:02 | | |
| PTEN R130G | HLA-A*29:02 | KGGTGVMICAY | 1448 |
| PTEN R130Q | HLA-C*12:03; HLA-C*03:03; | AGKGQTGVM | 1449 |
| | HLA-C*07:02 | | |
| PTEN R130Q | HLA-A*31:01 | AGKGQTGVMIC | 1450 |
| PTEN R130Q | HLA-C*03:03; HLA-C*12:03 | CKAGKGQTG | 1451 |
| PTEN R130Q | HLA-C*12:03; HLA-C*03:03 | GKGQTGVMI | 1452 |
| PTEN R130Q | HLA-C*12:03; HLA-C*03:03 | GQTGVMICA | 1453 |
| PTEN R130Q | HLA-B*15:01; HLA-A*29:02 | GQTGVMICAY | 1454 |
| PTEN R130Q | HLA-C*12:03; HLA-C*03:03; | HCKAGKGQT | 1455 |
| | HLA-C*07:02 | | |
| PTEN R130Q | HLA-C*12:03; HLA-C*03:03 | IHCKAGKGQ | 1456 |
| PTEN R130Q | HLA-C*12:03; HLA-C*15:02; | KAGKGQTGV | 1457 |
| | HLA-C*05:01; HLA-C*03:03 | | |
| PTEN R130Q | HLA-C*12:03; HLA-C*03:03; | KGQTGVMIC | 1458 |
| | HLA-C*07:02 | | |
| PTEN R130Q | HLA-A*29:02 | KGQTGVMICAY | 1459 |
| PTEN R130Q | HLA-A*24:02 | QTGVMICA | 1460 |
| PTEN R130Q | HLA-C*12:03; HLA-C*03:03; | QTGVMICAY | 1461 |
| | HLA-A*01:01; HLA-B*15:01; | | |
| | HLA-A*29:02 | | |
| RNF43 G659Vfs*41 | HLA-B*08:01 | ALGPRMQL | 1462 |
| RNF43 G659Vfs*41 | HLA-C*12:03; HLA-C*03:03; | ALGPRMQLC | 1463 |
| | HLA-C*07:01; HLA-C*07:02 | | |
| RNF43 G659Vfs*41 | HLA-B*08:01 | ALGPRMQLCTQ | 1464 |
| RNF43 G659Vfs*41 | HLA-A*31:01 | ALGPRMQLCTQLAR | 1465 |
| RNF43 G659Vfs*41 | HLA-C*12:03; HLA-C*03:03; | ARFFPITPP | 1466 |
| | HLA-C*07:01; HLA-C*07:02 | | |
| RNF43 G659Vfs*41 | HLA-B*27:05; HLA-A*30:01 | ARFFPITPPV | 1467 |
| RNF43 G659Vfs*41 | HLA-B*27:05 | ARFFPITPPVWHIL | 1468 |
| RNF43 G659Vfs*41 | HLA-C*12:03; HLA-C*03:03 | CTQLARFFP | 1469 |
| RNF43 G659Vfs*41 | HLA-B*27:05 | CTQLARFFPI | 1470 |
| RNF43 G659Vfs*41 | HLA-A*02:01 | CTQLARFFPITPPV | 1471 |
| RNF43 G659Vfs*41 | HLA-A*02:01 | FFPITPPV | 1472 |
| RNF43 G659Vfs*41 | HLA-C*12:03; HLA-C*07:02; | FFPITPPVW | 1473 |
| | HLA-C*03:03; HLA-A*24:02 | | |
| RNF43 G659Vfs*41 | HLA-C*03:03; HLA-C*12:03; | FPITPPVWH | 1474 |
| | HLA-B*35:01 | | |
| RNF43 G659Vfs*41 | HLA-B*51:01; HLA-A*02:01; | FPITPPVWHI | 1475 |
| | HLA-B*07:02 | | |
| RNF43 G659Vfs*41 | HLA-C*12:03 | GPRMQLCTQ | 1476 |
| RNF43 G659Vfs*41 | HLA-B*07:02; HLA-B*27:05; | GPRMQLCTQL | 1477 |
| | HLA-B*08:01 | | |
| RNF43 G659Vfs*41 | HLA-A*31:01 | GPRMQLCTQLAR | 1478 |
| RNF43 G659Vfs*41 | HLA-C*12:03; HLA-C*07:02 | GVPPSPPLA | 1479 |
| RNF43 G659Vfs*41 | HLA-B*15:01 | GVPPSPPLAL | 1480 |
| RNF43 G659Vfs*41 | HLA-C*03:03; HLA-C*12:03; | HILGPQRHT | 1481 |
| | HLA-C*07:02 | | |
| RNF43 G659Vfs*41 | HLA-B*08:01 | HPQRKRRG | 1482 |
| RNF43 G659Vfs*41 | HLA-C*12:03; HLA-B*07:02; | HPQRKRRGV | 1483 |
| | HLA-B*08:01; HLA-C*03:03 | | |
| RNF43 G659Vfs*41 | HLA-B*07:02 | HPQRKRRGVP | 1484 |
| RNF43 G659Vfs*41 | HLA-A*24:02 | ILGPQRHT | 1485 |
| RNF43 G659Vfs*41 | HLA-C*12:03; HLA-C*03:03; | ILGPQRHTP | 1486 |
| | HLA-C*07:02 | | |
| RNF43 G659Vfs*41 | HLA-C*12:03; HLA-C*16:01; | ITPPVWHIL | 1487 |
| | HLA-C*07:02; HLA-A*24:02 | | |
| RNF43 G659Vfs*41 | HLA-A*02:01 | KRRGVPPS | 1488 |
| RNF43 G659Vfs*41 | HLA-C*12:03; HLA-C*07:02; | KRRGVPPSP | 1489 |
| | HLA-A*30:01; HLA-C*07:01 | | |
| RNF43 G659Vfs*41 | HLA-C*03:03; HLA-C*03:04; | LALGPRMQL | 1490 |
| | HLA-C*16:01; HLA-C*12:03; | | |
| | HLA-B*07:02 | | |
| RNF43 G659Vfs*41 | HLA-B*27:05 | LALGPRMQLCTQL | 1491 |
| RNF43 G659Vfs*41 | HLA-C*12:03; HLA-C*03:03; | LARFFPITP | 1492 |
| | HLA-A*30:01 | | |
| RNF43 G659Vfs*41 | HLA-C*03:03; HLA-C*12:03 | LCTQLARFF | 1493 |
| RNF43 G659Vfs*41 | HLA-C*12:03; HLA-C*07:02 | LGPRMQLCT | 1494 |
| RNF43 G659Vfs*41 | HLA-B*27:05 | LGPRMQLCTQL | 1495 |
| RNF43 G659Vfs*41 | HLA-A*31:01 | LGPRMQLCTQLAR | 1496 |
| RNF43 G659Vfs*41 | HLA-A*31:01; HLA-C*12:03; | MQLCTQLAR | 1497 |
| | HLA-C*03:03 | | |
| RNF43 G659Vfs*41 | HLA-B*15:01; HLA-B*18:01; | MQLCTQLARF | 1498 |
| | HLA-B*35:01 | | |
| RNF43 G659Vfs*41 | HLA-B*15:01 | MQLCTQLARFF | 1499 |
| RNF43 G659Vfs*41 | HLA-B*13:02 | MQLCTQLARFFPI | 1500 |
| RNF43 G659Vfs*41 | HLA-C*12:03; HLA-C*03:03; | PITPPVWHI | 1501 |
| | HLA-C*07:02 | | |
| RNF43 G659Vfs*41 | HLA-A*31:01 | PITPPVWHILGPQR | 1502 |
| RNF43 G659Vfs*41 | HLA-C*03:03; HLA-C*12:03; | PLALGPRMQ | 1503 |
| | HLA-C*07:02 | | |
| RNF43 G659Vfs*41 | HLA-C*03:03; HLA-C*12:03; | PPLALGPRM | 1504 |
| | HLA-B*35:01 | | |
| RNF43 G659Vfs*41 | HLA-C*03:03; HLA-C*12:03 | PPSPPLALG | 1505 |
| RNF43 G659Vfs*41 | HLA-B*07:02 | PPSPPLALGPRMQL | 1506 |
| RNF43 G659Vfs*41 | HLA-C*12:03; HLA-C*03:03 | PPVWHILGP | 1507 |
| RNF43 G659Vfs*41 | HLA-B*08:01 | PQRKRRGV | 1508 |
| RNF43 G659Vfs*41 | HLA-C*12:03 | PQRKRRGVP | 1509 |
| RNF43 G659Vfs*41 | HLA-B*15:01 | PQRKRRGVPP | 1510 |
| RNF43 G659Vfs*41 | HLA-C*07:02; HLA-C*12:03; | PRMQLCTQL | 1511 |
| | HLA-C*06:02; HLA-C*03:03; | | |
| | HLA-C*07:01; HLA-B*27:05 | | |
| RNF43 G659Vfs*41 | HLA-B*27:05 | PRMQLCTQLA | 1512 |
| RNF43 G659Vfs*41 | HLA-A*31:01 | PRMQLCTQLAR | 1513 |
| RNF43 G659Vfs*41 | HLA-C*12:03; HLA-C*15:02 | PSPPLALGP | 1514 |
| RNF43 G659Vfs*41 | HLA-B*07:02 | PSPPLALGPRMQL | 1515 |
| RNF43 G659Vfs*41 | HLA-C*12:03; HLA-C*03:03 | PVWHILGPQ | 1516 |
| RNF43 G659Vfs*41 | HLA-B*08:01; HLA-A*02:01 | QLARFFPI | 1517 |
| RNF43 G659Vfs*41 | HLA-C*03:03; HLA-C*12:03 | QLARFFPIT | 1518 |
| RNF43 G659Vfs*41 | HLA-B*15:01 | QLARFFPITP | 1519 |
| RNF43 G659Vfs*41 | HLA-A*02:01 | QLARFFPITPPV | 1520 |
| RNF43 G659Vfs*41 | HLA-A*02:01 | QLARFFPITPPVW | 1521 |
| RNF43 G659Vfs*41 | HLA-C*12:03; HLA-B*15:01; | QLCTQLARF | 1522 |
| | HLA-C*03:03; HLA-C*05:01 | | |
| RNF43 G659Vfs*41 | HLA-B*15:01 | QLCTQLARFF | 1523 |
| RNF43 G659Vfs*41 | HLA-C*12:03; HLA-C*03:03; | QRKRRGVPP | 1524 |
| | HLA-C*07:02; HLA-B*14:02 | | |
| RNF43 G659Vfs*41 | HLA-B*27:05 | QRKRRGVPPS | 1525 |
| RNF43 G659Vfs*41 | HLA-B*27:05 | QRKRRGVPPSP | 1526 |
| RNF43 G659Vfs*41 | HLA-C*12:03; HLA-A*30:01; | RFFPITPPV | 1527 |
| | HLA-C*16:01; HLA-C*07:02; | | |
| | HLA-A*02:01; HLA-C*03:03; | | |
| | HLA-A*31:01; HLA-A*32:01 | | |
| RNF43 G659Vfs*41 | HLA-A*24:02; HLA-B*15:01; | RFFPITPPVW | 1528 |
| | HLA-A*29:02 | | |
| RNF43 G659Vfs*41 | HLA-A*24:02 | RFFPITPPVWHIL | 1529 |
| RNF43 G659Vfs*41 | HLA-C*03:03; HLA-C*07:02; | RGVPPSPPL | 1530 |
| | HLA-C*16:01; HLA-C*12:03; | | |
| | HLA-C*03:04; HLA-C*15:02; | | |
| | HLA-A*30:01 | | |
| RNF43 G659Vfs*41 | HLA-C*12:03; HLA-A*30:01; | RKRRGVPPS | 1531 |
| | HLA-C*07:02 | | |
| RNF43 G659Vfs*41 | HLA-B*07:02 | RKRRGVPPSP | 1532 |
| RNF43 G659Vfs*41 | HLA-C*12:03; HLA-A*30:01; | RMQLCTQLA | 1533 |
| | HLA-A*02:01 | | |
| RNF43 G659Vfs*41 | HLA-A*31:01; HLA-A*03:01; | RMQLCTQLAR | 1534 |
| | HLA-B*27:05; HLA-A*11:01 | | |
| RNF43 G659Vfs*41 | HLA-A*02:01; HLA-A*32:01; | RMQLCTQLARFFPI | 1535 |
| | HLA-B*27:05 | | |
| RNF43 G659Vfs*41 | HLA-A*24:02 | RRGVPPSP | 1536 |
| RNF43 G659Vfs*41 | HLA-C*12:03; HLA-C*03:03; | RRGVPPSPP | 1537 |
| | HLA-C*07:01; HLA-C*07:02 | | |
| RNF43 G659Vfs*41 | HLA-B*27:05 | RRGVPPSPPL | 1538 |
| RNF43 G659Vfs*41 | HLA-B*07:02 | SPPLALGP | 1539 |
| RNF43 G659Vfs*41 | HLA-C*12:03 | SPPLALGPR | 1540 |
| RNF43 G659Vfs*41 | HLA-B*07:02 | SPPLALGPRM | 1541 |
| RNF43 G659Vfs*41 | HLA-B*08:01; HLA-B*51:01 | SPPLALGPRMQ | 1542 |
| RNF43 G659Vfs*41 | HLA-B*07:02 | SPPLALGPRMQL | 1543 |
| RNF43 G659Vfs*41 | HLA-B*07:02 | SPPLALGPRMQLC | 1544 |
| RNF43 G659Vfs*41 | HLA-B*07:02 | SPPLALGPRMQLCT | 1545 |
| RNF43 G659Vfs*41 | HLA-B*07:02 | TPPVWHIL | 1546 |
| RNF43 G659Vfs*41 | HLA-C*12:03 | TPPVWHILG | 1547 |
| RNF43 G659Vfs*41 | HLA-B*08:01 | TPPVWHILGPQ | 1548 |
| RNF43 G659Vfs*41 | HLA-C*03:03; HLA-A*02:01; | TQLARFFPI | 1549 |
| | HLA-C*12:03; HLA-A*32:01; | | |
| | HLA-B*13:02; HLA-B*08:01; | | |
| | HLA-A*24:02; HLA-C*16:01; | | |
| | HLA-B*27:05; HLA-B*15:01 | | |
| RNF43 G659Vfs*41 | HLA-B*15:01 | TQLARFFPIT | 1550 |
| RNF43 G659Vfs*41 | HLA-A*02:01 | TQLARFFPITPPV | 1551 |
| RNF43 G659Vfs*41 | HLA-C*12:03; HLA-B*07:02; | VPPSPPLAL | 1552 |
| | HLA-B*35:01; HLA-C*03:03; | | |
| | HLA-C*07:02 | | |
| RNF43 G659Vfs*41 | HLA-C*12:03; HLA-C*03:03; | VWHILGPQR | 1553 |
| | HLA-A*31:01; HLA-C*07:02 | | |
| RNF43 G659Vfs*41 | HLA-C*03:03; HLA-C*12:03; | WHILGPQRH | 1554 |
| | HLA-C*07:02 | | |

In highly aggressive midline gliomas, a recurrent point mutation in the histone-3 gene (H3F3A) causes an amino acid change from lysine to methionine at position 27 (K27M). Ochs et al. (2017) Oncoimmunology 6(7): e1328340, incorporated by reference in its entirety herein) have shown that a peptide vaccine against K27M-mutant histone-3 is capable of inducing effective, mutation-specific, cytotoxic T-cell- and T-helper-1-cell-mediated immune responses in a major histocompatibility complex (MHC)-humanized mouse model. Accordingly, in some embodiments, the exogenous antigenic polypeptide comprises the neoantigen polypeptide H3F3A (K27M).

In some embodiments, the cancer is a cancer associated with an oncogenic virus, for example Epstein Barr virus (EBV), hepatitis B and C (HBV and HCV), human papilloma virus (HPV), Kaposi sarcoma virus (KSV), and polyoma viruses. In other certain embodiments, the cancer is a cancer where retrovirus epitopes are identified. Cancers which are associated with a virus and which may be treated using the methods of the disclosure include, but are not limited to, cervical cancer, head and neck cancer, lymphomas, and kidney clear cell carcinoma.

HLA molecules are required for the immune recognition and subsequent killing of neoplastic cells by the immune system, as tumor antigens must be presented in an HLA-restricted manner to be recognized by T-cell receptors. Some tumor cells use aberrant expression of non-classical HLA-I molecules (HLA-E and HLA-G), which function as inhibitor ligands for immune-competent cells, to escape immune recognition and facilitate tumor immune escape (Moreau et al. (2002) Cell. Mol. Life Sci. 59(9): 1460-6, incorporated by reference in its entirety herein). HLA class I histocompatibility antigen, alpha chain G (HLA-G) is a non-classical MHC class I molecule comprising a heavy a chain comprising one of more of alpha1, alpha2, and alpha3 domains.

In some embodiments, an engineered erythroid cell or enucleated cell comprises a wild-type or loadable antigen presenting polypeptide and an exogenous antigenic polypeptide, wherein the exogenous antigenic polypeptide is an HLA-G-derived polypeptide. In some embodiments, the HLA-G derived polypeptides are selected from the polypeptides shown below:

(SEQ ID NO: 1555)
HLA-G₁₄₆₋₁₅₄ - DYLALNEDL
(SEQ ID NO: 1556)
HLA-G₁₉₄₋₂₀₂ - RYLENGKEM
(SEQ ID NO: 1557)
HLA-G₁₃₉₋₁₄₈ - RYAYDGKDYL
(SEQ ID NO: 1558)
HLA-G₁₄₁₋₁₅₀ - AYDGKDYLAL

In some embodiments, the peptides shown above bind to the a specific HLA-A allele: HLA-A*24:02 (e.g., HLA-A*24:02:01:01). Therefore, in some embodiments, the wild-type or loadable antigen-presenting polypeptide comprises an HLA-A*24:02 allele (e.g., a HLA-A*24:02:01:01 allele). In some embodiments, the peptides shown above bind to other HLA class I or class II alleles described herein.

In one particular embodiment, an engineered erythroid cell or enucleated cell comprises at least one exogenous antigenic polypeptide, PR1, a HLA-A2 restricted peptide, fused to a wild-type or loadable exogenous antigen-presenting polypeptide comprising an HLA-A2 polypeptide, fused to the GPA transmembrane domain (PR1-HLA-A2-GPA). In some embodiments, the HLA-A2 polypeptide does not comprise an endogenous transmembrane domain. In some embodiments, the erythroid cell is an enucleated cell. In some embodiments, the erythroid cell is a nucleated cell.

In certain embodiments, the engineered erythroid cells or enucleated cells of the disclosure are used to treat highly vascularized tumors. Without being bound by theory, greater vascularization renders the tumors more accessible to the engineered erythroid cells or enucleated cells. Tumor vascularity can be measured, for example, by intercapillary distance (thought to reflect tumor oxygenation) and microvessel density (provides a histological assessment of tumor angiogenesis). A highly vascular tumor can be any tumor of vascular origin, for example a hemangioma, a lymphangioma, a hemangioendothelioma, Kaposi sarcoma, an angiosarcoma, a hemangioblastoma.

In other embodiments, the engineered erythroid cells or enucleated cells of the disclosure are used to treat tumors with leaky vasculature. There is general agreement that blood vessels in tumors are abnormal. One manifestation of this abnormality is a defective and leaky endothelium. Blood vessel leakiness not only influences the internal environment of tumors and perhaps the rate of angiogenesis, but it also governs access of therapeutics. Without being bound by theory, a more leaky blood vessel would provide more access to the engineered erythroid cells or enucleated cells.

Autoimmune Diseases

In certain embodiments, the engineered erythroid cells or enucleated cells of the present disclosure provide a novel and improved method of treating autoimmune diseases. The methods provided for treating autoimmune diseases have numerous advantages, including, for example, effective presentation of antigen on the surface of a cell (e.g., an engineered erythroid cell or enucleated cell) together with a wild-type or loadable exogenous antigen-presenting polypeptide, and the extremely long half-life of engineered erythroid cells or enucleated cells in circulation, thus providing extended exposure to the properly presented antigen.

In some embodiments, provided herein are methods of treating an autoimmune disease in a subject in need thereof, wherein the method comprises administering to a subject engineered erythroid cells or enucleated cells (or a pharmaceutical composition comprising the cells), wherein the engineered erythroid cells or enucleated cells comprise a first exogenous polypeptide comprising an wild-type or loadable exogenous antigen-presenting polypeptide and a second exogenous polypeptide comprising an exogenous antigenic polypeptide, wherein the exogenous antigenic polypeptide comprises or consists of an antigen associated with or that is the cause or trigger of the autoimmune disease, thereby treating the autoimmune disease. In some embodiments, provided herein are methods of treating an autoimmune disease in a subject in need thereof, wherein the method comprises administering to a subject engineered erythroid cells or enucleated cells (or a pharmaceutical composition comprising the cells), wherein the engineered erythroid cells or enucleated cells comprise a first exogenous polypeptide comprising an wild-type or loadable exogenous antigen-presenting polypeptide and a second exogenous polypeptide comprising an exogenous antigenic polypeptide, wherein the exogenous antigenic polypeptide comprises or consists of an amino acid sequence set forth in Tables 18, 19, 20 and B, or a fragment thereof, thereby treating the autoimmune disease. In some embodiments, the wild-type or loadable exogenous antigen-presenting polypeptide is bound (e.g., covalently or non-covalently attached) to the exogenous antigenic polypeptide. In some embodiments, the wild-type or loadable exogenous antigen-presenting polypeptide is not bound (e.g., covalently or non-covalently attached) to the exogenous antigenic polypeptide (e.g., are two independent polypeptides). In some embodiments, the engineered erythroid cell or enucleated cell further comprises an additional exogenous polypeptide, wherein the additional exogenous polypeptide provided herein (e.g., an exogenous polypeptide comprising at least one costimulatory polypeptide).

Any antigen associated (or fragment thereof) with or that is the cause or trigger of an autoimmune disease can be included in an exogenous antigenic polypeptide on the cells described herein. For example, the antigen may be a self-antigen to which the autoimmune response is directed. In some embodiments, the exogenous antigenic polypeptide comprises or consists of a self-antigen. In some embodiments, the exogenous antigenic polypeptide comprises or consists of antigen listed in Table 18, or an antigenic-portion thereof. In some embodiments, the exogenous antigenic polypeptide comprises or consists of an antigen listed in Table 19, or an antigenic-portion thereof. In some embodiments, the exogenous antigenic polypeptide comprises or consists of an antigens listed in Table 20, or an antigenic-portion thereof. In the provided methods for treating an autoimmune disorder, where the engineered erythroid cell or enucleated cell comprises a wild-type or loadable exogenous antigen-presenting polypeptide and an exogenous antigenic polypeptide comprising an antigen listed in Tables 18, 19 or 20, or antigenic-portion thereof, the engineered erythroid cell or enucleated cell may be administered to a subject to treat the autoimmune disorder corresponding to the antigen as provided in Tables 18, 19 or 20.

In one aspect, the engineered erythroid cells or enucleated cells are designed to suppress undesired T cell activity associated with or driving the autoimmune disorder. Thus, in some embodiments, the engineered erythroid cell or enucleated cell further comprises at least one exogenous co-inhibitory polypeptide as described herein. In some embodiments, the exogenous co-inhibitory polypeptide is selected from the co-inhibitory polypeptides listed in Table 10, or fragments thereof In some embodiments, the exogenous co-inhibitory polypeptide is selected from IL-35, IL-10, VSIG-3, and a LAG3 agonist, or a fragment thereof. In some embodiments, the co-inhibitory polypeptide suppresses an autoreactive T cell.

In another aspect, the engineered erythroid cells or enucleated cells are designed to stimulate T regulatory cells, thereby biasing the immune system back to a more tolerogenic state. Thus, in some embodiments, the engineered erythroid cell or enucleated cell further comprises at least one exogenous costimulatory polypeptide as described herein. In some embodiments, the at least one exogenous costimulatory polypeptide expands regulatory T-cells (Tregs) and is, e.g., an exogenous Treg costimulatory polypeptide as described herein. In some embodiments, the exogenous costimulatory polypeptide comprises or consists of a costimulatory polypeptide listed in Table 12, or fragments thereof. In some embodiments, the exogenous costimulatory polypeptide comprises or consists of a costimulatory polypeptide listed in Table 13, or fragments thereof.

In yet another aspect, the engineered erythroid cells or enucleated cells are designed to expand and stimulate T cells, e.g., cytotoxic CD8+ T cells. In this aspect, the autoimmune disorder is preferably an autoimmune disorder caused or triggered by an infectious agent. In some embodiments, the engineered erythroid cell or enucleated cell further comprises at least one exogenous costimulatory polypeptide as described herein. In some embodiments, the at least one exogenous costimulatory polypeptide expands cytotoxic CD8+ T cells. In some embodiments, the exogenous costimulatory polypeptide comprises or consists of a costimulatory polypeptides listed in Table 9, or fragments thereof. In some embodiments, the costimulatory polypeptides is selected from the group consisting of 4-1BBL, LIGHT, anti CD28, CD80, CD86, CD70, OX40L, IL-7, IL-12, GITRL, TIM4, SLAM, CD48, CD58, CD83, CD155, CD112, IL-15Ra fused to IL-15, IL-21, ICAM-1, a ligand for LFA-1, anti CD3, and fragments thereof, and a combination thereof.

In some aspects, the disclosure provides a method of treating a subject having an autoimmune disease, comprising administering to the subject an effective number of the erythroid cells described herein to the subject, thereby treating the autoimmune disease. In various embodiments, the autoimmune disease may be an autoimmune disease provided in Tables 18, 19, 20, or B. In such methods, the engineered erythroid cell or enucleated cell useful for treating the autoimmune disorder comprises a wild-type or loadable exogenous antigen-presenting polypeptide and an antigenic polypeptide, wherein the antigenic polypeptide may be an antigen listed in Tables 17, 18, 19, or B, or antigenic-portion thereof, wherein the engineered erythroid cell or enucleated cell comprising the antigen as provided in Tables, 17, 18, 19, or B is used to treat the corresponding autoimmune disorder listed in Tables 17, 18, 19, or B.

In some embodiments, the erythroid cell is an enucleated cell. In some embodiments, the erythroid cell is a nucleated cell.

In some embodiments, the autoimmune disease is a single antigen disease. Examples of single antigen diseases and their related antigens are shown in Table 18, below.

TABLE 18
Autoimmune diseases and associated single antigens

| Disease | Antigen |
|---|---|
| Neuromyelitis Optica (NMO) | Aquaporin 4 (AQP4) |
| Myasthenia Gravis (MG) | Acetylcholine receptor (AchR) |
| Membranous glomerulonephritis | Phospholipase A2 receptor (PLA2R) |
| Pemphigus Vulgaris (PV) | desmoglein 3 (DSG3) |
| Pemphigus Foliaceus (PF) | desmoglein 1 (DSG1) |

In another embodiments, the autoimmune disease is a multi-antigen disease. In some embodiments wherein the autoimmune disease is a multi-antigen disease, the subject may be treated with engineered erythroid cells or enucleated cells targeting more than one, e.g., two, three, four or more, of the antigens (e.g., including more than one exogenous antigenic polypeptide comprising the antigens). It will be recognized by the skilled artisan that the multiple antigens may be present on the same engineered erythroid cell or enucleated cell, or they may be present on distinct engineered erythroid cells or enucleated cells (e.g., a combination of the two, three, four or more distinct engineered erythroid cells or enucleated cells each comprising an exogenous antigenic polypeptide comprising a single antigen) are administered to the subject to treat the disorder. Examples of multi-antigen diseases and their related antigens are shown in Table 19, below.

TABLE 19
Autoimmune diseases and associated multi-antigens

| Disease | Antigen |
|---|---|
| Type I diabetes mellitus (T1DM) | Insulin/proinsulin/preproinsulin |
| Type I diabetes mellitus (T1DM) | glutamate decarboxylase (GAD65) |
| Type I diabetes mellitus (T1DM) | insulinoma antigen-2 (IA-2) |
| Multiple Sclerosis (MS) | myelin oligodendrocyte glycoprotein (MOG) |
| Multiple Sclerosis (MS) | myelin basic protein (MBP) |
| Multiple Sclerosis (MS) | proteolipid protein (PLP) |
| anti-phospholipid syndrome (APS)/ CAPS | beta-2 glycoprotein 1 (b2GP1) |
| celiac disease | A-gliadin |

In certain embodiments, the autoimmune disease is selected from the group consisting of pemphigus vulgaris, myasthenia gravis, neuromyelitis optica, bullous pemphigoid, celiac disease multiple sclerosis, type 1 diabetes, rheumatoid arthritis, and membranous glomerulonephritis.

In some embodiments, also provided are methods of treating celiac disease in a subject in need thereof, wherein the method comprises administering to a subject engineered erythroid cells or enucleated cells (or a pharmaceutical composition comprising the cells), wherein the engineered erythroid cells or enucleated cells comprise a first exogenous polypeptide comprising an wild-type or loadable exogenous antigen-presenting polypeptide and a second exogenous polypeptide comprising an exogenous antigenic polypeptide, wherein the exogenous antigenic polypeptide comprises or consists of an antigen amino acid sequence set forth in Table B, or a fragment thereof, thereby treating the celiac disease. In some embodiments, the wild-type or loadable exogenous antigen-presenting polypeptide comprises a HLA-DQα polypeptide, or a fragment thereof, and a HLA-DQβ polypeptide, or a fragment thereof, wherein the HLA-DQα polypeptide and the HLA-DQβ polypeptide comprise the following allele combinations, represented as HLA-DQα allele: HLA-DQβ allele: DQA1*05:01:DQB1*2:01; DQA1*2:01:DQB1*2:02; DQA1*03:02:DQB1*2:02; DQA1*3:01:DQB1*4:02; DQA1*03:02:DQB1*4:02; DQA1*4:01:DQB1*4:02; DQA1*1:01:DQB1*5:01; DQA1*1:02:DQB1*5:01; DQA1*1:03:DQB1*5:01; DQA1*1:04:DQB1*5:01; DQA1*1:02:DQB1*5:02; DQA1*1:03:DQB1*5:02; DQA1*1:04:DQB1*5:03; DQA1*1:02:DQB1*5:04; DQA1*1:03:DQB1*6:01; DQA1*1:02:DQB1*6:02; DQA1*1:03:DQB1*6:02; DQA1*1:04:DQB1*6:02; DQA1*1:02:DQB1*6:03; DQA1*1:03:DQB1*6:03; DQA1*1:02:DQB1*6:04; DQA1*1:02:DQB1*6:09; DQA1*2:01:DQB1*3:01; DQA1*3:01:DQB1*3:01; DQA1*03:03:DQB1*3:01; DQA1*3:01:DQB1*3:04; DQA1*03:02:DQB1*3:04; DQA1*4:01:DQB1*3:01; DQA1*05:05:DQB1*3:01; DQA1*6:01:DQB1*3:01; DQA1*3:01:DQB1*3:02; DQA1*03:02:DQB1*3:02; DQA1*02:01:DQB1*3:03; DQA1*3:02:DQB1*3:03; DQA1*03:01:DQB1*03:02; DQA1*03:02:DQB1*03:02; DQA1*04:01:DQB1*03:02 and DQA1*05:03:DQB1*03:02. In some embodiments, the wild-type or loadable exogenous antigen-presenting polypeptide comprises a HLA-DQα polypeptide, or a fragment thereof, and a HLA-DQβ polypeptide, or a fragment thereof, wherein the HLA-DQα polypeptide and the HLA-DQβ polypeptide comprise the allele combination provided in Table B, and the exogenous antigenic polypeptide comprises a corresponding antigen amino acids sequence provided in Table B

TABLE B
Antigens for Celiac

| Class II HLA | | |
|---|---|---|
| polypeptides | Antigen Source | Antigen |
| DQA1*05:01: DQB1*2:01 | α-gliadin | PFPQPELPY (SEQ ID NO: 1559); |
| (DQ2.5) | | PQPELPYPQ (SEQ ID NO: 1560); |
| | | PYPQPELPY (SEQ ID NO: 1561); |
| | | FRPEQPYPQ (SEQ ID NO: 1562) |
| | γ-gliadin | PQQSFPEQQ (SEQ ID NO: 1563); |
| | | QQPEQPYPQ (SEQ ID NO: 1564); |
| | | IQPEQPAQL (SEQ ID NO: 1565); |
| | | SQPEQEFPQ (SEQ ID NO: 1566); |
| | | PQPEQEFPQ (SEQ ID NO: 1567); |
| | | PQPEQPFCQ (SEQ ID NO: 1568); |
| | | QQPEQPFPQ (SEQ ID NO: 1569); |
| | | LQPEQPFPQ (SEQ ID NO: 1570); |
| | | QQPFPEQPQ (SEQ ID NO: 1571) |
| | ω-gliadin | PFPQPEQPF (SEQ ID NO: 1572); |
| | | PQPEQPFPW (SEQ ID NO: 1573) |
| | low molecular | PFSEQEQPV (SEQ ID NO: 1574) |
| | weight (LMW) | |
| | glutenin | |
| | LMW glutenin | FSQQQESPF (SEQ ID NO: 1575) |
| | hordein | PFPQPEQPF (SEQ ID NO: 1572); |
| | | PIPEQPQPY (SEQ ID NO: 1576); |
| | | PQPEQPFPQ (SEQ ID NO: 1577); |
| | | PYPEQPQPY (SEQ ID NO: 1578) |
| | secalin | PFPQPEQPF (SEQ ID NO: 1572); |
| | | PFPEQPEQI (SEQ ID NO: 1579); |
| | | PQPEQPFPQ (SEQ ID NO: 1577) |
| | avenin | PYPEQEEPF (SEQ ID NO: 1580); |
| | | PYPEQEQPI (SEQ ID NO: 1581); |
| | | PYPEQEQPF (SEQ ID NO: 1582) |
| DQA1*2:01: DQB1*2:02 | LMW glutenin | PFSEQEQPV (SEQ ID NO: 1574) |
| (DQ2.2) | α-gliadin | QGSVQPQQL (SEQ ID NO: 1583); |
| | | QYSQPEQPI (SEQ ID NO: 1584) |
| DQA1*02:01: DQB1*3:03; | α-gliadin | EGSFQPSQE (SEQ ID NO: 1585) |
| DQA1*3:02: DQB1*3:03; | | EQPQQPFPQ (SEQ ID NO: 1586); |
| DQA1*03:01: DQB1*03:02 | γ-gliadin | EQPQQPYPE (SEQ ID NO: 1587); |
| DQA1*03:02: DQB1*03:02 | | PQQSFPEQE (SEQ ID NO: 1588) |
| DQA1*04:01: DQB1*03:02 | high molecular | QGYYPTSPQ (SEQ ID NO: 1589) |
| DQA1*05:03: DQB1*03:02 | weight (HMW) | |
| (DQ8) | glutenin | |
| DQA1*05:03: DQB1*03:02 | α-gliadin | EGSFQPSQE (SEQ ID NO: 1585) |
| (DQ8.5) | γ-gliadin | PQQSFPEQE (SEQ ID NO: 1588) |
| | HMW glutenin | QGYYPTSPQ (SEQ ID NO: 1589) |

In some embodiments, the autoimmune disease is selected from those listed in Table 20, below.

TABLE 20
Antigens for MS, Type 1 Diabetes, RA, and Membranous Nephritis

| Disease | Known antigen |
|---|---|
| Acute rheumatic fever | cross reactive antibodies to cardiac muscle |
| alopecia areata | Trychohyalin, keratin 16 |
| ANCA-associated vasculitis | Neutrophil cytoplasmic antigen, proteinase 3, |
| | myeloperodixase, bacterial permeability increasing factor |
| autoimmune gastritis | H, K adenosine triphosphatase |
| Autoimmune hemolytic | Rh blood group antigens, 1 antigen |
| Anemia | |
| autoimmune hepatitis | nuclear protein, liver-kidney microsome type 1, liver cytosol |
| | type 1 |
| autoimmune myocarditis | cardiac myosin |
| Autoimmune thyroiditis | Thyroid peroxidase, thyroglobulin, thyroid-stimulating |
| | hormone receptor |
| Autoimmune uveitis | Retinal arrestin (S-antigen) |
| dermatomyositis | Mi2 ATPase |
| diabetes (type 1) | Pancreatic beta cell antigen |
| good pasture's syndrome | Noncollagenous domain of basement membrane collagen |
| | type IV |
| Graves' disease | Thyroid stimulating hormone receptor |
| Guillain-Barré syndrome | Neurofascin-18G, gliomedin, nodal adhesion molecules |
| Hypoglycemia | Insulin receptor |
| Idiopathic thrombocytopenic | Platelet integrin Gpllb, Gpllla |
| purpura | |
| Insulin resistant diabetes | Insulin receptor |
| Membranous nephritis | Phospholipase AZ |
| mixed essential | rheumatoid factor IgG complexes |
| cryoglobulinemia | |
| multiple sclerosis | Myelin basic protein, proteolipid protein, myelin |
| | oligodendrocyte glycoprotein |
| myasthenia gravis | Acetylcholine receptor |
| Myasthenia gravis-MUSC | Muscarinic receptor |
| pemphigus/pemphigoid | Epidermal cadherin |
| pernicious anemia | intrinsic factor (Gastric) |
| polymyositis | nuclear and nucleolar antigen |
| primary biliary cirrhosis | neutrophil nuclear antigen, mitochondrial multienzyme |
| | complex |
| psoriasis | PSO p27 |
| rheumatoid arthritis | rheumatoid factor IgG complexes, synovial joint antigen, |
| | citrullinated protein, carbamylated protein |
| scleroderma/systemic | Scl-86, nucleolar scleroderma antigen |
| sclerosis | |
| Sjogren's syndrome | SS-B, lupus La protein |
| systemic lupus erythematosus | DNAr histones, ribosomes, snRNP, scRNP |
| Vitiligo | VIT-90, VIT-75, VIT-40 |
| Wegener's granulomatosis | neutrophil nuclear antigen |
| Antiphospholipid syndrome | Beta-2 glycoprotein 1 |
| (APS) & catastrophic APS | |
| Chemotherapy induced | Neuronal antigens |
| peripheral neuropathy | |
| Atypical hemolytic uremic | Complement factor H |
| syndrome | |
| Thrombotic | ADAMTS13 |
| thrombocytopenic purpura | |

In certain embodiments, an engineered erythroid cell or enucleated cell described herein is used to drive tolerance induction in subjects with Type I diabetes. In one particular embodiment, an engineered erythroid cell or enucleated cell comprises a first exogenous polypeptide comprising an wild-type or loadable exogenous antigen-presenting polypeptide and a second exogenous polypeptide comprising an exogenous antigenic polypeptide, wherein the exogenous antigenic polypeptide comprises or consists of insulin B-chain, and in particular, a portion of the insulin B-chain. In certain embodiments, the portion of the insulin B-chain is amino acids 9-23 of insulin B-chain. In some embodiments, the erythroid cell is an enucleated cell. In some embodiments, the erythroid cell is a nucleated cell.

In some embodiments, the autoimmune disease is a disease of immune activation. Diseases of immune activation also include inflammatory diseases, such as, e.g. Crohn's disease, ulcerative colitis, celiac disease, or other idiopathic inflammatory bowel disease. Diseases of immune activation also include allergic diseases, such as, e.g. asthma, peanut allergy, shellfish allergy, pollen allergy, milk protein allergy, insect sting allergy, and latex allergy, animal dander allergy, black and English walnut allergy, brazil nut allergy, cashew nut allergy, chestnut allergy, dust mite allergy, egg allergy, fish allergy, hazelnut allergy, mold allergy, pollen allergy, grass allergy, shellfish allergy, soy allergy, tree nut allergy and wheat allergy.

Diseases of immune activation also include immune activation in response to a therapeutic protein administered to treat a primary condition, that lessens the efficacy of therapeutic protein, such as, e.g., clotting factor VIII in hemophilia A, clotting factor IX in hemophilia B, antitumor necrosis factor alpha (TNFa) antibodies in rheumatoid arthritis and other inflammatory diseases, glucocerebrosidase in Gaucher's disease, any recombinant protein used for enzyme replacement therapy, or asparaginase in acute lymphoblastic leukemia (ALL).

In some embodiments a subject is suffering from an autoimmune disease or condition or a self-antibody mediated disease or condition, in which the subject's immune system is active against an endogenous (self) molecule, for example a protein antigen, such that the immune system attacks the endogenous molecule, induces inflammation, damages tissue, and otherwise causes the symptoms of the autoimmune or self-antibody disease or conditions. The immune response might be driven by antibodies that bind to the endogenous molecule, or it may be driven by overactive T cells that attack cells expressing the endogenous molecule, or it may be driven by other immune cells such as regulatory T cells, NK cells, NKT cells, or B cells. In these embodiments, an antigenic protein or a fragment thereof corresponding to the endogenous (self) molecule may be expressed on an engineered erythroid cell or enucleated cell comprising an erythroid cell, presenting (e.g. comprising on the cell surface) one or more exogenous polypeptides, as described herein. The engineered erythroid cells or enucleated cells, when administered once or more to the subject suffering from the disease or condition, would be sufficient to induce tolerance to the antigenic protein such that it no longer induced activation of the immune system, and thus would treat or ameliorate the symptoms of the underlying disease or condition. In certain embodiments, the engineered erythroid cells or enucleated cells are used to stimulate T regulatory cells, thereby biasing the immune system back to a more tolerogenic state for the endogenous (self) molecule.

In some embodiments, a subject is suffering from an allergic disease, for example an allergy to animal dander, black walnut, brazil nut, cashew nut, chestnut, dust mites, egg, English walnut, fish , hazelnut, insect venom, latex, milk, mold, peanuts, pollen, grass, shellfish, soy, tree nuts, or wheat. A subject suffering from an allergy may mount an immune response upon contact with the antigenic fragment of the allergen, for example through diet, skin contact, injection, or environmental exposure. The immune response may involve IgE antibody, sensitized mast cells, degranulation, histamine release, and anaphylaxis, as well as canonical immune cells like T cells, B cells, DCs, T regulatory cells, NK cells, neutrophils, and NKT cells. The allergic reaction may cause discomfort or it may be severe enough to cause death, and thus requires constant vigilance on the part of the sufferer as well as his or her family and caretakers. In these embodiments, the antigenic protein or a fragment thereof may be presented on an erythroid cell of the engineered erythroid cell or enucleated cell (e.g., as part of an exogenous antigenic polypeptide described herein). A population of these cells, when administered once or more to the subject suffering from the allergic disease or condition, would be sufficient to induce tolerance to the antigenic protein such that it no longer induced activation of the immune system upon exposure, and thus would treat or ameliorate the symptoms of the underlying allergic disease or condition.

Autoimmune Diseases Associated with an Infectious Agent

In some embodiments, the disclosure provides methods for treating an autoimmune disease or disorder associated with or triggered by an infectious agent. Exemplary autoimmune diseases or disorders associated with or triggered by infectious agents are provided in Table 21.

TABLE 21
Autoimmune diseases and associated infectious agents.

| Autoimmune Disease | Infectious Agent(s) |
|---|---|
| Allergic encephalitis | Measles virus |
| Autoimmune kidney disease | Streptococcal infections |
| Chagas disease | Trypanosoma cruzi |
| Chronic autoimmune hepatitis | Hepatitis C virus |
| Guillain-Barré syndrome | Campylobacter jejuni, Cytomegalovirus, |
| | Zika virus |
| Herpetic stromal keratitis | Herpes simplex virus |
| HTLV-associated myelopathy | Human T-cell leukemia virus |
| Lyme arthritis | Borrelia burgdorferi |
| Mixed cryoglobulinemia | Hepatitis C virus |
| Myocarditis | Coxsackie virus B3 |
| Pediatric autoimmune | Streptococcal infections |
| neuropsychiatric disorders | |
| Polyarteritis nodosa | Hepatitis B virus |
| Primary biliary cirrhosis | Escherichia coli |
| Reactive arthritis | Yersinia enterocolitica |
| Reiter's syndrome | Chlamydia trachomatis, Shigella species |
| Rheumatic fever | Streptococcus pyogenes |
| Rheumatic heart disease | Streptococci |
| Rheumatoid arthritis | Normal gut flora |
| Scleroderma | Cytomegalovirus |
| Tourette syndrome | infections |
| Type 1 diabetes | Enterovirus, Rotavirus |
| Type 1 diabetes mellitus | Coxsackie virus B4 |

Any autoimmune disorder associated with an infectious agent, including without limitation those disorders presented in Table 21, is contemplated to be treated with an engineered erythroid cell or enucleated cell described herein.

As provided by the present disclosure, an autoimmune disease associated with an infectious agent may be treated by administering to the subject an engineered erythroid cell or enucleated cell comprising a first exogenous polypeptide comprising an wild-type or loadable exogenous antigen-presenting polypeptide and a second exogenous polypeptide comprising an exogenous antigenic polypeptide, wherein the exogenous antigenic polypeptide comprises or consists of an antigen or fragment thereof from the infectious agent (i.e., targeting the specific infectious agent triggering the disease).

In a particular embodiment, the engineered erythroid cell or enucleated cell useful for treating an autoimmune disorder associated with an infectious agent further comprises an exogenous costimulatory polypeptide. In some embodiments, the exogenous costimulatory polypeptide activates cytotoxic CD8+ T cells in order to target and eliminate cells infected with the infectious agent. For example, the cytotoxic CD8+ T cells may target, suppress and/or eliminate autoreactive B cells infected with the infectious agent. The exogenous costimulatory polypeptide may include costimulatory polypeptide described herein. In some embodiments, the exogenous costimulatory polypeptide expands cytotoxic CD8+ T cells. In some embodiments, the exogenous costimulatory polypeptide comprises or consists of a costimulatory polypeptides listed in Table 9. In some embodiments, the costimulatory polypeptides icomprises or consists of 4-1BBL, LIGHT, anti CD28, CD80, CD86, CD70, OX40L, IL-7, IL-12, GITRL, TIM4, SLAM, CD48, CD58, CD83, CD155, CD112, IL-15Ra fused to IL-15, IL-21, ICAM-1, a ligand for LFA-1, anti CD3, a fragment thereof, and a combination thereof.

In some embodiments, the autoimmune disease associated with an infectious agent is multiple sclerosis (MS). Several infectious agents have been associated with MS. Exemplary infectious agents associated with MS are provided in Table 22. In some embodiments, the infectious agent associated with the autoimmune disorder is a virus. In some embodiments, the engineered erythroid cell or enucleated cell for use in the treatment of MS comprise a first exogenous polypeptide comprising an wild-type or loadable exogenous antigen-presenting polypeptide and a second exogenous polypeptide comprising an exogenous antigenic polypeptide, wherein the exogenous antigenic polypeptide comprises or consists of an amino acid sequence set forth in Table 22.

TABLE 22
Infectious agents associated with multiple
sclerosis (MS)

| Infectious Agent | Antigenic polypeptides |
|---|---|
| Borna virus | |
| Coronavirus | |
| Cytomegalovirus | |
| EBV | VLQWASLAV (SEQ ID NO: 868), |
| | FMVFLQTHI (SEQ ID NO: 869), |
| | FLQTHIFAEV (SEQ ID NO: 870), |
| | or |
| | SIVCYFMVFL (SEQ ID NO: 871) |
| Hepatitis B virus | |
| Herpes simplex virus, | |
| type 1 | |
| Herpes simplex virus, | |
| type 2 | |
| HHV-6 | PRTPPPS (SEQ ID NO: 1590) |
| HTLV-1 | |
| Influenza A virus | |
| LM7 virus | |
| Measles virus | |
| MS1533 | |
| MSRV (HERV-W) | |
| Parainfluenza virus 1 | |
| Rabies virus | |
| Rubella virus | |
| Scrapie agent | |
| Simian virus 5 | |
| SMON-like | virus |
| Tick borne | |
| encephalitis virus | |
| Torque Teno virus | |
| VZV | |

In some embodiments, the present disclosure provides a method of treating a subject having MS, comprising administering to the subject an effective number of engineered erythroid cells or enucleated cells comprise a first exogenous polypeptide comprising an wild-type or loadable exogenous antigen-presenting polypeptide and a second exogenous polypeptide comprising an exogenous antigenic polypeptide, wherein the exogenous antigenic polypeptide comprises or consists of an antigen from an infectious agent listed in Table 22, or an immunogenic peptide thereof, thereby treating the MS. In some embodiments, the engineered erythroid cells or enucleated cells further comprise an exogenous costimulatory polypeptide.

In a particular embodiment, the infectious agent associated with MS is Epstein-Barr virus (EBV). While not wishing to be bound by any particular theory, it is believed that during primary infection, EBV infects autoreactive naive B cells in tonsils driving them to enter germinal centers where they proliferate intensely and differentiate into latently infected autoreactive memory B cells, which then exit from the tonsils and circulate in the blood. The number of EBV-infected B cells is normally controlled by EBV-specific cytotoxic CD8+ T cells, which kill proliferating and lytically infected B cells, but not if there is a defect in this defense mechanism. Surviving EBV-infected autoreactive memory B cells enter the CNS where they take up residence and produce oligoclonal IgG and pathogenic autoantibodies, which attack myelin and other components of the CNS. Autoreactive T cells that have been activated in peripheral lymphoid organs by common systemic infections circulate in the blood and enter the CNS where they are reactivated by EBV-infected autoreactive B cells presenting CNS peptides (Cp) bound to major histocompatibility complex (MEW) molecules. These EBV-infected B cells provide costimulatory survival signals (B7) to the CD28 receptor on the autoreactive T cells and thereby inhibit activation-induced T-cell apoptosis, which normally occurs when autoreactive T cells enter the CNS and interact with nonprofessional APCs such as astrocytes and microglia which do not express B7 costimulatory molecules. After the autoreactive T cells have been reactivated by EBV-infected autoreactive B cells, they produce cytokines such as interleukin-2 (IL-2) interferon-γ (IFNγ) and tumor necrosis factor (TNF) and orchestrate an autoimmune attack on the CNS with resultant oligodendrocyte and myelin destruction.

As provided herein, there are several alternative ways in which an engineered erythroid cell or enucleated cell may be designed and used to treat an autoimmune disease associated with or triggered by an infectious agent in a subject. For example, an autoimmune disease associated with an infectious agent may alternatively be treated by administering to the subject an engineered erythroid cell or enucleated cell comprising a first exogenous polypeptide comprising a wild-type or loadable exogenous antigen-presenting polypeptide, a second exogenous polypeptide comprising an exogenous antigenic polypeptide, and optionally a third exogenous polypeptide comprising at least one co-inhibitory polypeptide, or at least one Treg costimulatory polypeptide (also referred to herein as Treg expansion polypeptide). In some embodiments, the exogenous antigenic polypeptide comprises an antigen, or antigenic fragment thereof, from the infectious agent (i.e., targeting the infectious agent triggering the disease). In some embodiments, the antigen or antigenic fragment thereof is an antigen associated with the infection-induced autoimmune disorder (e.g., a self-polypeptide).

In some embodiments, as described above more generally for any autoimmune disorder, the engineered erythroid cell or enucleated cell useful for treating an autoimmune disorder associated with an infectious agent further comprises an exogenous Treg costimulatory polypeptide. The exogenous Treg costimulatory polypeptide may be any Treg expansion polypeptide described herein. In an embodiment, the exogenous Treg costimulatory polypeptide induces the expansion of Tregs that, in turn, suppress T cells generated in the subject in response to the infectious agent.

In some embodiments, as described above more generally for any autoimmune disorder, the engineered erythroid cell or enucleated cell useful for treating an autoimmune disorder associated with an infectious agent comprises an exogenous co-inhibitory polypeptide. The exogenous co-inhibitory polypeptide may comprise or consist of any co-inhibitory polypeptide described herein. In some embodiments, the exogenous co-inhibitory polypeptide suppresses T cells generated in the subject in response to the infectious agent.

In another embodiment, the autoimmune disease is not associated with an infectious agent. For an autoimmune disease that is not associated with an infectious agent, one of skill in the art will recognize, based on the disclosure elsewhere herein, that the autoimmune disease may be treated by administering to the subject an engineered erythroid cell or enucleated cell comprising a first exogenous polypeptide comprising a wild-type or loadable exogenous antigen-presenting polypeptide, a second exogenous polypeptide comprising an exogenous antigenic polypeptide comprising an antigen or fragment thereof from the endogenous (self) polypeptide to which the autoimmune activity is directed, and a third exogenous polypeptide comprising at least one coinhibitory polypeptide or at least one Treg expansion polypeptide. In these embodiments, the engineered erythroid cells or enucleated cells are administered to the subject in order to induce peripheral tolerance to the antigen triggering the autoimmune response.

Infectious Diseases

The engineered erythroid cells or enucleated cells provided herein may be used for the treatment of an infectious disease in a subject in need thereof. Accordingly, in some aspects, the disclosure provides a method of treating a subject having an infectious disease, comprising administering to the subject an effective number or amount of the engineered erythroid cells or enucleated cells described herein to the subject, thereby treating the infectious disease.

In some embodiments, provided herein are methods of treating an infectious disease in a subject in need thereof, wherein the method comprises administering to a subject engineered erythroid cells or enucleated cells (or a pharmaceutical composition comprising the cells) , wherein the engineered erythroid cells or enucleated cells comprise a first exogenous polypeptide comprising an wild-type or loadable exogenous antigen-presenting polypeptide and a second exogenous polypeptide comprising an exogenous antigenic polypeptide, wherein the exogenous antigenic polypeptide comprises or consists of an antigen from a pathogen or infectious agent, thereby treating the infectious disease. In various embodiments, the antigen is an antigen of a pathogen, e.g., a viral pathogen, a bacterial pathogen, a fungal pathogen, or a parasitic pathogen. In some embodiments, the engineered erythroid cell or enucleated cell comprises a first exogenous polypeptide comprising a wild-type or loadable exogenous antigen-presenting polypeptide, a second exogenous polypeptide comprising an exogenous antigenic polypeptide comprising a first antigen (e.g., first antigen of a pathogen), and the third exogenous polypeptide comprises a second exogenous antigenic polypeptide comprising a second antigen (e.g, second antigen from a pathogen). The two antigens can be derived from two different proteins, or they may be derived from the same protein. For example, in some embodiments, the first antigen or the second antigen is an HPV-E6 antigen. In some embodiments, the first antigen or the second antigen is an HPV-E7 antigen. In some embodiments, the first antigen is an HPV-E6 antigen and the second antigen is an HPV-E7 antigen. In some embodiments, the wild-type or loadable exogenous antigen-presenting polypeptide is bound (e.g., covalently or non-covalently attached) to the exogenous antigenic polypeptide. In some embodiments, the wild-type or loadable exogenous antigen-presenting polypeptide is not bound (e.g., covalently or non-covalently attached) to the exogenous antigenic polypeptide (e.g., are two independent polypeptides). In some embodiments, the engineered erythroid cell or enucleated cell further comprises an additional exogenous polypeptide, wherein the additional exogenous polypeptide provided herein (e.g., an exogenous polypeptide comprising at least one costimulatory polypeptide).

In certain embodiments, the present disclosure provides an engineered erythroid cell or enucleated cell comprising an erythroid cell (e.g. an enucleated erythroid cell) or an enucleated cell, comprising one or more exogenous polypeptides, wherein one of the one or more exogenous polypeptides comprises an infectious disease therapeutic. In some embodiments, the erythroid cell is an enucleated erythroid cell. In some embodiments, the erythroid cell is a nucleated erythroid cell.

“Infectious disease therapeutic” as used herein, refers to an exogenous polypeptide which inhibits an infectious disease, e.g., reduces or alleviates a cause or symptom of an infectious disease, or improves a value for a parameter associated with the infectious disease, e.g., viral load or bacterial load. In some embodiments, the infectious disease therapeutic is a first or second exogenous polypeptide, which when present or expressed with the other exogenous polypeptide, inhibits an infectious disease. In an embodiment, a first or second infectious disease therapeutic has activity in the absence of the other. In some embodiments, the infectious disease therapeutic inhibits the infectious disease directly, e.g., by killing pathogens. In some embodiments, the infectious disease therapeutic inhibits the infectious disease by stimulating a subject's immune response, e.g., as a vaccine.

In some embodiments, the engineered erythroid cell or enucleated cell comprises a first exogenous polypeptide comprising a first infectious disease therapeutic and a second exogenous polypeptide, comprising a second infectious disease therapeutic. In some embodiments, the engineered erythroid cell or enucleated cell comprises a first exogenous polypeptide comprising a first infectious disease therapeutic, a second exogenous polypeptide, comprising a second infectious disease therapeutic, and a third exogenous polypeptide, comprising a third infectious disease therapeutic.

The first, second and third infectious disease therapeutic can act on the same target, for example a cell surface receptor and/or an endogenous human protein. Alternatively, the first, second and third anti-cancer therapeutic can act on different targets. The first, second or third targets may be members of the same biological pathway, wherein optionally the targets are cell surface receptors, endogenous human proteins. The first, second or third targets may be on different cell types. In some embodiments, the first exogenous polypeptide localizes the engineered erythroid cell to a desired site, e.g., a human cell, and the second exogenous polypeptide has a therapeutic activity, e.g., antigen-presenting activity.

In certain preferred embodiments, the infectious disease therapeutic is an antigen, e.g., an antigen from a pathogen or infectious agent (where “pathogen” and “infectious agent” are used interchangeably herein). In some embodiments, the first therapeutic is an antigen, e.g., an antigen from a pathogen. In some embodiments, the first therapeutic and second therapeutic is an antigen, e.g., an antigen from a pathogen. In certain embodiments, the first, second and third therapeutic is an antigen, e.g., an antigen from a pathogen.

In some embodiments, the subject may be treated with engineered erythroid cells or enucleated cells comprising more than one, e.g., two, three, four, five or more, different exogenous antigenic polypeptides. It some embodiments the multiple exogenous antigenic polypeptides may be present on the same engineered enucleated erythroid cell. In some embodiments, the multiple exogenous antigenic polypeptides may be present on two or more distinct engineered enucleated erythroid cells, wherein a combination of the distinct engineered enucleated erythroid cells are administered to the subject to treat the disorder.

The exogenous antigenic polypeptide may include any pathogenic antigen, or antigenic-portion thereof, known in the art. Exemplary pathogenic antigens which may be included in one or more exogenous antigenic polypeptides on an erythroid cell or enucleated cell described herein are described in detail below, but are not intended to be limiting. In various embodiments, the exogenous antigenic polypeptide includes an antigen from a pathogen provided in any one of Tables 21, 22, 23 and 24 below. It will be recognized by the skilled artisan that an engineered erythroid cell or enucleated cell as provided herein, comprising an exogenous antigenic polypeptide comprising an antigen from a particular pathogen or infectious agent, can be administered to a subject to treat an infection in the subject caused by that pathogen or infectious agent. It will be further recognized by the skilled artisan that an engineered erythroid cell or enucleated cell as provided herein, comprising an exogenous antigenic polypeptide comprising an antigen from a particular pathogen or infectious agent, can be administered to a subject to treat a disease or disorder in the subject, wherein the disease, disorder is caused, directly or indirectly, by infection with that pathogen or infectious agent.

In some aspects, the disclosure provides an method of treating a subject having an infectious disease, comprising administering to the subject an effective number of the erythroid cells described herein to the subject, thereby treating the infectious disease.

In another aspect, the present disclosure provides a method of treating a subject having an infectious disease, comprising administering to the subject an effective number of engineered erythroid cells or enucleated cells comprising an erythroid cell (e.g., an enucleated erythroid cell) or an enucleated cell, comprising one or more exogenous polypeptides, wherein one of the one or more exogenous polypeptides comprises a pathogenic antigen, thereby treating the infectious disease.

In certain embodiments, the infectious disease therapeutic is selected from an antimicrobial polypeptide listed on a publicly available bioinformatic database, such as CAMP, CAMP release 2 (Collection of sequences and structures of antimicrobial peptides), the Antimicrobial Peptide Database (available on the world wide web at aps.unmc.edu/AP/main.php), LAMP, BioPD, and ADAM (A Database of Anti-Microbial peptides) (available on the world wide web at bioinformatics.cs.ntou.edu.tw/adam/). The Antimicrobial peptide databases may be divided into two categories on the basis of the source of peptides it contains, as specific databases and general databases. These databases have various tools for antimicrobial peptides analysis and prediction. For example, CAMP contains AMP prediction, feature calculator, BLAST search, clustalW, VAST, PRATT, Helical wheel etc. In addition, ADAM allows users to search or browse through AMP sequence-structure relationships.

In certain embodiments, the infectious disease therapeutic is selected from a viral polypeptide. In these embodiments, an engineered erythroid cell or enucleated cell comprises an erythroid cell (e.g., an enucleated erythroid cell) or an enucleated cell, comprising one or more exogenous polypeptides, wherein one of the one or more exogenous polypeptides comprises an antigenic viral polypeptide, and the engineered erythroid cell or enucleated cell is administered to a subject to treat an infection with the virus.

Viral infections include adenovirus, coxsackievirus, hepatitis A virus, poliovirus, Epstein-Barr virus, herpes simplex type 1, herpes simplex type 2, human cytomegalovirus, human herpesvirus type 8, varicella-zoster virus, hepatitis B virus, hepatitis C viruses, human immunodeficiency virus (HIV), influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza virus or thogotovirus), Parvoviridae (e.g., parvovirus), Picomaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). Specific examples of these viruses include human respiratory coronavirus, influenza viruses A-C, hepatitis viruses A to G, and herpes simplex viruses 1-9.

Exemplary viral pathogens are shown below in Table 23. In certain embodiments, the viral pathogen is selected from Hepatitis B virus, Hepatitis C virus, Epstein Barr virs, Cytomegalovirus (CMV).

TABLE 23
Viral Pathogens

| Baltimore | Examples, by Family | |
|---|---|---|
| classification | (Human Host) | Species/Pathology example |
| dsDNA viruses | Adenoviridae | Example - Respiratory infection |
| dsDNA viruses | Polyomaviridae | Example - progressive multifocal |
| | | leukoencephalopathy |
| dsDNA viruses | Papiliomaviradae | Example - betapapilloma virus (warts, malignant |
| | | tumors) |
| dsDNA viruses | Poxviridae | Example - Molluscum contagiosum (skin lesions) |
| dsDNA viruses | Herpesviridae | Example - Varicellovirus (Chickenpox) |
| ssDNA viruses (+strand | Anelloviridae | Asymptomatic, may be associate with hepatitis, |
| or “sense”) DNA | | pulmonary disease, hematological disorders, |
| | | myopathy, lupus |
| ssDNA viruses (+strand | Parvoviridae | Example - Fifth disease |
| or “sense”) DNA | | |
| dsRNA viruses | Reoviridae | Example - Colorado tick fever |
| (e.g. Reoviruses) | | |
| (+)ssRNA viruses | Coronaviridae | Example - Pneumonia, gastroenteritis |
| (+strand or sense) RNA | | |
| (+)ssRNA viruses | Picornaviridae | Example - Myocarditis |
| (+strand or sense) RNA | | |
| (+)ssRNA viruses | Astoviridae | Example - infantile gastroenteritis |
| (+strand or sense) RNA | | |
| (+)ssRNA viruses | Caliciviridae | Example - Norovirus - gastroenteritis |
| (+strand or sense) RNA | | |
| (+)ssRNA viruses | Flaviviridae | Example - Dengue, Zika |
| (+strand or sense) RNA | | |
| (+)ssRNA viruses | Hepeviridae | Example - Hepatitis |
| (+strand or sense) RNA | | |
| (+)ssRNA viruses | Togaviridae | Example - Rubella |
| (+strand or sense) RNA | | |
| (−)ssRNA viruses | Rhabdoviridae | Example - Rabies |
| (−strand or antisense) RNA | | |
| (−)ssRNA viruses | Filoviridae | Example - Ebola, Marburg |
| (−strand or antisense) RNA | | |
| (−)ssRNA viruses | Paramyxoviridae | Example - Mumps |
| (−strand or antisense) RNA | | |
| (−)ssRNA viruses | Pneumoviridae | Example - Respiratory tract infection |
| (−strand or antisense) RNA | | |
| (−)ssRNA viruses | Arenaviridae | Example - Encephalitis, Hemorrhagic fever |
| (−strand or antisense) RNA | | |
| (−)ssRNA viruses | Bunyaviridae | Example - Hantavirus pulmonary syndrome/ |
| (−strand or antisense) RNA | | hemorrhagic fever |
| (−)ssRNA viruses | Deltavirus | Example - hepatitis, cirrhosis |
| (−strand or antisense) RNA | | |
| (−)ssRNA viruses | Orthomyxoviridae | Example - Influenza A, Influenza B |
| (−strand or antisense) RNA | | |
| ssRNA-RT viruses | Retroviridae | Example - lentivirus - HIV |
| (+strand or sense) RNA | | |
| with DNA intermediate | | |
| in life-cycle | | |
| dsDNA-RT viruses | Hepadnaviridae | Example - hepatitis, cirrhosis, hepatocellular |
| | | carcinoma |

In certain embodiments, the exogenous antigenic polypeptides for use in treating an infectious disease comprise antigens selected from viral, retroviral, and testes antigens. For example, in certain embodiments, the virus is selected from Epstein Barr virus (EBV), hepatitis B (HBV), hepatitis C (HCV), human papilloma virus (HPV), Kaposi sarcoma virus (KSV), and polyoma viruses. In some embodiments, the virus is hepatitis B (HBV).

In some embodiments, the present disclosure provides a method of treating a subject having a cancer associated with an oncogenic virus (e.g. HPV), comprising administering to the subject an effective number of engineered erythroid cells or enucleated cells, comprising one or more exogenous antigenic polypeptides, wherein one of the one or more exogenous antigenic polypeptides comprises an HPV antigen from Table 8. In some embodiments, the present disclosure provides a method of treating a subject having a cancer associated with an oncogenic virus (e.g. HPV), comprising administering to the subject an effective number of engineered erythroid cells or enucleated cells, comprising one or more exogenous antigenic polypeptides, wherein one of the one or more exogenous antigenic polypeptides comprises an HPV-E7 antigen. In some embodiments, the present disclosure provides a method of treating a subject having a cancer associated with an oncogenic virus (e.g. HPV), comprising administering to the subject an effective number of engineered erythroid cells or enucleated cells, comprising two or more exogenous antigenic polypeptides, wherein the two or more exogenous antigenic polypeptides comprises an HPV-E7 antigen and an HPV-E6 antigen.

In some embodiments, the present disclosure provides a method of treating a subject having a viral infection, comprising administering to the subject an effective number of engineered erythroid cells or enucleated cells, comprising one or more exogenous antigenic polypeptides, wherein one of the one or more exogenous antigenic polypeptides comprises an antigen from an infectious agent listed in Table 23, or an immunogenic peptide thereof, thereby treating the viral infection. In some embodiments, the engineered erythroid cells or enucleated cells further comprise a wild-type or loadable exogenous antigen-presenting polypeptide, and optionally an exogenous costimulatory polypeptide.

In some embodiments, the present disclosure provides methods for treating hepatitis B infection, e.g., chronic HBV infection. It is estimated that up to 250 million people worldwide have chronic hepatitis B, which is primarily transmitted in utero or during childbirth. Chronic HBV causes cirrhosis and hepatocellular carcinoma. The pathology of HBV may be due to hepatitis B antigen-specific T cell recruitment of nonantigen-specific T cells, which secrete cytokines causing liver damage. It has been proposed that exhausted T cells are key to the etiology of chronic hepatitis. HBV-specific T cells have been reported to be reactivated by PD1 blockade, e.g., by anti-PD1, and/or IL-12 (Schirdewahn et al. (2017) J. Infect. Dis. 215(1): 139-49; Fisicaro etal. (2010) Gastroenterology 138(2): 682-93, 693.e1-4; and Schurich etal. (2013) PLOS Pathogens 9(3): e1003208; the entire contents of each of which is incorporated herein by reference). Thus, it is envisioned by the present disclosure that engineered erythroid cells or enucleated cells provided herein may be used to treat chronic HBV by reactivating HBV-specific T cells, e.g., for cytolytic or non-cytolytic anti-viral activity.

Accordingly, in some embodiments, the present disclosure provides a method of treating a subject having a hepatitis B viral (HBV) infection, e.g., chronic hepatitis B, comprising administering to the subject an effective number of engineered erythroid cells or enucleated cells, comprising one or more exogenous antigenic polypeptides, wherein one of the one or more exogenous antigenic polypeptides comprises an HBV-specific antigen, or an immunogenic peptide thereof, thereby treating the HBV infection. In some embodiments, the HBV infection is a chronic HBV infection. In some embodiments, the engineered erythroid cells or enucleated cells further comprise a wild-type or loadable exogenous antigen-presenting polypeptide. In some embodiments, the engineered erythroid cell or enucleated cell further comprises at least one exogenous costimulatory polypeptide. In some embodiments, the at least one exogenous costimulatory polypeptide comprises 4-1BBL, IL-2, IL-12, IL-15, IL-18, IL-21, a fragment thereof, and any combination thereof, e.g., IL-12 and IL-15, or 4-1BBL and IL-15. In some embodiments, the engineered erythroid cell or enucleated cell further comprises an additional exogenous polypeptide, wherein the additional exogenous polypeptide comprises a checkpoint inhibitor. In some embodiments, the checkpoint inhibitor is an antibody molecule to PD1. In a particular embodiment, the engineered erythroid cell or enucleated cell comprises an erythroid cell comprising one or more exogenous polypeptides, wherein the one or more exogenous polypeptides comprise: an exogenous antigenic polypeptide comprising an HBV-specific antigen, or an immunogenic peptide thereof, a wild-type or loadable exogenous antigen-presenting polypeptide, an exogenous costimulatory polypeptide, e.g., comprising IL-12 or 4-1BBL, and an exogenous polypeptide comprising a checkpoint inhibitor, e.g., antibody to PD1.

In certain embodiments, the infectious disease therapeutic is selected from a bacterial polypeptide. In these embodiments, an engineered erythroid cell or enucleated cell comprises an erythroid cell (e.g., an enucleated erythroid cell) or an enucleated cell, comprising one or more exogenous antigenic polypeptides, wherein one of the one or more exogenous antigenic polypeptides comprises an antigenic bacterial polypeptide, and the engineered erythroid cell or enucleated cell is administered to a subject to treat an infection with the bacteria.

Bacterial infections include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Eschericia coli, Bacillus anthracis, Rickettsia, Bartonella henselae, Bartonella quintana, Coxiella burnetii, chlamydia, Mycobacterium leprae, Salmonella; shigella; Yersinia enterocolitica; Yersinia pseudotuberculosis; Legionella pneumophila; Mycobacterium tuberculosis; Listeria monocytogenes; Mycoplasma spp.; Pseudomonas fluorescens; Vibrio cholerae; Haemophilus influenzae; Bacillus anthracis; Treponema pallidum; Leptospira; Borrelia; Corynebacterium diphtheriae; Francisella; Brucella melitensis; Campylobacter jejuni; Enterobacter; Proteus mirabilis; Proteus; and Klebsiella pneumoniae.

Exemplary bacterial pathogens are shown below in Table 24.

TABLE 24
Bacterial Pathogens

| Phylum | By Genus | Species/Pathology Example |
|---|---|---|
| Proteobacteria | Erlichia | Erlichosis (Pain, malaise, fever gastrointestinal disorder, |
| | | confusion rash. Can be fatal) |
| Proteobacteria | Rickettsia | Two groups of diseases from different subspecies - |
| | | Spotted fever; Typhus |
| Proteobacteria | Brucella | Brucellosis. 4 known species cause disease in humans. |
| | | Can be sporadic or chronic. Fever, malaise, lesions |
| Proteobacteria | Bartonella | Multiple species. Pathophysiology includes endocarditis, |
| | | neuroretinitis, cat scratch disease, peliosis hepititus, |
| | | general bactermia angiomatosis, Carrion's disease, |
| | | Trench fever |
| Proteobacteria | Bordetella | Multiple species. Adherence to ciliated ephilium drives |
| | | respiratory tract infections. B. pertussis = subspecies |
| | | example |
| Proteobacteria | Neisseria | Includes N meningitidis (bacterial meningitis and |
| | | septicemia) and N gonorrheae (Gonorrhea) - colonize |
| | | mucosal surfaces |
| Proteobacteria | Francisella | Examples include f. tularensis (tularemia), F novicida, |
| | | F philomiragia (septicemia) |
| Proteobacteria | Legionella | Examples include L pneumophila - legionnaires disease |
| | | and pontiac fever |
| Proteobacteria | Coxiella | Coxiella burnetii - Q Fever |
| Proteobacteria | Moraxella | Examples include M. catarrhalis (lower respiratory tract |
| | | infection) and Moraxella lacunata |
| | | (blepharoconjunctivitis) |
| Proteobacteria | Pseudomonas | Multiple groups and subspecies therein. Examples |
| | | include P. aeruginosa - opportunistic infection in cystic |
| | | fobrosis, burn and immunocompromised subjects. |
| | | Colonizes skin, lungs, kidney, urinary tract. Found on |
| | | most medical equipment - develops enduring biofilms |
| Proteobacteria | Vibrio | Multiple species - foodborne disease generally |
| | | implicated with gastroenteritis - example - V. cholerae. |
| | | Can also cause septicemia in open wounds |
| Proteobacteria | Plesimonas | Example P. Shigelloides - causes gastrointestinal disease |
| Proteobacteria | Aeromonas | Cornucopia of human disease including and not limited to |
| | | gastrointestinal disease, wound and soft tissue infection, |
| | | blood borne dyscrasias |
| Proteobacteria | Citrobacter | Examples: C. freundii, C. koseri, C. amalonaticus. Infect |
| | | urinary tract, can cause infant meningitis and sepsis |
| Proteobacteria | Enterobacter | Multiple species - Urinary and respiratory tract |
| | | infections are most common. Example E. aerogenes are a |
| | | common source of opportunistic and/or nosocomial |
| | | infections |
| Proteobacteria | Escerichia | Common cause of gastrointestinal infections. Example |
| | | E. coli |
| Proteobacteria | Klebsiella | Multiple species and sub-species. Cause a variety of |
| | | opporuntistic infections: Pneumonia, UTI, septicemia, |
| | | meningitis, diarrhea, soft tissue infection |
| Proteobacteria | Proteus | Urinary tract infections (including kidney) - P. vulgaris, |
| | | P. mirabilis, P. penneri |
| Proteobacteria | Providencia | Multiple species cause. Urinary tract infections. |
| | | Example: P. stuartii |
| Proteobacteria | Morganella | Single species with 2 subspecies. M. morganii. Cause |
| | | opportunistic infections (wound, UTI) |
| Proteobacteria | Salmonella | Two species S. bongori and S. enterica, along with |
| | | multiple subspecies. Typical non-typhoidal salmonella |
| | | causes gastrointestinal disease. In developing countries it |
| | | also causes blood infections. Typhiidal disease causes |
| | | typhoid fever, hypovolemic shock, septic shock |
| Proteobacteria | Serratia | Multiple species: Opportunisitc infection that often |
| | | develops biofilms. Colonizes respiratory and urinary |
| | | tract. Responsible for 2% of nosocomial infections of |
| | | blood, LRT, UT, surgical wounds, skin and soft tissue |
| | | infection. Example s. marcescens |
| Proteobacteria | Shigella | 4 species. Causes shigellosis (leading bacterial cause of |
| | | diarrhea). Example : S. dysenteriae |
| Proteobacteria | Yersinia | Multiple species and sub-species. Example: Y. pestis |
| | | causes the plague |
| Proteobacteria | Pasteurella | Multiple species. P. multocida species is the most |
| | | frequent example of human infection ( - symptoms |
| | | include swelling, cellulitis, wound drainage and arthritis) |
| Proteobacteria | Hemophilus | Multiple species. Example H influenzae (Hib) causes |
| | | penumonia, sepsis and bacterial meningitis in young |
| | | children |
| Proteobacteria | Campylobacter | Multiple species. Cause campylobacteriosis |
| | | (gastrointestinal disease - inflammatory diarrhea/ |
| | | dysentery). Example C. rectus |
| Proteobacteria | Heliobacter | Example H pylori. Causes gastritis and gastric ulcers |
| Firmicutes | Clostridia | Multiple Species. Examples: C. Difficile, C. botulinum, |
| | | C. tetani. Cause a variety of serious conditions, from |
| | | colitis to paralysis |
| Firmicutes | Mycoplasmas | Multiple species. Examples: M. genitalium, |
| | | M pneumoniae. Ureaplasma species. Associated with |
| | | sexually transmited disease, infertility and infant |
| | | respiratory distress and brain hemorrhage. P1 antigen is |
| | | primary virulence factor, which is also expressed on |
| | | erthyrocytes. This can lead to autoantibody agglutination |
| Firmicutes | Bacillus | One of the most diverse genus from a speciation |
| | | perspective. Examples B. anthracis (anthrax), B. cereus |
| | | (food poisoning) |
| Firmicutes | Listeria | 15 identified species. Example: L. monocytogenes food |
| | | poisining. Less frequently seen disease manifestation = |
| | | listeriosis (sepsis and meningitis with a 20% fatality rate) |
| Firmicutes | Staphylococcus | Multiple species and sub. Example: S. aureus. Disease |
| | | can range from folliculitis to necrotizing pneumonia and |
| | | endocarditis. Commonly drug resistant (MRSA) |
| Firmicutes | Enterococcus | Multiple species. Cause UTI, Bacteremia, endocarditis, |
| | | diverticulitis, meningitis, prostatitis. Example: |
| | | E. faecium. Increasingly drug resistant (VRE) |
| Firmicutes | Lactobacillus | Often considered beneficial, but can cause septicemia, |
| | | endocarditis, rheumatic vascular disease and dental |
| | | caries(particularly in immuno-compromised subjects). |
| | | Example L. rhamnosus |
| Firmicutes | Streptococcus | Multiple species. Cause strep throat, pink eye, |
| | | meningitis, bacterial pneumonia, sepsis, endocarditis, |
| | | erysipelas, necrotizing fasciitis. Examples: S. pyogenes, |
| | | S. pneumoniae, S. sanguinis |
| Actinobacteria | Nocardia | Multiple species. Low virulence and generally infect the |
| | | immunocompromised only. Cause penumonia, |
| | | endocarditis, encephalitis, cellulitis. Example |
| | | N. asteroids |
| Actinobacteria | Mycobacterium | Multiple species. Cause tuberculosis, leprosy. Examples |
| | | M. tuberculosis, M. leprae |
| Actinobacteria | Corynebacterium | Multiple species. Cause diphtheria, colonizes prosthetics, |
| | | and can cause skin infections, endocarditis, pneumonitis. |
| | | Nosocomial |
| Actinobacteria | Actinomyces | Multiple species. Cause periodontal abscesses, |
| | | lymphadenopathy, thoracic disease and abdominal |
| | | abscess. Example: Aggregatibacter |
| | | actinomycetemcomitans |
| Chlamydia | Chlamydia | 4 species. Most common bacterial STD and can cause |
| | | blindness. Example Chlamydia trachomatis. |
| Spirochetes | Borrelia | 52 species. Cause Lyme disease and relapsing fever |
| | | (severe bacteremia). Example: Borrelia burgdorferi |
| Spirochetes | Leptospira | Multiple species (13 cause disease in humans). Cause |
| | | leptospirosis - symptoms range from headaches and |
| | | fatigue to meningitis, kidney failure and pulmonary |
| | | hemorrhage. |
| Spirochetes | Treponema | Multiple species. Causes syphilis Example: Treponema |
| | | pallidum. |
| Bacteroides | Bacteroides | Multiple species. Colonize the gut and cause infections |
| | | associated with surgery, appendicitis etc. Example: |
| | | B. fragilis |

In certain embodiments, the infectious disease therapeutic is selected from a fungal polypeptide. In these embodiments, an engineered erythroid cell or enucleated cell comprises an erythroid cell (e.g., an enucleated erythroid cell) or an enucleated cell, comprising one or more exogenous antigenic polypeptides, wherein one of the one or more exogenous antigenic polypeptides comprises an antigenic fungal polypeptide, and the engineered erythroid cell or enucleated cell is administered to a subject to treat an infection with the fungus.

Exemplary fungal pathogens are shown below in Table 25.

TABLE 25
Fungal Pathogens

| Fungal | | |
|---|---|---|
| Group | Pathogen Species | Pathology example |
| Yeast | Candida albicans | Oral thrush, onychomycosis |
| Yeast | Candida glabrata | Vaginitis, esophageal candidiasis |
| Yeast | Candida krusei | Invasive candidiasis, neutropenia |
| Yeast | Candida | Catheter and central line infections, biofilms, UTI, |
| | parapsilosis | endocarditis, meningitis |
| Yeast | Candida tropicalis | Candidemia, oral thrush |
| Yeast | Rhodotorula | Sepsis, catheter infections, fungemia, meningitis, peritonitis |
| | mucilaginosa | |
| Yeast | Sporothrix | Sporotrichosis (lympocutaneous tissue, skin ulcerations) |
| | schenckii complex | |
| Yeast | Cryptococcus | Cryptococcal meningitis, pulmonary infection, osteomyelitis |
| | neoformans | |
| Yeast | Cryptococcus | Cryptococcal meningitis, pulmonary infection, osteomyelitis |
| | gattii | (more common in immunocompromised subjects that |
| | | C. neoformans, which is more prevalent in immunocompetent |
| | | subjects) |
| Moulds | Alternaria | Allergic fungal rhinosinusitis, Keratitis, peritonitis, allergic |
| | alternata | respiratory disease |
| Moulds | Apophysomyces | Cutaneous and subcutaneous infection |
| | variabilis | |
| Moulds | Aspergillus | Chronic pulmonary aspergillosis, Asthma exacerbation with |
| | fumigatus | fungal sensitization, chronic invasive sinusitis, invasive and |
| | | disseminated aspergillosis |
| Moulds | Aspergillus flavus | Chronic cavity pulmonary aspergillosis, Cutaneous and |
| | | wound infection, endocarditis, pericarditis and CNS |
| | | infection, UTI |
| Moulds | Aspergillus niger | Otomycosis, SAFS, Allergic bronchopulmonary |
| | | aspergillosis, invasive pulmonary aspergillosis, disseminated |
| | | aspergillosis |
| Moulds | Aspergillus terreus | ABPA, aspergillus bronchitis, invasive aspergillosis, |
| | | disseminated aspergillosis |
| Moulds | Cladosphialophora | Chromoblastomycosis, mycetoma, phaehyphomycosis |
| | spp. | |
| Moulds | Exserohilum | Skin, corneal infection, invasive infection of sinus heart and |
| | | lung |
| Moulds | Fonsecaea | Chromoblastomycosis |
| | pedrosoi | |
| Moulds | Fusarium | Keratitis, Onychomycosis, endophthalmitis, skin infection, |
| | oxysporum | sinusitis, disseminated infection |
| Moulds | Fusarium solani | Keratitis, Onychomycosis, endophthalmitis, skin infection, |
| | | sinusitis, disseminated infection |
| Moulds | Lichtheimia | cutaneous, pulmonary, rhinocerebral CNS and disseminated |
| | corymbifera | infection (rare) |
| Moulds | Lichtheimia | cutaneous, pulmonary, rhinocerebral CNS and disseminated |
| | ramosa | infection (rare) |
| Moulds | Rhizopus | Mucormycosis |
| | microsporus | |
| Moulds | Stachybotrys | idiopathic pulmonary hemorrhage (infants) |
| Moulds | Trichophyton | Tinea pedis, corporis, cruris, onychomycosis and |
| | interdigitale | occasionally Tinea capitis |
| Moulds | Trichophyton | Tinea of the groin, glabrous skin, feet, hands, and the nails. |
| | rubrum | Tinea cruris, tinea corporis, tinea pedis, tinea manuum, and |
| | | onychomycosis |
| Dimorphic | Histoplasma | Pneumonia, multi-organ failure |
| Fungi | capsulatum | |
| Dimorphic | Pneumocystis | Pneumonia (mostly in immune suppressed subjects) |
| Fungi | jirovecii | |
| Dimorphic | Paracoccidioides | Disseminated systemic infection |
| Fungi | brasiliensis | |
| Dimorphic | Penicillium | Disseminated infection with prominent skin lesions |
| Fungi | marneffei | (immunocompromised subjects) |
| Dimorphic | Blastomyces | Skin infections, rarely disseminated |
| Fungi | | |
| Dimorphic | Coccidioides | Meningitis, disseminated disease, lung nodule, chronic |
| Fungi | | cavitary pulmonary coccidioidmycosis |

In certain embodiments, the infectious disease therapeutic is selected from a parasitic polypeptide. In these embodiments, an engineered erythroid cell or enucleated cell comprises an erythroid cell (e.g., an enucleated erythroid cell) or an enucleated cell, comprising one or more exogenous antigenic polypeptides, wherein one of the one or more exogenous antigenic polypeptides comprises an antigenic parasitic polypeptide, and the engineered erythroid cell or enucleated cell is administered to a subject to treat an infection with the parasite.

Exemplary parasitic pathogens are shown below in Table 26.

TABLE 26
Parasitic Pathogens

| Parasitic | | |
|---|---|---|
| Group | Pathogen Species | Disease/Pathology Example |
| Amoeba/ | Acanthamoeba | Acanthamoeba Infection |
| Protozoa | | |
| Amoeba/ | Acanthamoeba | Acanthamoeba Keratitis Infection |
| Protozoa | | |
| Amoeba/ | Trypanosoma brucei | African Sleeping Sickness (African trypanosomiasis) |
| Protozoa | | |
| Amoeba/ | Entamoeba histolytica | Amebiasis (Entamoeba histolytica Infection) |
| Protozoa | | |
| Amoeba/ | Trypanosoma cruzi | American Trypanosomiasis (Chagas Disease) |
| Protozoa | | |
| Amoeba/ | Balantidium coli | Balantidiasis (Balantidium Infection) |
| Protozoa | | |
| Amoeba/ | Balamuthia mandrillaris | Balamuthia (Granulomatous Amebic Encephalitis (GAE)) |
| Protozoa | | |
| Amoeba/ | Cryptosporidium | Cryptosporidiosis (Cryptosporidium Infection) |
| Protozoa | | |
| Amoeba/ | Cyclospora | Cyclosporiasis (Cyclospora Infection) |
| Protozoa | | |
| Amoeba/ | Taenia solium | Cysticercosis (Neurocysticercosis) |
| Protozoa | | |
| Amoeba/ | Cystoisospora belli | Cystoisospora Infection (Cystoisosporiasis) formerly |
| Protozoa | | Isospora Infection |
| Amoeba/ | Dientamoeba fragilis | Dientamoeba fragilis Infection |
| Protozoa | | |
| Amoeba/ | Entamoeba histolytica | Entamoeba histolytica Infection (Amebiasis) |
| Protozoa | | |
| Amoeba/ | Giardia intestinalis, | Giardiasis (Giardia Infection) |
| Protozoa | Giardia lamblia, or | |
| | Giardia duodenalis | |
| Amoeba/ | Leishmania | Kala-azar (Leishmaniasis, Leishmania Infection) |
| Protozoa | | |
| Amoeba/ | Acanthamoeba | Keratitis (Acanthamoeba Infection) |
| Protozoa | | |
| Amoeba/ | Multiple species. | Malaria (Plasmodium Infection) |
| Protozoa | Examples: P. vivax and | |
| | P. ovale | |
| Amoeba/ | Naegleria fowleri | Naegleria Infection |
| Protozoa | “brain-eating amoeba” | |
| Amoeba/ | Sappinia ameoba | Sappinia (amebic encephalitis) |
| Protozoa | | |
| Amoeba/ | Sarcocystis | Sarcocystosis (Sarcocystosis Infection) |
| Protozoa | | |
| Amoeba/ | Toxoplasma gondii | Toxoplasmosis (Toxoplasma Infection) - protozoa |
| Protozoa | | |
| Amoeba/ | Trichomonas vaginalis | Trichomoniasis (Trichomonas Infection) - protozoa |
| Protozoa | | |
| Amoeba/ | Trypanosoma brucei | Trypanosomiasis, African (African Sleeping Sickness, |
| Protozoa | | Sleeping Sickness) - protozoa |
| Amoeba/ | Babesia (various | Babesiosis (Babesia Infection) - similar lifecycle to |
| Protozoa | species) | malaria, taken up by RBCs |
| Arthropod | Cimex lectulariusand | Bed Bugs |
| | C. hemipterus | |
| Arthropod | Pediculus humanus | Body Lice Infestation (Pediculosis) |
| | humanus | |
| Arthropod | Phthirus pubis | Crabs (Pubic Lice) |
| Arthropod | Pediculus humanus | Head Lice Infestation (Pediculosis) |
| | capitis | |
| Arthropod | Sarcoptes | Mite Infestation (Scabies) |
| | scabiei var. hominis | |
| Arthropod | infection of a fly larva. | Myiasis |
| | Examples: Dermatobia | |
| | hominis | |
| Other | group of obligate | Microsporidiosis (Microsporidia Infection) |
| | intracellular parasitic | |
| | fungi. Examples: M. | |
| | africanum, Nosema | |
| | ocularum | |
| Other | Pneumocystis jirovecii | Pneumocystis jirovecii Pneumonia |
| | (considered a parasitic | |
| | fungi) | |
| Protist | Blastocystis hominis | Blastocystis hominis Infection |
| Worm | Echinococcus | Alveolar Echinococcosis (Echinococcosis, Hydatid |
| | granulosus | Disease) |
| Worm | Hookworm (various | Ancylostomiasis (Hookworm) |
| | species) | |
| Worm | Angiostrongylus | Angiostrongyliasis (Angiostrongylus Infection) |
| | (various species) | |
| | nematode | |
| Worm | anisakid nematodes | Anisakiasis (Anisakis Infection, Pseudoterranova |
| | | Infection) |
| Worm | Ascaris lumbricoides | Ascariasis (Ascaris Infection, Intestinal Roundworms) |
| Worm | Baylisascaris procyonis | Baylisascariasis (Baylisascaris Infection, Raccoon |
| | | Roundworm) |
| Worm | Schistosoma mansoni | Bilharzia (Schistosomiasis) |
| Worm | Capillaria | Capillariasis (Capillaria Infection) |
| | hepatica and Capillaria | |
| | philippinensis | |
| Worm | Various species, | Cercarial Dermatitis (Swimmer's Itch) |
| | example: | |
| | Austrobilharzia | |
| | variglandis | |
| Worm | Clonorchis liver fluke | Clonorchiasis (Clonorchis Infection) |
| Worm | Diphyllobothrium | Diphyllobothriasis (Diphyllobothrium Infection) |
| | latum (tapeworm) | |
| Worm | Dipylidiu tapeworm | Dipylidium caninum Infection (dog or cat tapeworm |
| | | infection) |
| Worm | Dirofilaria roundworms | Dirofilariasis (Dirofilaria Infection) |
| Worm | Guinea worm | Dracunculiasis (Guinea Worm Disease) |
| Worm | Echinococcus | Echinococcosis (Cystic, Alveolar Hydatid Disease) |
| | granulosus & | |
| | Echinococcus | |
| | multilocularis | |
| Worm | Wuchereria | Elephantiasis (Filariasis, Lymphatic Filariasis) |
| | bancrofti, Brugia malayi | |
| | and Brugia timori | |
| Worm | Enterobius vermicularis | Enterobiasis (Pinworm Infection) |
| Worm | Fasciola hepatica | Fascioliasis (Fasciola(liver fluke) Infection) |
| Worm | Fasciolopsis buski | Fasciolopsiasis (Fasciolopsis Infection) |
| Worm | Gnathostoma: several | Gnathostomiasis (Gnathostoma Infection) |
| | species of parasitic | |
| | nematodes | |
| Worm | trematode Heterophyes | Heterophyiasis (Heterophyes Infection) |
| | heterophyes | |
| Worm | Multiple species. | Hookworm Infection, Human |
| | Example: Ancylostoma | |
| | duodenale and Necator | |
| | americanus | |
| Worm | Multiple species. | Hookworm Infection, Zoonotic (Ancylostomiasis, |
| | Example: Ancylostoma | Cutaneous Larva Migrans [CLM]) |
| | brazilense, A. caninum, | |
| | A. ceylanicum | |
| Worm | Hymenolepis nana | Hymenolepiasis (Hymenolepis Infection) |
| | (dwarf tapeworm) | |
| Worm | Various species, | Intestinal Roundworms (Ascariasis, Ascaris Infection) |
| | example: A. | |
| | lumbricoides | |
| Worm | Loa: parasitic worm | Loiasis (Loa loa Infection) |
| Worm | Taenia solium | Neurocysticercosis (Cysticercosis) |
| Worm | Toxocara canis and | Ocular Larva Migrans (Toxocariasis, Toxocara Infection, |
| | Toxocara cati | Visceral Larva Migrans) |
| Worm | Onchocerca volvulus | Onchocerciasis (River Blindness) |
| Worm | Opisthorchis (liver | Opisthorchiasis (Opisthorchis Infection) |
| | fluke). Example: O | |
| | Opisthorchis felineus | |
| Worm | Paragonimus (lung | Paragonimiasis (Paragonimus Infection) |
| | fluke) | |
| Worm | anisakid nematodes | Pseudoterranova Infection (Anisakiasis, Anisakis |
| | | Infection) |
| Worm | Strongyloides | Strongyloidiasis (Strongyloides Infection) |
| | nematodes. Example: | |
| | Strongyloides | |
| | stercoralis | |
| Worm | Taenia saginata | Taeniasis (Taenia Infection, Tapeworm Infection) |
| Worm | Trichinella | Trichinellosis (Trichinosis) |
| Worm | Trichuris trichiura | Trichuriasis (Whipworm Infection, Trichuris Infection) |

In some embodiments, the infectious disease is a multi-drug resistant Staphylococcus infection (e.g., a Staphylococcus aureus infection. In another embodiments, the infectious diseases is Pseudomonas infection. In another embodiment, the infectious disease is a nosocomial infections (i.e. any systemic or localized conditions that result from the reaction by an infectious agent or toxin) that are difficult to treat, for example infections caused by Clostridium difficile.

Other

Other diseases and disorders are contemplated for treatment by the engineered erythroid cells or enucleated cells of the present disclosure. Examples include, but are not limited to cardiovascular diseases and immune diseases.

### Subjects

The methods described herein are intended for use with any subject that may experience the benefits of these methods. Thus, “subjects,” “subjects,” and “individuals” (used interchangeably) include humans as well as non-human subjects, particularly domesticated animals.

In some embodiments, the subject and/or animal is a mammal, e g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal. In some embodiments, the subject and/or animal is a human. In some embodiments, the human is a pediatric human In other embodiments, the human is an adult human. In other embodiments, the human is a geriatric human. In other embodiments, the human may be referred to as a subject.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the disclosure pertains to veterinary use. In a specific embodiment, the non-human animal is a household pet. In another specific embodiment, the non-human animal is a livestock animal In certain embodiments, the subject is a human cancer subject that cannot receive chemotherapy, e.g. the subject is unresponsive to chemotherapy or too ill to have a suitable therapeutic window for chemotherapy (e.g. experiencing too many dose- or regimen-limiting side effects). In certain embodiments, the subject is a human cancer subject having advanced and/or metastatic disease.

In some embodiments, the subject is selected for treatment with an engineered erythroid cell or enucleated cell comprising one or more exogenous polypeptides of the present disclosure. In some embodiments, the subject is selected for treatment of cancer with an engineered erythroid cell or enucleated cell comprising one or more exogenous polypeptides of the present disclosure. In some embodiments, the subject is selected for treatment of an autoimmune disease with an engineered erythroid cell or enucleated cell comprising one or more exogenous polypeptides of the present disclosure. In some embodiments, the subject is selected for treatment of an infectious disease with an engineered erythroid cell or enucleated cell comprising one or more exogenous polypeptides of the present disclosure.

In some embodiments of the above aspects and embodiments, the engineered erythroid cell is an enucleated erythroid cell. In some embodiments of the above aspects and embodiments, the engineered erythroid cell is a nucleated erythroid cell.

### IV. Pharmaceutical Compositions

The present disclosure encompasses the preparation and use of pharmaceutical compositions comprising an engineered erythroid cell or enucleated cell of the disclosure as an active ingredient. Such a pharmaceutical composition may consist of the active ingredient alone, as a combination of at least one active ingredient (e.g., an effective dose of an engineered erythroid cell or enucleated cell) in a form suitable for administration to a subject, or the pharmaceutical composition may comprise the active ingredient and one or more pharmaceutically acceptable carriers, one or more additional (active and/or inactive) ingredients, or some combination of these.

In some embodiments, the disclosure features a pharmaceutical composition comprising a plurality of the engineered erythroid cells or enucleated cells described herein, and a pharmaceutical carrier. In other embodiments, the disclosure features a pharmaceutical composition comprising a population of engineered erythroid cells or enucleated cells as described herein, and a pharmaceutical carrier. It will be understood that any single engineered erythroid cells or enucleated cells , plurality of engineered erythroid cells or enucleated cells , or population of engineered erythroid cells or enucleated cells as described elsewhere herein may be present in a pharmaceutical composition of the disclosure.

In some embodiments, the pharmaceutical compositions provided herein comprise engineered (i.e. modified) erythroid cells and unmodified erythroid cells. For example, a single unit dose of engineered erythroid cells or enucleated cells can comprise, in various embodiments, about, at least, or no more than 10%, 20%, 30%, 40%, 50%, 60%, 70%, 75%, 80%), 85%, 90%, 95%, or 99% engineered erythroid cells, wherein the remaining erythroid cells in the composition are not engineered.

In some embodiments, the pharmaceutical compositions provided herein comprise engineered erythroid cells or enucleated cells comprising engineered erythroid cells or enucleated cells and nucleated erythroid cells. For example, a single unit dose of engineered erythroid cells (e.g., enucleated and nucleated erythroid cells) can comprise, in various embodiments, about, or at least 10%, 20%, 30%, 40%, 50%, 60%, 70%, 75%, 80%, 85%, 90%, 91%, 92%, 93%, 94%, 95%, 96%, 97%, 98%, or 99% enucleated erythroid cells, wherein the remaining erythroid cells in the composition are nucleated.

Pharmaceutical compositions of the present disclosure may be administered in a manner appropriate to the disease to be treated (or prevented). The quantity and frequency of administration will be determined by such factors as the condition of the subject, and the type and severity of the subject's disease, although appropriate dosages may be determined by clinical trials.

The administration of the pharmaceutical compositions may be carried out in any convenient manner, including by aerosol inhalation, injection, ingestion, transfusion, implantation or transplantation. The compositions of the present disclosure may be administered to a subject subcutaneously, intradermally, intramuscularly, by intravenous (i.v.) injection, or intraperitoneally. The pharmaceutical compositions may be injected directly into a tumor or lymph node.

As used herein, the term “pharmaceutically acceptable carrier” means a chemical composition with which the active ingredient may be combined and which, following the combination, can be used to administer the active ingredient to a subject.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs, birds including commercially relevant birds such as chickens, ducks, geese, and turkeys, fish including farm-raised fish and aquarium fish, and crustaceans such as farm-raised shellfish.

Pharmaceutical compositions that are useful in the methods of the disclosure may be prepared, packaged, or sold in formulations suitable for oral, rectal, vaginal, parenteral, topical, pulmonary, intranasal, intra-lesional, buccal, ophthalmic, intravenous, intra-organ or another route of administration. Other contemplated formulations include projected nanoparticles, liposomal preparations, resealed erythrocytes containing an exogenous polypeptide described herein, and immunologically-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the disclosure may further comprise one or more additional pharmaceutically active agents. Particularly contemplated additional agents include anti-emetics and scavengers such as cyanide and cyanate scavengers and AZT, protease inhibitors, reverse transcriptase inhibitors, interleukin-2, interferons, cytokines, and the like.

Controlled- or sustained-release formulations of a pharmaceutical composition of the disclosure may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

The engineered erythroid cell or enucleated cell of the disclosure and/or T cells expanded using the engineered erythroid cell or enucleated cell, can be administered to an animal, preferably a human. When the T cells expanded using an engineered erythroid cell or enucleated cell of the disclosure are administered, the amount of cells administered can range from about 1 million cells to about 300 billion. Where the engineered erythroid cells or enucleated cells themselves are administered, either with or without T cells expanded thereby, they can be administered in an amount ranging from about 100,000 to about one billion cells wherein the cells are infused into the animal, preferably, a human subject in need thereof. While the precise dosage administered will vary depending upon any number of factors, including but not limited to, the type of animal and type of disease state being treated, the age of the animal and the route of administration.

The engineered erythroid cell or enucleated cell may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

An engineered erythroid cell or enucleated cell (or cells expanded thereby) may be co-administered with the various other compounds (cytokines, chemotherapeutic and/or antiviral drugs, among many others). Alternatively, the compound(s) may be administered an hour, a day, a week, a month, or even more, in advance of the engineered erythroid cell or enucleated cell (or cells expanded thereby), or any permutation thereof. Further, the compound(s) may be administered an hour, a day, a week, or even more, after administration of engineered erythroid cell or enucleated cell (or cells expanded thereby), or any permutation thereof. The frequency and administration regimen will be readily apparent to the skilled artisan and will depend upon any number of factors such as, but not limited to, the type and severity of the disease being treated, the age and health status of the animal, the identity of the compound or compounds being administered, the route of administration of the various compounds and the engineered erythroid cell or enucleated cell (or cells expanded thereby), and the like.

Further, it would be appreciated by one skilled in the art, based upon the disclosure provided herein, that where the engineered erythroid cell or enucleated cell is to be administered to a mammal, the cells are treated so that they are in a “state of no growth”; that is, the cells are incapable of dividing when administered to a mammal. As disclosed elsewhere herein, the cells can be irradiated to render them incapable of growth or division once administered into a mammal. Other methods, including haptenization (e.g., using dinitrophenyl and other compounds), are known in the art for rendering cells to be administered, especially to a human, incapable of growth, and these methods are not discussed further herein. Moreover, the safety of administration of engineered erythroid cell or enucleated cell that have been rendered incapable of dividing in vivo has been established in Phase I clinical trials using engineered erythroid cell or enucleated cell transfected with plasmid vectors encoding some of the molecules discussed herein.

### Combination Therapies

In some embodiments, the disclosure provides methods that further comprise administering an additional agent to a subject. In some embodiments, the disclosure pertains to co-administration and/or co-formulation.

In some embodiments, administration of the engineered erythroid cell or enucleated cell acts synergistically when co-administered with another agent and is administered at doses that are lower than the doses commonly employed when such agents are used as monotherapy.

In some embodiments, inclusive of, without limitation, cancer applications, the present disclosure pertains to chemotherapeutic agents as additional agents. Examples of chemotherapeutic agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores), aclacinomy sins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel, and TAXOTERE doxetaxel; chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB); inhibitors of PKC-a., Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of radiation.

Some human tumors can be eliminated by a subject's immune system. For example, administration of a monoclonal antibody targeted to an immune “checkpoint” molecule can lead to complete response and tumor remission. A mode of action of such antibodies is through inhibition of an immune regulatory molecule that the tumors have co-opted as protection from an anti-tumor immune response. By inhibiting these “checkpoint” molecules (e.g., with an antagonistic antibody), a subject's CD8+ T cells may be allowed to proliferate and destroy tumor cells.

For example, administration of a monoclonal antibody targeted to by way of example, without limitation, CTLA-4 or PD-1 can lead to complete response and tumor remission. The mode of action of such antibodies is through inhibition of CTLA-4 or PD-1 that the tumors have co-opted as protection from an anti-tumor immune response. By inhibiting these “checkpoint” molecules (e.g., with an antagonistic antibody), a subject's CD8+ T cells may be allowed to proliferate and destroy tumor cells.

Thus, the engineered erythroid cells or enucleated cells comprising an enucleated cell or erythroid cell presenting (e.g. comprising on the cell surface) one or more exogenous polypeptides provided herein can be used in combination with one or more blocking antibodies targeted to an immune “checkpoint” molecule. For instance, in some embodiments, the compositions provided herein can be used in combination with one or more blocking antibodies targeted to a molecule such as CTLA-4 or PD-1. For example, the compositions provided herein may be used in combination with an agent that blocks, reduces and/or inhibits PD-1 and PD-L1 or PD-L2 and/or the binding of PD-1 with PD-L1 or PD-L2 (by way of non-limiting example, one or more of nivolumab (ONO-4538/BMS-936558, MDX1106, OPDIVO, BRISTOL MYERS SQUIBB), pembrolizumab (KEYTRUDA, Merck), pidilizumab (CT-011, CURE TECH), MK-3475 (MERCK), BMS 936559 (BRISTOL MYERS SQUIBB), MPDL328OA (ROCHE)). In an embodiment, the compositions provided herein may be used in combination with an agent that blocks, reduces and/or inhibits the activity of CTLA-4 and/or the binding of CTLA-4 with one or more receptors (e.g. CD80, CD86, AP2M1, SHP-2, and PPP2R5A). For instance, in some embodiments, the immune-modulating agent is an antibody such as, by way of non-limitation, ipilimumab (MDX-010, MDX-101, Yervoy, BMS) and/or tremelimumab (Pfizer). Blocking antibodies against these molecules can be obtained from, for example, Bristol Myers Squibb (New York, N.Y.), Merck (Kenilworth, N.J.), Medlmmune (Gaithersburg, Md.), and Pfizer (New York, N.Y.).

Further, the engineered erythroid cell or enucleated cell compositions provided herein can be used in combination with one or more blocking antibodies targeted to an immune “checkpoint” molecule such as for example, BTLA, HVEM, TIM3, GALS, LAG3, VISTA, KIR, 2B4, CD160 (also referred to as BY55), CGEN-15049, CHK 1 and CHK2 kinases, A2aR, CEACAM (e.g., CEACAM-1, CEACAM-3 and/or CEACAM-5), GITR, GITRL, galectin-9, CD244, CD160, TIGIT, SIRPa, ICOS, CD172a, and TMIGD2 and various B-7 family ligands (including, but are not limited to, B7-1, B7-2, B7-DC, B7-H1, B7-H2, B7-H3, B7-H4, B7-H5, B7-H6 and B7-H7).

In some embodiments of the above aspects and embodiments, the erythroid cell is an enucleated erythroid cell. In some embodiments of the above aspects and embodiments, the erythroid cell is a nucleated erythroid cell.

In some embodiments, the disclosure features a pharmaceutical composition comprising a plurality of the engineered erythroid cells described herein, and a pharmaceutical carrier. In other embodiments, the disclosure features a pharmaceutical composition comprising a population of engineered erythroid cells as described herein, and a pharmaceutical carrier. It will be understood that any single engineered erythroid cell, plurality of engineered erythroid cells, or population of engineered erythroid cells as described elsewhere herein may be present in a pharmaceutical composition of the disclosure.

In some embodiments, the pharmaceutical compositions provided herein comprise engineered (i.e. modified) erythroid cells and unmodified erythroid cells. For example, a single unit dose of erythroid cells (e.g., modified and unmodified erythroid cells) can comprise, in various embodiments, about, at least, or no more than 10%, 20%, 30%, 40%, 50%, 60%, 70%, 75%, 80%), 85%, 90%, 95%, or 99% engineered erythroid cells, wherein the remaining erythroid cells in the composition are not engineered.

In some embodiments, the pharmaceutical compositions provided herein comprise engineered erythroid cells or enucleated cells and nucleated erythroid cells. For example, a single unit dose of engineered erythroid cells (e.g., enucleated and nucleated erythroid cells) can comprise, in various embodiments, about, or at least 10%, 20%, 30%, 40%, 50%, 60%, 70%, 75%, 80%, 85%, 90%, 91%, 92%, 93%, 94%, 95%, 96%, 97%, 98%, or 99% enucleated erythroid cells, wherein the remaining erythroid cells in the composition are nucleated.

### EXAMPLES

### Example 1

### Engineered Enucleated Erythroid Cells Including Loadable Antigen-presenting Polypeptides Can Be Loaded with Antigenic Polypeptide and Activate Antigen-specific T-Cell Receptors

To determine whether engineered enucleated erythroid cells comprising an exogenous loadable antigen-presenting polypeptide can be loaded (e.g., specifically bound to exogenous antigenic polypeptides) and whether once loaded they are capable of activating antigen-specific T-cell receptors (TCRs), the following experiments were performed.

Briefly, engineered enucleated erythroid cells were generated which included either: (1) an antigen-presenting polypeptide comprising wild-type HLA*02:01 polypeptide, β2M polypeptide, GPA, and FLAG-tag (“wt HLA-A2”), as shown in SEQ ID NO:38 (below),

(SEQ ID NO: 38)
MSRSVALAVLALLSLSGLEAIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDMGGGGSGGGGSGGGGSGGGGSGSHSMRYFFTS
VSRPGRGEPRFIAVGYVDDTQFVRFDSDAASQRMEPRAPWIEQEGPEYWD
GETRKVKAHSQTHRVDLGTLRGYYNQSEAGSHTVQRMYGCDVGSDWRFLR
GYHQYAYDGKDYIALKEDLRSWTAADMAAQTTKHKWEAAHVAEQLRAYLE
GTCVEWLRRYLENGKETLQRTDAPKTHMTHHAVSDHEATLRCWALSFYPA
EITLTWQRDGEDQTQDTELVETRPAGDGTFQKWAAVVVPSGQEQRYTCHV
QHEGLPKPLTLRWEPSSQPTIPIGSGSGSGSDYKDDDDKGSGSGSGSLST
TEVAMHTSTSSSVTKSYISSQTNDTHKRDTYAATPRAHEVSEISVRTVYP
PEEETGERVQLAHHFSEPEITLIIFGVMAGVIGTILLISYGIRRLIKKSP
SDVKPLPSPDTDVPLSSVEIENPETSDQ;

(2) a loadable antigen-presenting polypeptide comprising mutant HLA*02:01 polypeptide having the amino acid substitutions Y84C and A139C, β2M polypeptide, GPA, and a FLAG-tag, (“ds HLA-A2”), as shown in SEQ ID NO:39 (below),

(SEQ ID NO: 39)
MSRSVALAVLALLSLSGLEAIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDMGGGGSGGGGSGGGGSGGGGSGSHSMRYFFTS
VSRPGRGEPRFIAVGYVDDTQFVRFDSDAASQRMEPRAPWIEQEGPEYWD
GETRKVKAHSQTHRVDLGTLRGCYNQSEAGSHTVQRMYGCDVGSDWRFLR
GYHQYAYDGKDYIALKEDLRSWTAADMCAQTTKHKWEAAHVAEQLRAYLE
GTCVEWLRRYLENGKETLQRTDAPKTHMTHHAVSDHEATLRCWALSFYPA
EITLTWQRDGEDQTQDTELVETRPAGDGTFQKWAAVVVPSGQEQRYTCHV
QHEGLPKPLTLRWEPSSQPTIPIGSGSGSGSDYKDDDDKGSGSGSGSLST
TEVAMHTSTSSSVTKSYISSQTNDTHKRDTYAATPRAHEVSEISVRTVYP
PEEETGERVQLAHHFSEPEITLIIFGVMAGVIGTILLISYGIRRLIKKSP
SDVKPLPSPDTDVPLSSVEIENPETSDQ;
or

(3) a fusion polypeptide comprising an HPV E7 peptide, mutant HLA*02:01 polypeptide having the amino acid substitution Y84A, β2M polypeptide, GPA, and FLAG-tag (“sc trimer”), as shown in SEQ ID NO: 40 (below),

(SEQ ID NO: 40)
MSRSVALAVLALLSLSGLEAYMLDLQPETGGGGSGGGGSGGGGSIQRTPK
IQVYSRHPAENGKSNFLNCYVSGFHPSDIEVDLLKNGERIEKVEHSDLSF
SKDWSFYLLYYTEFTPTEKDEYACRVNHVTLSQPKIVKWDRDMGGGGSGG
GGSGGGGSGGGGSGSHSMRYFFTSVSRPGRGEPRFIAVGYVDDTQFVRFD
SDAASQRMEPRAPWIEQEGPEYWDGETRKVKAHSQTHRVDLGTLRGAYNQ
SEAGSHTVQRMYGCDVGSDWRFLRGYHQYAYDGKDYIALKEDLRSWTAAD
MAAQTTKHKWEAAHVAEQLRAYLEGTCVEWLRRYLENGKETLQRTDAPKT
HMTHHAVSDHEATLRCWALSFYPAEITLTWQRDGEDQTQDTELVETRPAG
DGTFQKWAAVVVPSGQEQRYTCHVQHEGLPKPLTLRWEPSSQPTIPIGSG
SGSGSEDGSGSGSGSLSTTEVAMHTSTSSSVTKSYISSQTNDTHKRDTYA
ATPRAHEVSEISVRTVYPPEEETGERVQLAHHFSEPEITLIIFGVMAGVI
GTILLISYGIRRLIKKSPSDVKPLPSPDTDVPLSSVEIENPETSDQ,

To generate the engineered enucleated erythroid cells, human CD34+ erythroid precursor cells derived from mobilized peripheral blood cells from normal human donors were obtained. The expansion/differentiation procedure comprised 3 stages. In the first stage, thawed CD34+ erythroid precursor cells were cultured in Iscove's MDM medium supplemented with albumin, recombinant human insulin, human transferrin, recombinant human fms-like tyrosine kinase 3 ligand, recombinant human stem cell factor, recombinant human interleukin 3, and recombinant human interleukin 6 at a seeding density of 1E5 cells/mL for 7 days. In the second stage, erythroid cells were cultured in Iscove's MDM medium supplemented with recombinant human insulin, human transferrin, dexamethasone, lipid mixture, recombinant human interleukin 3, human recombinant stem cell factor, human recombinant erythropoietin, and L-glutamine at a starting density of 1E5 cells/mL for 7 days. In the third stage, erythroid cells were cultured in Iscove's MDM medium supplemented with human transferrin, recombinant human insulin, human recombinant erythropoietin, recombinant human stem cell factor, human AB serum, human peripheral blood plasma, and heparin sodium salt at a starting density of 1E5 cells/mL for 9 days. Fresh differentiation medium was added to the cultures on various days. The cultures were maintained at 37° C. in 5% CO2 incubator. On day 7 of the culture process described above, precursor cells were transduced with a lentiviral vector including the gene encoding the polypeptide of interest (e.g., a loadable antigen-presenting polypeptide). Transduction reactions were incubated overnight at 37° C. The following day, erythroid cells were gently spun down at 2,000 rpm for 5 minutes, supernatant removed, and the cells were re-suspended in fresh erythroid differentiation medium and cultured further at 37° C.

The fusion polypeptide described in “(3)” above was used as control since it already includes the antigenic polypeptide and does not need to be loaded with an additional antigenic polypeptide. To load the antigen-presenting polypeptides, the engineered erythroid cells described above were contacted with HPV E7 antigenic polypeptide (YMLDLQPET; SEQ ID NO:41) in serum-free Iscove's modified Dulbecco's media (IMDM) including 1 μg/mL peptide for 90 minutes at 37° C. Following this period, cells were briefly washed twice with phosphate-buffered saline (PBS), followed by a single wash with IMDM with serum, and cultured in Iscove's MDM medium supplemented with human serum albumin, recombinant human insulin, human transferrin, human recombinant erythropoietin, and human plasma, and then the cells were cultured in the latter media at 37° C. for up to 6 days (as indicated).

To assess the ability of the engineered enucleated erythroid cells to activate TCRs with specificity for the loaded HPV E7 peptide, TCR activity assays were performed. Briefly, NFAT-Luc reporter T lymphocyte cell line (InvivoGenTM) engineered to express an HPV E7-specific TCR were contacted for 18-22 hours with different amounts of engineered enucleated erythroid cells that had been cultured for 0, 3, or 6 days. The reporter T lymphocyte cell line was used to assess the ability of the engineered erythroid cells to activate signaling pathways following TCR engagement, which was measured by determining the levels of luciferase in the cell culture suspension using QUANTI-Luc™ (Invivogen™), a luciferase detection reagent, as instructed by the manufacturer. Measured signal was normalized to the baseline luminescence activity of the T lymphocyte reporter cells and expressed as fold increase in luminescense.

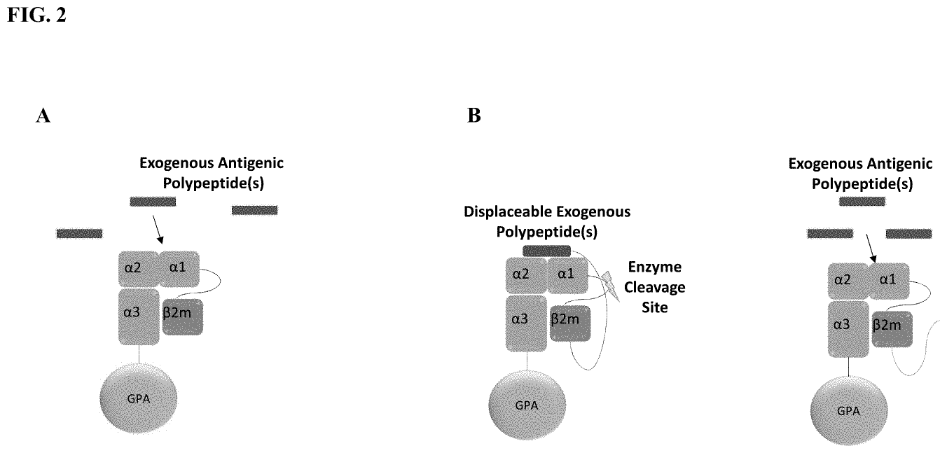

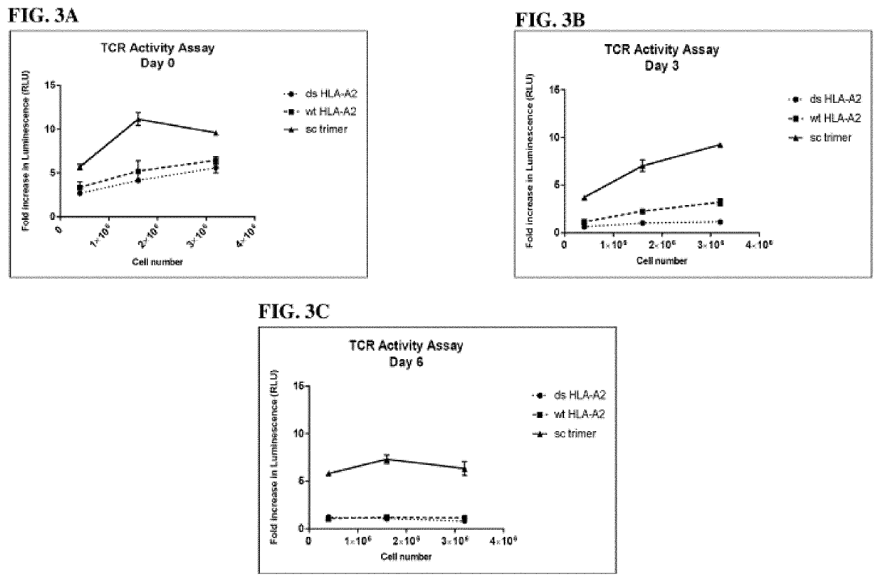

As shown in FIG. 3A, a dose-dependent response was observed when the reporter T lymphocytes were contacted with antigen-loaded engineered enucleated erythroid cells including either the wt HLA-A2 or the ds HLA-A2 antigen-presenting polypeptides which had been cultured for 0 days.

A dose-dependent response was also observed when the reporter T lymphocytes were contacted with antigen-loaded engineered enucleated erythroid cells including wt HLA-A2 after 3 days of culture (FIG. 3B). However, none or minimal activity was observed when the reporter T lymphocytes were contacted with antigen-loaded engineered enucleated erythroid cells including ds HLA-A2 after 3 days of culture. Without wishing to be bound by any particular theory, it is believed that the lack of activity observed using cells that had been cultured for 3 days may be attributed to HPV E7 antigenic polypeptide unloading from the ds HLA-A2 exogenous antigen-presenting polypeptide.

Finally, none or minimal activity was observed when the reporter T lymphocytes were contacted with antigen-loaded engineered enucleated erythroid cells including either ds HLA-A2 or wt HLA-A2 after 6 days of culture, while the engineered enucleated erythroid cells including sc trimer were capable of inducing a response (FIG. 3C).

### Example 2

### Antigen-Presenting Polypeptide Cell Surface Levels are Stable on Engineered Enucleated Erythroid Cells Regardless of the Loading Status of the Polypeptide

To examine the stability of the antigen-presenting polypeptides on the cell surface of engineered enucleated erythroid cells over time, the level of the polypeptides was monitored on cells that included either the wt HLA-A2 or the ds HLA-A2 antigen-presenting polypeptides (described in Example 1 above), in either an unloaded state or after being loaded with the HPV E7 peptide. Briefly, the engineered enucleated erythroid cells were either unloaded or were loaded with HPV E7 peptide as described in Example 1. The cells were cultured in Iscove's MDM medium supplemented with human serum albumin, recombinant human insulin, human transferrin, human recombinant erythropoietin, and human plasma for a period of 10 days at 37° C. At days 0, 3, 5, 7, and 10, cells were analyzed by flow cytometry after staining with either anti-β2M antibody, or anti-HLA-A2 antibody.

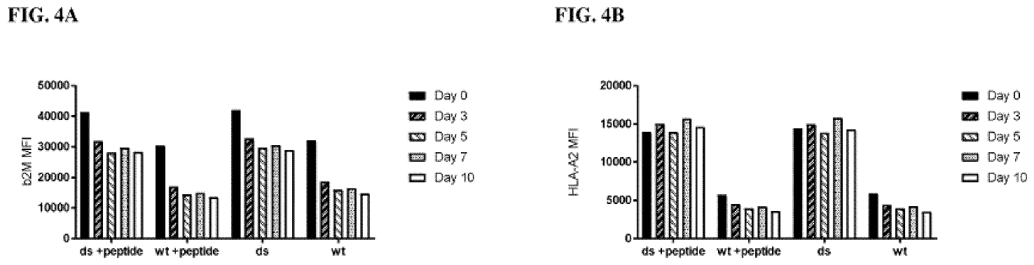

As shown in FIG. 4A (cells stained with anti-β2M antibody), a minimal reduction in signal intensity (expressed in Mean Fluorescence Intensity (MFI)) was observed in cells stained with anti-β2M antibody from day 0 to day 3; however, the signal intensity remained stable from days 3 through 10. Further, as shown in FIG. 4B (cells stained with anti-HLA-A2 antibody), a reduction in signal intensity was not observed when the cells were stained with anti-HLA-A2 antibody. This data demonstrates that the cell surface levels of both the wt HLA-A2 or the ds HLA-A2 antigen-presenting polypeptides were stable on the engineered enucleated erythroid cells regardless of the loading status of the antigen-presenting polypeptides.

### Example 3

### Engineered Enucleated Erythroid Cells Including Antigen-Presenting Polypeptides Can Be Loaded with Multiple Antigenic Polypeptides and Specifically Activate TCRs

To determine whether engineered enucleated erythroid cells including a loadable antigen-presenting polypeptide are loadable with multiple antigenic polypeptides and capable of activating multiple antigen-specific TCRs, the following experiment was performed. Briefly, engineered enucleated erythroid cells including either the wt HLA-A2 or the ds HLA-A2 antigen-presenting polypeptides (described in Example 1 above), or a loadable antigen-presenting polypeptide comprising mutant HLA*02:01 polypeptide having the amino acid substitutions Y84C and A139C, β2M polypeptide, and GPA (“ds HLA-A2 without FLAG”), as shown in SEQ ID NO:42 (below), were used.

(SEQ ID NO: 42)
MSRSVALAVLALLSLSGLEAIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDMGGGGSGGGGSGGGGSGGGGSGSHSMRYFFTS
VSRPGRGEPRFIAVGYVDDTQFVRFDSDAASQRMEPRAPWIEQEGPEYWD
GETRKVKAHSQTHRVDLGTLRGCYNQSEAGSHTVQRMYGCDVGSDWRFLR
GYHQYAYDGKDYIALKEDLRSWTAADMCAQTTKHKWEAAHVAEQLRAYLE
GTCVEWLRRYLENGKETLQRTDAPKTHMTHHAVSDHEATLRCWALSFYPA
EITLTWQRDGEDQTQDTELVETRPAGDGTFQKWAAVVVPSGQEQRYTCHV
QHEGLPKPLTLRWEPSSQPTIPIGSGSGSGSEDGSGSGSGSLSTTEVAMH
TSTSSSVTKSYISSQTNDTHKRDTYAATPRAHEVSEISVRTVYPPEEETG
ERVQLAHHFSEPEITLIIFGVMAGVIGTILLISYGIRRLIKKSPSDVKPL
PSPDTDVPLSSVEIENPETSDQ

The cells were loaded simultaneously with both HPV E7 antigenic polypeptide (YMLDLQPET; SEQ ID NO:41) and HPV E6 antigenic polypeptide (TIHDIILECV; SEQ ID NO:43) by contacting the cells with serum-free IMDM including either 10 μg/mL, 1 μg/mL, or 100 ng/mL of each antigenic polypeptide, as described in Example 1. As positive control, engineered erythroid cells including the sc trimer fusion polypeptide (see Example 1) were used. As negative control, erythroid cells generated from untransduced erythroid precursor cells were used.

The ability of the engineered enucleated erythroid cells to activate TCRs with specificity for either the loaded HPV E7 peptide or the loaded HPV E6 peptide was assessed using TCR activity assays. The TCR activity assays were performed as described in Example 1 using NFAT-Luc reporter T lymphocyte cell lines (InvivoGen™) engineered to express either an HPV E7-specific TCR or an HPV E6-specific TCR.

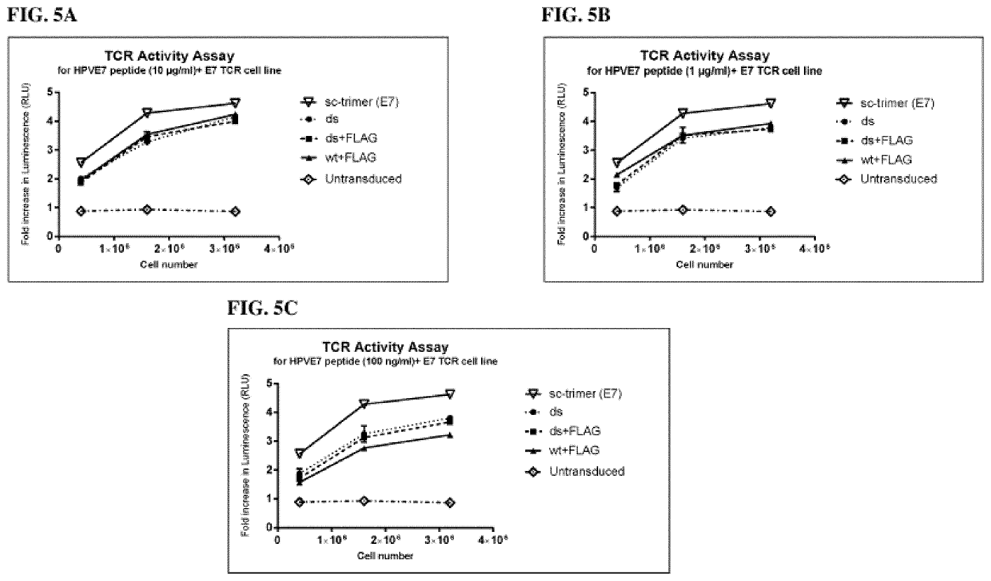

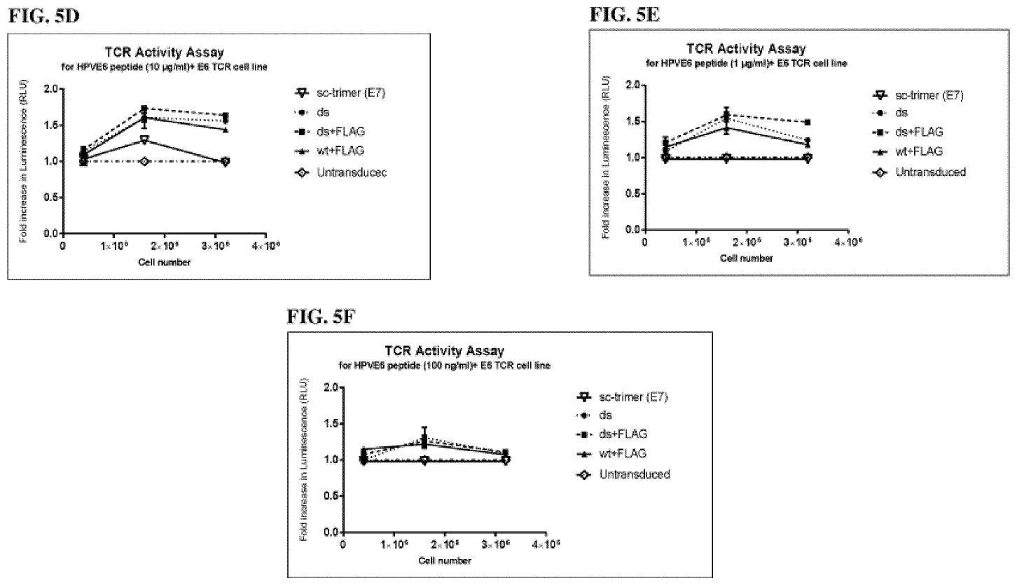

As shown in FIGS. 5A, 5B, and 5C, a dose-dependent response was observed when the reporter T lymphocytes including an HPV E7-specific TCR were contacted with engineered erythroid cells including the HPV E7 antigenic polypeptide loaded-wt HLA-A2, ds HLA-A2, or ds HLA-A2 without FLAG antigen-presenting polypeptides at all used loading concentrations of antigenic polypeptide. Further, as shown in FIGS. 5D, 5E, and 5F, engineered erythroid cells including the HPV E6 antigenic polypeptide loaded-wt HLA-A2, ds HLA-A2, and ds HLA-A2 without FLAG antigen-presenting polypeptides activated HPV E6-specific TCRs at all loading concentrations of HPV E6 antigenic polypeptide used. Moreover, engineered erythroid cells including the sc trimer fusion polypeptide did not induce a response in reporter T lymphocytes that expressed the HPV E6-specific TCR since the fusion polypeptide includes the HPV E7 antigenic polypeptide (and not the HPV E6 antigenic polypeptide).

To confirm that the response observed when reporter T lymphocytes were contacted with engineered enucleated erythroid cells including loadable antigen-presenting polypeptides was specific, cells including either the wt HLA-A2, the ds HLA-A2, or the ds HLA-A2 without FLAG antigen-presenting polypeptides were used. The cells were either unloaded (i.e., were not contacted with antigenic polypeptide to load the antigen-presenting polypeptides), or were loaded with either HPV E7 antigenic polypeptide (YMLDLQPET; SEQ ID NO:41) or HPV E6 antigenic polypeptide (TIHDIILECV; SEQ ID NO:43) as described in Example 1. Cells including the unloaded antigen-presenting polypeptides were used to perform TCR activity assays using reporter T lymphocytes that expressed either an HPV E7-specific TCR or an HPV E6-specific TCR. In addition, cells including antigen-presenting polypeptides that were loaded with HPV E6 antigenic polypeptide were used to perform TCR activity assays using the reporter T lymphocytes that expressed an HPV E7-specific TCR, while cells that were loaded with HPV E7 antigenic polypeptide were used in TCR activity assays using reporter T lymphocytes that expressed an HPV E6-specific TCR. As positive control, engineered enucleated erythroid cells including the sc trimer fusion polypeptide were used to perform TCR activity assays using reporter T lymphocytes that expressed either an HPV E7-specific TCR or an HPV E6-specific TCR.

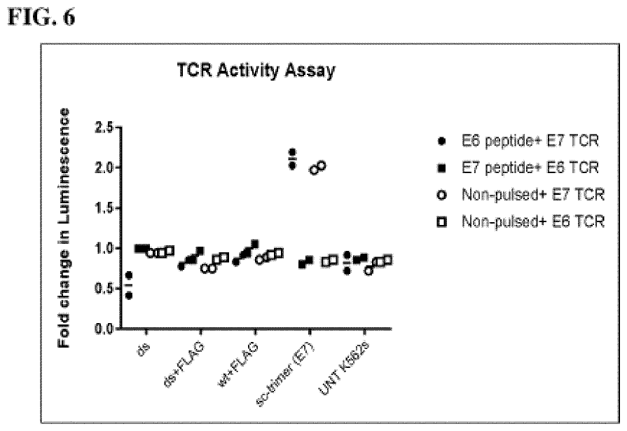

As shown in FIG. 6, no activity was observed when cells including unloaded antigen-presenting polypeptides were used (see data corresponding to “Non-pulsed+E7 TCR” and “Non-pulsed+E6 TCR”). Engineered enucleated erythroid cells including the sc trimer fusion polypeptide solely activated reporter T lymphocytes that expressed HPV E7-specific TCR, but not reporter T lymphocytes that expressed HPV E6-specific TCR. Additionally, no activity was observed when either i) cells including antigen-presenting polypeptides loaded with HPV E6 antigenic polypeptide were contacted with the reporter T lymphocytes that expressed an HPV E7-specific TCR; or ii) cells including antigen-presenting polypeptides loaded with HPV E7 antigenic polypeptide were contacted with the reporter T lymphocytes that expressed an HPV E6-specific TCR. Thus, the engineered enucleated cells including loadable antigen-presenting polypeptides are capable of activating TCRs that are specific for the loaded antigenic polypeptide.

### Example 4

### Loadable Antigen-Presenting Polypeptide Including Mutant HLA*02:01 Exhibits Faster Antigenic Polypeptide Loading as Compared to Antigen-Presenting Polypeptides Including Wild-Type HLA*02:01

To examine the antigenic polypeptide loading kinetics of loadable antigen-presenting polypeptides, the following experiment was performed. Briefly, engineered enucleated erythroid cells including either the wt HLA-A2, or the mutant ds HLA-A2 antigen-presenting polypeptides were labeled as described in Example 1 with 10 ng/mL of one of two tetramethylrhodamine (TAMRA)-labeled versions of the HPV E7 antigenic polypeptides: HPV E7-GGK (YMLDLQPETGGK, where the lysine (K) residue was TAMRA-labeled (SEQ ID NO:44) or HPV E7-E18K (YMLDLQPKT, where the lysine (K) residue was TAMRA-labeled (SEQ ID NO:45) over about 90 minutes at 4° C., room temperature (RT), or 37° C. Binding kinetics were measured by detecting the intensity of fluorescent signal on the cells after antigenic polypeptide loading using flow cytometry.

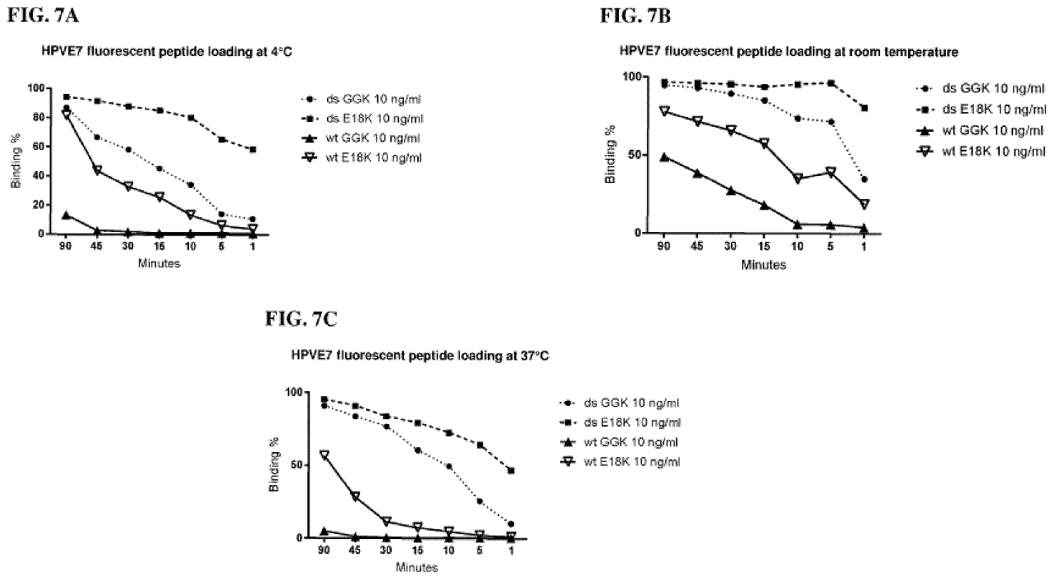

As shown in FIGS. 7A-7C, at all temperatures tested, the HPV E7-E18K fluorescent peptide loaded more rapidly than the HPV E7-GGK fluorescent peptide onto cells including the wt HLA-A2 antigen-presenting polypeptide, and onto cells including the ds HLA-A2 loadable antigen-presenting polypeptide. However, the on-rate kinetics were observed to be faster on cells including the ds HLA-A2 loadable antigen-presenting polypeptide as compared to the on-rate kinetics for cells including the wt HLA-A2 antigen-presenting polypeptide. Without wishing to be bound by any particular theory, it is believed that the differences in on-rate kinetics may be due to increased affinity for the HPV E7 antigenic polypeptide.

### Example 5

### Engineered Enucleated Erythroid Cells Including Loadable Antigen-Presenting Polypeptides Loaded with a CMV Antigenic Polypeptide are Capable of Activating and Inducing the Expansion of CMV-Specific T-Cells

To assess the ability of engineered enucleated erythroid cells including a loadable antigen-presenting polypeptide loaded with an exogenous antigenic polypeptide to activate antigenic-peptide-specific T cells, the following experiments were performed using human engineered enucleated erythroid cells including either the wt HLA-A2 or the mutant ds HLA-A2 antigen-presenting polypeptides, that were generated as described in Example 1.

First, to assess the ability of the CMV antigenic polypeptide-loaded engineered enucleated erythroid cells to activate CMV-specific TCRs the following experiment was performed. The engineered enucleated erythroid cells were loaded with a CMV antigenic polypeptide by resuspending 2×10′ cells/mL in RPMI supplemented with 10% fetal bovine serum containing 1 μg/ml of a CMV antigenic polypeptide (NLVPMVATV; SEQ ID NO: 155) for 90 minutes at 37° C. The cells were subsequently washed twice with PBS and once with RPMI supplemented with 10% fetal bovine serum. Subsequently, the engineered enucleated erythroid cells were contacted with CMV-specific CD8+ T cells obtained from an HLA-A2-restricted donor for 4 hours at 10:1, 2:1, or 0.4:1 erythroid cell to T-cell ratio. As negative controls, erythroid cells generated from untransduced erythroid precursor cells, contacted with CMV antigenic polypeptide (UNT-pulsed) or not contacted with CMV antigenic polypeptide (UNT-non-pulsed) were used.

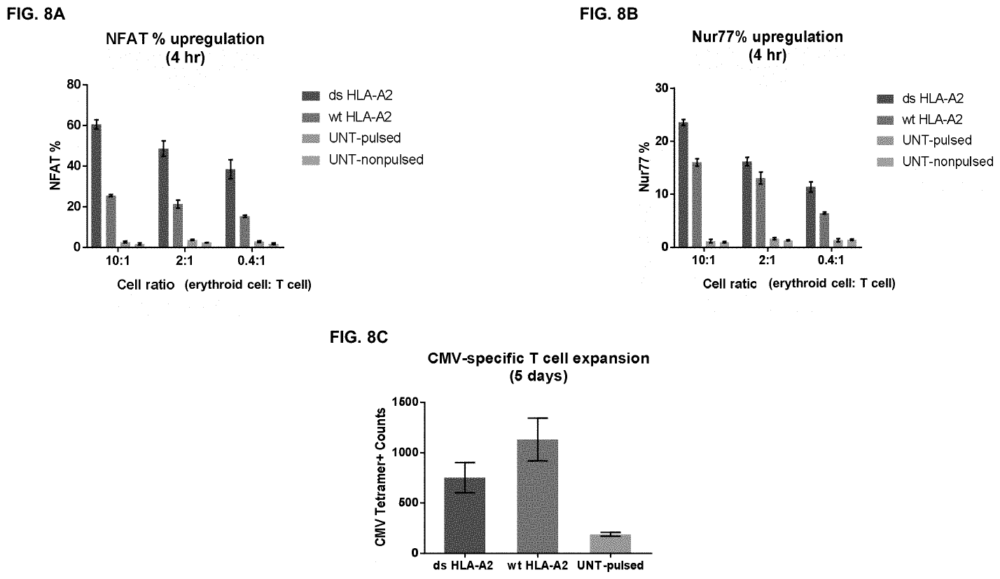

The extent of TCR activation for each condition was measured by performing flow cytometry to identify the frequency of T cells having elevated expression levels of intracellular NFAT (FIG. 8B) or intracellular Nur77 (FIG. 8C). Briefly, the cells were fixed with IC Fixation Buffer (Invitrogen™), permeabilized with Permabilization Buffer (InvitrogenTM) containing both PE anti-NFATc1 (BioLegend®) and Alexa Fluor® 647 anti-Nur77 monoclonal antibodies (BD BIOSCIENCES) and analyzed using flow cytometry.

As shown in FIGS. 8A and 8B, increased expression levels of NFAT and Nur77 was observed when the CMV-specific CD8+ T cells were contacted with engineered enucleated erythroid cells including the CMV antigenic polypeptide-loaded-wt HLA-A2, or ds HLA-A2 polypeptides, which was indicative of robust activation of CMV-specific CD8+ T cells. In contrast, T cells contacted with the control enucleated erythroid cells exhibited background expression levels of NFAT and Nur77, which was indicative of minimal TCR activation.

Second, to assess the ability of the CMV antigenic polypeptide-loaded engineered enucleated erythroid cells to induce the expansion of CMV-specific CD8⁺ T cells, the following experiment was performed. The engineered enucleated were loaded with the CMV antigenic polypeptide by resuspeding the 2 ×10⁶ cells/mL in serum free X-VIVO™ 15 media contiaing 1 μg/m of a CMV antigenic polypeptide (NLVPMVATV; SEQ ID NO: 155) for 90 minutes at 37° C. The cells were subsequently washed twice with PBS and once with serum-free X-VIVO™ 15. Subsequently, the CMV antigenic polypeptide-loaded engineered enucleated erythroid cells were contacted with PBMCs obtained from the same HLA-A2-restricted donor as the above experiment at 4:1 erythroid cell to T-cell ratio, in the presence of a second population of engineered enucleated erythroid cells having a two exogenous polypeptides on the cell surface: a first exogenous polypeptide comprising IL-12 and a second exogenous polypeptide comprising 4-1BBL. As negative controls, erythroid cells generated from untransduced erythroid precursor cells, contacted with CMV antigenic polypeptide (UNT-pulsed) were used. Following 5 days of incubation, the cell mixture was stained with fluorescently labelled CMV-HLA-A2 tetramer and analyzed by flow cytometry to quantify CMV-specific T cell expansion.

As shown in FIG. 8D, robust expansion of CMV-specific T cells was observed when the PBMCs were contacted with engineered enucleated erythroid cells including the CMV antigenic polypeptide-loaded-wt HLA-A2, or ds HLA-A2 polypeptides, as compared to PBMCs contacted with control cells (UNT-pulsed).

These experiments demonstrated that engineered enucleated erythroid cells including a loadable antigen-presenting polypeptide which was loaded with CMV antigenic polypeptide were capable achieve robust TCR activation and expansion of primary T cells.

### Example 6

### Loadable Antigen-Presenting Polypeptides Including HLA Class II Polypeptides Can Be Stably Expressed on the Surface of Mammalian Cells

To determine whether antigenic polypeptide-unloaded exogenous loadable antigen-presenting polypeptides comprising HLA class II polypeptides could be stably expressed on the surface of mammalian cells, the following experiments were performed.

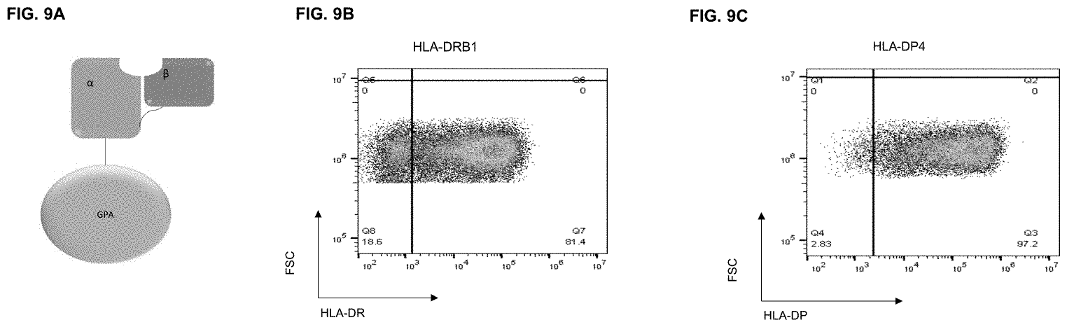

K562 cells comprising a loadable antigen-presenting polypeptide including HLA class II polypeptides (as depicted in FIG. 9A) were generated by transducing K562 cells with lentivirus encoding the following:
(1) an antigen-presenting polypeptide comprising wild-type HLA-DRA*01:01 polypeptide (excluding the native transmembrane domain and cytosolic region), a GlySer linker, HLA-DRB1*01:01 polypeptide (excluding the native transmembrane domain and cytosolic region) and a GPA transmembrane domain, as shown in SEQ ID NO: 1591 (below):

MSRSVALAVLALLSLSGLEAGDTRPRFLWQLKFECHFFNGTERVRLLERC
IYNQEESVRFDSDVGEYRAVTELGRPDAEYWNSQKDLLEQRRAAVDTYCR
HNYGVGESFTVQRRVEPKVTVYPSKTQPLQHHNLLVCSVSGFYPGSIEVR
WFRNGQEEKAGVVSTGLIQNGDWTFQTLVMLETVPRSGEVYTCQVEHPSV
TSPLTVEWRARSESAQSKTGSGGGGSGGGGSGGGGSGGGGSGTIKEEHVI
IQAEFYLNPDQSGEFMFDFDGDEIFHVDMAKKETVWRLEEFGRFASFEAQ
GALANIAVDKANLEIMTKRSNYTPITNVPPEVTVLTNSPVELREPNVLIC
FIDKFTPPVVNVTWLRNGKPVTTGVSETVFLPREDHLFRKFHYLPFLPST
EDVYDCRVEHWGLDEPLLKHWEFDAPSPLPETTELSTTEVAMHTSTSSSV
TKSYISSQTNDTHKRDTYAATPRAHEVSETSVRTVYPPEEETGERVQLAH
HFSEPEITLIIFGVMAGVIGTILLISYGIRRLIKKSPSDVKPLPSPDTDV
PLSSVEIENPETSDQ (signal sequence underlined);

or
(2) a loadable antigen-presenting polypeptide comprising HLA-DPA1*01:03 polypeptide (excluding the native transmembrane domain and cytosolic region), a GlySer linker, HLA-DPB1*04:01 polypeptide (excluding the native transmembrane domain and cytosolic region), and a GPA transmembrane domain, as shown in SEQ ID NO: 1592 (below),

MSRSVALAVLALLSLSGLEARATPENYLFQGRQECYAFNGTQRFLERYIY
NREEFARFDSDVGEFRAVTELGRPAAEYWNSQKDILEEKRAVPDRMCRHN
YELGGPMTLQRRVQPRVNVSPSKKGPLQHHNLLVCHVTDFYPGSIQVRWF
LNGQEETAGVVSTNLIRNGDWTFQILVMLEMTPQQGDVYTCQVEHTSLDS
PVTVEWKAQSDSARSKTLTGAGGTGSGGGGSGGGGSGGGGSGGGGSGTAG
AIKADHVSTYAAFVQTHRPTGEFMFEFDEDEMFYVDLDKKETVWHLEEFG
QAFSFEAQGGLANIAILNNNLNTLIQRSNHTQATNDPPEVTVFPKEPVEL
GQPNTLICHIDKFFPPVLNVTWLCNGELVTEGVAESLFLPRTDYSFHKFH
YLTFVPSAEDFYDCRVEHWGLDQPLLKHWEAQEPIQMPETTELSTTEVAM
HTSTSSSVTKSYISSQTNDTHKRDTYAATPRAHEVSEISVRTVYPPEEET
GERVQLAHHFSEPEITLIIFGVMAGVIGTILLISYGIRRLIKKSPSDVKP
LPSPDTDVPLSSVEIENPETSDQ (signal sequence
underlined).

Expression of the loadable antigen-presenting polypeptide including HLA class II polypeptides was detected by staining the cells with either anti-HLA-DR/DP monoclonal antibody (MEM-136)—APC (Thermo Fisher Scientific) or anti-HLA-DR mouse antibody (LN3)—PE/Cy5® (ABCAM). As shown in FIGS. 9B and 9C, expression of both of the loadable antigen-presenting polypeptide including HLA class II polypeptides was observed on the transduced K562 cells. These results demonstrate that exogenous loadable antigen-presenting polypeptides comprising HLA class II polypeptides which have not been loaded with antigenic polypeptide can be successfully expressed on the surface of mammalian cells.

The contents of International Patent Publication No. WO 2019/126818 are hereby incorporated by reference in its entirety.

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

1. An engineered enucleated erythroid cell comprising a loadable exogenous antigen-presenting polypeptide on its cell surface, wherein the loadable exogenous antigen-presenting polypeptide comprises one or more amino acid substitutions which stabilize the loadable exogenous antigen-presenting polypeptide on the cell surface.

2. The engineered enucleated erythroid cell of claim 1, wherein the loadable exogenous antigen-presenting polypeptide is stabilized in the absence of a polypeptide bound to the exogenous antigen-presenting polypeptide.

3. The engineered enucleated erythroid cell of claim 1, wherein the engineered enucleated erythroid cell further comprises an exogenous antigenic polypeptide bound to the loadable exogenous antigen-presenting polypeptide.

4. The engineered enucleated erythroid cell of claim 1, wherein the loadable exogenous antigen-presenting polypeptide has a higher affinity for an exogenous antigenic polypeptide than for an exogenous displaceable polypeptide.

5. (canceled)

6. The engineered enucleated erythroid cell of claim 3, wherein the exogenous antigenic polypeptide comprises an amino acid sequence provided in any one of Tables 7-8, 16-26, and B.

7. (canceled)

8. The engineered enucleated erythroid cell of claim 3, wherein the exogenous antigenic polypeptide is non-covalently attached to the loadable exogenous antigen-presenting polypeptide.

9. The engineered enucleated erythroid cell of claim 3, wherein the exogenous antigenic polypeptide is covalently attached to the loadable exogenous antigen-presenting polypeptide.

10. (canceled)

11. The engineered enucleated erythroid cell of claim 9 or 10, wherein exogenous antigenic polypeptide comprises a reactive functional group that forms a covalent bond with an amino acid residue of the loadable exogenous antigen-presenting polypeptide, wherein the reactive functional group is a diazirine group or a thiol-reactive functional group selected from the group consisting of (i) 2-cyanobenzothiazole (CBT); (ii) maleimide or a maleimide derivative (iii) an arylpropionitrile; (iv) a sulfone; (v) an allenamide; (v) a dibromopyridazinedione; (vi) a disulfide; and (vii) a haloacetamide.

12. The engineered enucleated erythroid cell of claim 11, wherein the reactive functional group is a thiol-reactive functional group and the exogenous antigenic polypeptide is covalently linked to a cysteine residue of the loadable exogenous antigen-presenting polypeptide.

13. The engineered enucleated erythroid cell of claim 1, wherein the loadable exogenous antigen-presenting polypeptide comprises a linker, wherein the linker comprises an acceptor sequence for conjugation of an exogenous antigenic polypeptide.

14.-15. (canceled)

16. The engineered enucleated erythroid cell of claim 1, wherein the loadable exogenous antigen-presenting polypeptide comprises a transmembrane domain.

17.-18. (canceled)

19. The engineered enucleated erythroid cell of claim 1, wherein the engineered enucleated erythroid cell further comprises a displaceable exogenous polypeptide bound to the loadable exogenous antigen-presenting polypeptide.

20.-22. (canceled)

23. The engineered enucleated erythroid cell of claim 19, wherein the exogenous displaceable polypeptide comprises an amino acid sequence provided in Table 6.

24. The engineered enucleated erythroid cell of claim 19, wherein the loadable exogenous antigen-presenting polypeptide and the exogenous displaceable polypeptide are comprised in a single-chain fusion protein.

25.-34. (canceled)

35. The engineered enucleated erythroid cell of claim 24, wherein the single chain fusion protein comprises a transmembrane domain.

36.-37. (canceled)

38. The engineered enucleated erythroid cell of claim 1, wherein the loadable exogenous antigen-presenting polypeptide comprises a human leukocyte antigen a (HLA) heavy chain polypeptide and a beta-2-microglobulin (β2M) polypeptide, or a fragment thereof.

39.-42. (canceled)

43. The engineered enucleated erythroid cell of claim 38, wherein the HLA heavy chain polypeptide is derived from an HLA class I polypeptide.

44.-49. (canceled)

50. The engineered enucleated erythroid cell of claim 43, wherein the loadable exogenous antigen-presenting polypeptide comprises:
(i) an amino acid substitution to an alanine at an amino acid residue corresponding to position 84 of an HLA heavy chain polypeptide;
(ii) an amino acid substitution to a cysteine at amino acid residues corresponding to positions 84 and 139 of an HLA heavy chain polypeptide;
(iii) an amino acid substitution to a cysteine at amino acid residues corresponding to positions 51 and 175 of an HLA heavy chain polypeptide;
(iv) at least one pair of amino acid substitutions to cysteine at amino acid residues corresponding to positions 84 and 139, 51 and 175, 5 and 168, 130 and 157, 135 and 140, 11 and 74, 45 and 63, or 33 and 49 of an HLA heavy chain polypeptide;
(v) an amino acid substitution to a cysteine at an amino acid residue corresponding to position 84 of an HLA heavy chain polypeptide, a displaceable exogenous polypeptide, and a linker disposed between the β2M polypeptide and the displaceable exogenous polypeptide, wherein the linker comprises a cysteine; or
(vi) an HLA heavy chain polypeptide derived from an HLA-A*02:01 allele, wherein the HLA heavy chain polypeptide comprises an amino acid substitution to a glutamic acid at an amino acid residue corresponding to position 115 of an HLA heavy chain polypeptide.

51.-55. (canceled)

56. The engineered enucleated erythroid cell of claim 38, wherein the HLA heavy chain polypeptide is derived from an HLA class II polypeptide.

57.-62. (canceled)

63. A method of treating a subject in need of an altered immune response, the method comprising:
a) determining an HLA status of the subject;
b) selecting an engineered enucleated erythroid cell comprising a loadable exogenous antigen-presenting polypeptide on its cell surface, wherein the antigen-presenting polypeptide is immunologically-compatible with the subject, and the exogenous antigen-presenting polypeptide comprises one or more amino acid substitutions which stabilize the loadable exogenous antigen-presenting polypeptide on the cell surface;
c) contacting the engineered enucleated erythroid cell with an exogenous antigenic polypeptide; and
d) administering the engineered enucleated erythroid cell to the subject, thereby treating the subject.

64.-72. (canceled)

73. A method of making an engineered enucleated erythroid cell comprising an antigen-loaded exogenous antigen-presenting polypeptide, the method comprising:
a) obtaining an engineered enucleated erythroid cell comprising a loadable exogenous antigen-presenting polypeptide on its the cell surface, wherein the loadable exogenous antigen-presenting polypeptide comprises one or more amino acid substitutions which stabilize the loadable exogenous antigen-presenting polypeptide on the cell surface; and
b) contacting the engineered enucleated erythroid cell with an exogenous antigenic polypeptide,
thereby generating the engineered enucleated erythroid cell comprising the antigen-loaded exogenous antigen-presenting polypeptide.

74.-78. (canceled)

79. A method of making an engineered enucleated erythroid cell comprising a loadable exogenous antigen-presenting polypeptide on its cell surface, the method comprising:
introducing an exogenous nucleic acid encoding the loadable exogenous antigen-presenting polypeptide into a nucleated erythroid precursor cell, wherein the loadable exogenous antigen-presenting polypeptide comprises one or more amino acid substitutions which stabilize the loadable exogenous antigen-presenting polypeptide on the cell surface; and
culturing the nucleated erythroid precursor cell under conditions suitable for enucleation and production of the loadable exogenous antigen-presenting polypeptide, thereby making the engineered enucleated erythroid cell.

80.-111. (canceled)
